(19) United States
(12) Patent Application Publication
(10) Pub. No.: US 2023/0303496 A1
(43) Pub. Date: Sep. 28, 2023

(54) SELECTIVE ANDROGEN RECEPTOR COVALENT ANTAGONISTS (SARCAs) AND METHODS OF USE THEREOF

(71) Applicant: University of Tennessee Research Foundation, Knoxville, TN (US)

(72) Inventors: Ramesh Narayanan, Cordova, TN (US); Thamarai Ponnusamy, Memphis, TN (US); Duane D. Miller, Collierville, TN (US); Yali He, Germantown, TN (US); Dong-Jin Hwang, Arlington, TN (US)

(21) Appl. No.: 17/821,952

(22) Filed: Aug. 24, 2022

Related U.S. Application Data
(63) Continuation of application No. PCT/US2021/019490, filed on Feb. 24, 2021.
(60) Provisional application No. 62/981,516, filed on Feb. 25, 2020.

(51) Int. Cl. C07D 231/14; C07D 231/12; C07D 403/06; A61P 35/00

(52) U.S. Cl. CPC C07D 231/14; C07D 231/12; C07D 403/06; A61P 35/00

(57) ABSTRACT

This invention relates to selective androgen receptor covalent antagonists, synthetic intermediates and by-products, and related compounds, and compositions comprising the same, and uses thereof in treating androgen receptor dependent diseases and conditions such as hyperproliferations of the prostate including pre-malignancies and benign prostatic hyperplasia, prostate cancer, advanced prostate cancer, castration resistant prostate cancer, triple negative breast cancer, other cancers expressing the androgen receptor, androgenic alopecia or other hyperandrogenic dermal diseases, Kennedy's disease, amyotrophic lateral sclerosis (ALS), abdominal aortic aneurysm (AAA), and uterine fibroids, and to methods for reducing the levels of androgen receptor-full length (AR-FL) including pathogenic or resistance mutations, AR-splice variants (AR-SV), and pathogenic polyglutamine (polyQ) polymorphisms of AR.

### CROSS REFERENCE TO RELATED APPLICATIONS

This application is a continuation of PCT Application No. PCT/US2021/019490, filed Feb. 24, 2021, and claims the benefit of U.S. Provisional Application No. 62/981,516, filed Feb. 25, 2020, which are incorporated in their entirety herein by reference.

### GOVERNMENT INTEREST STATEMENT

This invention was made with government support under R01 CA229164, awarded by the National Cancer Institute. The government has certain rights in the invention.

### REFERENCE TO SEQUENCE LISTING

The present application is filed with a Sequence Listing in Electronic format. The Sequence Listing is provided as a file entitled ONCTG.01OC1_ST_26.xml, created Jun. 7, 2023, which is approximately 170 kb in size. The information in the electronic format of the sequence listing is incorporated herein by reference in its entirety.

### FIELD OF THE INVENTION

This invention relates to selective androgen receptor covalent antagonist (SARCA) compounds, synthetic intermediates and by-products, and related compounds, and compositions comprising the same, and uses thereof for treating androgen receptor dependent diseases and conditions such as hyperproliferations of the prostate including pre-malignancies and benign prostatic hyperplasia, prostate cancer, advanced prostate cancer, castration resistant prostate cancer, triple negative breast cancer, other cancers expressing the androgen receptor, androgenic alopecia or other hyperandrogenic dermal diseases, Kennedy's disease, amyotrophic lateral sclerosis (ALS), abdominal aortic aneurysm (AAA), and uterine fibroids, and to methods for reducing the levels of androgen receptor-full length (AR-FL) including pathogenic or resistance mutations, AR-splice variants (AR-SV), and pathogenic polyglutamine (polyQ) polymorphisms of AR.

### BACKGROUND OF THE INVENTION

Prostate cancer (PCa) is one of the most frequently diagnosed noncutaneous cancers among men in the US and is the second most common cause of cancer deaths with more than 200,000 new cases and over 30,000 deaths each year in the United States. PCa therapeutics market is growing at an annual rate of 15-20% globally.

Androgen-deprivation therapy (ADT) is the standard of treatment for advanced PCa. Patients with advanced prostate cancer undergo ADT, either by luteinizing hormone releasing hormone (LHRH) agonists, LHRH antagonists or by bilateral orchiectomy. Despite initial response to ADT, 25 disease progression is inevitable, and the cancer emerges as castration-resistant prostate cancer (CRPC). Up to 30% of patients with prostate cancer that undergo primary treatment by radiation or surgery will develop metastatic disease within 10 years of the primary treatment. Approximately 50,000 patients a year will develop metastatic disease, which is termed metastatic CRPC (mCRPC).

Patients with CRPC have a median survival of 12-18 months. Though castration-resistant, CRPC is still dependent on the androgen receptor (AR) signaling axis for continued growth. The primary reason for CRPC re-emergence is re-activation of AR by alternate mechanisms such as: 1) intracrine androgen synthesis, 2) AR splice variants (AR-SV), e.g., that lack ligand binding domain (LBD), 3) AR-LBD mutations with potential to resist AR antagonists (i.e., mutants that are not sensitive to inhibition by AR antagonists, and in some cases AR antagonists act as agonists of the AR bearing these LBD mutations), 4) amplifications of the AR gene within the tumor (e.g., as driven by the fusion of other genes such as the ETS family of transcription factors (see for example PMID: 20478527, 30033370), and 5) rearrangements of the AR gene within the tumor, e.g., as described in PMID: 27897170. A critical barrier to progress in treating CRPC is that AR signaling inhibitors such as darolutamide, enzalutamide, bicalutamide, and abiraterone, acting through the LBD, fail to inhibit growth driven by the N-terminal domain (NTD)-dependent constitutively active AR-SV such as AR-V7, the most prominent AR-SV. Recent high-impact clinical trials with enzalutamide and abiraterone in CRPC patients demonstrated that just 13.9% of AR-V7-positive patients among 202 patients starting treatment with enzalutamide (Xtandi) or abiraterone acetate (Zytiga) had PSA responses to either of the treatments (Antonarakis E S, et al. J Clin. Oncol. 2017 April 6. doi: 10.1200/JCO.2016.70.1961), indicating the requirement for next generation AR antagonists that target AR-SVs. In addition, a significant number of CRPC patients are becoming refractory to abiraterone, enzalutamide, apalutamide, darolutamide, etc., emphasizing the need for next generation AR antagonists.

Current evidences demonstrate that CRPC growth is dependent on constitutively active AR including AR-SV's that lack the LBD such as AR-V7 and therefore cannot be inhibited by conventional antagonists. AR inhibition and degradation through binding to a domain that is distinct from the AR LBD provides alternate strategies to manage CRPC.

As described herein the AF-1 region of the NTD of AR is characterized to be bound irreversibly by the SARCAs of the invention. Covalently modified peptides from tryptic digests of AF-1 incubated with SARCAs of the invention were isolated and characterized by mass spectrometry, incontrovertibly establishing that the SARCAs produced stable covalent adducts of the AF-1 of AR. Further, the functional activity of AF-1 is inhibited as revealed by inhibition of AR-V7 dependent activation of transcription, i.e., AR-V7 transactivation, by the SARCAs of this invention. Both AF-1 and AR-V7 lack the LBD required for traditional AR antagonists. Moreover, SARCA compounds possessed AR full length (AR FL) and AR SV degradation activities. This is in addition to standard metrics of AR antagonists such as the inhibition of wtAR (i.e., AR FL) (see IC₅₀ values of Tables 1 and 2), binding to the LBD (see Kᵢ values of Tables 1 and 2), and inhibition of AR-dependent proliferation in vitro, e.g., in PCa cell lines or in vivo in androgen-dependent organs (see Example 15), and these criteria were comparable to LBD mediated inhibition. The report of irreversible or covalent binding of small molecules antagonists to AR via NTD or LBD binding sites was only previously seen for marine natural products that possessed poor pharmacokinetic properties and proved to be instable in clinical trials (see EPI-506). The SARCA activity incorporated into an acrylamide linker to mimic the highly prolific propanamide AR ligands which include flutamide, bicalutamide, enobosarm, UT-69, UT-155, and UT-34 provided improved AR affinity/selectivity and tunable warhead reactivity, helping to explain the unprecedented AR-V7 inhibitory potency, while maintaining the prodigiously broad AR antagonism profiles seen with the SARCAs of this invention. These SARCAs have the potential to evolve as new therapeutics to treat CRPCs that are untreatable with any other antagonists. These unique properties of irreversibly binding and inhibiting AF-1 provides the unique ability to inhibit constitutively active AR SVs lacking the LBD such as AR-V7. These unique properties have extreme importance in overcoming the health consequence that AR SVs pose for prostate cancer patients. SARCAs that irreversibly bind to the LBD would also have novel characteristics to overcome many of the known mechanisms of CRPC such as those itemized above.

Molecules that irreversibly inhibit or degrade the AR prevent any inadvertent AR activation through growth factors or signaling pathways, or promiscuous ligand-dependent activation. In addition, molecules that inhibit the constitutive activation of AR-SVs are extremely important to provide extended benefit to CRPC patients.

Currently no irreversible AR antagonists are available in clinical practice. No irreversible inhibitors of the LBD are known and only a single AR antagonist, 5N-bicalutamide (PMID: 28981251), has been characterized by mutational analysis to be consistent with reversible covalent inhibition by a reversible alkylation of C784 by the aryl nitrile A-ring of 5N-bicalutamide. Moreover, only a few AF-1 binding chemotypes have been reported such as EPI-001, EPI-506, sintokamides, glycerol ether Naphetenone B, 3E10-AR441 BSAb (bispecific antibodies), etc. Some of these AF-1 binding chemotypes from marine sponges such as the niphatenones (e.g., niphatenone A and niphatenone B), bisphenol A derivatives (e.g., EPI-001, EPI-506 and EPI-002), polychlorinated small peptide such as sintokamides (e.g., sintokamide A) and dysamides (e.g., dysamide A), etc., possessed an alkylation warhead, as reviewed in PMID: 30565725 H; however, none of the AF-1 binding chemotypes was reported as possessing SARD activity. Though these prior art agents are reported to bind to AR-NTD and inhibit AR function and PCa cell growth, they possessed lower affinity and inability to degrade the receptor. The SARCAs as described herein also bind to AR-NTD and inhibit NTD-driven (e.g., ligand independent) AR activity but exert potent inhibition of AR in the nM range and importantly possessed SARD activity. Only a few chemotypes are known to degrade AR which include the SARDs niclosamide, mahanine, ARN-509, AZD-3514, and ASC-J9. However, these molecules degrade AR indirectly at much higher concentrations than their binding coefficient and they fail to degrade the AR-SVs that have become in recent years the primary reason for resurgence of treatment-resistant CRPC.

This invention describes novel AR antagonists with unique pharmacology that strongly and irreversibly bind to AR, antagonize AR and degrade AR. Such selective AR covalent antagonists (SARCAs) possess dual degradation and (irreversible) inhibitory functions and hence are distinct from any available CRPC therapeutics in use or previously reported. These SARCA compounds will inhibit the growth of PCa cells and tumors that are dependent of AR FL and SV for proliferations, as well as treat a wide variety of AR-dependent or androgen dependent diseases or conditions as would be known by the skilled in the art and are outlined in part herein.

The positive correlation between AR and PCa and the lack of an infallible AR antagonist capable of inhibiting the broad spectrum of CRPC resistance mechanisms, emphasizes the need for molecules that inhibit AR function through novel or alternate mechanisms and/or binding sites, and that can elicit antagonistic activities within an altered cellular environment.

Although traditional antiandrogens such as darolutamide, enzalutamide, bicalutamide and flutamide and androgen deprivation therapies (ADT) were approved for use in prostate cancer, there is significant evidence that antiandrogens could also be used in a variety of other hormone dependent and hormone independent cancers. For example, antiandrogens have been tested in breast cancer (enzalutamide in Breast Cancer Res. (2014) 16(1): R7; darolutamide in ClinicalTrials.gov Identifier: NCT03004534), non-small cell lung cancer (shRNAi AR), renal cell carcinoma (ASC-J9), partial androgen insensitivity syndrome (PAIS) associated malignancies such as gonadal tumors and seminoma, advanced pancreatic cancer (World J. Gastroenterology 20(29), 9229), cancer of the ovary, fallopian tubes, or peritoneum, cancer of the salivary gland (Head and Neck (2016) 38, 724-731; ADT was tested in AR-expressing recurrent/metastatic salivary gland cancers and was confirmed to have benefit on progression free survival and overall survival endpoints), bladder cancer (Oncotarget 6(30), 29860-29876); Int J. Endocrinol (2015), Article ID 384860), pancreatic cancer, lymphoma (including mantle cell), and hepatocellular carcinoma. Use of a more potent antiandrogen such as a SARCA in these cancers may more efficaciously treat the progression of these and other cancers. Other cancers may also benefit from SARCA treatment such as breast cancer (e.g., triple negative breast cancer (TNBC)), testicular cancer, cancers associated with partial androgen insensitivity syndromes (PAIS) such as gonadal tumors and seminoma, uterine cancer, ovarian cancer, cancer of the fallopian tubes or peritoneum, salivary gland cancer, bladder cancer, urogenital cancer, brain cancer, skin cancer, lymphoma, mantle cell lymphoma, liver cancer, hepatocellular carcinoma, renal cancer, renal cell carcinoma, osteosarcoma, pancreatic cancer, endometrial cancer, lung cancer, non-small cell lung cancer (NSCLC), gastric cancer, colon cancer, perianal adenoma, or central nervous system cancer.

Triple negative breast cancer (TNBC) is a type of breast cancer lacking the expression of the estrogen receptor (ER), progesterone receptor (PR), and HER2 receptor kinase. As such, TNBC lacks the hormone and kinase therapeutic targets used to treat other types of primary breast cancers. Correspondingly, chemotherapy is often the initial pharmacotherapy for TNBC. Interestingly, AR is often still expressed in TNBC and may offer a hormone targeted therapeutic alternative to chemotherapy. In ER-positive breast cancer, AR is a positive prognostic indicator as it is believed that activation of AR limits and/or opposes the effects of the ER in breast tissue and tumors. However, in the absence of ER, it is possible that AR actually supports the growth of breast cancer tumors. Though the role of AR is not fully understood in TNBC, there is evidence that certain TNBC's may be supported by androgen independent activation of AR-SVs lacking the LBD or androgen-dependent activation of AR full length. As such, enzalutamide and other LED-directed traditional AR antagonists would not be able to antagonize AR-SVs in these TNBC's. However, SARCAs of this invention are AR antagonists (Example 3) which are capable of destroying AR-SVs (see Tables 1 and 2, and Examples 2 and 13) and inhibiting AR SV (see Examples 6 and 12) through a binding site in the NTD of AR (see Examples 4, 5, 9, and 10) were able to antagonize AR in AR-dependent prostate cancer cells (see Examples 8 and 14) including AR SV dependent cells (see Example 8) and in vivo in AR-dependent target organs (Example 16); as would be necessary to provide an anti-tumor effects in the heavily pre-treated anti-androgen resistant CRPC patient population and other AR-expressing cancers, and treat a wide variety of AR-dependent diseases and conditions.

Traditional antiandrogens such as bicalutamide and flutamide were approved for use in prostate cancer. Subsequent studies have demonstrated the utility of antiandrogens (e.g., flutamide, spironolactone, cyproterone acetate, finasteride and chlormadinone acetate) in androgen-dependent dermatological conditions such as androgenic alopecia (male pattern baldness), acne vulgaris, and hirsutism (e.g., in female facial hair). Prepubertal castration prevents sebum production and androgenic alopecia but this can be reversed by use of testosterone, suggesting its androgen-dependence.

The AR gene has a polymorphism of glutamine repeats (polyQ) within exon 1 which when shortened may augment AR transactivation (i.e., hyperandrogenism). It has been found that shortened polyQ polymorphisms are more common in people with alopecia, hirsutism, and acne. Classic antiandrogens are undesirable for these purposes because they are ineffective through dermal dosing and their long-term systemic use raises the risks of untoward sexual effects such as gynecomastia and impotence. Further, similar to CPRC discussed above, inhibition of ligand-dependent AR activity alone may not be sufficient as AR can be activated by various cellular factors other than the endogeneous androgens testosterone (T) and dihydrotestosterone (DHT), such as growth factors, kinases, co-activator overexpression and/or promiscuous activation by other hormones (e.g., estrogens or glucocorticoids). Consequently, blocking the binding of T and DHT to AR with a classical antiandrogen may not be sufficient to have the desired efficacy.

An emerging concept is the topical application of a SARCAs to irreversibly inhibit or destroy the AR locally to the affected areas of the skin or other tissue without exerting any systemic antiandrogenism. For this use, a SARCA that does not penetrate the skin or is rapidly metabolized would be preferrable.

Supporting this approach is the observation that cutaneous wound healing has been demonstrated to be suppressed by androgens. Castration of mice accelerates cutaneous wound healing while attenuating the inflammation in the wounds. The negative correlation between androgen levels and cutaneous healing and inflammation, in part, explains another mechanism by which high levels of endogenous androgens exacerbate hyperandrogenic dermatological conditions. Further, it provides a rationale for the treatment of wounds such as diabetic ulcers or even trauma, or skin disorders with an inflammatory component such as acne or psoriasis, with a topical SARCA.

Androgenic alopecia occurs in ˜50% of Caucasian males by midlife and up to 90% by 80 years old. Minoxidil (a topical vasodilator) and finasteride (a systemic 5alpha reductase type II inhibitor) are FDA approved for alopecia but require 4-12 months of treatment to produce a therapeutic effect and only arrest hair loss in most with mild to moderate hair regrowth in 30-60%. Since currently available treatments have slow and limited efficacy that varies widely between individuals, and produce unwanted sexual side effects, it is important to find a novel approach to treat androgenic alopecia and other hyperandrogenic dermatologic diseases.

Amyotrophic lateral sclerosis (ALS) is a fatal neurodegenerative disease characterized by selective loss of upper and lower motor neurons and skeletal muscle atrophy. Epidemiologic and experimental evidence suggest the involvement of androgens in ALS pathogenesis (“Anabolic/androgenic steroid nandrolone exacerbates gene expression modifications induced by mutant SOD1 in muscles of mice models of amyotrophic lateral sclerosis.” Galbiati M, et al. Pharmacol. Res. 2012, 65(2), 221-230), but the mechanism through which androgens modify the ALS phenotype is unknown. A transgenic animal model of ALS demonstrated improved survival upon surgical castration (i.e., androgen ablation). Treatment of these castrated animals with the androgen agonist nandrolone decanoate worsened disease manifestations. Castration reduces the AR level, which may be the reason for extended survival. The survival benefit is reversed by androgen agonist (“Androgens affect muscle, motor neuron, and survival in a mouse model of SOD1-related amyotrophic lateral sclerosis.” Aggarwal T, et al. Neurobiol. Aging. 2014 35(8), 1929-1938). Notably, stimulation with nandrolone decanoate promoted the recruitment of endogenous androgen receptor into biochemical complexes that were insoluble in sodium dodecyl sulfate, a finding consistent with protein aggregation. Overall, these results shed light on the role of androgens as modifiers of ALS pathogenesis via dysregulation of androgen receptor homeostasis. Antiandrogens should block the effects of nandrolone undecanoate or endogeneous androgens and reverse the toxicities due to AR aggregation. Further, an antiandrogen that can block action of LBD-dependent AR agonists and concomitantly lower AR protein levels, such as the SARCAs of this invention, would be therapeutic in ALS. Riluzole is an available drug for ALS treatment, however, it only provides short-term effects. There is an urgent need for drugs that extend the survival of ALS patients.

Androgen receptor action promotes uterine proliferation. Hyperandrogenicity of the short polyQ AR has been associated with increased leiomyoma or uterine fibroids. (Hsieh Y Y, et al. J. Assist. Reprod. Genet. 2004, 21(12), 453-457). A separate study of Brazilian women found that shorter and longer [CAG](n) repeat alleles of AR were exclusive to the leiomyoma group in their study (Rosa F E, et al. Clin. Chem. Lab. Med. 2008, 46(6), 814-823). Similarly, in Asian Indian women long polyQ AR was associated with endometriosis and leiomyoma and can be regarded as high-risk markers. SARCAs could be used in women with uterine fibroids, especially those expressing shorter and longer [CAG](n) repeat alleles, to treat existing uterine fibroids, prevent worsening of fibroids and/or ameliorate carcinogenicity associated with fibroids.

An abdominal aortic aneurysm (AAA) is an enlarged area in the lower part of the aorta, the major blood vessel that supplies blood to the body. The aorta, about the thickness of a garden hose, runs from your heart through the center of your chest and abdomen. Because the aorta is the body's main supplier of blood, a ruptured abdominal aortic aneurysm can cause life-threatening bleeding. Depending on the size and the rate at which your abdominal aortic aneurysm is growing, treatment may vary from watchful waiting to emergency surgery. Once an abdominal aortic aneurysm is found, doctors will closely monitor it so that surgery can be planned if it is necessary. Emergency surgery for a ruptured abdominal aortic aneurysm can be risky. AR blockade (pharmacologic or genetic) reduces AAA. Davis et al. (Davis J P, et al. J Vase Surg (2016) 63(6):1602-1612) showed that flutamide (50 mg/kg) or ketoconazole (150 mg/kg) attenuated porcine pancreatic elastase (0.35 U/mL) induced AAA by 84.2% and 91.5% compared to vehicle (121%). Further AR−/− mice showed attenuated AAA growth (64.4%) compared to wildtype (both treated with elastase). Correspondingly, administration of a SARCA to a patient suffering from an AAA may help reverse, treat or delay progression of AAA to the point where surgery is needed.

X-linked spinal-bulbar muscular atrophy (SBMA—also known as Kennedy's disease) is a muscular atrophy that arises from a defect in the androgen receptor gene on the X chromosome. Proximal limb and bulbar muscle weakness results in physical limitations including dependence on a wheelchair in some cases. The mutation results in a protracted polyglutamine tract added to the N-terminal domain of the androgen receptor (polyQ AR). Binding and activation of this lengthened polyQ AR by endogeneous androgens (testosterone and DHT) results in unfolding and nuclear translocation of the mutant androgen receptor. The androgen-induced toxicity and androgen-dependent nuclear accumulation of polyQ AR protein seems to be central to the pathogenesis. Therefore, the inhibition of the androgen-activated polyQ AR might be a therapeutic option (A. Baniahmad. Inhibition of the androgen receptor by antiandrogens in spinobulbar muscle atrophy. J. Mol. Neurosci. 2016 58(3), 343-347). These steps are required for pathogenesis and result in partial loss of transactivation function (i.e., an androgen insensitivity) and a poorly understood neuromuscular degeneration. Support of use antiandrogen comes in a report in which the antiandrogen flutamide protects male mice from androgen-dependent toxicity in three models of spinal bulbar muscular atrophy (Renier K J, et al. Endocrinology 2014, 155(7), 2624-2634).

More than 70% of the drugs that have been approved function as competitive antagonist or inhibitor. Efficacy of such competitive antagonists can be reduced by increasing agonist levels. All AR antagonists in clinical use are competitive that bind to the AR-LBD by hydrogen bonds and inhibit the AR activity. However, increasing levels of agonists will displace the antagonists by breaking the weak hydrophobic and hydrogen bonds. This competition between the antagonist and the agonist will result in a dynamic equilibrium, providing the cancers an opportunity to find alternate pathways to increase the intratumoral androgens and displace the antagonists. Irreversible antagonists of a protein such as the AR will bind to the AR through covalent bonding, which has 10-20 fold higher energy than the hydrogen bond, thereby thwarting any competition from agonist surge. It is highly desirable to discover covalent binders to proteins that will permanently bind to the protein and lock them in an inactive conformation. E.g., CRPC and breast cancer (BC) and many other AR-dependent diseases and conditions could benefit from selective AR covalent antagonists (SARCAs).

Covalent irreversible antagonists bind permanently to a protein that can be displaced only due to recycling of the protein and not by any endogenous substrates. Advantages of covalent irreversible antagonists include a) improved biochemical efficacy as competition with endogenous substrate is reduced; b) lower, less frequent dosing, resulting in a lower overall patient burden; c) potential prevention of drug resistance due to continuous target suppression. About 30% of the drugs approved by the FDA are covalent binders. Although covalent-binding drugs have been discovered and approved for several other targets, nuclear receptor family does not have any drug that binds covalently to the target. The closest covalent-binding drugs targeting hormonal cancers are abiraterone (Cyp17A1 inhibitor) and finasteride (Sa reductase inhibitor), but these inhibit enzymes in the androgen biosynthetic pathways, not nuclear receptors.

The unique property of degrading AR-SV has extremely important health consequences. Only few molecules are reported to bind and inhibit the AR-NTD or DNA binding domains (DBD). No irreversible AR antagonists have been approved yet. Most small molecule antagonists or inhibitors bind to the target protein by hydrophobic and hydrogen bonds and function as competitive antagonists. The bonds are weak and can be easily displaced by excess competitors. It is highly desirable to discover molecules that bind covalently (covalent bonds have at least 10 fold higher energy than hydrogen bonds) and irreversibly. It is important to discover irreversible AR antagonists that can provide sustained inhibition of the AR, for example, the inhibition of enzalutamide (Enza)-resistant-AR and -PCa tumors and treatment-refractory BC with selective AR covalent antagonists (SARCAs) as described herein. Furthermore, a wide variety of androgen-dependent diseases and conditions are described herein to be susceptible to treatment with AR antagonists. The SARCAs of this invention, in addition to alkylating the AR, further provide potent inhibition of wtAR in vitro (see IC₅₀ values in Tables 1 and 2) and hence will be effective in the same scope of diseases as traditional AR antagonists. I.e., the novel properties possessed by the SARCAs of this invention, e.g., binding of AF-1 in the NTD, alkylation of AR at NTD or LBD, or degradation of AR do not limit the scope of diseases susceptible to the AR antagonists of this invention. Instead, these novel AR antagonistic properties serve to expand the scope of androgen dependent diseases and conditions that are susceptible as fewer resistance mechanisms will be able to overcome treatment with the SARCAs of this invention.

### SUMMARY OF THE INVENTION

In one aspect, the invention provides a compound represented by the structure of formula I

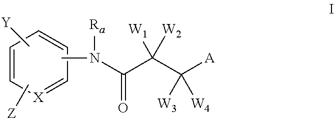

wherein
X is CH or N;
Y is H, CF₃, F, Br, Cl, I, CN, or C(R)₃;
Z is H, NO₂, CN, F, Br, Cl, I, COOH, COR, NHCOR, or CONHR;
or Y and Z form a 5 to 8 membered fused ring;
R is H, alkyl, alkenyl, CH₂CH₂OH, CF₃, CH₂Cl, CH₂CH₂Cl, aryl, F, Cl, Br, I, or OH; Rₐ is H, alkyl-NCO, alkyl-NCS, alkyl-SCN, alkyl-OCN, alkyl-N₃, alkyl-SO₂F, alkyl-CH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂, wherein halide is F, Cl, Br, or I;
W₁ is H or OR_{d}, wherein R_{d} is H, alkyl-NCO, alkyl-NCS, alkyl-SCN, alkyl-OCN, alkyl-N₃, alkyl-SO₂F, alkyl-CH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
W₂ is CH₃, CH₂F, CHF₂, CF₃, CH₂CH₃, CF₂CF₃, or CH₂A;
or W₁ and W₂, together with the carbon atom to which they are attached, form a C═CW₅W₆ group, wherein W₅ and W₆ are each H or alkyl;
W₃ and W₄ are individually H, OH, alkyl, wherein the alkyl is optionally substituted with OR, NO₂, CN, F, Br, Cl, I, COR, NHCOR, CONHR, —NCO, —NCS, —SCN, —OCN, —N₃, —SO₂F, —CH₂halide, —NHCOCH₂halide, —NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
or one of W₁ and W₂ with one of W₃ and W₄, together with the carbon atoms to which they are attached, form a C═C bond;
A is NR_{b}R_{c} or a 5 to 10-membered aryl or heteroaryl group, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
R_{b} is H or alkyl, wherein the alkyl is optionally substituted with OR, NO₂, CN, F, Br, Cl, I, COR, NHCOR, or CONHR;
R_{c} is alkyl, haloalkyl, cycloalkyl, heterocycloalkyl, aryl or heteroaryl, wherein said alkyl, haloalkyl, cycloalkyl, heterocycloalkyl, aryl and heteroaryl groups are optionally substituted with CN, NO₂, CF₃, F, Cl, Br, I NHCOOR, N(R)₂, NHCOR, COR, alkyl, or alkoxy;
or R_{b} and R_{c}, together with the nitrogen atom to which they are attached, form a 5 to 10-membered saturated or unsaturated heterocyclic ring having at least one nitrogen atom and 0, 1, or 2 double bonds, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
or its isomer, optical isomer, racemic mixture, pharmaceutically acceptable salt, pharmaceutical product, hydrate or any combination thereof.

In one embodiment, the compound of the invention is represented by the structure of formula II

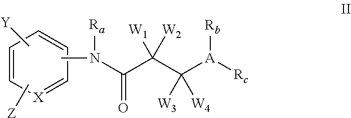

wherein
X is CH or N;
Y is H, CF₃, F, Br, Cl, I, CN, or C(R)₃;
Z is H, NO₂, CN, F, Br, Cl, I, COOH, COR, NHCOR, or CONHR;
or Y and Z form a 5 to 8 membered fused ring;
R is H, alkyl, alkenyl, CH₂CH₂OH, CF₃, CH₂Cl, CH₂CH₂Cl, aryl, F, Cl, Br, I, or OH;
Rₐ is H, alkyl-NCO, alkyl-NCS, alkyl-SCN, alkyl-OCN, alkyl-N₃, alkyl-SO₂F, alkylCH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂, wherein halide is F, Cl, Br, or I;
W₁ is H or OR_{d}, wherein R_{d} is H, alkyl-NCO, alkyl-NCS, alkyl-SCN, alkyl-OCN, alkylN₃, alkyl-SO₂F, alkyl-CH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
W₂ is CH₃, CH₂F, CHF₂, CF₃, CH₂CH₃, CF₂CF₃, or CH₂A;
or W₁ and W₂, together with the carbon atom to which they are attached, form a C═CW₅W₆ group, wherein W₅ and W₆ are each H or alkyl;
W₃ and W₄ are individually H, OH, alkyl, wherein the alkyl is optionally substituted with OR, NO₂, CN, F, Br, Cl, I, COR, NHCOR, CONHR, —NCO, —NCS, —SCN, —OCN, —N₃, —SO₂F, —CH₂halide, —NHCOCH₂halide, —NHSO₂CH₂halide, —CH₂—CH═CH—COO R, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
or one of W₁ and W₂ with one of W₃ and W₄, together with the carbon atoms to which they are attached, form a C═C bond;
A is NR_{b}R_{c} or a 5 to 10-membered aryl or heteroaryl group, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
R_{b} is H or alkyl, wherein the alkyl is optionally substituted with OR, NO₂, CN, F, Br, Cl, I, COR, NHCOR, or CONHR;
R_{c} is alkyl, haloalkyl, cycloalkyl, heterocycloalkyl, aryl or heteroaryl, wherein said alkyl, haloalkyl, cycloalkyl, heterocycloalkyl, aryl and heteroaryl groups are optionally substituted with CN, NO₂, CF₃, F, Cl, Br, I NHCOOR, N(R)₂, NHCOR, COR, alkyl, or alkoxy;
or R_{b} and R_{c}, together with the nitrogen atom to which they are attached, form a 5 to 10-membered saturated or unsaturated heterocyclic ring having at least one nitrogen atom and 0, 1, or 2 double bonds, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHR)═CH₂, —CH₂—CH═CH—CON(R)₂, or —CH₂—C(CON(R)₂)═CH₂;
or its isomer, optical isomer, racemic mixture, pharmaceutically acceptable salt, pharmaceutical product, hydrate or any combination thereof.

In one embodiment, the compound of the invention represented by the structure of formula I or formula II contains at least one nucleophile acceptor group. In one embodiment, the compound of the invention represented by the structure of formula I or formula II contains at least one functional group with an α, β-unsaturated carbonyl. In one embodiment, such α, β-unsaturated carbonyl functional groups include but are not limited to α, β-unsaturated ketones, amides, esters, thioesters, acid anhydrides, carboxylic acids, carboxylates, acid halides, imides, and the like. In one embodiment, the α, β-unsaturated functional group serves as a Michael addition reaction acceptor for nucleophiles within the AR.

In one embodiment, the compound of the invention represented by the structure of formula I or formula II contains at least one nucleophile acceptor group. In one embodiment, the nucleophile acceptor group is at least one of isocyanato (—NCO), isothiocyanato (—NCS), cyanato (—CNO), thiocyanato (—CNS), azido (N₃), sulfonyl fluoride (—SO₂F), halomethyl (—CH₂-halide), 2-haloacetyl (—NHCOCH₂-halide), halosulfonyl (—NHSO₂CH₂-halide), and the like. In one embodiment, the nucleophile acceptor group serves as a nucleophile acceptor for nucleophiles within the AR. In one embodiment, said AR nucleophile is within the NTD. In another embodiment, said AR nucleophile is within the AF-1 domain. In another embodiment, said AR nucleophile is within the LBD. In one embodiment, the nucleophile acceptor group is present in the Rₐ group. In one embodiment, the nucleophile acceptor group is present in the W₁ group. In one embodiment, the nucleophile acceptor group is present in the W₃ or W₄ group. In one embodiment, the nucleophile acceptor group is present in any one of the Q¹, Q², Q³ or Q⁴ groups.

In one embodiment, the compound of the invention is represented by the structure of any one of the following compounds:

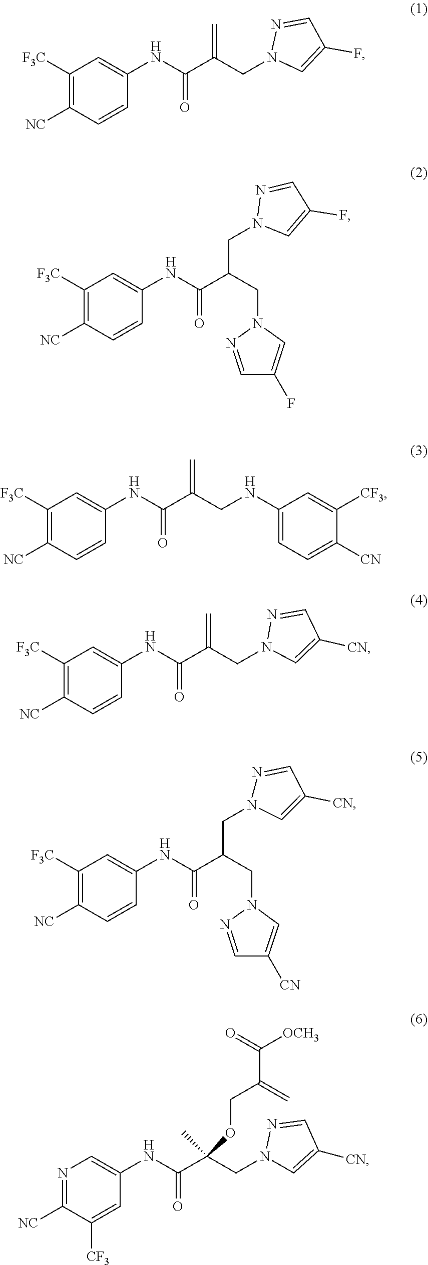 [SMILES: C=C(Cn1cc(F)cn1)C(=O)Nc1ccc(C#N)c(C(F)(F)F)c1 ; N#Cc1ccc(NC(=O)C(Cn2cc(F)cn2)Cn2cc(F)cn2)cc1C(F)(F)F ; C=C(CNc1ccc(C#N)c(C(F)(F)F)c1)C(=O)Nc1ccc(C#N)c(C(F)(F)F)c1 ; C=C(Cn1cc(C#N)cn1)C(=O)Nc1ccc(C#N)c(C(F)(F)F)c1 ; N#Cc1cnn(CC(Cn2cc(C#N)cn2)C(=O)Nc2ccc(C#N)c(C(F)(F)F)c2)c1 ; C=C(CO[C@@](C)(Cn1cc(C#N)cn1)C(=O)Nc1cnc(C#N)c(C(F)(F)F)c1)C(=O)OC]

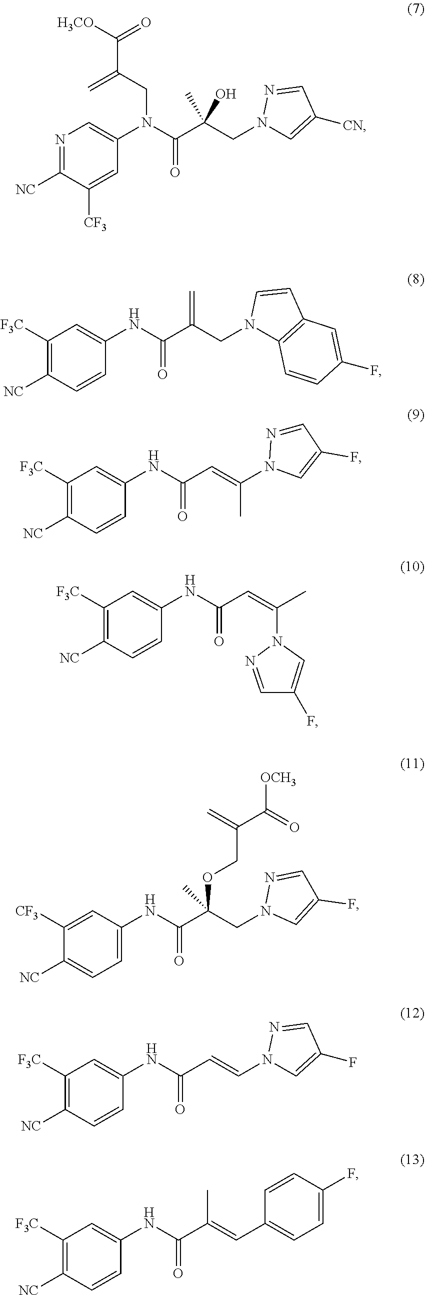

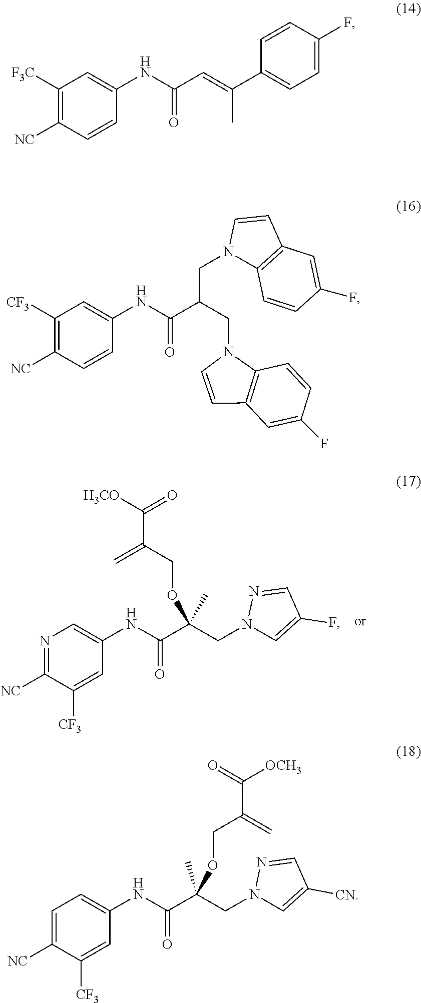 [SMILES: C/C(=C\C(=O)Nc1ccc(C#N)c(C(F)(F)F)c1)c1ccc(F)cc1 ; N#Cc1ccc(NC(=O)C(Cn2ccc3cc(F)ccc32)Cn2ccc3cc(F)ccc32)cc1C(F)(F)F ; C=C(CO[C@@](C)(Cn1cc(F)cn1)C(=O)Nc1cnc(C#N)c(C(F)(F)F)c1)C(=O)OC ; C=C(CO[C@@](C)(Cn1cc(C#N)cn1)C(=O)Nc1ccc(C#N)c(C(F)(F)F)c1)C(=O)OC]

In one embodiment, the compound of the invention is represented by the structure of compound 15

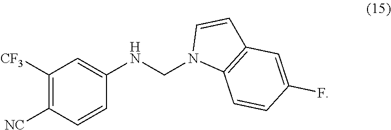 [SMILES: N#Cc1ccc(NCn2ccc3cc(F)ccc32)cc1C(F)(F)F]

In one aspect, the invention provides a pharmaceutical composition comprising a compound of the invention, or its isomer, optical isomer, or any mixture of optical isomers, pharmaceutically acceptable salt, pharmaceutical product, hydrate or any combination thereof, and a pharmaceutically acceptable carrier. In one embodiment, the composition is formulated for topical use. In one embodiment, the composition is in the form of a solution, lotion, salve, cream, ointment, liposome, spray, gel, foam, roller stick, cleansing soap or bar, emulsion, mousse, aerosol, or shampoo. In another embodiment, the composition is formulated for oral use.

In another aspect, the invention provides a method of treating an androgen receptor dependent disease or condition in a subject in need thereof, comprising administering to the subject a therapeutically effective amount of a compound of the invention as described herein. In one embodiment, the compound of the invention binds irreversibly to androgen receptor (AR).

In one embodiment, the androgen receptor dependent disease or condition in the subject responds to at least one of AR-splice variant (AR-SV) degradation activity, AR full length (AR-FL) degradation activity, irreversible or reversible AR-SV inhibitory activity, or irreversible or reversible AR-FL inhibitory activity.

In one embodiment, the androgen receptor dependent disease or condition is breast cancer.

In one embodiment, the subject has AR expressing breast cancer, AR-SV expressing breast cancer, and/or AR-V7 expressing breast cancer.

In one embodiment, the androgen receptor dependent disease or condition is Kennedy's disease.

In one embodiment, the androgen receptor dependent disease or condition is acne. In one embodiment, the acne is acne vulgaris.

In one embodiment, the androgen receptor dependent disease or condition is overproduction of sebum. In one embodiment, reducing the overproduction of sebum treats at least one of seborrhea, seborrheic dermatitis, or acne.

In one embodiment, the androgen receptor dependent disease or condition is hirsutism or alopecia.

In one embodiment, the alopecia is at least one of androgenic alopecia, alopecia areata, alopecia secondary to chemotherapy, alopecia secondary to radiation therapy, alopecia induced by scarring, or alopecia induced by stress.

In one embodiment, the androgen receptor dependent disease or condition is a hormonal disease or condition in a female. In one embodiment, the hormonal disease or condition in a female is at least one of precocious puberty, dysmenorrhea, amenorrhea, multilocular uterus syndrome, endometriosis, hysteromyoma, abnormal uterine bleeding, early menarche, fibrocystic breast disease, fibroids of the uterus, ovarian cysts, polycystic ovary syndrome, pre-eclampsia, eclampsia of pregnancy, pre term labor, premenstrual syndrome, or vaginal dryness.

In one embodiment, the androgen receptor dependent disease or condition is hormonal disease or condition in a male. In one embodiment, the hormonal disease or condition in a male is at least one of hypergonadism, hypersexuality, sexual dysfunction, gynecomastia, precocious puberty in a male, alterations in cognition and mood, depression, hair loss, hyperandrogenic dermatological disorders, pre-cancerous lesions of the prostate, benign prostate hyperplasia, prostate cancer and/or other androgen-dependent cancers.

In one embodiment, the androgen receptor dependent disease or condition is sexual perversion, hypersexuality, or paraphilias.

In one embodiment, the androgen receptor dependent disease or condition is androgen psychosis.

In one embodiment, the androgen receptor dependent disease or condition is virilization.

In one embodiment, the androgen receptor dependent disease or condition is androgen insensitivity syndrome.

In one embodiment, the androgen receptor dependent disease or condition is AR-expressing cancer in said subject. In one embodiment, the AR-expressing cancer is at least one of breast cancer, testicular cancer, cancers associated with partial androgen insensitivity syndromes (PAIS) such as gonadal tumors and seminoma, uterine cancer, ovarian cancer, cancer of the fallopian tubes or peritoneum, salivary gland cancer, bladder cancer, urogenital cancer, brain cancer, skin cancer, lymphoma, mantle cell lymphoma, liver cancer, hepatocellular carcinoma, renal cancer, renal cell carcinoma, osteosarcoma, pancreatic cancer, endometrial cancer, lung cancer, non-small cell lung cancer (NSCLC), gastric cancer, colon cancer, perianal adenoma, or central nervous system cancer.

In one embodiment, the androgen receptor dependent disease or condition is amyotrophic lateral sclerosis (ALS).

In one embodiment, the androgen receptor dependent disease or condition is uterine fibroids.

In one embodiment, the androgen receptor dependent disease or condition is abdominal aortic aneurysm (AAA).

In one embodiment, the androgen receptor dependent disease or condition is caused by polyglutamine (polyQ) AR polymorphs in a subject. In one embodiment, the polyQ-AR is a short polyQ polymorph or a long polyQ polymorph. In one embodiment, the polyQ-AR is a short polyQ polymorph and the method further treats dermal disease. In one embodiment, the dermal disease is at least one of alopecia, seborrhea, seborrheic dermatitis, or acne. In another embodiment, the polyQ-AR is a long polyQ polymorph and the method further treats Kennedy's disease.

In another aspect, the invention encompasses a method of treating prostate cancer (PCa) or increasing survival in a male subject in need of treatment comprising administering to the subject a therapeutically effective amount of a compound of the invention as described herein. The prostate cancer includes, but is not limited to, advanced prostate cancer, castration resistant prostate cancer (CRPC), metastatic CRPC (mCRPC), non-metastatic CRPC (nmCRPC), high-risk nmCRPC or any combination thereof. Another embodiment of the invention encompasses the method further comprising administering androgen deprivation therapy (ADT). Alternatively, the method may treat a prostate or other cancer that is resistant to treatment with known androgen receptor antagonist(s) or ADT. In another embodiment, the method may treat enzalutamide resistant prostate cancer. In another embodiment, the method may treat apalutamide resistant prostate cancer. In another embodiment, the method may treat abiraterone resistant prostate cancer. In another embodiment, the method may treat darolutamide resistant prostate cancer. Yet another embodiment of the invention encompasses a method of treating prostate or other AR antagonist resistant cancer with a compound of the invention as described herein, wherein the androgen receptor antagonist(s) is at least one of darolutamide, apalutamide, enzalutamide, bicalutamide, abiraterone, EPI-001, EPI-506, AZD-3514, galeterone, ASC-J9, flutamide, hydroxyflutamide, nilutamide, cyproterone acetate, ketoconazole, or spironolactone.

Yet another embodiment of the invention encompasses a method of treating prostate or other AR-expressing cancers using a compound of the invention wherein the other cancers are selected from breast cancer such as triple negative breast cancer (TNBC), testicular cancer, cancers associated with partial androgen insensitivity syndromes (PAIS) such as gonadal tumors and seminoma, uterine cancer, ovarian cancer, cancer of the fallopian tubes or peritoneum, salivary gland cancer, bladder cancer, urogenital cancer, brain cancer, skin cancer, lymphoma, mantle cell lymphoma, liver cancer, hepatocellular carcinoma, renal cancer, renal cell carcinoma, osteosarcoma, pancreatic cancer, endometrial cancer, lung cancer, non-small cell lung cancer (NSCLC), gastric cancer, colon cancer, perianal adenoma, or central nervous system cancer. In another embodiment, the breast cancer is triple negative breast cancer (TNBC).

The invention encompasses a method of reducing the levels of AR-splice variants in a subject comprising administering to the subject a therapeutically effective amount of a compound of this invention, or its isomer, optical isomer, or any mixture of optical isomers, pharmaceutically acceptable salt, pharmaceutical product, polymorph, hydrate or any combination thereof. The method may comprise further reducing the levels of AR-full length (AR-FL) in the subject.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIG. 1 depicts AR antagonist effects of compounds 1 and 4. AR transactivation assay was performed in COS cells with AR, GRE-LUC, and CMV-renilla-LUC.

FIG. 2 depicts 1 and 4 are covalent irreversible antagonists using Schild's plot. AR transactivation was performed with a dose-response of R1881 and three doses of AR antagonists. Enzalutamide, a competitive antagonist, showed a shift in the curves to the right with a Hill slope of 1. 1 and 4 both reduced the Eₘₐₓ with Hill's slope not near 1.

FIG. 3 depicts covalent binding of 1 using proteomic mass spectrometry. 1 was incubated with AR AF-1 protein and the protein complex was trypsin digested. Mass spectrometry was performed to determine the binding of 1 to AF-1. 1 bound to the peptides indicated in the panel. The M.Wt. shift by 338.08 Dalton of the top peptide corresponds to the M.Wt. of 1. Similarly, three molecules of 1 covalently interacted with the bottom peptide with M.Wt. corresponding shift of 98.75.

FIG. 4 depicts that 1 inhibited AR-V7 transactivation. Transactivation studies were performed with AR-V7 and GRE-LUC and p65 and NFkB-LUC. Cells were treated with 1 or enzalutamide. Luciferase assay was performed twenty-four hours after treatment. 1 inhibited AR-V7 transactivation, but not NFkB transactivation.

FIG. 5 depicts that 1 inhibited PCa cell proliferation. PCa cells were plated in 96 well plates and treated as indicated in the figure. Three days later, medium was replaced and the cells were retreated. At the end of six days of treatment, SRB assay was performed to measure the number of viable cells. 1 inhibited LNCaP and 22RV1 cells proliferation. At higher doses, 1 inhibited COS cell proliferation.

FIG. 6 depicts that the SARCA compounds of the invention are inhibitory of full length wildtype AR in vitro but the potency of the compounds is comparable 9 or less potent (10 and all others in the figure) compared to enzalutamide (˜300 nM) which is a LBD binding antiandrogen. AR transactivation: COS7 cells were plated in 24 well plates at 40,000 cells/well in DME+5% csFBS without phenol red. Twenty-four hours after plating, the cells were transfected with 0.25 μg GRE-LUC, 0.01 μg CMV-LUC, 0.025 μg CMV-hAR using Lipofectamine reagents in optiMEM medium. Twenty-four hours after transfection, the cells were treated with a dose-response of the compounds in the presence of 0.1 nM R1881. Twenty-four hours after treatment, the cells were harvested, and luciferase assay was performed using Dual-luciferase reagent. Firefly values were divided by Renilla numbers and the values are represented as relative light units (RLU).

FIG. 7 depicts that 6 is a SARCA compound which binds irreversibly to the tryptic peptides. Mass Spec Study: AR AF-1 was incubated with 6 (covalent binder) alone or 6+UT-34 (UT-34 is a noncovalent binder of AF-1). AF-1 was pre-incubated for 2 h with 200 μM UT-34 and then with 6 (100 μM).

FIG. 8 depicts that enzalutamide was a reversible AR inhibitor whereas the SARCAs 6 and 8 were irreversible AR inhibitors using a Schild's plot analysis.

FIG. 9 depicts the selectivity of inhibition of 6 across steroid receptors. RU486, a known superpotent steroid antagonist, inhibits both GR and PR in the sub-nM range. SARCA 6 in the same assay demonstrated low efficacy GR activity (about 20%) and no PR activity was observed until 10 μM. There was very little cross-reactivity of this SARCA with the other steroid receptors tested. GR and PR transactivation. COS7 cells were plated in 24 well plates at 40,000 cells/well in DME+5% csFBS without phenol red. Twenty-four hours after plating, the cells were transfected with 0.25 μg GRE-LUC, 0.01 μg CMV-LUC, 0.025 μg pCR3.1 rat GR or rat PR using Lipofectamine reagents in optiMEM medium. Twenty-four hours after transfection, the cells were treated with a dose-response of the compounds in the presence of 0.1 nM R1881. Twenty-four hours after treatment, the cells were harvested, and luciferase assay was performed using Dual-luciferase reagent. Firefly values were divided by Renilla numbers and the values are represented as relative light units (RLU).

FIG. 10 depicts that SARCAs that irreversibly bound to the NTD (present in AR-V7) such as 1 and 6 were able to significantly inhibited the transcriptional activation of AR-V7.

FIG. 11 depicts 6 and 8 bind irreversibly to AR using a Schild's plot analysis. Enzalutamide shifts the EC₅₀ of R1881 suggesting competitive binding, whereas 6 and 8 decreased the Eₘₐₓ of R1881 suggesting irreversible binding.

FIG. 12 depicts that compound UT-34 (a noncovalent binder of AF-1) did not alkylate the AF-1 protein; whereas the SARCA 1 bound irreversibly with AF-1. The UT-34 experiment serves as a negative control to demonstrate that not all AF-1 binding agents bind irreversibly to AF-1. This is in contrast to 1 which bound to the cysteine C18 as shown in the 2^{nd} row (or C20 (see 4^{th} row) if digested peptide is cut slightly differently) of the ‘LENPLADYGSA . . . ’ peptide. 1 also binds at C9 to the ‘GLEGESLGCS . . . ’ peptide. 1 additionally bound to C3 of the ‘GDC . . . ’ peptide near the bottom of the slide (not seen for 6).

FIG. 13 depicts a mass spec study with SARCA 4 showing alkylation of the ‘GLEGESLGSC . . . ’ and ‘LENPLDYGSA . . . ’ peptides (like 6 and 1), and also the ‘GDC . . . ’ peptide (like 1), but additionally K5 was alkylated in peptide GGYTK (unique).

FIG. 14 depicts that 1 and 4 did not alkylate the LBD, and hence their irreversible activity is solely based on AF-1 alkylation.

FIG. 15 depicts that 4 and 6 were stable to in vitro metabolism by mouse liver microsomes.

FIG. 16 depicts that SARCA 1 had antiproliferative activity in LNCaP cells (like non-SARCA AF-1 binding compound 155 [(S)—N-(4-cyano-3-(trifluoromethyl)phenyl)-3-(5-fluoro-1H-indol-1-yl)-2-hydroxy-2-methylpropanamide] and enzalutamide but improved potency) and 22RV1 cells (more potent than 155; enza failed), but also has some nonspecific toxicity in the COS7 cell line whose growth is not dependent on the AR. Improved antiproliferative potency and efficacy in 22RV1 cells is another advantage of SARCA which is consistent with the improved inhibition of AR-V7 transactivation (FIG. 10) as 22RV1 cells highly express AR-V7. Improved antiproliferative potency and efficacy was also seen in LNCaP cells that only express AR FL.

FIG. 17 depicts that 1 and 4 at 10 μM acted as degraders of AR (full length) and AR SV (AR-V7). AR degradation activity of 2 and 5 is also shown.

FIG. 18 depicts that 1, 4, and enzalutamide dose-dependently displaced tritiated R1881, whereas the vehicle (negative control) did not displace tritiated R1881. Negligible binding of tritiated R1881 was observed in the absence of LBD (vector). This experiment demonstrated that in addition to irreversible NTD binding (MS and Schild's analysis), these SARCAs also reversibly and competitively bind to the LBD. COS cells were plated in 24 well plates. Cells were transfected with AR LBD. Cells were treated as indicated in the figure in the presence of 1 nM³H-R1881 for 4 h. Cells were washed with cold PBS and intracellular radioactivity and cellular proteins were extracted using ice-cold 100% ethanol. Scintillation cocktail was added and the incorporated radioactivity was counted in a scintillation counter.

FIG. 19 depicts that LNCaP-V7 cells inducibly expressed AR-V7 by the addition of doxycycline (Dox). FIG. 19 (top left) demonstrates that in the absence of Dox, no AR-V7 was expressed (left panel), but upon addition of Dox then AR-V7 expression was seen. 1 and 4 degraded AR and AR-V7. FIG. 19 (top right) demonstrates that 1 degraded AR (see top band) and AR-V7 (see middle band) at 1 and 3 μM in 22RV1 cells. In 22RV1 cells where AR-V7 was endogenously co-expressed with AR, 1 and 4 both demonstrated AR degradation activity of AR FL and ARV7. FIG. 19 (bottom) shows degradation by 1 and 4 of AR FL (T877A) in the parental LNCaP cell line lacking expression of AR-V7.

FIG. 20 depicts that 1 was stable in rat liver microsome (RLM) for >60 minutes. Estimated half-life for Phase I stability was about 84 min.

FIG. 21 depicts that 1 had a half-life of 41 min in mouse liver microsomes (MLM).

FIG. 22 depicts that SARCAs 1 and 4 degraded both AR and AR-V7. LNCaP-V7 (LNCaP cells stably transfected with AR-V7) cells were plated in 60 mm dishes. Cells were treated in growth medium for 24 h. Cells were harvested, protein extracted, and Western blot for AR and AR-V7 was performed.

FIG. 23 depicts that 4 (630 nM) and 1 (776 nM) were moderate to weak inhibitors of GR, whereas 2 and 6 did not demonstrate significant inhibition of GR. This suggests some cross-reactivity of 4 and 1 in other steroid receptors. The GR and AR co-antagonism of SARCAs 1 and 4 is favorable for the treatment of prostate cancer whose AR-axis is reactivated by GR. It is unexpected in view of the structural differences between 1 and 4 vs. 2 and 6 that 1 and 4 would have nM level potency GR antagonism.

FIG. 24 depicts diagrammatically where the three alkylated cysteine residues map in the AF-1 domain and the AR FL as a whole. C267 and C327 lie within transcriptional activation unit-1 (Tau-1) and C407 lies within Tau-5.

FIGS. 25A and 25B depict that SARCA 4 (FIG. 25A) lowered Eₘₐₓ values (irreversible) whereas UT-34 (a noncovalent binder of AF-1 binder) (FIG. 25B) increased EC₅₀ values (reversible competitive). These results are as expected given that 4 alkylated AF-1 but UT-34 did not alkylate the AF-1.

FIG. 26 depicts that 4 was a weak antagonist of GR (1431 nM) and a moderate potency PR (125 nM) antagonist. These results are unexpected in view of the prior art and favorable for the treatment of prostate cancer whose AR-axis is reactivated by PR or GR. PR, GR and AR co-antagonism is an unexpectedly advantageous feature of 4 in these prostate cancers.

FIG. 27 depicts a Schild's analysis of 11. Trends toward right shift and decreased Eₘₐₓ like other SARCAs suggest a mixture of irreversible NTD binding and reversible LBD binding like other SARCAs of this invention.

FIG. 28 depicts significant inhibition with 1 at 3 (first number in column labels is the concentration in M, e.g., 10 Enza is 10 μM of enzalutamide and 3-1 is 3 μM of compound 1, etc.) and 10 μM, partial inhibition with 11 and 6 at 10 μM, and significant inhibition with 7 at 10 μM in an AR-V7 transactivation experiment. This demonstrates that AR-V7 inhibition is a generalizable activity of SARCAs whereas enzalutamide and vehicle fail, and no activation is seen in the absence of AR-V7 (vector). Further, enzalutamide failed to inhibit AR-V7 which lacks the LBD required for enzalutamide binding.

FIG. 29 depicts that 11, 6, and enzalutamide inhibited AR in vitro in an AR transactivation assay.

FIG. 30 depicts that 6 (164 nM) was almost equipotent to enzalutamide (149 nM) whereas 7 was slightly less potent (256 nM).

FIG. 31 depicts that enzalutamide (top left) failed to inhibit AR-V7 but SARCA 7 (top right), 1 (bottom left), and 6 (bottom right) each dose-dependently inhibited AR-V7. 1 was most potent (as low as 0.3 μM) but 6 and 7 demonstrated greater maximum efficacy at 10 M.

FIG. 32 depicts the three cysteine residues alkylated by 1 and maps them to the AF-1 domain. FIG. 32 reports the same results as in FIG. 24 and presents the data in a graphical way. The data incontrovertibly demonstrated irreversible binding of SARCAs (1 in this example) to the AR-1 of the NTD of AR.

FIG. 33 depicts the three cysteine residues alkylated by 1 and maps them to the AF-1 domain.

FIG. 34 depicts the three cysteines alkylated by 4.

FIG. 35 depicts that 4 and 1 alkylated the same three cysteine residues of AF-1, whereas UT-34 (a noncovalent binder of AF-1) did not alkylate AF-1. Additionally, 1 and 4 alkylated cysteine residues in GST.

FIG. 36 depicts that for 6, two of the cysteines in AF-1 were alkylated, C327 and C407.

FIG. 37 depicts that the same two cysteines of AF-1 were alkylated in the presence or absence of UT-34, a noncovalent binder of AF-1; and further demonstrates for 6 that both cysteines in GST were alkylated.

FIG. 38 depicts that 1 and 6, and to some extent enzalutamide, were able to overcome 0.1 nM R1881 induced AR-dependent LNCaP proliferation. 1 and 6 demonstrated dose-dependent inhibition with full efficacy antiproliferation at 1 μM and 10 μM, respectively, whereas enzalutamide only reached approximately 40% efficacy at 1, 3, and 10 μM.

FIG. 39 depicts that AR dependent gene expressions of PSA and FKBP5 in LNCaP cells were dose-dependently decreased by 1 and 6, like enzalutamide. This data confirms that AR antagonism observed in transcriptional activation assays translated into AR antagonism in AR dependent prostate cancer cells.

FIGS. 40A and 40B depict that in vivo AR antagonism was demonstrated in intact Sprague Dawley rats with SARCA 6. Prostate and seminal vesicles weights were reduced by ˜45% and 60% relative to intact control which is shown as vehicle (0% reduction). S.D. rats were treated for 14 days with 20 mg/kg/day oral dose of 6. Avg.+/−S.D. *=0.05; **=0.01; ***=0.001).

FIG. 41 depicts AR antagonist effects of 13 and 14. The top left panel was a positive control experiment that demonstrated that known agonist R1881 activated transcription in this transcriptional activation experiment. The top right panel demonstrated that 13 and 14 both inhibited AR transactivation. The bottom left panel demonstrated that neither 13 or 14 possessed any intrinsic agonist activity in vitro. The bottom right panel is the raw data for the graphs. This data demonstrates that although 13 and 14 lack the nitrogen atom in or near the left side aromatic ring, they are still potent inhibitors of wt AR.

FIG. 42 depicts a mass spec study with SARCA 7 showing alkylation of the ‘GLEGESLGSC . . . ’ and ‘LENPLDYGSA . . . ’ peptides (like 6 and 1), and also a novel peptide ‘EASGA . . . ’ (unique).

FIG. 43 depicts antagonist effects of 15, 8 and 4 which inhibited wtAR with IC₅₀ values of 2852 nM, 6525 nM, and 850.7 nM, respectively.

FIG. 44 depicts that compound 18 bound covalently to AR AF-1.

FIG. 45 depicts AR antagonist activity of compounds 1 and 6.

FIGS. 46A and 46B depict that compounds 1 and 6 inhibited AR-V7 (FIG. 46A), but not NFkB (FIG. 46B), transactivation.

FIG. 47 depicts that compound 6 inhibited AR-target gene expression in prostate cancer cells.

FIG. 48 depicts that compound 6 inhibited prostate cancer cell proliferation.

FIG. 49 depicts that compounds 1 and 6 inhibited proliferation of prostate cancer cells that expressed AR-splice variants (AR-SVs).

FIGS. 50A-50C depict that compounds 1 and 6 inhibited proliferation of prostate cancer cells that expressed AR-SVs, but not non-cancerous cells. FIG. 50A: 22RV1 proliferation (compound 6); Figure SOB: 22RV1 proliferation (compound 1); and Figure SOC: COS7 proliferation (compound 6).

FIG. 51 depicts that compounds 6 inhibited wildtype AR-V7 transactivation, but not transactivation of AR-V7 where three cysteines (C267, C327, and C406) were mutated.

FIG. 52 depicts that mutating individual cysteines did not affect compound 6 activity, but affected AR-V7 function. Mutating the cysteines individually to alanines, reduced AR-V7 activity, but had minimum to no effect on SARCA (e.g., compound 6) inhibitory activity.

FIGS. 53A and 53B depict that compounds 1 and 6 inhibited AR-target tissues prostate and seminal vesicles. FIG. 53A: S.V. weight normalized to body weight; and FIG. 53B: prostate weight normalized to body weight.

FIG. 54 depicts that compound 6 had long half-life in rats. Male Sprague Dawley rats (n=3/timepoint; 80-100 gms) were treated orally with 20 mg/kg SARCA. Blood was collected at the indicated timepoints. Amount of drug present in serum was measured using LC-MS/MS.

FIGS. 55A and 55B depict that compound 6 inhibited growth of prostate cancer and triple-negative breast cancer xenograft growth in NSG mice. FIG. 55A: LNCaP-AR xenograft in intact NSG mice; and FIG. 55B: MDA-MB-453 TNBC xenograft in NSG mice.

FIGS. 56A-56D provide quantification of peptides modified by compounds 1 and 6. FIG. 56A: modification of AR AF-1 by compound 6; FIG. 56B: modification of AR AF-1 by compound 1; FIG. 56C: modification of AR AF-1 & LBD by compound 6; and FIG. 56D: modification of AR AF-1 & LBD by compound 1.

FIGS. 57A-57C depict that C406, C327, and C267 were important for the AR-V7 stability.

FIGS. 58A and 58B depict that compounds 1 and 6 minimally cross-reacted with GST.

FIGS. 59A-59D depict that UT-105 and UT-34 competed with 1 and 6 for binding to AF-1. FIG. 59A: compound 6 alone or in combination with UT-34 (C406); FIG. 59B: compound 6 alone or in combination with UT-34 (C327); FIG. 59C: compound 6 alone or in combination with UT-105; and FIG. 59D: compound 6 alone or in combination with UT-105.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Androgens act in cells by binding to the AR, a member of the steroid receptor superfamily of transcription factors. As the growth and maintenance of prostate cancer (PCa) is largely controlled by circulating androgens, treatment of PCa heavily relies on therapies that target AR. Treatment with AR antagonists such as darolutamide, apalutamide, enzalutamide, abiraterone (an indirect antagonist), bicalutamide or hydroxyflutamide to disrupt receptor activation has been successfully used in the past to reduce PCa growth. All currently available direct AR antagonists competitively bind AR and recruit corepressors such as NCoR and SMRT to repress transcription of target genes. However, altered intracellular signaling, AR mutations, and increased expression of coactivators lead to functional impairment of antagonists or even transformation of antagonists into agonists. Studies have demonstrated that mutation of W741 and T877 within AR converts bicalutamide and hydroxyflutamide, respectively, to agonists. Similarly, increased intracellular cytokines recruit coactivators instead of corepressors to AR-responsive promoters subsequently converting bicalutamide to an agonist. Similarly, mutations that have been linked to enzalutamide, apalutamide and abiraterone resistance include F876, H874, T877, and di-mutants T877/S888, T877/D890, F876/T877 (i.e., MR49 cells), and H874/T877 (Genome Biol. (2016) 17:10 (doi: 10.1186/si3059-015-0864-1)). Abiraterone resistance mutations include L702H mutations which results in activation of the AR by glucocorticoids such as prednisone, causing resistance to abiraterone because abiraterone is usually prescribed in combination with prednisone. If resistance develops to enzalutamide then often the patient is refractory to abiraterone and apalutamide also and vice versa; or the duration of response is very short. Darolutamide also has limited efficacy and duration of action in CRPC. This situation highlights the need for a definitive androgen ablation therapy to prevent AR reactivation in advanced prostate cancers.

Despite initial response to androgen deprivation therapy (ADT), PCa disease progression is inevitable and the cancer emerges as castration-resistant prostate cancer (CRPC). The primary reason for castration resistant prostate cancer (CRPC) re-emergence is re-activation of androgen receptor (AR) by alternate mechanisms such as:
(a) intracrine androgen synthesis;
(b) expression of AR splice variants (AR-SV), e.g., that lack ligand binding domain (LBD);
(c) AR-LBD mutations with potential to resist antagonists;
(d) hyper-sensitization of AR to low androgen levels, e.g., due to AR gene amplification or AR mutation;
(e) amplification of the AR gene within the tumor; and
(f) over expression of coactivators and/or altered intracellular signal transduction.

The invention encompasses novel selective androgen receptor covalent antagonists (SARCA) compounds encompassed by formula I, which inhibit the growth of prostate cancer (PCa) cells and tumors that are dependent on AR full length (AR-FL) including pathogenic and resistance mutations and wildtype, and/or AR splice variants (AR-SV) for proliferation.

As used herein, unless otherwise defined, a “selective androgen receptor covalent antagonist” (SARCA) compound is an androgen receptor antagonist capable of inhibiting the growth of PCa cells and tumors that are dependent on AR-full length (AR-FL) and/or AR splice variants (AR-SV) for proliferation. Alternatively, a “selective androgen receptor covalent antagonist” (SARCA) compound is an androgen receptor antagonist capable of causing degradation of a variety of pathogenic mutant variant ARs and wildtype AR and hence are capable of exerting anti-androgenism is a wide variety of pathogenic altered cellular environments found in the disease states embodied in this invention.

The selective androgen receptor covalent antagonists (SARCA) bind covalently to the AR and inhibit its activity irreversibly. Some SARCA compounds bind irreversibly and covalently to the AR AF-1 domain, which is demonstrated by mass spectrometry experiments as described herein. Other SARCA compounds may bind to the LBD of AR.

The SAR CA compound may bind to the N-terminal domain (NTD) of the AR; to an alternate binding and degradation domain (BDD) of the AR; to both the AR ligand binding domain (LBD) and to an alternate binding and degradation domain (BDD); or to both the N-terminal domain (NTD) and to the ligand binding domain (LBD) of the AR. In one embodiment, the BDD may be located in the NTD. In one embodiment, the BDD is located in the AF-1 region of the NTD. Alternatively, the SARCA compound may be capable of: inhibiting growth driven by the N-terminal domain (NTD)-dependent constitutively active AR-SV; or inhibiting the AR through binding to a domain that is distinct from the AR LBD. Also, the SARCA compound may be a strong (i.e., highly potent and highly efficacious) selective androgen receptor antagonist, which antagonizes the AR stronger than other known AR antagonists (e.g., darolutamide, apalutamide, enzalutamide, bicalutamide and abiraterone).

The SAR CA compound may be a selective androgen receptor antagonist, which targets AR-SVs, which cannot be inhibited by conventional antagonists. The SARCA compound may exhibit any one of several activities including, but not limited to: AR-SV degradation activity; AR-FL degradation activity; AR-SV inhibitory activity (i.e., is an AR-SV antagonist); AR-FL inhibitory activity (i.e., is an AR-FL antagonist); inhibition of the constitutive activation of AR-SVs; or inhibition of the constitutive activation of AR-FLs. Alternatively, the SARCA compound may possess dual AR-SV degradation and AR-SV inhibitory functions, and/or dual AR-FL degradation and AR-FL inhibitory functions; or alternatively possess all four of these activities.

The SARCA compound may also degrade AR-FL and AR-SV. The SARCA compound may degrade the AR through binding to a domain that is distinct from the AR LBD. The SARCA compound may possess dual degradation and AR-SV inhibitory functions that are distinct from any available CRPC therapeutics. The SARCA compound may inhibit the re-activation of the AR by alternate mechanisms such as: intracrine androgen synthesis, expression of AR-SV that lack ligand binding domain (LBD) and AR-LBD mutations with potential to resist antagonists, or inhibit reactivated androgen receptors present in pathogenic altered cellular environments.

Examples of AR-splice variants include, but are not limited to, AR-V7 and ARv567es (a.k.a. AR-V12; S. Sun, et al. Castration resistance in human prostate cancer is conferred by a frequently occurring androgen receptor splice variant. J Clin Invest. (2010) 120(8), 2715-2730). Nonlimiting examples of AR mutations conferring antiandrogen resistance are: W741L, T877A, and F876L (J. D. Joseph et al. A clinically relevant androgen receptor mutation confers resistance to seconds generation antiandrogens enzalutamide and ARN-509. Cancer Discov. (2013) 3(9), 1020-1029) mutations. Many other LBD resistance conferring mutations are known in the art and will continue to be discovered. AR-V7 is a splice variant of AR that lacks the LBD (A. H. Bryce & E. S. Antonarakis. Androgen receptor splice variant 7 in castration-resistant prostate cancer: Clinical considerations. Int J Urol. (2016 June 3) 23(8), 646-53. doi: 10.1111/iju.13134). It is constitutively active and has been demonstrated to be responsible for aggressive PCa and resistance to endocrine therapy.

The invention encompasses novel selective androgen receptor covalent antagonist (SARCA) compounds of formulas I-XX which bind to the AR through an alternate binding and degradation domain (BDD), e.g., the NTD or AF-1. The SARCAs may further bind the AR ligand binding domain (LBD). SARCA compounds possess nucleophile acceptor groups intended to acceptor a nucleophile from within the AR. Either NTD binding or LBD binding may be irreversible.

The SARCA compounds may be used in treating CRPC that cannot be treated with any other antagonist. The SARCA compounds may treat CRPC by irreversibly inhibiting the AR-SVs or degrading AR-SVs. The SARCA compounds may maintain their antagonistic activity in AR mutants that normally convert AR antagonists to agonists. For instance, the SARCA compounds are expected to maintain their antagonistic activity to AR mutants W₇₄₁L, T877A, and F876L (J. D. Joseph et al. A clinically relevant androgen receptor mutation confers resistance to second-generation antiandrogens enzalutamide and ARN-509. Cancer Discov. (2013) 3(9), 1020-1029). Alternatively, the SARCA compounds elicit antagonistic activity within an altered cellular environment in which LED-targeted agents are not effective or in which NTD-dependent AR activity is constitutively active.

Selective Androgen Receptor Covalent Antagonist (SARCA) Compounds

The compounds of the invention as described herein are selective androgen receptor covalent antagonist (SARCA) compounds. The SARCA compounds as described herein may irreversibly bind FL or SV androgen receptors, degrade FL or SV androgen receptors, or bind reversibly to NTD and/or LBD.

In one aspect, the invention encompasses a compound represented by the structure of formula I

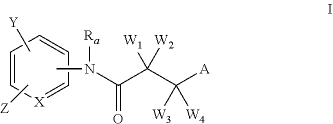

wherein
X is CH or N;
Y is H, CF₃, F, Br, Cl, I, CN, or C(R)₃;
Z is H, NO₂, CN, F, Br, Cl, I, COOH, COR, NHCOR, or CONHR;
or Y and Z form a 5 to 8 membered fused ring;
R is H, alkyl, alkenyl, CH₂CH₂OH, CF₃, CH₂Cl, CH₂CH₂Cl, aryl, F, Cl, Br, I, or OH;
Rₐ is H, alkyl-NCO, alkyl-NCS, alkyl-SCN, alkyl-OCN, alkyl-N₃, alkyl-SO₂F, alkyl-CH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂, wherein halide is F, Cl, Br, or I;
W₁ is H or OR_{d}, wherein R_{d} is H, alkyl-NCO, alkyl-NCS, alkyl-SCN, alkyl-OCN, alkyl-N₃, alkyl-SO₂F, alkyl-CH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
W₂ is CH₃, CH₂F, CHF₂, CF₃, CH₂CH₃, CF₂CF₃, or CH₂A;
or W₁ and W₂, together with the carbon atom to which they are attached, form a C═CW₅W₆ group, wherein W₅ and W₆ are each H or alkyl;
W₃ and W₄ are individually H, OH, alkyl, wherein the alkyl is optionally substituted with OR, NO₂, CN, F, Br, Cl, I, COR, NHCOR, CONHR, —NCO, —NCS, —SCN, —OCN, —N₃, —SO₂F, —CH₂halide, —NHCOCH₂halide, —NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
or one of W₁ and W₂ with one of W₃ and W₄, together with the carbon atoms to which they are attached, form a C═C bond;
A is NR_{b}R_{c} or a 5 to 10-membered aryl or heteroaryl group, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
R_{b} is H or alkyl, wherein the alkyl is optionally substituted with OR, NO₂, CN, F, Br, Cl, I, COR, NHCOR, or CONHR;
R_{c} is alkyl, haloalkyl, cycloalkyl, heterocycloalkyl, aryl or heteroaryl, wherein said alkyl, haloalkyl, cycloalkyl, heterocycloalkyl, aryl and heteroaryl groups are optionally substituted with CN, NO₂, CF₃, F, Cl, Br, I NHCOOR, N(R)₂, NHCOR, COR, alkyl, or alkoxy;
or R_{b} and R_{c}, together with the nitrogen atom to which they are attached, form a 5 to 10-membered saturated or unsaturated heterocyclic ring having at least one nitrogen atom and 0, 1, or 2 double bonds, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
or its isomer, optical isomer, racemic mixture, pharmaceutically acceptable salt, pharmaceutical product, hydrate or any combination thereof.

In one embodiment, the compound of the invention is represented by the structure of formula II

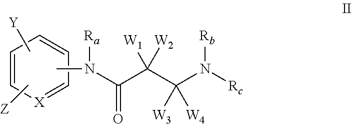

wherein
X is CH or N;
Y is H, CF₃, F, Br, Cl, I, CN, or C(R)₃;
Z is H, NO₂, CN, F, Br, Cl, I, COOH, COR, NHCOR, or CONHR;
or Y and Z form a 5 to 8 membered fused ring;
R is H, alkyl, alkenyl, CH₂CH₂OH, CF₃, CH₂Cl, CH₂CH₂Cl, aryl, F, Cl, Br, I, or OH;
Rₐ is H, alkyl-NCO, alkyl-NCS, alkyl-SCN, alkyl-OCN, alkyl-N₃, alkyl-SO₂F, alkyl-CH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂, wherein halide is F, Cl, Br, or I;
W₁ is H or OR_{d}, wherein R_{d} is H, alkyl-NCO, alkyl-NCS, alkyl-SCN, alkyl-OCN, alkyl-N₃, alkyl-SO₂F, alkyl-CH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
W₂ is CH₃, CH₂F, CHF₂, CF₃, CH₂CH₃, CF₂CF₃, or CH₂A;
or W₁ and W₂, together with the carbon atom to which they are attached, form a C═CW₅W₆ group, wherein W₅ and W₆ are each H or alkyl; W₃ and W₄ are individually H, OH, alkyl, wherein the alkyl is optionally substituted with OR, NO₂, CN, F, Br, Cl, I, COR, NHCOR, CONHR, —NCO, —NCS, —SCN, —OCN, —N₃, —SO₂F, —CH₂halide, —NHCOCH₂halide, —NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
or one of W₁ and W₂ with one of W₃ and W₄, together with the carbon atoms to which they are attached, form a C═C bond;
A is NR_{b}R_{c} or a 5 to 10-membered aryl or heteroaryl group, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
R_{b} is H or alkyl, wherein the alkyl is optionally substituted with OR, NO₂, CN, F, Br, Cl, I, COR, NHCOR, or CONHR;
R_{c} is alkyl, haloalkyl, cycloalkyl, heterocycloalkyl, aryl or heteroaryl, wherein said alkyl, haloalkyl, cycloalkyl, heterocycloalkyl, aryl and heteroaryl groups are optionally substituted with CN, NO₂, CF₃, F, Cl, Br, I NHCOOR, N(R)₂, NHCOR, COR, alkyl, or alkoxy;
or R_{b} and R_{c}, together with the nitrogen atom to which they are attached, form a 5 to 10-membered saturated or unsaturated heterocyclic ring having at least one nitrogen atom and 0, 1, or 2 double bonds, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
or its isomer, optical isomer, racemic mixture, pharmaceutically acceptable salt, pharmaceutical product, hydrate or any combination thereof.

In some embodiments of the structure of formula I or II, at least one of Rₐ, W₁, W₂, W₃, W₄, or Q¹-Q⁴ contain an α, β-unsaturated carbonyl such as a ketone, amide, ester, acid halide, acid anhydride, imide, or the like, or another nucleophile acceptor group which acts as an acceptor of a nucleophile from within the AR.

In some embodiments of the structure of formula I or II, Rₐ and R_{d} are not H at the same time.

In some embodiments, the compound of the invention represented by the structure of formula I or formula II contains at least one nucleophile acceptor group. In one embodiment, the compound of the invention represented by the structure of formula I or formula II contains at least one functional group with an α, β-unsaturated carbonyl. In one embodiment, such α, β-unsaturated carbonyl functional groups include but are not limited to α, β-unsaturated ketones, amides, esters, thioesters, acid anhydrides, carboxylic acids, carboxylates, acid halides, imides, and the like. In one embodiment, the α, β-unsaturated functional group serves as a Michael addition acceptor for nucleophiles within the AR.

In one embodiment, the compound of the invention represented by the structure of formula I or formula II contains at least one nucleophile acceptor group. In one embodiment, the nucleophile acceptor group is at least one of isocyanato (—NCO), isothiocyanato (—NCS), cyanato (—CNO), thiocyanato (—CNS), azido (N₃), sulfonyl fluoride (—SO₂F), halomethyl (—CH₂-halide), 2-haloacetyl (—NHCOCH₂-halide), halosulfonyl (—NHSO₂CH₂-halide), and the like. In one embodiment, the nucleophile acceptor group serves as a nucleophile acceptor for nucleophiles within the AR. In one embodiment, said AR nucleophile is within the NTD. In another embodiment, said AR nucleophile is within the AF-1 domain. In an embodiment, said AR nucleophile is within the LBD. In one embodiment, the nucleophile acceptor group is present in the Rₐ group. In one embodiment, the nucleophile acceptor group is present in the W₁ group. In one embodiment, the nucleophile acceptor group is present in the W₃ or W₄ group. In one embodiment, the nucleophile acceptor group is present in any one of the Q¹, Q², Q³ or Q⁴ groups.

In one embodiment, the compound of the invention is represented by the structure of formula III:

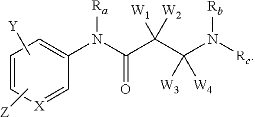

In one embodiment, X, Y, Z, Rₐ, R_{b}, R_{c}, W₁, W₂, W₃, and W₄ are defined as anywhere herein.

In one embodiment, the compound of the invention is represented by the structure of formula IV:

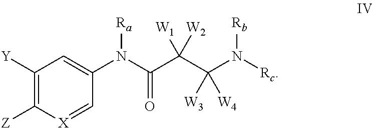

In one embodiment, X, Y, Z, Rₐ, R_{b}, R_{c}, W₁, W₂, W₃, and W₄ are defined as anywhere herein.

In one embodiment, the compound of the invention is represented by the structure of formula V:

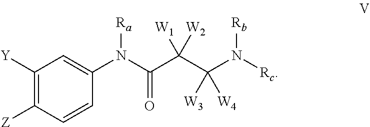

In one embodiment, Y, Z, Rₐ, R_{b}, R_{c}, W₁, W₂, W₃, and W₄ are defined as anywhere herein.

In one embodiment, the compound of the invention is represented by the structure of formula VI:

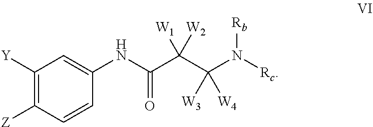

In one embodiment, Y, Z, R_{b}, R_{c}, W₁, W₂, W₃, and W₄ are defined as anywhere herein.

In one embodiment, the compound of the invention is represented by the structure of formula VII:

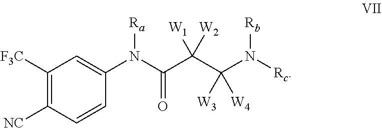

In one embodiment, Rₐ, R_{b}, R_{c}, W₁, W₂, W₃, and W₄ are defined as anywhere herein.

In one embodiment, in the compound of the invention, W₁ and W₂, together with the carbon atom to which they are attached, form a C═CW₅W₆ group. In one embodiment, W₁ is OR_{d}. In one embodiment, one of W₁ and W₂ with one of W₃ and W₄, together with the carbon atoms to which they are attached, form a C═C bond.

In one embodiment, the compound of the invention is represented by the structure of formula VIII:

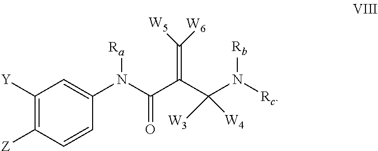

In one embodiment, Y, Z, Rₐ, R_{b}, R_{c}, W₅, W₆, W₃, and W₄ are defined as anywhere herein.

In one embodiment, the compound of the invention is represented by the structure of formula IX:

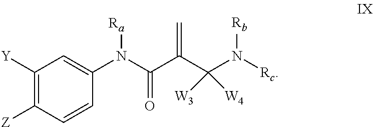

In one embodiment, Y, Z, Rₐ, R_{b}, R_{c}, W₃, and W₄ are defined as anywhere herein.

In one embodiment, in the compound of formula IX, R_{b} and R_{c}, together with the nitrogen atom to which they are attached, form a 5 or 6 membered unsaturated heterocyclic ring, optionally substituted with CN, NO₂, CF₃, F, Cl, Br, I, NHCOOR, N(R)₂, NHCOR, COR, alkyl, alkoxy, or substituted or unsubstituted phenyl. In one embodiment, R_{b} and R_{c}, together with the nitrogen atom to which they are attached, form an optionally substituted indole group. In one embodiment, the indole group is substituted with halogen or CN.

In one embodiment, in the compound of formula IX, R_{b} is H and R_{c} is aryl or heteroaryl, optionally substituted with CN, NO₂, CF₃, F, Cl, Br, I, NHCOOR, N(R)₂, NHCOR, COR, alkyl, or alkoxy.

In one embodiment, the compound of the invention is represented by the structure of formula X:

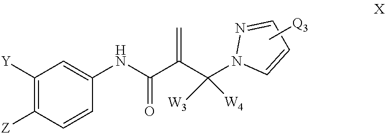

wherein Q₃ is hydrogen, CN, NO₂, CF₃, F, Cl, Br, I, NHCOOR, N(R)₂, NHCOR, COR, alkyl, alkoxy, or substituted or unsubstituted phenyl.

In one embodiment, Y, Z, W₃, and W₄ are defined as anywhere herein.

In one embodiment, in the compound of formula X, Q₃ is F. In one embodiment, Q₃ is CN. In one embodiment, W₃ and W₄ are H.

In one embodiment, the compound of the invention is represented by the structure of formula XI:

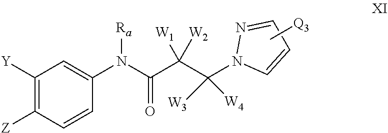

wherein Q₃ is hydrogen, CN, N₀₂, CF₃, F, Cl, Br, I, NHCOOR, N(R)₂, NHCOR, COR, alkyl, alkoxy, or substituted or unsubstituted phenyl.

In one embodiment, Y, Z, Rₐ, W₁, W₂, W₃, and W₄ are defined as anywhere herein.

In one embodiment, in the compound of formula XI, W₃ and W₄ are H. In one embodiment, Rₐ is —CH₂—C(COOR)═CH₂. In one embodiment, W₁ is OR_{d}, wherein R_{d} is H, —CH₂—CH═CH—COOR or —CH₂—C(COOR)═CH₂.

In one embodiment, the compound of the invention is represented by the structure of formula XII:

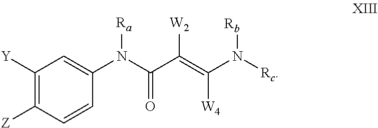

In one embodiment, Y, Z, Rₐ, R_{b}, R_{c}, W₂, and W₄ are defined as anywhere herein.

In one embodiment, the compound of the invention is represented by the structure of formula XIII:

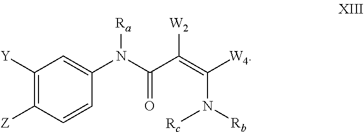

In one embodiment, Y, Z, Rₐ, R_{b}, R_{c}, W₂, and W₄ are defined as anywhere herein.

In one embodiment, in the compound of formula XIII, W₂ is H. In one embodiment, W₄ is CH₃. In one embodiment, in the compound of formula XIII, W₂ and W₄ are H.

In one embodiment, the compound of the invention is represented by the structure of formula XIV:

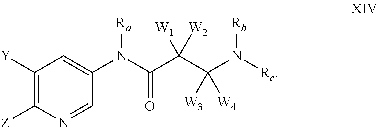

In one embodiment, Y, Z, Rₐ, R_{b}, R_{c}, W₁, W₂, W₃, and W₄ are defined as anywhere herein.

In one embodiment, in the compound of formula XIV, W₁ is OR_{d}. In one embodiment, R_{d} is H, —CH₂—CH═CH—COOR or —CH₂—C(COOR)═CH₂. In one embodiment, W₂ is CH₃. In one embodiment, Y is CF₃ and Z is CN.

In one embodiment, the compound of the invention is represented by the structure of formula XV:

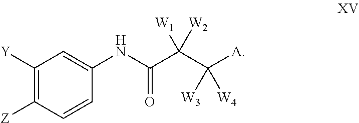

In one embodiment, Y, Z, A, W₁, W₂, W₃, and W₄ are defined as anywhere herein.

In one embodiment, the compound of the invention is represented by the structure of formula XVI:

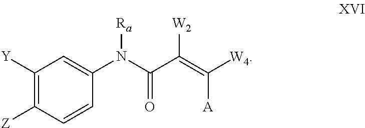

In one embodiment, Y, Z, Rₐ, A, W₂, and W₄ are defined as anywhere herein.

In one embodiment, the compound of the invention is represented by the structure of formula XVII:

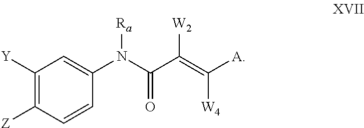

In one embodiment, Y, Z, Rₐ, A, W₂, and W₄ are defined as anywhere herein.

In one embodiment, the compound of the invention is represented by the structure of formula XVIII:

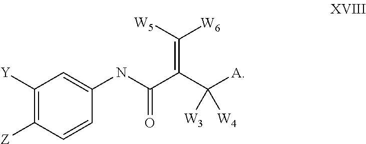

In one embodiment, Y, Z, A, W₅, W₆, W₃, and W₄ are defined as anywhere herein.

In one embodiment, the compound of the invention is represented by the structure of formula XIX:

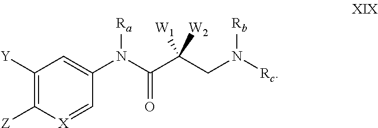

In one embodiment, X, Y, Z, Rₐ, R_{b}, R_{c}, W₁, and W₂ are defined as anywhere herein. In some embodiments of the structure of formula XIX, Rₐ and R_{d} are not H at the same time.

In one embodiment, X is CH. In another embodiment, X is N.

In one embodiment, Y is CF₃. In one embodiment, Z is CN.

In one embodiment, Rₐ is H. In one embodiment, Rₐ is —CH₂—C(COOR)═CH₂.

In one embodiment, W₁ is H. In one embodiment, W₁ is OR_{d}. In one embodiment, R_{d} is H, —CH₂—CH═CH—COOR or —CH₂—C(COOR)═CH₂. In one embodiment, W₂ is CH₃. In one embodiment, W₃ is H. In one embodiment, W₄ is H.

In one embodiment, R_{b} and R_{c}, together with the nitrogen atom to which they are attached, form a 5 to 10-membered unsaturated heterocyclic ring, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR or COR. In one embodiment, R_{b} and R_{c}, together with the nitrogen atom to which they are attached, form a 5 to 10-membered unsaturated heterocyclic ring, optionally substituted with substituted or unsubstituted alkyl, substituted or unsubstituted cycloalkyl, haloalkyl, F, Cl, Br, I, CN, NO₂, or OR. In one embodiment, R_{b} and R_{c}, together with the nitrogen atom to which they are attached, form a 5-membered unsaturated heterocyclic ring, optionally substituted with CF₃, F, Cl, Br, I, CN, NO₂, OH, or OCH3. In one embodiment, the 5-membered unsaturated heterocyclic ring is pyrrole, pyrazole, pyrazolidine, imidazole, or triazole.

In one embodiment, the compound of the invention is represented by the structure of formula XX:

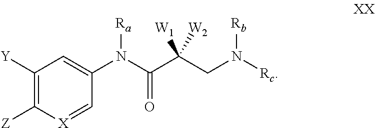

In one embodiment, X, Y, Z, Rₐ, R_{b}, R_{c}, W₁, and W₂ are defined as anywhere herein. In some embodiments of the structure of formula XX, Rₐ and R_{d} are not H at the same time.

In one embodiment, X is CH. In another embodiment, X is N.

In one embodiment, Y is CF₃. In one embodiment, Z is CN.

In one embodiment, Rₐ is H. In one embodiment, Rₐ is —CH₂—C(COOR)═CH₂.

In one embodiment, W₁ is H. In one embodiment, W₁ is OR_{d}. In one embodiment, R_{d} is H, —CH₂—CH═CH—COOR or —CH₂—C(COOR)═CH₂. In one embodiment, W₂ is CH₃. In one embodiment, W₃ is H. In one embodiment, W₄ is H.

In one embodiment, R_{b} and R_{c}, together with the nitrogen atom to which they are attached, form a 5 to 10-membered unsaturated heterocyclic ring, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR or COR. In one embodiment, R_{b} and R_{c}, together with the nitrogen atom to which they are attached, form a 5 to 10-membered unsaturated heterocyclic ring, optionally substituted with substituted or unsubstituted alkyl, substituted or unsubstituted cycloalkyl, haloalkyl, F, Cl, Br, I, CN, NO₂, or OR. In one embodiment, R_{b} and R_{c}, together with the nitrogen atom to which they are attached, form a 5-membered unsaturated heterocyclic ring, optionally substituted with CF₃, F, Cl, Br, I, CN, NO₂, OH, or OCH₃. In one embodiment, the 5-membered unsaturated heterocyclic ring is pyrrole, pyrazole, pyrazolidine, imidazole, or triazole.

In one embodiment, the compound of the invention is represented by the structure of any one of the following compounds:

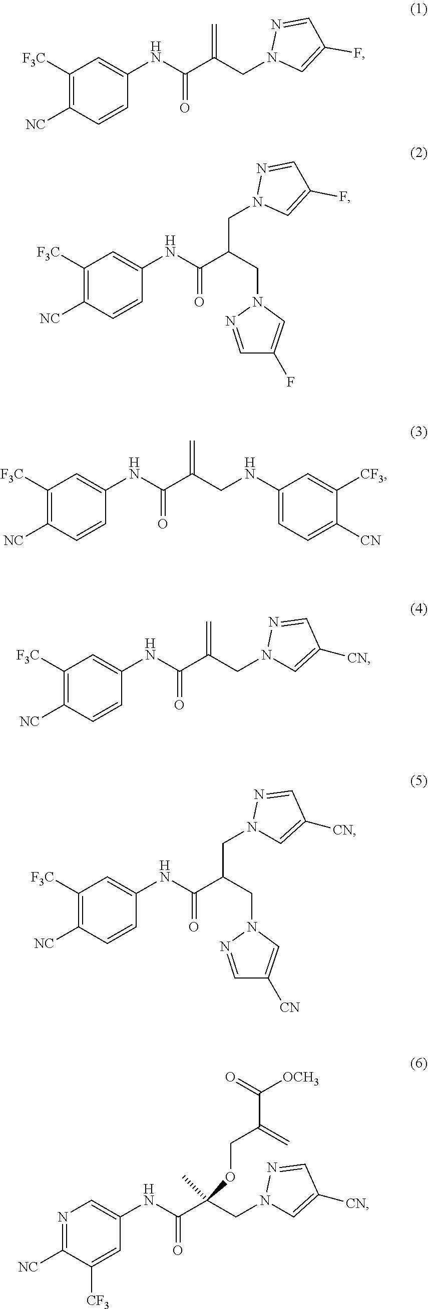 [SMILES: C=C(Cn1cc(F)cn1)C(=O)Nc1ccc(C#N)c(C(F)(F)F)c1 ; N#Cc1ccc(NC(=O)C(Cn2cc(F)cn2)Cn2cc(F)cn2)cc1C(F)(F)F ; C=C(CNc1ccc(C#N)c(C(F)(F)F)c1)C(=O)Nc1ccc(C#N)c(C(F)(F)F)c1 ; C=C(Cn1cc(C#N)cn1)C(=O)Nc1ccc(C#N)c(C(F)(F)F)c1 ; N#Cc1cnn(CC(Cn2cc(C#N)cn2)C(=O)Nc2ccc(C#N)c(C(F)(F)F)c2)c1 ; C=C(CO[C@@](C)(Cn1cc(C#N)cn1)C(=O)Nc1cnc(C#N)c(C(F)(F)F)c1)C(=O)OC]

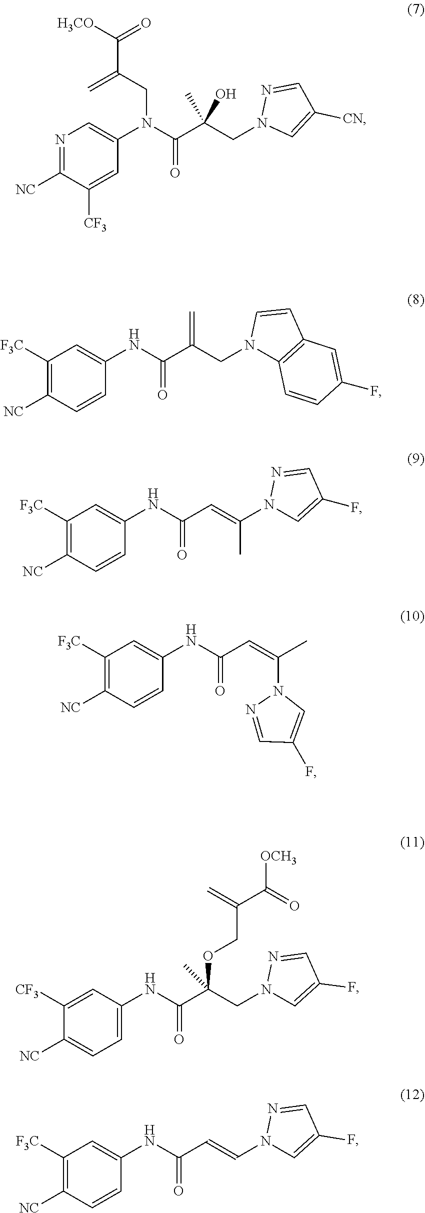 [SMILES: C=C(CN(C(=O)[C@@](C)(O)Cn1cc(C#N)cn1)c1cnc(C#N)c(C(F)(F)F)c1)C(=O)OC ; C=C(Cn1ccc2cc(F)ccc21)C(=O)Nc1ccc(C#N)c(C(F)(F)F)c1 ; C/C(=C\C(=O)Nc1ccc(C#N)c(C(F)(F)F)c1)n1cc(F)cn1 ; C/C(=C/C(=O)Nc1ccc(C#N)c(C(F)(F)F)c1)n1cc(F)cn1 ; C=C(CO[C@@](C)(Cn1cc(F)cn1)C(=O)Nc1ccc(C#N)c(C(F)(F)F)c1)C(=O)OC ; N#Cc1ccc(NC(=O)/C=C/n2cc(F)cn2)cc1C(F)(F)F]

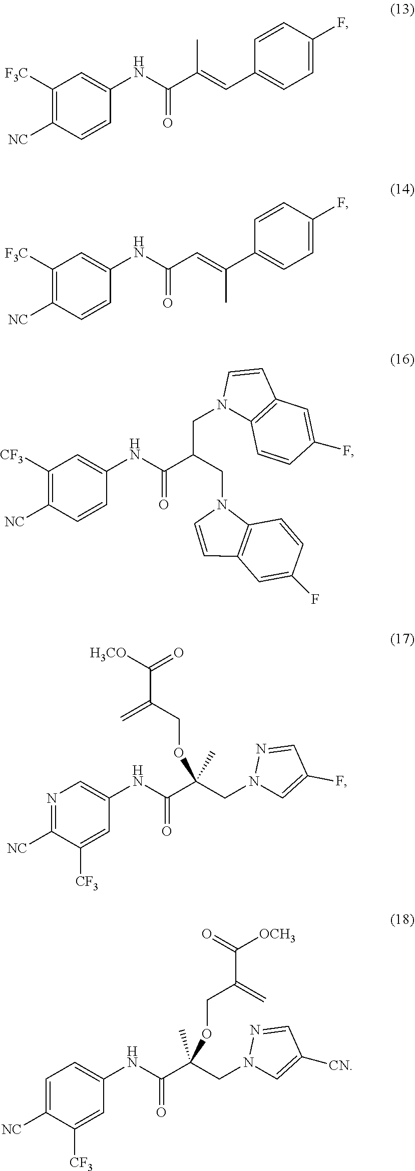 [SMILES: C/C(=C\c1ccc(F)cc1)C(=O)Nc1ccc(C#N)c(C(F)(F)F)c1 ; C/C(=C\C(=O)Nc1ccc(C#N)c(C(F)(F)F)c1)c1ccc(F)cc1 ; N#Cc1ccc(NC(=O)C(Cn2ccc3cc(F)ccc32)Cn2ccc3cc(F)ccc32)cc1C(F)(F)F ; C=C(CO[C@@](C)(Cn1cc(F)cn1)C(=O)Nc1cnc(C#N)c(C(F)(F)F)c1)C(=O)OC ; C=C(CO[C@@](C)(Cn1cc(C#N)cn1)C(=O)Nc1ccc(C#N)c(C(F)(F)F)c1)C(=O)OC]

In one embodiment, the compound of the invention is represented by the structure of compound 15

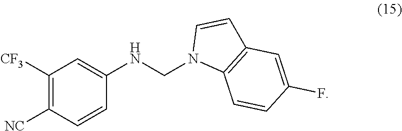 [SMILES: N#Cc1ccc(NCn2ccc3cc(F)ccc32)cc1C(F)(F)F]

In some embodiments of the compounds of the invention, X is CH. In some embodiments, X is N.

In some embodiments of the compounds of the invention, Y is H. In some embodiments, Y is CF₃. In some embodiments, Y is F. In some embodiments, Y is I. In some embodiments, Y is Br. In some embodiments, Y is Cl. In some embodiments, Y is CN. In some embodiments, Y is C(R)₃.

In some embodiments of the compounds of the invention, Z is H. In some embodiments, Z is NO₂. In some embodiments, Z is CN. In some embodiments, Z is a halide. In some embodiments, Z is F. In some embodiments, Z is Cl. In some embodiments, Z is Br. In some embodiments, Z is I. In some embodiments, Z is COOH. In some embodiments, Z is COR. In some embodiments, Z is NHCOR. In some embodiments, Z is CONHR.

In some embodiments, Y and Z forms a fused ring with the phenyl. In other embodiments, the fused ring with the phenyl is a 5 to 8 membered ring. In other embodiments, the fused ring with the phenyl is a 5 or 6 membered ring. In other embodiments, the ring is a carbocyclic or heterocyclic. In other embodiments, Y and Z form together with the phenyl to form a naphthyl, quinolinyl, benzimidazolyl, indazolyl, indolyl, isoindolyl, indenyl, or quinazolinyl.

In some embodiments of the compounds of the invention, A is a five or six-membered saturated or unsaturated ring having at least one nitrogen atom. In another embodiment, A is a substituted or unsubstituted pyrrole, pyrroline, pyrrolidine, pyrazole, pyrazoline, pyrazolidine, imidazole, imidazoline, imidazolidine, triazole, tetrazole, pyridine, morpholine, or other heterocyclic ring. Each represents a separate embodiment of this invention. In another embodiment, A is a five or six-membered heterocyclic ring. In another embodiment, a nitrogen atom of the five or six membered saturated or unsaturated ring is attached to the backbone structure of the molecule. In another embodiment, a carbon atom of the five or six membered saturated or unsaturated ring is attached to the backbone structure of the molecule. In some embodiments, of the compounds of the invention, A is a 5-10 membered aryl or heteroaryl group, optionally substituted with at least one of Q¹, Q2, Q³ or Q4, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, or COR.

In some embodiments of the compounds of the invention, A of the compound of the invention is NR_{b}R_{c}. In one embodiment, R_{b} is H. In another embodiment, R_{b} is alkyl, wherein the alkyl is optionally substituted with OR, NO₂, CN, F, Br, Cl, I, COR, NHCOR, or CONHR. In one embodiment, R_{c} is alkyl, haloalkyl, cycloalkyl, heterocycloalkyl, aryl or heteroaryl, wherein said alkyl, haloalkyl, cycloalkyl, heterocycloalkyl, aryl and heteroaryl groups are optionally substituted with CN, NO₂, CF₃, F, Cl, Br, I NHCOOR, N(R)₂, NHCOR, COR, alkyl, or alkoxy. In one embodiment, R_{b} and R_{c}, together with the nitrogen atom to which they are attached, form a 5 to 10-membered saturated or unsaturated heterocyclic ring having at least one nitrogen atom and 0, 1, or 2 double bonds, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, or COR.

In some embodiments of the compounds of the invention, R_{b} and R_{c}, together with the nitrogen atom to which they are attached, form a substituted or unsubstituted pyrrole, pyrroline, pyrrolidine, pyrazole, pyrazoline, pyrazolidine, imidazole, imidazoline, imidazolidine, triazole, tetrazole, pyridine, morpholine, or other heterocyclic ring. Each represents a separate embodiment of this invention.

In some embodiments, one of Q¹, Q², Q³ and Q⁴ is hydrogen. In some embodiments, one of Q¹, Q², Q³ and Q⁴ is CN. In other embodiments, one of Q¹, Q², Q³ and Q⁴ is F. In some embodiments, one of Q¹, Q², Q³ and Q⁴ is NCS. In some embodiments, one of Q¹, Q², Q³ and Q⁴ is maleimide. In some embodiments, Q¹ is NHCOOR. In some embodiments, one of Q¹, Q², Q³ and Q⁴ is N(R)₂. In some embodiments, one of Q¹, Q², Q³ and Q⁴ is CONHR. In some embodiments, one of Q¹, Q², Q³ and Q⁴ is NHCOR. In some embodiments, one of Q¹, Q², Q³ and Q⁴ is Cl. In some embodiments, one of Q¹, Q², Q³ and Q⁴ is Br. In some embodiments, one of Q¹, Q², Q³ and Q⁴ is I. In some embodiments, one of Q¹, Q², Q³ and Q⁴ is NO₂. In some embodiments, one of Q¹, Q², Q³ and Q⁴ is phenyl. In some embodiments, one of Q¹, Q², Q³ and Q⁴ is 4-fluorophenyl In some embodiments, one of Q¹, Q², Q³ and Q⁴ is CF₃. In some embodiments, one of Q¹, Q², Q³ and Q⁴ is substituted or unsubstituted alkyl. In some embodiments, one of Q¹, Q², Q³ and Q⁴ is substituted or unsubstituted cycloalkyl. In some embodiments, one of Q¹, Q², Q³ and Q⁴ is substituted or unsubstituted heterocycloalkyl. In some embodiments, one of Q¹, Q², Q³ and Q⁴ is haloalkyl. In some embodiments, one of Q¹, Q², Q³ and Q⁴ is substituted or unsubstituted aryl. In some embodiments, Q¹ is hydroxyl. one of Q¹, Q², Q³ and Q⁴ is alkoxy. In some embodiments, one of Q¹, Q², Q³ and Q⁴ is OR. In some embodiments, one of Q¹, Q², Q³ and Q⁴ is arylalkyl. In some embodiments, one of Q¹, Q², Q³ and Q⁴ is amine. In some embodiments, one of Q¹, Q², Q³ and Q⁴ is amide. In some embodiments, one of Q¹, Q², Q³ and Q⁴ is COOR. In some embodiments, one of Q¹, Q², Q³ and Q⁴ is COR. In some embodiments, one of Q¹, Q², Q³ and Q⁴ is keto.

In some embodiments, Q³ is CN. In some embodiments, Q³ is F. In some embodiments, Q³ is NCS. In some embodiments, Q³ is maleimide. In some embodiments, Q³ is NHCOOR. In some embodiments, Q³ is N(R)₂. In some embodiments, Q³ is CONHR. In some embodiments, Q³ is NHCOR. In some embodiments, Q³ is hydrogen. In some embodiments, Q³ is keto. In some embodiments, Q³ is CL In some embodiments, Q³ is Br. In some embodiments, Q³ is I. In some embodiments, Q³ is NO₂. In some embodiments, Q³ is phenyl. In some embodiments, Q³ is 4-fluorophenyl. In some embodiments, Q³ is CF₃. In some embodiments, Q³ is substituted or unsubstituted alkyl. In some embodiments, Q³ is substituted or unsubstituted cycloalkyl. In some embodiments, Q³ is substituted or unsubstituted heterocycloalkyl. In some embodiments, Q³ is haloalkyl. In some embodiments, Q³ is substituted or unsubstituted aryl. In some embodiments, Q³ is hydroxyl. In some embodiments, Q³ is alkoxy. In some embodiments, Q³ is OR. In some embodiments, Q³ is arylalkyl. In some embodiments, Q³ is amine. In some embodiments, Q³ is amide. In some embodiments, Q³ is COOR. In some embodiments, Q³ is COR.

In some embodiments of the compounds of the invention, Q¹ is H, CN, CF₃, phenyl, 4-fluorophenyl, F, Br, Cl, I, COMe, NHCOOMe, NHCOMe or NHCOOC(CH₃)₃.

In some embodiments of the compounds of the invention, Q² is H, CN, CF₃, phenyl, 4-fluorophenyl, F, Br, Cl, I, COMe, NHCOOMe, NHCOMe or NHCOOC(CH₃)₃.

In some embodiments of the compounds of the invention, Q³ is H, CN, CF₃, phenyl, 4-fluorophenyl, F, Br, Cl, I, COMe, NHCOOMe, NHCOMe or NHCOOC(CH₃)₃.

In some embodiments of the compounds of the invention, Q⁴ is H, CN, CF₃, phenyl, 4-fluorophenyl, F, Br, Cl, I, COMe, NHCOOMe, NHCOMe or NHCOOC(CH₃)₃.

In some embodiments of the compounds of the invention, R is H. In some embodiments, R is alkyl. In some embodiments, R is alkenyl. In some embodiments, R is haloalkyl. In some embodiments, R is an alcohol. In some embodiments, R is CH₂CH₂OH. In some embodiments, R is CF₃. In some embodiments, R is CH₂Cl. In some embodiments, R is CH₂CH₂Cl. In some embodiments, R is aryl. In some embodiments, R is F. In some embodiments, R is Cl. In some embodiments, R is Br. In some embodiments, R is I. In some embodiments, R is OH.

In some embodiments of the compounds of the invention, Rₐ is H. In some embodiments, Rₐ is —CH₂—CH═CH—COOR. In some embodiments, Rₐ is —CH₂—C(COOR)═CH₂. In some embodiments, Rₐ is —CH₂—CH═CH—CONHR. In some embodiments, Rₐ is —CH₂—C(CONHR)═CH₂. In some embodiments, Rₐ is —CH₂—CH═CH—CON(R)₂. In some embodiments, Rₐ is —CH₂—C(CON(R)₂)═CH₂.

In some embodiments of the compounds of the invention, W₁ is H. In some embodiments, W₁ is OR_{d}. In some embodiments, R_{d} is H. In some embodiments, R_{d} is —CH₂—CH═CH—COOR. In some embodiments, R_{d} is —CH₂—C(COOR)═CH₂. In some embodiments, R_{d} is —CH₂—CH═CHCONHR. In some embodiments, R_{d} is —CH₂—C(CONHR)═CH₂. In some embodiments, R_{d} is —CH₂—CH═CH—CON(R)₂. In some embodiments, R_{d} is —CH₂—C(CON(R)₂)═CH₂.

In some embodiments of the compounds of the invention, W₂ is CH₃. In some embodiments, W₂ is CH₂F. In some embodiments, W₂ is CHF₂. In some embodiments, W₂ is CF₃. In some embodiments, W₂ is CH₂CH₃. In some embodiments, W₂ is CF₂CF₃. In some embodiments, W₂ is CH₂A.

In some embodiments of the compounds of the invention, W₁ and W₂, together with the carbon atom to which they are attached, form a C═CW₅W₆ group, wherein W₅ and W₆ are each H or alkyl. In some embodiments, W₅ is H. In some embodiments, W₅ is alkyl. In some embodiments, W₆ is H. In some embodiments, W₆ is alkyl. In some embodiments, W₅ and W₆ are both H. In some embodiments, W₅ is H and W₆ is alkyl. In some embodiments, W₅ is alkyl and W₆ is H. In some embodiments, W₅ and W₆ are both alkyl.

In some embodiments of the compounds of the invention, W₃ and W₄ are individually H, OH, or alkyl, wherein the alkyl is optionally substituted with OR, N₀₂, CN, F, Br, Cl, I, COR, NHCOR, or CONHR. In some embodiments, W₃ is H. In some embodiments, W₃ is OH. In some embodiments, W₃ is alkyl. In some embodiments, W₄ is H. In some embodiments, W₄ is alkyl. In some embodiments, W₃ and W₄ are both H. In some embodiments, W₃ is H and W₄ is alkyl. In some embodiments, W₃ is alkyl and W₄ is H. In some embodiments, W₃ is OH and W₄ is alkyl. In some embodiments, W₃ is alkyl and W₄ is OH. In some embodiments, W₃ and W₄ are both alkyl. In some embodiments, when W₃ is alkyl and/or W₄ is alkyl, the alkyl is optionally substituted with OR, N₀₂, CN, F, Br, Cl, I, COR, NHCOR, or CONHR.

In some embodiments of the compounds of the invention, one of W₁ and W₂ with one of W₃ and W₄, together with the carbon atoms to which they are attached, form a C═C bond. For example, W₁ and W₃, or W₁ and W₄, or W₂ and W₃, or W₂ and W₄, together with the carbon atoms to which they are attached, form a C═C bond.

In one embodiment, the compound of the invention represented by the structure of formula I or formula II contains at least one nucleophile acceptor group. In one embodiment, the compound of the invention represented by the structure of formula I or formula II contains at least one functional group with an α, β-unsaturated carbonyl. In one embodiment, such α, β-unsaturated carbonyl functional groups include but are not limited to α, β-unsaturated ketones, amides, esters, thioesters, acid anhydrides, carboxylic acids, carboxylates, acid halides, imides, and the like. In one embodiment, the α, β-unsaturated functional group serves as a Michael Addition reaction acceptor for nucleophiles within the AR.

In one embodiment, the compound of the invention represented by the structure of formula I or formula II contains at least one nucleophile acceptor group. In one embodiment, the nucleophile acceptor group is at least one of isocyanato (—NCO), isothiocyanato (—NCS), cyanato (—CNO), thiocyanato (—CNS), azido (N₃), sulfonyl fluoride (—SO₂F), halomethyl (—CH₂-halide), 2-haloacetyl (—NHCOCH₂-halide), halosulfonyl (—NHSO₂CH₂-halide), and the like. In one embodiment, the nucleophile acceptor group serves as a nucleophile acceptor for nucleophiles within the AR. In one embodiment, said AR nucleophile is within the NTD. In another embodiment, said AR nucleophile is within the AF-1 domain. In another embodiment, said AR nucleophile is within the LBD. In one embodiment, the nucleophile acceptor group is present in the Rₐ group. In one embodiment, the nucleophile acceptor group is present in the W₁ group. In one embodiment, the nucleophile acceptor group is present in the W₃ or W₄ group. In one embodiment, the nucleophile acceptor group is present in any one of the Q¹, Q², Q³, or Q⁴ groups.

The invention encompasses a selective androgen receptor covalent antagonist (SARCA) compound selected from any one of the following structures:

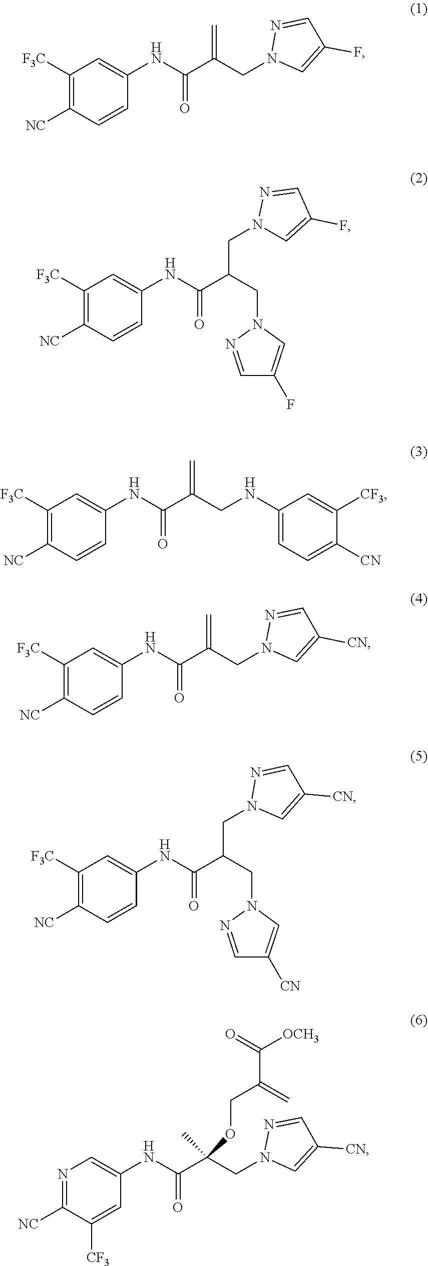 [SMILES: C=C(Cn1cc(F)cn1)C(=O)Nc1ccc(C#N)c(C(F)(F)F)c1 ; N#Cc1ccc(NC(=O)C(Cn2cc(F)cn2)Cn2cc(F)cn2)cc1C(F)(F)F ; C=C(CNc1ccc(C#N)c(C(F)(F)F)c1)C(=O)Nc1ccc(C#N)c(C(F)(F)F)c1 ; C=C(Cn1cc(C#N)cn1)C(=O)Nc1ccc(C#N)c(C(F)(F)F)c1 ; N#Cc1cnn(CC(Cn2cc(C#N)cn2)C(=O)Nc2ccc(C#N)c(C(F)(F)F)c2)c1 ; C=C(CO[C@@](C)(Cn1cc(C#N)cn1)C(=O)Nc1cnc(C#N)c(C(F)(F)F)c1)C(=O)OC]

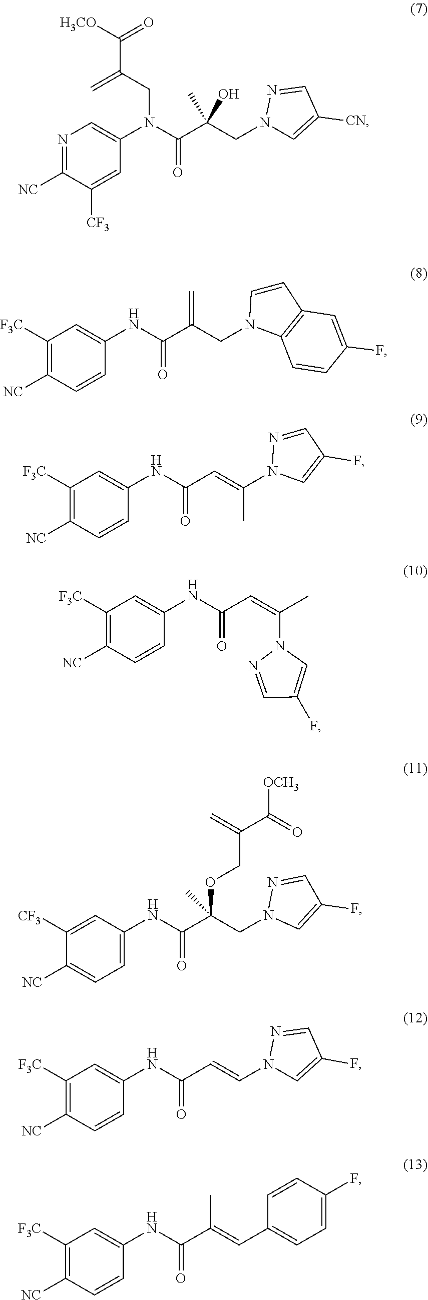

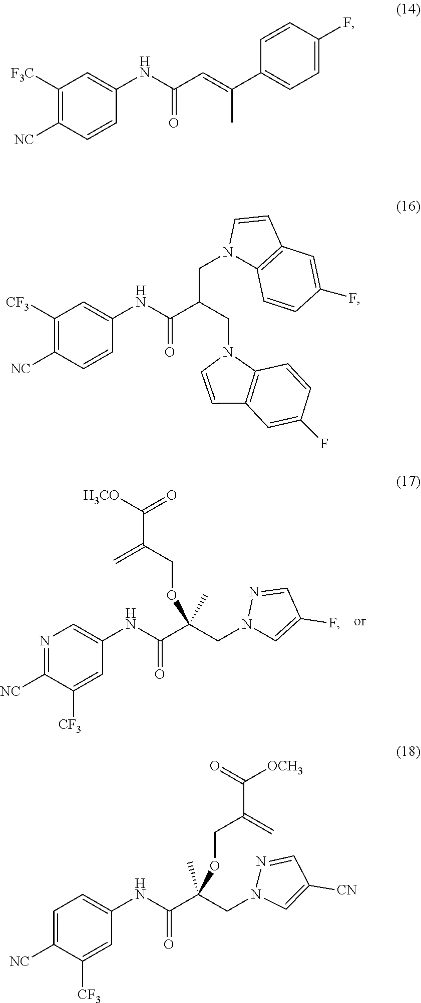 [SMILES: C/C(=C\C(=O)Nc1ccc(C#N)c(C(F)(F)F)c1)c1ccc(F)cc1 ; N#Cc1ccc(NC(=O)C(Cn2ccc3cc(F)ccc32)Cn2ccc3cc(F)ccc32)cc1C(F)(F)F ; C=C(CO[C@@](C)(Cn1cc(F)cn1)C(=O)Nc1cnc(C#N)c(C(F)(F)F)c1)C(=O)OC ; C=C(CO[C@@](C)(Cn1cc(C#N)cn1)C(=O)Nc1ccc(C#N)c(C(F)(F)F)c1)C(=O)OC]

In one embodiment, the compound of the invention is represented by the structure of compound 15

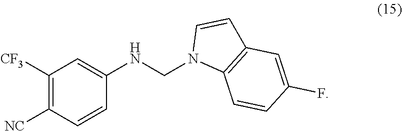 [SMILES: N#Cc1ccc(NCn2ccc3cc(F)ccc32)cc1C(F)(F)F]

As used herein, the term “heterocycle” or “heterocyclic ring” group refers to a ring structure comprising in addition to carbon atoms, at least one atom of sulfur, oxygen, nitrogen or any combination thereof, as part of the ring. The heterocycle may be a 3-12 membered ring; 4-8 membered ring; a 5-7 membered ring; or a 6 membered ring. Preferably, the heterocycle is a 5 to 6 membered ring. Typical examples of heterocycles include, but are not limited to, piperidine, pyridine, furan, thiophene, pyrrole, pyrrolidine, pyrazole, pyrazine, piperazine or pyrimidine. Examples of C₅-C₈ heterocyclic rings include pyran, dihydropyran, tetrahydropyran, dihydropyrrole, tetrahydropyrrole, pyrazine, dihydropyrazine, tetrahydropyrazine, pyrimidine, dihydropyrimidine, tetrahydropyrimidone, pyrazole, dihydropyrazole, tetrahydropyrazole, triazole, tetrazole, piperidine, piperazine, pyridine, dihydropyridine, tetrahydropyridine, morpholine, thiomorpholine, furan, dihydrofuran, tetrahydrofuran, thiophene, dihydrothiophene, tetrahydrothiophene, thiazole, imidazole, isoxazole, and the like. The heterocycle ring may be fused to another saturated or unsaturated cycloalkyl or a saturated or unsaturated heterocyclic ring. When the heterocycle ring is substituted, the substituents include at least one of halogen, haloalkyl, hydroxyl, alkoxy, carbonyl, amido, alkylamido, dialkylamido, cyano, nitro, CO₂H, amino, alkylamino, dialkylamino, carboxyl, thiol, or thioalkyl.

The term “aniline ring system” refers to the conserved ring represented to the left of the structures in this document which is substituted by X, Y, and/or Z.

The term “cycloalkyl” refers to a non-aromatic, monocyclic or polycyclic ring comprising carbon and hydrogen atoms. A cycloalkyl group can have one or more carbon-carbon double bonds in the ring so long as the ring is not rendered aromatic by their presence. Examples of cycloalkyl groups include, but are not limited to, (C₃-C₇) cycloalkyl groups, such as cyclopropyl, cyclobutyl, cyclopentyl, cyclohexyl, and cycloheptyl, and saturated cyclic and bicyclic terpenes and (C₃-C₇) cycloalkenyl groups, such as cyclopropenyl, cyclobutenyl, cyclopentenyl, cyclohexenyl, and cycloheptenyl, and unsaturated cyclic and bicyclic terpenes. Examples of C₅-C₈ carbocyclic include cyclopentane, cyclopentene, cyclohexane, and cyclohexene rings. A cycloalkyl group can be unsubstituted or substituted by at least one substituent. Preferably, the cycloalkyl group is a monocyclic ring or bicyclic ring.

The term “alkyl” refers to a saturated aliphatic hydrocarbon, including straight-chained and branched-chained. Typically, the alkyl group has 1-12 carbons, 1-7 carbons, 1-6 carbons, or 1-4 carbon atoms. A branched alkyl is an alkyl substituted by alkyl side chains of 1 to 5 carbons. The branched alkyl may have an alkyl substituted by a C₁-C₅ haloalkyl. Additionally, the alkyl group may be substituted by at least one of halogen, haloalkyl, hydroxyl, alkoxy carbonyl, amido, alkylamido, dialkylamido, nitro, CN, amino, alkylamino, dialkylamino, carboxyl, thio or thioalkyl.

An “arylalkyl” group refers to an alkyl bound to an aryl, wherein alkyl and aryl are as defined herein. An example of an arylalkyl group is a benzyl group.

An “alkenyl” group refers to an unsaturated hydrocarbon, including straight chain and branched chain having one or more double bonds. The alkenyl group may have 2-12 carbons, preferably the alkenyl group has 2-6 carbons or 2-4 carbons. Examples of alkenyl groups include, but are not limited to, ethenyl, propenyl, butenyl, cyclohexenyl, etc. The alkenyl group may be substituted by at least one halogen, hydroxy, alkoxy carbonyl, amido, alkylamido, dialkylamido, nitro, amino, alkylamino, dialkylamino, carboxyl, thio, or thioalkyl.

As used herein the term “aryl” group refers to an aromatic group having at least one carbocyclic aromatic group or heterocyclic aromatic group, which may be unsubstituted or substituted. When present, substituents include, but are not limited to, at least one halogen, haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like. The aryl group may be a 4-12 membered ring, preferably the aryl group is a 4-8 membered ring. Also, the aryl group may be a 6 or 5 membered ring.

The term “heteroaryl” refers to an aromatic group having at least one heterocyclic aromatic ring. In one embodiment, the heteroaryl comprises at least one heteroatom such as sulfur, oxygen, nitrogen, silicon, phosphorous or any combination thereof, as part of the ring. In another embodiment, the heteroaryl may be unsubstituted or substituted by one or more groups selected from halogen, aryl, heteroaryl, cyano, haloalkyl, hydroxy, alkoxy carbonyl, amido, alkylamido, dialkylamido, nitro, amino, alkylamino, dialkylamino, carboxy or thio or thioalkyl. Nonlimiting examples of heteroaryl rings are pyranyl, pyrrolyl, pyrazinyl, pyrimidinyl, pyrazolyl, pyridinyl, furanyl, thiophenyl, thiazolyl, indolyl, imidazolyl, isoxazolyl, and the like. In one embodiment, the heteroaryl group is a 5-12 membered ring. In one embodiment, the heteroaryl group is a five membered ring. In one embodiment, the heteroaryl group is a six membered ring. In another embodiment, the heteroaryl group is a 5-8 membered ring. In another embodiment, the heteroaryl group comprises of 1-4 fused rings. In one embodiment, the heteroaryl group is 1,2,3-triazole. In one embodiment the heteroaryl is a pyridyl. In one embodiment the heteroaryl is a bipyridyl. In one embodiment the heteroaryl is a terpyridyl.

As used herein, the term “haloalkyl” group refers to an alkyl group that is substituted by one or more halogen atoms, e.g., by F, Cl, Br or I.

A “hydroxyl” group refers to an OH group. It is understood by a person skilled in the art that when T, Q¹, Q², Q³ or Q⁴, in the compounds of the present invention is OR, then R is not OH.

The term “halogen” or “halo” or “halide” refers to a halogen; F, Cl, Br or I.

In one embodiment, this invention provides the compounds and/or its use and/or its derivative, and/or its synthetic intermediates, and/or its synthetic by-products, or their isomer, optical isomer, isomer, metabolite, pharmaceutically acceptable salt, pharmaceutical product, hydrate, N-oxide, prodrug, polymorph, crystal or combinations thereof.

In one embodiment, the methods of this invention make use of “pharmaceutically acceptable salts” of the compounds, which may be produced, by reaction of a compound of this invention with an acid or base.

The compounds of the invention may be converted into pharmaceutically acceptable salts. A pharmaceutically acceptable salt may be produced by reaction of a compound with an acid or base.

Suitable pharmaceutically acceptable salts of amines may be prepared from an inorganic acid or from an organic acid. Examples of inorganic salts of amines include, but are not limited to, bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphates, sulfates, sulfamates, sulfanilates, sulfonic acids (alkylsulfonates, arylsulfonates, halogen substituted alkylsulfonates, halogen substituted arylsulfonates), sulfonates, or thiocyanates.

Examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, carboxylates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates, gluconates, glutamates, glycolates, glucorates, glucoheptanoates, glycerophosphates, gluceptates, glycollylarsanilates, glutarates, glutamates, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, nitrates, naphthalenesulfonates, 2-naphthalenesulfonates, nicotinates, napsylates, N-methylglucamines, oxalates, octanoates, oleates, pamoates, phenylacetates, picrates, phenylbenzoates, pivalates, propionates, phthalates, pectinates, phenylpropionates, palmitates, pantothenates, polygalacturates, pyruvates, quinates, salicylates, succinates, stearates, sulfanilates, subacetates, tartarates, theophyllineacetates, p-toluenesulfonates (tosylates), trifluoroacetates, terephthalates, tannates, teoclates, trihaloacetates, triethiodide, tricarboxylates, undecanoates and valerates. Examples of inorganic salts of carboxylic acids or phenols may be selected from ammonium, alkali metals, and alkaline earth metals. Alkali metals include, but are not limited to, lithium, sodium, potassium, or cesium. Alkaline earth metals include, but are not limited to, calcium, magnesium, aluminium; zinc, barium, cholines, or quaternary ammoniums. Examples of organic salts of carboxylic acids or phenols may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, methylamines, ethanolamines, meglamines, nicotinamides, organic amines, tris(hydroxymethyl)methylamines, tromethamines and ureas, ethylenediamines, hydrabamines, imidazoles, lysines, N-methyl-D-glucamines, N,N′-dibenzylethylenediamines, ornithines, pyridines, picolines, piperazines, procaine, triethylamines, triethanolamines, trimethylamines.

In various embodiments, the pharmaceutically acceptable salts of the compounds of this invention include: HCl salt, oxalic acid salt, L-(+)-tartaric acid salt, HBr salt and succinic acid salt. Each represents a separate embodiment of this invention.

Salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of an existing salt for another ion or suitable ion-exchange resin.

The methods of the invention may use an uncharged compound or a pharmaceutically acceptable salt of the compound. In particular, the methods use pharmaceutically acceptable salts of compounds of the invention as described herein. The pharmaceutically acceptable salt may be an amine salt or a salt of a phenol of the compounds of the invention as described herein.

In one embodiment, the methods of this invention make use of a free base, free acid, non charged or non-complexed compounds of the invention as described herein, and/or its isomer, pharmaceutical product, hydrate, polymorph, or combinations thereof.

In one embodiment, the methods of this invention make use of an optical isomer of a compound of the invention as described herein. In one embodiment, the methods of this invention make use of an isomer of a compound of the invention as described herein. In one embodiment, the methods of this invention make use of a pharmaceutical product of a compound of the invention as described herein. In one embodiment, the methods of this invention make use of a hydrate of a compound of the invention as described herein. In one embodiment, the methods of this invention make use of a polymorph of a compound of the invention as described herein. In one embodiment, the methods of this invention make use of a metabolite of a compound of the invention as described herein. In another embodiment, the methods of this invention make use of a composition comprising a compound of the invention as described herein, or, in another embodiment, a combination of isomer, metabolite, pharmaceutical product, hydrate, polymorph of a compound of the invention as described herein.

As used herein, the term “synthetic by-product” is a compound synthesized together with the SARCA compound that contains a nucleophile acceptor group which itself has no nucleophile acceptor group. It will be appreciated by those skilled in the art that synthetic by-products can themselves possess significant and useful properties including potent inhibition of wtAR or degradation of the AR or AR SV.

As used herein, the term “isomer” includes, but is not limited to, optical isomers, structural isomers, or conformational isomers.

The term “isomer” is meant to encompass optical isomers of the SARCA compound. It will be appreciated by those skilled in the art that the SARCA s of the present invention contain at least one chiral center. Accordingly, the compounds may exist as optically-active (such as an (R) isomer or (S) isomer) or racemic forms. Optically active compounds may exist as enantiomerically enriched mixtures. Some compounds may also exhibit polymorphism. It is to be understood that the present invention encompasses any racemic, optically active, polymorphic, or stereroisomeric form, or mixtures thereof. Thus, the invention may encompass SARCA compounds as pure (R)isomers or as pure (S)-isomers. It is known in the art how to prepare optically active forms. For example, by resolution of the racemic form by recrystallization techniques, by synthesis from optically active starting materials, by chiral synthesis, or by chromatographic separation using a chiral stationary phase.

Compounds of the invention may be hydrates of the compounds. As used herein, the term “hydrate” includes, but is not limited to, hemihydrate, monohydrate, dihydrate, or trihydrate. The invention also includes use of N-oxides of the amino substituents of the compounds described herein.

This invention provides, in other embodiments, use of metabolites of the compounds as herein described. In one embodiment, “metabolite” means any substance produced from another substance by metabolism or a metabolic process.

In one embodiment, the compounds of this invention are prepared as described herein, for example, according to Example 1.

Biological Activity of Selective Androgen Receptor Covalent Antagonists

The compounds of the invention are selective androgen receptor covalent antagonists (SARCAs) that bind covalently and irreversibly to AR AF-1 or LBD and inhibit the function of AR and AR-SVs and/or degrade AR and AR-SVs.

The SAR CA compounds of the invention can be used for treating prostate cancer (PCa) or increasing the survival of a male subject suffering from prostate cancer, the method comprising administering to the subject a therapeutically effective amount of a compound or its pharmaceutically acceptable salt, represented by the structure of formula I:

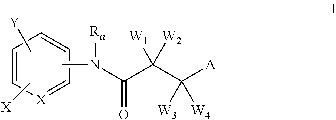

wherein
X is CH or N;
Y is H, CF₃, F, Br, Cl, I, CN, or C(R)₃;
Z is H, NO₂, CN, F, Br, Cl, I, COOH, COR, NHCOR, or CONHR;
or Y and Z form a 5 to 8 membered fused ring;
R is H, alkyl, alkenyl, CH₂CH₂OH, CF₃, CH₂Cl, CH₂CH₂Cl, aryl, F, Cl, Br, I, or OH;
Rₐ is H, alkyl-NCO, alkyl-NCS, alkyl-SCN, alkyl-OCN, alkyl-N₃, alkyl-SO₂F, alkyl-CH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂, wherein halide is F, Cl, Br, or I;
W₁ is H or OR_{d}, wherein R_{d} is H, alkyl-NCO, alkyl-NCS, alkyl-SCN, alkyl-OCN, alkyl-N₃, alkyl-SO₂F, alkyl-CH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
W₂ is CH₃, CH₂F, CHF₂, CF₃, CH₂CH₃, CF₂CF₃, or CH₂A;
or W₁ and W₂, together with the carbon atom to which they are attached, form a C═CW₅W₆ group, wherein W₅ and W₆ are each H or alkyl;
W₃ and W₄ are individually H, OH, alkyl, wherein the alkyl is optionally substituted with OR, NO₂, CN, F, Br, Cl, I, COR, NHCOR, CONHR, —NCO, —NCS, —SCN, —OCN, —N₃, —SO₂F, —CH₂halide, —NHCOCH₂halide, —NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
or one of W₁ and W₂ with one of W₃ and W₄, together with the carbon atoms to which they are attached, form a C═C bond;
A is NR_{b}R_{c} or a 5 to 10-membered aryl or heteroaryl group, optionally substituted with at least one of Q¹, Q₂, Q₃ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
R_{b} is H or alkyl, wherein the alkyl is optionally substituted with OR, NO₂, CN, F, Br, Cl, I, COR, NHCOR, or CONHR;
R_{c} is alkyl, haloalkyl, cycloalkyl, heterocycloalkyl, aryl or heteroaryl, wherein said alkyl, haloalkyl, cycloalkyl, heterocycloalkyl, aryl and heteroaryl groups are optionally substituted with CN, NO₂, CF₃, F, Cl, Br, I NHCOOR, N(R)₂, NHCOR, COR, alkyl, or alkoxy;
or R_{b} and R_{c}, together with the nitrogen atom to which they are attached, form a 5 to 10-membered saturated or unsaturated heterocyclic ring having at least one nitrogen atom and 0, 1, or 2 double bonds, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CO NHCOR, —CH₂—C(CONHCOR)═CH₂, —CH₂—CH═CH—CON(R)₂, or —CH₂—C(CON(R)₂)═CH₂;
or its isomer, optical isomer, racemic mixture, pharmaceutically acceptable salt, pharmaceutical product, synthetic by-product, hydrate or any combination thereof.

In one embodiment, the compound of the invention is represented by the structure of formula II:

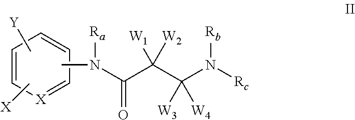

wherein
X is CH or N;
Y is H, CF₃, F, Br, Cl, I, CN, or C(R)₃;
Z is H, NO₂, CN, F, Br, Cl, I, COOH, COR, NHCOR, or CONHR;
or Y and Z form a 5 to 8 membered fused ring;
R is H, alkyl, alkenyl, CH₂CH₂OH, CF₃, CH₂Cl, CH₂CH₂Cl, aryl, F, Cl, Br, I, or OH;
Rₐ is H, alkyl-NCO, alkyl-NCS, alkyl-SCN, alkyl-OCN, alkyl-N₃, alkyl-SO₂F, alkyl-CH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CO NHCOR, —CH₂—C(CONHCOR)═CH₂, —CH₂—CH═CH—CON(R)₂, or —CH₂—C(CON(R)₂)═CH₂, wherein halide is F, Cl, Br, or I;
W₁ is H or OR_{d}, wherein R_{d} is H, alkyl-NCO, alkyl-NCS, alkyl-SCN, alkyl-OCN, alkyl-N₃, alkyl-SO₂F, alkyl-CH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CH—CON(R)₂, or —CH₂—C(CON(R)₂)═CH₂;
W₂ is CH₃, CH₂F, CHF₂, CF₃, CH₂CH₃, CF₂CF₃, or CH₂A;
or W₁ and W₂, together with the carbon atom to which they are attached, form a C═CW₅W₆ group, wherein W₅ and W₆ are each H or alkyl;
W₃ and W₄ are individually H, OH, alkyl, wherein the alkyl is optionally substituted with OR, NO₂, CN, F, Br, Cl, I, COR, NHCOR, CONHR, —NCO, —NCS, —SCN, —OCN, —N₃, —SO₂F, —CH₂halide, —NHCOCH₂halide, —NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
or one of W₁ and W₂ with one of W₃ and W₄, together with the carbon atoms to which they are attached, form a C═C bond;
A is NR_{b}R_{c} or a 5 to 10-membered aryl or heteroaryl group, optionally substituted with at least one of Q¹, Q², Q₃ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
R_{b} is H or alkyl, wherein the alkyl is optionally substituted with OR, NO₂, CN, F, Br, Cl, I, COR, NHCOR, or CONHR;
R_{c} is alkyl, haloalkyl, cycloalkyl, heterocycloalkyl, aryl or heteroaryl, wherein said alkyl, haloalkyl, cycloalkyl, heterocycloalkyl, aryl and heteroaryl groups are optionally substituted with CN, NO₂, CF₃, F, Cl, Br, I NHCOOR, N(R)₂, NHCOR, COR, alkyl, or alkoxy;
or R_{b} and R_{c}, together with the nitrogen atom to which they are attached, form a 5 to 10-membered saturated or unsaturated heterocyclic ring having at least one nitrogen atom and 0, 1, or 2 double bonds, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
or its isomer, optical isomer, racemic mixture, pharmaceutically acceptable salt, pharmaceutical product, synthetic by-product, hydrate or any combination thereof.

In one embodiment, the compound of the invention represented by the structure of formula I or formula II contains at least one nucleophile acceptor group. In one embodiment, the compound of the invention represented by the structure of formula I or formula II contains at least one functional group with an α, β-unsaturated carbonyl. In one embodiment, such α, β-unsaturated carbonyl functional groups include but are not limited to α, β-unsaturated ketones, amides, esters, thioesters, acid anhydrides, carboxylic acids, carboxylates, acid halides, imides, and the like. In one embodiment, the α, β-unsaturated functional group serves as a Michael Addition reaction acceptor for nucleophiles within the AR.

In one embodiment, the compound of the invention represented by the structure of formula I or formula II contains at least one nucleophile acceptor group. In one embodiment, the nucleophile acceptor group is at least one of isocyanato (—NCO), isothiocyanato (—NCS), cyanato (CNO), thiocyanato (—CNS), azido (N₃), sulfonyl fluoride (—SO₂F), halomethyl (—CH₂-halide), 2-haloacetyl (—NHCOCH₂-halide), halosulfonyl (—NHSO₂CH₂-halide), and the like. In one embodiment, the nucleophile acceptor group serves as a nucleophile acceptor for nucleophiles within the AR. In one embodiment, said AR nucleophile is within the NTD. In another embodiment, said AR nucleophile is within the AF-1 domain. In another embodiment, said AR nucleophile is within the LBD. In one embodiment, the nucleophile acceptor group is present in the Rₐ group. In one embodiment, the nucleophile acceptor group is present in the W₁ group. In one embodiment, the nucleophile acceptor group is present in the W₃ or W₄ group. In one embodiment, the nucleophile acceptor group is present in any one of the Q¹, Q², Q³, or Q⁴ groups.

The present invention provides a method of treating prostate cancer (PCa) or increasing the survival of a male subject suffering from prostate cancer comprising administering to the subject a therapeutically effective amount of a compound or its pharmaceutically acceptable salt, or isomer, represented by a compound of the invention as described herein.

The prostate cancer may be advanced prostate cancer, refractory prostate cancer, castration resistant prostate cancer (CRPC), metastatic CRPC (mCRPC), non-metastatic CRPC (nmCRPC), high-risk nmCRPC or any combination thereof.

The prostate cancer may depend on AR-FL and/or AR-SV for proliferation. The prostate or other cancer may be resistant to treatment with an androgen receptor antagonist. The prostate or other cancer may be resistant to treatment with enzalutamide, bicalutamide, abiraterone, ARN-509, ODM-201, EPI-001, EPI-506, AZD-3514, galeterone, ASC-J9, flutamide, hydroxyflutamide, nilutamide, cyproterone acetate, ketoconazole, spironolactone, or any combination thereof. The method may also reduce the levels of AR, AR-FL, AR-FL with antiandrogen resistance-conferring AR-LBD mutations, AR-SV, gene-amplified AR, or any combination thereof.

In one embodiment, this invention provides a method of treating enzalutamide resistant prostate cancer comprising administering to the subject a therapeutically effective amount of a compound of this invention, or its isomer, optical isomer, isomer, pharmaceutically acceptable salt, pharmaceutical product, polymorph, hydrate or any combination thereof.

In one embodiment, this invention provides a method of treating abiraterone resistant prostate cancer comprising administering to the subject a therapeutically effective amount of a compound of this invention, or its isomer, optical isomer, isomer, pharmaceutically acceptable salt, pharmaceutical product, polymorph, hydrate or any combination thereof.

In one embodiment, this invention provides a method of treating triple negative breast cancer (TNBC) comprising administering to the subject a therapeutically effective amount of a compound of this invention, or its isomer, optical isomer, isomer, pharmaceutically acceptable salt, pharmaceutical product, polymorph, hydrate or any combination thereof.

The method may further comprise a second therapy such as androgen deprivation therapy (ADT) or LHRH agonist or antagonist. LHRH agonists include, but are not limited to, leuprolide acetate.

The invention encompasses a method of treating or inhibiting the progression of prostate cancer (PCa) or increasing the survival of a male subject suffering from prostate cancer comprising administering to the subject a therapeutically effective amount of a SARCA compound or pharmaceutically acceptable salt, wherein the compound is at least one of compounds 1-18.

The invention encompasses a method of treating or inhibiting the progression of refractory prostate cancer (PCa) or increasing the survival of a male subject suffering from refractory prostate cancer comprising administering to the subject a therapeutically effective amount of a SARCA compound or pharmaceutically acceptable salt, wherein the compound is represented by a compound of formulas I-XX, or the compound is at least one of compounds 1-18.

The invention encompasses a method of treating or increasing the survival of a male subject suffering from castration resistant prostate cancer (CRPC) comprising administering to the subject a therapeutically effective amount of a SARCA wherein the compound is represented by a compound of formulas I-XX, or at least one of compounds 1-18.

The method may further comprise administering androgen deprivation therapy to the subject.

The invention encompasses a method of treating or inhibiting the progression of enzalutamide resistant prostate cancer (PCa) or increasing the survival of a male subject suffering from enzalutamide resistant prostate cancer comprising administering to the subject a therapeutically effective amount of a SARCA compound or pharmaceutically acceptable salt, wherein the compound is represented by a compound of formulas I-XX, or the compound is at least one of compounds 1-18.

The method may further comprise administering androgen deprivation therapy to the subject.

The invention encompasses a method of treating or inhibiting the progression of triple negative breast cancer (TNBC) or increasing the survival of a female subject suffering from triple negative breast cancer comprising administering to the subject a therapeutically effective amount of a SARCA compound or pharmaceutically acceptable salt, wherein the compound is represented by a compound of formulas I-XX, or the compound is at least one of compounds 1-18.

The invention encompasses a method of treating breast cancer in a subject in need thereof, wherein said subject has AR expressing breast cancer, AR-SV expressing breast cancer, and/or ARV7 expressing breast cancer, comprising administering to the subject a therapeutically effective amount of a selective androgen receptor covalent antagonist (SARCA) compound, or its isomer, pharmaceutically acceptable salt, pharmaceutical product, polymorph, hydrate or any combination thereof, wherein said SARCA compound is represented by the structure of formula formulas I-XX, or the compound is at least one of compounds 1-18.

The invention encompasses a method of treating AR expressing breast cancer in a subject in need thereof, comprising administering to the subject a therapeutically effective amount of a selective androgen receptor covalent antagonist (SARCA) compound, or its isomer, pharmaceutically acceptable salt, pharmaceutical product, polymorph, hydrate or any combination thereof, wherein said SARCA compound is represented by the structure of formula formulas I-XX, or the compound is at least one of compounds 1-18.

The invention encompasses a method of treating AR-SV expressing breast cancer in a subject in need thereof, comprising administering to the subject a therapeutically effective amount of a selective androgen receptor covalent antagonist (SARCA) compound, or its isomer, pharmaceutically acceptable salt, pharmaceutical product, polymorph, hydrate or any combination thereof, wherein said SARCA compound is represented by the structure of formula formulas I-XX, or the compound is at least one of compounds 1-18.

The invention encompasses a method of treating AR-V7 expressing breast cancer in a subject in need thereof, comprising administering to the subject a therapeutically effective amount of a selective androgen receptor covalent antagonist (SARCA) compound, or its isomer, pharmaceutically acceptable salt, pharmaceutical product, polymorph, hydrate or any combination thereof, wherein said SARCA compound is represented by the structure of formula formulas I-XX, or the compound is at least one of compounds 1-18.

As used herein, the term “increase the survival” refers to a lengthening of time when describing the survival of a subject. Thus in this context, the compounds of the invention may be used to increase the survival of men with advanced prostate cancer, refractory prostate cancer, castration resistant prostate cancer (CRPC); metastatic CRPC (mCRPC); non-metastatic CRPC (nmCRPC); or high-risk nmCRPC; or women with TNBC.

Alternatively, as used herein, the terms “increase”, increasing”, or “increased” may be used interchangeably and refer to an entity becoming progressively greater (as in size, amount, number, or intensity), wherein for example the entity is sex hormone-binding globulin (SHBG) or prostate-specific antigen (PSA).

The compounds and compositions of the invention may be used for increasing metastasis-free survival (MPS) in a subject suffering from non-metastatic prostate cancer. The non-metastatic prostate cancer may be non-metastatic advanced prostate cancer, non-metastatic CRPC (nmCRPC), or high-risk nmCRPC.

The SARCA compounds described herein may be used to provide a dual action. For example, the SARCA compounds may treat prostate cancer and prevent metastasis. The prostate cancer may be refractory prostate cancer; advanced prostate cancer; castration resistant prostate cancer (CRPC); metastatic CRPC (mCRPC); non-metastatic CRPC (nmCRPC); or high-risk nmCRPC.

The SARCA compounds described herein may be used to provide a dual action. For example, the SARCA compounds may treat TNBC and prevent metastasis.

Men with advanced prostate cancer who are at high risk for progression to castration resistant prostate cancer (CRPC) are men on ADT with serum total testosterone concentrations greater than 20 ng/dL or men with advanced prostate cancer who at the time of starting ADT had either (1) confirmed Gleason pattern 4 or 5 prostate cancer, (2) metastatic prostate cancer, (3) a PSA doubling time <3 months, (4) a PSA ≥20 ng/mL, or (5) a PSA relapse in <3 years after definitive local therapy (radical prostatectomy or radiation therapy).

Normal levels of prostate specific antigen (PSA) are dependent on several factors, such as age and the size of a male subject's prostate, among others. PSA levels in the range between 2.5-10 ng/mL are considered “borderline high” while levels above 10 ng/mL are considered “high.” A rate change or “PSA velocity” greater than 0.75/year is considered high. PSA levels may increase despite ongoing ADT or a history of ADT, surgical castration or despite treatment with antiandrogens and/or LHRH agonist.

Men with high risk non-metastatic castration resistant prostate cancer (high-risk nmCRPC) may include those with rapid PSA doubling times, having an expected progression-free survival of approximately 18 months or less (Miller K, Moul J W, Gleave M, et al. 2013. “Phase III, randomized, placebo-controlled study of once-daily oral zibotentan (ZD4054) in patients with non-metastatic castration-resistant prostate cancer,” Prostate Cane Prost Dis. February; 16:187-192). This relatively rapid progression of their disease underscores the importance of novel therapies for these individuals.

The methods of the invention may treat subjects with PSA levels greater than 8 ng/mL where the subject suffers from high-risk nmCRPC. The patient population includes subjects suffering from nmCRPC where PSA doubles in less than 8 months or less than 10 months. The method may also treat patient populations where the total serum testosterone levels are greater than 20 ng/mL in a subject suffering from high-risk nmCRPC. In one case, the serum free testosterone levels are greater than those observed in an orchiectomized male in a subject suffering from high-risk nmCRPC.

The pharmaceutical compositions of the invention may further comprise at least one LHRH agonist or antagonist, antiandrogen, anti-programmed death receptor 1 (anti-PD-1) drug or anti-PD-1 drug. LHRH agonists include, but are not limited to, leuprolide acetate (Lupron®) (U.S. Pat. Nos. 5,480,656; 5,575,987; 5,631,020; 5,643,607; 5,716,640; 5,814,342; 6,036,976 hereby incorporated by reference) or goserelin acetate (Zoladex©) (U.S. Pat. Nos. 7,118,552; 7,220,247; 7,500,964 hereby incorporated by reference). LHRH antagonists include, but are not limited to, degarelix or abarelix. Antiandrogens include, but are not limited to, bicalutamide, flutamide, apalutamide, finasteride, dutasteride, enzalutamide, nilutamide, chlormadinone, abiraterone, or any combination thereof. Anti-PD-1 drugs include, but are not limited to, AMP-224, nivolumab, pembrolizumab, pidilizumab, and AMP-554. Anti-PD-1 drugs include, but are not limited to, BMS-936559, atezolizumab, durvalumab, avelumab, and MPDL3280A. Anti-CTLA-4 drugs include, but are not limited to, ipilimumab and tremelimumab.

Treatment of prostate cancer, advanced prostate cancer, CRPC, mCRPC and/or nmCRPC may result in clinically meaningful improvement in prostate cancer related symptoms, function and/or survival. Clinically meaningful improvement can be determined by an increase in radiographic progression free survival (rPFS) if cancer is metastatic, or an increase metastasis-free survival (MFS) if cancer is non-metastatic, among others.

The invention encompasses methods of lowering serum prostate specific antigen (PSA) levels in a male subject suffering from prostate cancer, advanced prostate cancer, metastatic prostate cancer or castration resistant prostate cancer (CRPC) comprising administering a therapeutically effective amount of a SARCA compound, wherein the compound is represented by the structure of formulas I-XX or the compound is at least one of compounds 1-18.

The invention encompasses a method of secondary hormonal therapy that reduces serum PSA in a male subject suffering from castration resistant prostate cancer (CRPC) comprising administering a therapeutically effective amount of a compound of formulas I-XX or the compound is at least one of compounds 1-18 that reduces serum PSA in a male subject suffering from castration resistant prostate cancer.

The invention encompasses a method of reducing levels of AR, AR-full length (AR-FL), AR-FL with antiandrogen resistance-conferring AR-LBD mutations, AR-splice variant (AR-SV), and/or amplifications of the AR gene within the tumor in the subject in need thereof comprising administering a therapeutically effective amount of a compound of formulas I-XX or the compound is at least one of compounds 1-18 to reduce the level of AR, AR-full length (AR-FL), AR-FL with antiandrogen resistance-conferring AR-LBD or other AR mutations, AR-splice variant (AR-SV), and/or amplifications of the AR gene within the tumor.

The method may increase radiographic progression free survival (rPFS) or metastasis-free survival (MPS).

Subjects may have non-metastatic cancer; failed androgen deprivation therapy (ADT), undergone orchidectomy, or have high or increasing prostate specific antigen (PSA) levels; subjects may be a patient with prostate cancer, advanced prostate cancer, refractory prostate cancer, CRPC patient, metastatic castration resistant prostate cancer (mCRPC) patient, or non-metastatic castration resistant prostate cancer (nmCRPC) patient. In these subjects, the refractory may be enzalutamide resistant prostate cancer. In these subjects, the nmCRPC may be high-risk nmCRPC. Further the subject may be on androgen deprivation therapy (ADT) with or without castrate levels of total T.

As used herein, the phrase “a subject suffering from castration resistant prostate cancer” refers to a subject with at least one of the following characteristics: has been previously treated with androgen deprivation therapy (ADT); has responded to the ADT and currently has a serum PSA >2 ng/mL or >2 ng/mL and representing a 25% increase above the nadir achieved on the ADT; a subject which despite being maintained on androgen deprivation therapy is diagnosed to have serum PSA progression; a castrate level of serum total testosterone (<50 ng/dL) or a castrate level of serum total testosterone (<20 ng/dL). The subject may have rising serum PSA on two successive assessments at least 2 weeks apart; been effectively treated with ADT; or has a history of serum PSA response after initiation of ADT.

As used herein, the term “serum PSA progression” refers to a 25% or greater increase in serum PSA and an absolute increase of 2 ng/ml or more from the nadir; or to serum PSA >2 ng/mL, or >2 ng/mL and a 25% increase above the nadir after the initiation of androgen deprivation therapy (ADT). The term “nadir” refers to the lowest PSA level while a patient is undergoing ADT.

The term “serum PSA response” refers to at least one of the following: at least 90% reduction in serum PSA value prior to the initiation of ADT; to <10 ng/mL undetectable level of serum PSA (<0.2 ng/mL) at any time; at least 50% decline from baseline in serum PSA; at least 90% decline from baseline in serum PSA; at least 30% decline from baseline in serum PSA; or at least 10% decline from baseline in serum PSA.

The methods of this invention comprise administering a combination of forms of ADT and a compound of this invention. Forms of ADT include a LHRH agonist. LHRH agonist includes, but is not limited to, leuprolide acetate (Lupron®)(U.S. Pat. Nos. 5,480,656; 5,575,987; 5,631,020; 5,643,607; 5,716,640; 5,814,342; 6,036,976 hereby incorporated by reference) or goserelin acetate (Zoladex®) (U.S. Pat. Nos. 7,118,552; 7,220,247; 7,500,964 hereby incorporated by reference). Forms of ADT include, but are not limited to LHRH antagonists, reversible antiandrogens, or bilateral orchidectomy. LHRH antagonists include, but are not limited to, degarelix and abarelix. Antiandrogens include, but are not limited to, bicalutamide, flutamide, apalutamide, finasteride, dutasteride, enzalutamide, EPI-001, EPI-506, ARN-509, ODM-201, nilutamide, chlormadinone, abiraterone, or any combination thereof.

The methods of the invention encompass administering at least one compound of the invention and a lyase inhibitor (e.g., abiraterone).

The term “advanced prostate cancer” refers to metastatic cancer having originated in the prostate, and having widely metastasized to beyond the prostate such as the surrounding tissues to include the seminal vesicles the pelvic lymph nodes or bone, or to other parts of the body. Prostate cancer pathologies are graded with a Gleason grading from 1 to 5 in order of increasing malignancy. Patients with significant risk of progressive disease and/or death from prostate cancer should be included in the definition and any patient with cancer outside the prostate capsule with disease stages as low as IIB clearly has “advanced” disease. “Advanced prostate cancer” can refer to locally advanced prostate cancer. Similarly, “advanced breast cancer” refers to metastatic cancer having originated in the breast and having widely metastasized to beyond the breast to surrounding tissues or other parts of the body such as the liver, brain, lungs, or bone.

The term “refractory” may refer to cancers that do not respond to treatment. E.g., prostate or breast cancer may be resistant at the beginning of treatment or it may become resistant during treatment. “Refractory cancer” may also be referred to herein as “resistant cancer”.

The term “castration resistant prostate cancer” (CRPC) refers to advanced prostate cancer that is worsening or progressing while the patient remains on ADT or other therapies to reduce testosterone, or prostate cancer which is considered hormone refractory, hormone naive, androgen independent or chemical or surgical castration resistant. CRPC may be the result of AR activation by intracrine androgen synthesis; expression of AR splice variants (AR-SV) that lack ligand binding domain (LBD); or expression of AR-LBD or other AR mutations with potential to resist antagonists. Castration resistant prostate cancer (CRPC) is an advanced prostate cancer which developed despite ongoing ADT and/or surgical castration. Castration resistant prostate cancer is defined as prostate cancer that continues to progress or worsen or adversely affect the health of the patient despite prior surgical castration, continued treatment with gonadotropin releasing hormone agonists (e.g., leuprolide) or antagonists (e.g., degarelix or abarelix), antiandrogens (e.g., bicalutamide, flutamide, apalutamide, enzalutamide, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®), cabozantinib (Cometriq™, also known as XL184)) or other prostate cancer therapies (e.g., vaccines (sipuleucel-T (Provenge®), GV AX, etc.), herbal (PC-SPES) and lyase inhibitor (abiraterone)) as evidenced by increasing or higher serum levels of prostate specific antigen (PSA), metastasis, bone metastasis, pain, lymph node involvement, increasing size or serum markers for tumor growth, worsening diagnostic markers of prognosis, or patient condition.

Castration resistant prostate cancer may be defined as hormone naive prostate cancer. In men with castration resistant prostate cancer, the tumor cells may have the ability to grow in the absence of androgens (hormones that promote the development and maintenance of male sex characteristics).

Many early prostate cancers require androgens for growth, but advanced prostate cancers are androgen-independent, or hormone naive.

The term “androgen deprivation therapy” (ADT) may include orchiectomy; administering luteinizing hormone-releasing hormone (LHRH) analogs; administering luteinizing hormone-releasing hormone (LHRH) antagonists; administering 5α-reductase inhibitors; administering antiandrogens; administering inhibitors of testosterone biosynthesis; administering estrogens; or administering 17α-hydroxylase/C17,20 lyase (CYP17A1) inhibitors. LHRH drugs lower the amount of testosterone made by the testicles. Examples of LHRH analogs available in the United States include leuprolide (Lupron®, Viadur®, Eligard®), goserelin (Zoladex®), triptorelin (Trelstar®), and histrelin (Vantas®). Antiandrogens block the body's ability to use any androgens. Examples of antiandrogens drugs include enzalutamide (Xtandi®), flutamide (Eulexin®), apalutamide (Erleada®), bicalutamide (Casodex®), and nilutamide (Nilandron®). Luteinizing hormone-releasing hormone (LHRH) antagonists include abarelix (Plenaxis®) or degarelix (Firmagon®) (approved for use by the FDA in 2008 to treat advanced prostate cancer). 5α-Reductase inhibitors block the body's ability to convert testosterone to the more active androgen, 5α-dihydrotestosterone (DHT) and include drugs such as finasteride (Proscar®) and dutasteride (Avodart®). Inhibitors of testosterone biosynthesis include drugs such as ketoconazole (Nizoral®). Estrogens include diethylstilbestrol or 17α-estradiol. 17α-Hydroxylase/C17,20 lyase (CYP17A1) inhibitors include abiraterone (Zytiga*).

The invention encompasses a method of treating antiandrogen-resistant prostate cancer. The antiandrogen may include, but is not limited to, bicalutamide, hydroxyflutamide, flutamide, apalutamide, enzalutamide, darolutamide, or abiraterone.

The invention encompasses a method of treating prostate cancer in a subject in need thereof, wherein said subject has a rearranged AR, AR overexpressing prostate cancer, castration-resistant prostate cancer, castration-sensitive prostate cancer, AR-V7 expressing prostate cancer, or d567ES expressing prostate cancer, comprising administering to the subject a therapeutically effective amount of a selective androgen receptor covalent antagonist (SARCA) compound, or its isomer, pharmaceutically acceptable salt, pharmaceutical product, polymorph, hydrate or any combination thereof, wherein said SARCA compound is represented by the structure of formula formulas I-XX, or the compound is at least one of compounds 1-18.

In one embodiment, the castration-resistant prostate cancer is a rearranged AR, AR overexpressing castration-resistant prostate cancer, F876L mutation expressing castration-resistant prostate cancer, F876L_T877A double mutation expressing castration-resistant prostate cancer, AR-V7 expressing castration-resistant prostate cancer, d567ES expressing castration-resistant prostate cancer, and/or castration-resistant prostate cancer characterized by intratumoral androgen synthesis.

In one embodiment, the castration-sensitive prostate cancer is F876L mutation expressing castration-sensitive prostate cancer, F876L_T877A double mutation castration-sensitive prostate cancer, and/or castration-sensitive prostate cancer characterized by intratumoral androgen synthesis.

In one embodiment, the treating of castration-sensitive prostate cancer is conducted in a non-castrate setting, or as monotherapy, or when castration-sensitive prostate cancer tumor is resistant to enzalutamide, apalutamide, and/or abiraterone.

The invention encompasses a method of treating AR overexpressing prostate cancer in a subject in need thereof, comprising administering to the subject a therapeutically effective amount of a selective androgen receptor covalent antagonist (SARCA) compound, or its isomer, pharmaceutically acceptable salt, pharmaceutical product, polymorph, hydrate or any combination thereof, wherein said SARCA compound is represented by the structure of formula formulas I-XX, or the compound is at least one of compounds 1-18.

The invention encompasses a method of treating castration-resistant prostate cancer in a subject in need thereof, comprising administering to the subject a therapeutically effective amount of a selective androgen receptor covalent antagonist (SARCA) compound, or its isomer, pharmaceutically acceptable salt, pharmaceutical product, polymorph, hydrate or any combination thereof, wherein said SARCA compound is represented by the structure of formula formulas I-XX, or the compound is at least one of compounds 1-18. In one embodiment, the castration-resistant prostate cancer is a rearranged AR, AR overexpressing castration-resistant prostate cancer, F876L mutation expressing castration-resistant prostate cancer, F876L_T877A double mutation expressing castration-resistant prostate cancer, AR-V7 expressing castration-resistant prostate cancer, d567ES expressing castration-resistant prostate cancer, and/or castration-resistant prostate cancer characterized by intratumoral androgen synthesis.

The invention encompasses a method of treating castration-sensitive prostate cancer in a subject in need thereof, comprising administering to the subject a therapeutically effective amount of a selective androgen receptor covalent antagonist (SARCA) compound, or its isomer, pharmaceutically acceptable salt, pharmaceutical product, polymorph, hydrate or any combination thereof, wherein said SARCA compound is represented by the structure of formula formulas I-XX, or the compound is at least one of compounds 1-18. In one embodiment, the castration-sensitive prostate cancer is F876L mutation expressing castration-sensitive prostate cancer, F876L_T877A double mutation castration-sensitive prostate cancer, and/or castration-sensitive prostate cancer characterized by intratumoral androgen synthesis. In one embodiment, the treating of castration-sensitive prostate cancer is conducted in a non-castrate setting, or as monotherapy, or when castration-sensitive prostate cancer tumor is resistant to enzalutamide, apalutamide, and/or abiraterone.

The invention encompasses a method of treating AR-V7 expressing prostate cancer in a subject in need thereof, comprising administering to the subject a therapeutically effective amount of a selective androgen receptor covalent antagonist (SARCA) compound, or its isomer, pharmaceutically acceptable salt, pharmaceutical product, polymorph, hydrate or any combination thereof, wherein said SARCA compound is represented by the structure of formula formulas I-XX, or the compound is at least one of compounds 1-18.

The invention encompasses a method of treating d567ES expressing prostate cancer in a subject in need thereof, comprising administering to the subject a therapeutically effective amount of a selective androgen receptor covalent antagonist (SARCA) compound, or its isomer, pharmaceutically acceptable salt, pharmaceutical product, polymorph, hydrate or any combination thereof, wherein said SARCA compound is represented by the structure of formula formulas I-XX, or the compound is at least one of compounds 1-18.

Treatment of Triple Negative Breast Cancer (TNBC)

Triple negative breast cancer (TNBC) is a type of breast cancer lacking the expression of the estrogen receptor (ER), progesterone receptor (PR), and HER2 receptor kinase. As such, TNBC lacks the hormone and kinase therapeutic targets used to treat other types of primary breast cancers. Correspondingly, chemotherapy is often the initial pharmacotherapy for TNBC. Interestingly, AR is often still expressed in TNBC and may offer a hormone targeted therapeutic alternative to chemotherapy. In ER-positive breast cancer, AR is a positive prognostic indicator as it is believed that activation of AR limits and/or opposes the effects of the ER in breast tissue and tumors. However, in the absence of ER, it is possible that AR actually supports the growth of breast cancer tumors. Though the role of AR is not fully understood in TNBC, there is evidence that certain TNBC's may be supported by androgen independent activation of AR-SVs lacking the LBD or androgen-dependent activation of AR full length. As such, enzalutamide and other LED-directed traditional AR antagonists would not be able to antagonize AR-SVs in these TNBC's. However, SARCAs of this invention through a binding site in the NTD of AR would be able to antagonize AR in these TNBC's and provide an anti-tumor effect.

Treatment of Kennedy's Disease

Muscle atrophy (MA) is characterized by wasting away or diminution of muscle and a decrease in muscle mass. For example, post-polio MA is muscle wasting that occurs as part of the post-polio syndrome (PPS). The atrophy includes weakness, muscle fatigue, and pain. Another type of MA is X-linked spinal-bulbar muscular atrophy (SBMA—also known as Kennedy's Disease). This disease arises from a defect in the androgen receptor gene on the X chromosome, affects only males, and its onset is in late adolescence to adulthood. Proximal limb and bulbar muscle weakness results in physical limitations including dependence on a wheelchair in some cases. The mutation results in an extended polyglutamine tract at the N-terminal domain of the androgen receptor (polyQ AR).

Binding and activation of the polyQ AR by endogeneous androgens (testosterone and DHT) results in unfolding and nuclear translocation of the mutant androgen receptor. The androgen-induced toxicity and androgen-dependent nuclear accumulation of polyQ AR protein seems to be central to the pathogenesis. Therefore, the inhibition of the androgen-activated polyQ AR might be a therapeutic option (A. Baniahmad. Inhibition of the androgen receptor by antiandrogens in spinobulbar muscle atrophy. J. Mol. Neurosci. 2016 58(3), 343-347). These steps are required for pathogenesis and result in partial loss of transactivation function (i.e., an androgen insensitivity) and a poorly understood neuromuscular degeneration. Peripheral polyQ AR anti-sense therapy rescues disease in mouse models of SBMA (Cell Reports 7, 774-784, May 8, 2014). Further support of use antiandrogen comes in a report in which the antiandrogen flutamide protects male mice from androgen-dependent toxicity in three models of spinal bulbar muscular atrophy (Renier K J, Troxell-Smith S M, Johansen J A, Katsuno M, Adachi H, Sobue G, Chua J P, Sun Kim H, Lieberman A P, Breedlove S M, Jordan C L. Endocrinology 2014, 155(7), 2624-2634). These steps are required for pathogenesis and result in partial loss of transactivation function (i.e., an androgen insensitivity) and a poorly understood neuromuscular degeneration. Currently there are no disease-modifying treatments, but rather only symptom directed treatments. Efforts to target the polyQ AR as the proximal mediator of toxicity by harnessing cellular machinery to promote its degradation hold promise for therapeutic intervention.

Selective androgen receptor covalent Antagonists such as those reported herein bind to, inhibit transactivation, and degrade all androgen receptors tested to date (full length, splice variant, antiandrogen resistance mutants, etc.), indicating that they are promising leads for treatment diseases whose pathogenesis is androgen-dependent such as SBMA.

The invention encompasses methods of treating Kennedy's disease comprising administering a therapeutically effective amount of a compound of formulas I-XX or the compound is at least one of compounds 1-18.

The term “androgen receptor dependent disease or condition” refers to diseases or conditions that have pathological origins or propagated by the altered, increased, dysregulated, or aberrant activity of an androgen receptor. In some embodiments, the androgen receptor is a full-length androgen receptor. In another embodiment, the androgen receptor is a wildtype full-length androgen receptor (AR-FL). In another embodiment, the androgen receptor is a point mutation of the full-length androgen receptor. In another embodiment, the androgen receptor is a polyQ polymorph. In another embodiment, the androgen receptor is a splice-variant of the androgen receptor (AR-SV). In another embodiment, the androgen receptor is any of the above or a combination thereof. In another embodiment, the androgen receptor is any of the above and is additionally overexpressed. In another embodiment, the androgen receptor is any of the above and further recombined with another gene to form a fusion protein. Examples of common AR fusion proteins include but are not limited to TMPRSS2 or ETS-family of transcription factors. In some embodiments, the androgen receptor is any of the above and presence in a pathologically changed cellular milieau. In another embodiment, the altered, increased, dysregulated or aberrant activity of an androgen receptor is caused by endogeneous androgens acting at the androgen receptor. In another embodiment, the altered, increased, dysregulated, or aberrant activity of an androgen receptor is caused by exogeneously administered compounds acting at the androgen receptor. In another embodiment, the altered, increased, dysregulated, or aberrant activity of an androgen receptor is ligand-independent. In another embodiment, the ligand-independent activity is caused by the constitutive activity of the androgen receptor. In another embodiment, the ligand-independent activity is caused by constitutively active mutants of the androgen receptor. In another embodiment, the ligand-independent activity is caused by pathologic cellular milieau. In another embodiment, these androgen receptor dependent diseases and conditions are improved by the administration of androgen receptor antagonists. In another embodiment, these androgen receptor dependent diseases and conditions are improved by the administration of androgen deprivation therapies (ADT) as described herein. In another embodiment, these androgen receptor dependent diseases and conditions are made worse by the administration of androgen receptor agonists. In another embodiment, these androgen receptor dependent diseases and conditions are improved by decreasing androgen receptor expression by biochemical treatments. In another embodiment, these androgen receptor dependent diseases and conditions are the result of hormonal imbalances. In another embodiment, the hormonal imbalance in a subject is a result of ageing, or in the other embodiments, the result of disease. In another embodiment, these androgen receptor dependent diseases and conditions are responsive to the administration of androgen receptor antagonists such as antiandrogens. In another embodiment, these androgen receptor dependent diseases and conditions are conditions, diseases, or disorders that are modulated by or whose pathogenesis is dependent upon the activity of the androgen receptor.

In one embodiment, an “androgen receptor dependent disease or condition” is a medical condition that is, in part or in full, dependent on, or is sensitive to, the presence of androgenic activity or activation of the AR-axis in the body. In another embodiment, an “androgen receptor dependent disease or condition” is any disease or condition which is known to be treated, inhibited, prevented, or suppressed by an AR antagonist.

In some embodiments, the androgen receptor dependent diseases and conditions are improved by administration of the selective androgen receptor covalent antagonists of the invention. In some embodiments, the benefit of selective androgen receptor covalent antagonists of the invention is their degradation of at least one form of the androgen receptor. In some embodiments, the benefit of selective androgen receptor covalent antagonists of the invention is their inhibition of at least one form of the androgen receptor. In some embodiments, the benefit of selective androgen receptor covalent antagonists of the invention is their degradation and inhibition of at least one form of the androgen receptor.

Many examples of androgen receptor dependent diseases and conditions are described herein, and these include but are not limited to prostate cancers, breast cancers, hormone-dependent cancers, hormone-independent cancers, AR-expressing cancers, and precursors to hormone-dependent cancers as are each described in detail herein below; dermatological disorders, hormonal conditions of a male or hormonal conditions of a female as are each described in detail herein below; androgen insufficiency syndromes as are described in detail below; uterine fibroids, Kennedy's disease (SBMA), amyotrophic lateral sclerosis (ALS), abdominal aortic aneurysm (AAA), improving wound healing, sexual perversion, hypersexuality, paraphilias, androgen psychosis, and virilization and the like.

As used herein, the term “androgen receptor associated conditions” or “androgen sensitive diseases or disorders” or “androgen-dependent diseases or disorders” are conditions, diseases, or disorders that are modulated by or whose pathogenesis is dependent upon the activity of the androgen receptor. The androgen receptor is expressed in most tissues of the body however it is overexpressed in, inter alia, the prostate and skin. ADT has been the mainstay of prostate cancer treatment for many years, and SARCAs may also be useful in treating various prostate cancers, benign prostatic hypertrophy, prostamegaly, and other maladies of the prostate.

The invention encompasses methods of treating benign prostatic hypertrophy comprising administering a therapeutically effective amount of at least one compound of formulas I-XX or the compound is at least one of compounds 1-18.

The invention encompasses methods of treating prostamegaly comprising administering a therapeutically effective amount of at least one compound of formulas I-XX or the compound is at least one of compounds 1-18.

The invention encompasses methods of treating hyperproliferative prostatic disorders and diseases comprising administering a therapeutically effective amount of a compound of formulas I-XX or the compound is at least one of compounds 1-18.

The effect of the AR on the skin is apparent in the gender dimorphism and puberty related dermatological problems common to teens and early adults. The hyperandrogenism of puberty stimulates terminal hair growth, sebum production, and predisposes male teens to acne, acne vulgaris, seborrhea, excess sebum, hidradenitis suppurativa, hirsutism, hypertrichosis, hyperpilosity, androgenic alopecia, male pattern baldness, and other dermatological maladies. Although antiandrogens theoretically should prevent the hyperandrogenic dermatological diseases discussed, they are limited by toxicities, sexual side effects, and lack of efficacy when topically applied. The SARCAs of this invention potently inhibit ligand-dependent and ligand-independent AR activation, and (in some cases) have short biological half-lives in the serum, suggesting that topically formulated SARCAs of this invention could be applied to the areas affected by acne, seborrheic dermatitis, and/or hirsutism without risk of systemic side effects.

The invention encompasses methods of treating acne, acne vulgaris, seborrhea, seborrheic dermatitis, hidradenitis supporativa, hirsutism, hypertrichosis, hyperpilosity, or alopecia comprising administering a therapeutically effective amount of a compound of formulas I-XX, or any of compounds 1-18.

The compounds and/or compositions described herein may be used for treating hair loss, alopecia, androgenic alopecia, alopecia areata, alopecia secondary to chemotherapy, alopecia secondary to radiation therapy, alopecia induced by scarring or alopecia induced by stress. Generally “hair loss” or “alopecia” refers to baldness as in the very common type of male-pattern baldness. Baldness typically begins with patch hair loss on the scalp and sometimes progresses to complete baldness and even loss of body hair. Hair loss affects both males and females.

The invention encompasses methods of treating androgenic alopecia comprising administering a therapeutically effective amount of a compound of formulas I-XX, or any of compounds 1-18.

The invention encompasses methods of treating, suppressing, reducing the incidence, reducing the severity, or inhibiting the progression of a hormonal condition in a male in need thereof, comprising administering to the subject a therapeutically effective amount of a selective androgen receptor covalent antagonist (SARCA) compound, or its isomer, pharmaceutically acceptable salt, pharmaceutical product, polymorph, hydrate or any combination thereof, wherein said SARCA compound is represented by the structure of formulas I-XX, or the compound is at least one of compounds 1-18.

In one embodiment, the condition is hypergonadism, hypersexuality, sexual dysfunction, gynecomastia, precocious puberty in a male, alterations in cognition and mood, depression, hair loss, hyperandrogenic dermatological disorders, pre-cancerous lesions of the prostate, benign prostate hyperplasia, prostate cancer and/or other androgen-dependent cancers.

SARCAs of this invention may also be useful in the treatment of hormonal conditions in females which can have hyperandrogenic pathogenesis such as precocious puberty, early puberty, dysmenorrhea, amenorrhea, multilocular uterus syndrome, endometriosis, hysteromyoma, abnormal uterine bleeding, early menarche, fibrocystic breast disease, fibroids of the uterus, ovarian cysts, polycystic ovary syndrome, pre-eclampsia, eclampsia of pregnancy, preterm labor, premenstrual syndrome, and/or vaginal dryness.

The invention encompasses methods of treating precocious puberty or early puberty, dysmenorrhea or amenorrhea, multilocular uterus syndrome, endometriosis, hysteromyoma, abnormal uterine bleeding, hyper-androgenic diseases (such as polycystic ovary syndrome (PCOS)), fibrocystic breast disease, fibroids of the uterus, ovarian cysts, polycystic ovary syndrome, preeclampsia, eclampsia of pregnancy, preterm labor, premenstrual syndrome, or vaginal dryness comprising administering a therapeutically effective amount of a compound of formulas I-XX, or any of compounds 1-18.

SARCAs of this invention may also find utility in treatment of sexual perversion, hypersexuality, paraphilias, androgen psychosis, virilization, androgen insensitivity syndromes (AIS) (such as complete AIS (CAIS) and partial AIS (PAIS)), and improving ovulation in an animal.

The invention encompasses methods of treating sexual perversion, hypersexuality, paraphilias, androgen psychosis, virilization androgen, insensitivity syndromes, increasing or modulating or improving ovulation comprising administering a therapeutically effective amount of a compound of formulas I-XX, or any of compounds 1-18.

SARCAs of this invention may also be useful for treating hormone-dependent cancers such as prostate cancer, breast cancer, testicular cancer, ovarian cancer, hepatocellular carcinoma, urogenital cancer, etc. In another embodiment, the breast cancer is triple negative breast cancer. Further, local or systemic SARCA administration may be useful for treatment of precursors of hormone-dependent cancers such as prostatic intraepithelial neoplasia (PIN) and atypical small acinar proliferation (ASAP).

The invention encompasses methods of treating breast cancer, testicular cancer, uterine cancer, ovarian cancer, urogenital cancer, precursors of prostate cancer, or AR related or AR expressing solid tumors, comprising administering a therapeutically effective amount of a compound of formulas I-XX or the compound is at least one of compounds 1-18. A precursor of prostate cancers may be prostatic intraepithelial neoplasia (PIN) or atypical small acinar proliferation (ASAP). The tumor may be hepatocellular carcinoma (HCC) or bladder cancer. Serum testosterone may be positively linked to the development of HCC. Based on epidemiologic, experimental observations, and notably the fact that men have a substantially higher risk of bladder cancer than women, androgens and/or the AR may also play a role in bladder cancer initiation.

Although traditional antiandrogens such as enzalutamide, bicalutamide and flutamide and androgen deprivation therapies (ADT) such as leuprolide were approved for use in prostate cancer, there is significant evidence that antiandrogens could also be used in a variety of other hormone-dependent and hormone-independent cancers. For example, antiandrogens may be used in a wide variety of AR-expressing cancers as described below. For example, antiandrogens have been successfully tested in breast cancer (enzalutamide; Breast Cancer Res (2014) 16(1): R7), non-small cell lung cancer (shRNAi AR), renal cell carcinoma (ASC-J9), partial androgen insensitivity associated malignancies such as gonadal tumors and seminoma, advanced pancreatic cancer (World J Gastroenterology 20(29):9229), cancer of the ovary, fallopian tubes, or peritoneum, cancer of the salivary gland (Head and Neck (2016) 38:724-731; ADT was tested in AR-expressing recurrent/metastatic salivary gland cancers and was confirmed to have benefit on progression free survival and overall survival endpoints), bladder cancer (Oncotarget 6(30): 29860-29876); Int J Endocrinol (2015), Article ID 384860), pancreatic cancer, lymphoma (including mantle cell), and hepatocellular carcinoma. Use of a more potent antiandrogen such as a SARCA in these cancers may treat the progression of these and other cancers. Other cancers may also benefit from SARCA treatment such as testicular cancer, uterine cancer, ovarian cancer, urogenital cancer, breast cancer, brain cancer, skin cancer, lymphoma, liver cancer, renal cancer, osteosarcoma, pancreatic cancer, endometrial cancer, lung cancer, non-small cell lung cancer (NSCLC), colon cancer, perianal adenoma, or central nervous system cancer.

SARCAs of this invention may also be useful for treating other cancers containing AR such as breast, brain, skin, ovarian, bladder, lymphoma, liver, kidney, pancreas, endometrium, lung (e.g., NSCLC), colon, perianal adenoma, osteosarcoma, CNS, melanoma, hypercalcemia of malignancy and metastatic bone disease, etc.

Thus, the invention encompasses methods of treating hypercalcemia of malignancy, metastatic bone disease, brain cancer, skin cancer, bladder cancer, lymphoma, liver cancer, renal cancer, osteosarcoma, pancreatic cancer, endometrial cancer, lung cancer, central nervous system cancer, gastric cancer, colon cancer, melanoma, amyotrophic lateral sclerosis (ALS), and/or uterine fibroids comprising administering a therapeutically effective amount of a compound of formulas I-XX, or any of compounds 1-18. The lung cancer may be non-small cell lung cancer (NSCLC).

SARCAs of this invention may also be useful for the treating of non-hormone-dependent cancers. Non-hormone-dependent cancers include liver, salivary duct, etc.

In another embodiment, the SARCAs of this invention are used for treating gastric cancer. In another embodiment, the SARCAs of this invention are used for treating salivary duct carcinoma. In another embodiment, the SARCAs of this invention are used for treating bladder cancer. In another embodiment, the SARCAs of this invention are used for treating esophageal cancer. In another embodiment, the SARCAs of this invention are used for treating pancreatic cancer. In another embodiment, the SARCAs of this invention are used for treating colon cancer. In another embodiment, the SARCAs of this invention are used for treating non-small cell lung cancer. In another embodiment, the SARCAs of this invention are used for treating renal cell carcinoma.

AR plays a role in cancer initiation in hepatocellular carcinoma (HCC). Therefore, targeting AR may be an appropriate treatment for patients with early stage HCC. In late-stage HCC disease, there is evidence that metastasis is suppressed by androgens. In another embodiment, the SARCAs of this invention are used for treating hepatocellular carcinoma (HCC).

Locati et al. in Head & Neck, 2016, 724-731 demonstrated the use of androgen deprivation therapy (ADT) in AR-expressing recurrent/metastatic salivary gland cancers and confirmed improved progression free survival and overall survival endpoints with ADT. In another embodiment, the SARCAs of this invention are used for treating salivary gland cancer.

Kawahara et al. in Oncotarget, 2015, Vol 6(30), 29860-29876 demonstrated that ELK1 inhibition, together with AR inactivation, has the potential of being a therapeutic approach for bladder cancer. McBeth et al. Int J Endocrinology, 2015, Vol 2015, Article ID 384860 suggested that the combination of antiandrogen therapy plus glucocorticoids as treatment of bladder cancer as this cancer is believed to have an inflammatory etiology. In another embodiment, the SARCAs of this invention are used for treating bladder cancer, optionally in combination with glucocorticoids.

Abdominal Aortic Aneurysm (AAA)

An abdominal aortic aneurysm (AAA) is an enlarged area in the lower part of the aorta, the major blood vessel that supplies blood to the body. The aorta, about the thickness of a garden hose, runs from your heart through the center of your chest and abdomen. Because the aorta is the body's main supplier of blood, a ruptured abdominal aortic aneurysm can cause life-threatening bleeding. Depending on the size and the rate at which your abdominal aortic aneurysm is growing, treatment may vary from watchful waiting to emergency surgery. Once an abdominal aortic aneurysm is found, doctors will closely monitor it so that surgery can be planned if it is necessary. Emergency surgery for a ruptured abdominal aortic aneurysm can be risky. AR blockade (pharmacologic or genetic) reduces AAA. Davis et al. (Davis J P, et al. J Vase Surg (2016) 63(6):1602-1612) showed that flutamide (50 mg/kg) or ketoconazole (150 mg/kg) attenuated AAA induced by porcine pancreatic elastase (0.35 U/mL) by 84.2% and 91.5% compared to vehicle (121%). Further AR−/−mice showed attenuated AAA growth (64.4%) compared to wildtype (both treated with elastase). Correspondingly, administration of a SARCA to a patient suffering from an AAA may help reverse, treat or delay progression of AAA to the point where surgery is needed.

Treatment of Wounds

Wounds and/or ulcers are normally found protruding from the skin or on a mucosal surface or as a result of an infarction in an organ. A wound may be a result of a soft tissue defect or a lesion or of an underlying condition. The term “wound” denotes a bodily injury with disruption of the normal integrity of tissue structures, sore, lesion, necrosis, and/or ulcer. The term “sore” refers to any lesion of the skin or mucous membranes and the term “ulcer” refers to a local defect, or excavation, of the surface of an organ or tissue, which is produced by the sloughing of necrotic tissue. “Lesion” generally includes any tissue defect. “Necrosis” refers to dead tissue resulting from infection, injury, inflammation, or infarctions. All of these are encompassed by the term “wound,” which denotes any wound at any particular stage in the healing process including the stage before any healing has initiated or even before a specific wound like a surgical incision is made (prophylactic treatment).

Examples of wounds which can be treated in accordance with the present invention are aseptic wounds, contused wounds, incised wounds, lacerated wounds, non-penetrating wounds (i.e., wounds in which there is no disruption of the skin but there is injury to underlying structures), open wounds, penetrating wounds, perforating wounds, puncture wounds, septic wounds, subcutaneous wounds, etc. Examples of sores include, but are not limited to, bed sores, canker sores, chrome sores, cold sores, pressure sores, etc. Examples of ulcers include, but are not limited to, peptic ulcer, duodenal ulcer, gastric ulcer, gouty ulcer, diabetic ulcer, hypertensive ischemic ulcer, stasis ulcer, ulcus cruris (venous ulcer), sublingual ulcer, submucous ulcer, symptomatic ulcer, trophic ulcer, tropical ulcer, veneral ulcer, e.g., caused by gonorrhoea (including urethritis, endocervicitis and proctitis). Conditions related to wounds or sores which may be successfully treated according to the invention include, but are not limited to, burns, anthrax, tetanus, gas gangrene, scalatina, erysipelas, sycosis barbae, folliculitis, impetigo contagiosa, impetigo bullosa, etc. It is understood, that there may be an overlap between the use of the terms “wound” and “ulcer,” or “wound” and “sore” and, furthermore, the terms are often used at random.

The kinds of wounds to be treated according to the invention include also: i) general wounds such as, e.g., surgical, traumatic, infectious, ischemic, thermal, chemical and bullous wounds; ii) wounds specific for the oral cavity such as, e.g., post-extraction wounds, endodontic wounds especially in connection with treatment of cysts and abscesses, ulcers and lesions of bacterial, viral or autoimmunological origin, mechanical, chemical, thermal, infectious and lichenoid wounds; herpes ulcers, stomatitis aphthosa, acute necrotising ulcerative gingivitis and burning mouth syndrome are specific examples; and iii) wounds on the skin such as, e.g., neoplasm, burns (e.g., chemical, thermal), lesions (bacterial, viral, autoimmunological), bites and surgical incisions. Another way of classifying wounds is by tissue loss, where: i) small tissue loss (due to surgical incisions, minor abrasions, and minor bites) or ii) significant tissue loss. The latter group includes ischemic ulcers, pressure sores, fistulae, lacerations, severe bites, thermal burns and donor site wounds (in soft and hard tissues) and infarctions. Other wounds include ischemic ulcers, pressure sores, fistulae, severe bites, thermal burns, or donor site wounds.

Ischemic ulcers and pressure sores are wounds, which normally only heal very slowly and especially in such cases an improved and more rapid healing is of great importance to the patient. Furthermore, the costs involved in the treatment of patients suffering from such wounds are markedly reduced when the healing is improved and takes place more rapidly.

Donor site wounds are wounds which e.g., occur in connection with removal of hard tissue from one part of the body to another part of the body e.g., in connection with transplantation. The wounds resulting from such operations are very painful and an improved healing is therefore most valuable.

In one case, the wound to be treated is selected from the group consisting of aseptic wounds, infarctions, contused wounds, incised wounds, lacerated wounds, non-penetrating wounds, open wounds, penetrating wounds, perforating wounds, puncture wounds, septic wounds, and subcutaneous wounds.

The invention encompasses methods of treating a subject suffering from a wound comprising administering to the subject a therapeutically effective amount of a compound of formulas I-XX, or the compound is at least one of compounds 1-18; or pharmaceutically acceptable salt thereof, or a pharmaceutical composition thereof.

The invention encompasses methods of treating a subject suffering from a burn comprising administering to the subject a therapeutically effective amount of a compound of formulas I-XX, or the compound is at least one of compounds 1-18; or pharmaceutically acceptable salt thereof, or a pharmaceutical composition thereof.

The term “skin” is used in a very broad sense embracing the epidermal layer of the skin and in those cases where the skin surface is more or less injured also the dermal layer of the skin. Apart from the stratum corneum, the epidermal layer of the skin is the outer (epithelial) layer and the deeper connective tissue layer of the skin is called the dermis.

Since the skin is the most exposed part of the body, it is particularly susceptible to various kinds of injuries such as, e.g., ruptures, cuts, abrasions, burns and frostbites or injuries arising from various diseases. Furthermore, much skin is often destroyed in accidents. However, due to the important barrier and physiologic function of the skin, the integrity of the skin is important to the well-being of the individual, and any breach or rupture represents a threat that must be met by the body in order to protect its continued existence.

Apart from injuries on the skin, injuries may also be present in all kinds of tissues (i.e., soft and hard tissues). Injuries on soft tissues including mucosal membranes and/or skin are especially relevant in connection with the present invention.

Healing of a wound on the skin or on a mucosal membrane undergoes a series of stages that results either in repair or regeneration of the skin or mucosal membrane. In recent years, regeneration and repair have been distinguished as the two types of healing that may occur. Regeneration may be defined as a biological process whereby the architecture and function of lost tissue are completely renewed. Repair, on the other hand, is a biological process whereby continuity of disrupted tissue is restored by new tissues which do not replicate the structure and function of the lost ones.

The majority of wounds heal through repair, meaning that the new tissue formed is structurally and chemically unlike the original tissue (scar tissue). In the early stage of the tissue repair, one process which is almost always involved is the formation of a transient connective tissue in the area of tissue injury. This process starts by formation of a new extracellular collagen matrix by fibroblasts. This new extracellular collagen matrix is then the support for a connective tissue during the final healing process. The final healing is, in most tissues, a scar formation containing connective tissue. In tissues which have regenerative properties, such as, e.g., skin and bone, the final healing includes regeneration of the original tissue. This regenerated tissue has frequently also some scar characteristics, e.g., a thickening of a healed bone fracture.

Under normal circumstances, the body provides mechanisms for healing injured skin or mucosa in order to restore the integrity of the skin barrier or the mucosa. The repair process for even minor ruptures or wounds may take a period of time extending from hours and days to weeks. However, in ulceration, the healing can be very slow and the wound may persist for an extended period of time, i.e., months or even years.

Burns are associated with reduced testosterone levels, and hypogonadism is associated with delayed wound healing. The invention encompasses methods for treating a subject suffering from a wound or a burn by administering at least one SARCA compound according to this invention. The SARCA may promote resolving of the burn or wound, participates in the healing process of a burn or a wound, or, treats a secondary complication of a burn or wound.

The treatment of burns or wounds may further use at least one growth factor such as epidermal growth factor (EGF), transforming growth factor-α (TGF-α), platelet derived growth factor (PDGF), fibroblast growth factors (FGFs) including acidic fibroblast growth factor (α-FGF) and basic fibroblast growth factor (β-FGF), transforming growth factor-O (TGF-β) and insulin like growth factors (IGF-1 and IGF-2), or any combination thereof, which promote wound healing.

Wound healing may be measured by many procedures known in the art, including, but not limited to, wound tensile strength, hydroxyproline or collagen content, procollagen expression, or re-epithelialization. As an example, a SARCA as described herein may be administered orally or topically at a dosage of about 0.1-100 mg per day. Therapeutic effectiveness is measured as effectiveness in enhancing wound healing as compared to the absence of the SARCA compound. Enhanced wound healing may be measured by known techniques such as decrease in healing time, increase in collagen density, increase in hydroxyproline, reduction in complications, increase in tensile strength, and increased cellularity of scar tissue.

The term “reducing the pathogenesis” is to be understood to encompass reducing tissue damage, or organ damage associated with a particular disease, disorder or condition. The term may include reducing the incidence or severity of an associated disease, disorder or condition, with that in question or reducing the number of associated diseases, disorders or conditions with the indicated, or symptoms associated thereto.

Pharmaceutical Compositions

The compounds of the invention may be used in pharmaceutical compositions. As used herein, “pharmaceutical composition” means either the compound or pharmaceutically acceptable salt of the active ingredient with a pharmaceutically acceptable carrier or diluent. A “therapeutically effective amount” as used herein refers to that amount which provides a therapeutic effect for a given indication and administration regimen.

As used herein, the term “administering” refers to bringing a subject in contact with a compound of the present invention. As used herein, administration can be accomplished in vitro, i.e., in a test tube, or in vivo, i.e., in cells or tissues of living organisms, for example humans. The subjects may be a male or female subject or both.

Numerous standard references are available that describe procedures for preparing various compositions or formulations suitable for administration of the compounds of the invention. Examples of methods of making formulations and preparations can be found in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

The mode of administration and dosage form are closely related to the therapeutic amounts of the compounds or compositions which are desirable and efficacious for the given treatment application.

The pharmaceutical compositions of the invention can be administered to a subject by any method known to a person skilled in the art. These methods include, but are not limited to, orally, parenterally, intravascularly, paracancerally, transmucosally, transdermally, intramuscularly, intranasally, intravenously, intradermally, subcutaneously, sublingually, intraperitoneally, intraventricularly, intracranially, intravaginally, by inhalation, rectally, or intratumorally. These methods include any means in which the composition can be delivered to tissue (e.g., needle or catheter). Alternatively, a topical administration may be desired for application to dermal, ocular, or mucosal surfaces. Another method of administration is via aspiration or aerosol formulation. The pharmaceutical compositions may be administered topically to body surfaces, and are thus formulated in a form suitable for topical administration. Suitable topical formulations include gels, ointments, creams, lotions, drops and the like. For topical administrations, the compositions are prepared and applied as solutions, suspensions, or emulsions in a physiologically acceptable diluent with or without a pharmaceutical carrier.

Suitable dosage forms include, but are not limited to, oral, rectal, sub-lingual, mucosal, nasal, ophthalmic, subcutaneous, intramuscular, intravenous, transdermal, spinal, intrathecal, intraarticular, intra-arterial, sub-arachinoid, bronchial, lymphatic, and intra-uterile administration, and other dosage forms for systemic delivery of active ingredients. Depending on the indication, formulations suitable for oral or topical administration are preferred.

Topical Administration: The compounds of formulas I-XX or at least one of compounds 1-18 may be administered topically. As used herein, “topical administration” refers to application of the compounds of formulas I-XX or the compound is at least one of compounds 1-18 (and optional carrier) directly to the skin and/or hair. The topical composition can be in the form of solutions, lotions, salves, creams, ointments, liposomes, sprays, gels, foams, roller sticks, and any other formulation routinely used in dermatology.

Topical administration is used for indications found on the skin, such as hirsutism, alopecia, acne, and excess sebum. The dose will vary, but as a general guideline, the compound will be present in a dermatologically acceptable carrier in an amount of from about 0.01 to 50 w/w %, and more typically from about 0.1 to 10 w/w %. Typically, the dermatological preparation will be applied to the affected area from 1 to 4 times daily. “Dermatologically acceptable” refers to a carrier which may be applied to the skin or hair, and which will allow the drug to diffuse to the site of action. More specifically “site of action”, it refers to a site where inhibition of androgen receptor or degradation of the androgen receptor is desired.

The compounds of formulas I-XX, or at least one of compounds 1-18, may be used topically to relieve alopecia, especially androgenic alopecia. Androgens have a profound effect on both hair growth and hair loss. In most body sites, such as the beard and pubic skin, androgens stimulate hair growth by prolonging the growth phase of the hair cycle (anagen) and increasing follicle size. Hair growth on the scalp does not require androgens but, paradoxically, androgens are necessary for the balding on the scalp in genetically predisposed individuals (androgenic alopecia) where there is a progressive decline in the duration of anagen and in hair follicle size. Androgenic alopecia is also common in women where it usually presents as a diffuse hair loss rather than showing the patterning seen in men.

While the compounds of formulas I-XX or at least one of compounds 1-18 will most typically be used to alleviate androgenic alopecia, the compounds may be used to alleviate any type of alopecia. Examples of non-androgenic alopecia include, but are not limited to, alopecia areata, alopecia due to radiotherapy or chemotherapy, scarring alopecia, or stress related alopecia.

The compounds of formulas I-XX or at least one of compounds 1-18 can be applied topically to the scalp and hair to prevent or treat balding. Further, the compound of formulas I-XX or at least one of compounds 1-18 can be applied topically in order to induce or promote the growth or regrowth of hair on the scalp.

The invention also encompasses topically administering a compound of formulas I-XX or the compound is at least one of compounds 1-18 to treat or prevent the growth of hair in areas where such hair growth in not desired. One such use will be to alleviate hirsutism. Hirsutism is excessive hair growth in areas that typically do not have hair (e.g., a female face). Such inappropriate hair growth occurs most commonly in women and is frequently seen at menopause. The topical administration of the compounds of formulas I-XX or at least one of compounds 1-18 will alleviate this condition leading to a reduction, or elimination of this inappropriate, or undesired, hair growth.

The compounds of formulas I-XX or at least one of compounds 1-18 may also be used topically to decrease sebum production. Sebum is composed of triglycerides, wax esters, fatty acids, sterol esters and squalene. Sebum is produced in the acinar cells of the sebaceous glands and accumulates as these cells age. At maturation, the acinar cells lyse, releasing sebum into the luminal duct so that it may be deposited on the surface of the skin.

In some individuals, an excessive quantity of sebum is secreted onto the skin. This can have a number of adverse consequences. It can exacerbate acne, since sebum is the primary food source for Propionbacterium acnes, the causative agent of acne. It can cause the skin to have a greasy appearance, typically considered cosmetically unappealing.

Formation of sebum is regulated by growth factors and a variety of hormones including androgens. The cellular and molecular mechanism by which androgens exert their influence on the sebaceous gland has not been fully elucidated. However, clinical experience documents the impact androgens have on sebum production. Sebum production is significantly increased during puberty when androgen levels are their highest. The compounds of formulas I-XX or at least one of compounds 1-18 inhibit the secretion of sebum and thus reduce the amount of sebum on the surface of the skin. The compounds of formulas I-XX or at least one of compounds 1-18 can be used to treat a variety of dermal diseases such as acne or seborrheic dermatitis.

In addition to treating diseases associated with excess sebum production, the compounds of formulas I-XX or at least one of compounds 1-18 can also be used to achieve a cosmetic effect. Some consumers believe that they are afflicted with overactive sebaceous glands. They feel that their skin is oily and thus unattractive. These individuals may use the compounds of formulas I-XX or at least one of compounds 1-18 to decrease the amount of sebum on their skin. Decreasing the secretion of sebum will alleviate oily skin in individuals afflicted with such conditions.

To treat these topical indications, the invention encompasses cosmetic or pharmaceutical compositions (such as dermatological compositions), comprising at least one of the compounds of formulas I-XX or the compound is at least one of compounds 1-18. Such dermatological compositions will contain from 0.001% to 10% w/w % of the compound(s) in admixture with a dermatologically acceptable carrier, and more typically, from 0.1 to 5 w/w % of the compounds. Such compositions will typically be applied from 1 to 4 times daily. The reader's attention is directed to Remington's Pharmaceutical Science, Edition 17, Mark Publishing Co., Easton, PA for a discussion of how to prepare such formulations.

The compositions of the invention may also include solid preparations such as cleansing soaps or bars. These compositions are prepared according to methods known in the art.

Formulations such as aqueous, alcoholic, or aqueous-alcoholic solutions, or creams, gels, emulsions or mousses, or aerosol compositions with a propellant may be used to treat indications that arise where hair is present. Thus, the composition can also be a hair care composition. Such hair care compositions include, but are not limited to, shampoo, a hair-setting lotion, a treating lotion, a styling cream or gel, a dye composition, or a lotion or gel for preventing hair loss. The amounts of the various constituents in the dermatological compositions are those conventionally used in the fields considered.

Medicinal and cosmetic agents containing the compounds of formulas I-XX or at least one of compounds 1-18 will typically be packaged for retail distribution (i.e., an article of manufacture). Such articles will be labeled and packaged in a manner to instruct the patient how to use the product. Such instructions will include the condition to be treated, duration of treatment, dosing schedule, etc.

Antiandrogens, such as finasteride or flutamide, have been shown to decrease androgen levels or block androgen action in the skin to some extent but suffer from undesirable systemic effects. An alternative approach is to topically apply a selective androgen receptor covalent antagonist (SARCA) compound to the affected areas. Such SARCA compound would exhibit potent but local inhibition of AR activity, and local degradation of the AR, would not penetrate to the systemic circulation of the subject, or would be rapidly metabolized upon entry into the blood, limiting systemic exposure.

To prepare such pharmaceutical dosage forms, the active ingredient may be mixed with a pharmaceutical carrier according to conventional pharmaceutical compounding techniques. The carrier may take a wide variety of forms depending on the form of preparation desired for administration.

As used herein “pharmaceutically acceptable carriers or diluents” are well known to those skilled in the art. The carrier or diluent may be a solid carrier or diluent for solid formulations, a liquid carrier or diluent for liquid formulations, or mixtures thereof.

Solid carriers/diluents include, but are not limited to, a gum, a starch (e.g., corn starch, pregeletanized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

Oral and Parenteral Administration: In preparing the compositions in oral dosage form, any of the usual pharmaceutical media may be employed. Thus, for liquid oral preparations, such as, suspensions, elixirs, and solutions, suitable carriers and additives include water, glycols, oils, alcohols, flavoring agents, preservatives, coloring agents, and the like. For solid oral preparations such as, powders, capsules, and tablets, suitable carriers and additives include starches, sugars, diluents, granulating agents, lubricants, binders, disintegrating agents, and the like. Due to their ease in administration, tablets and capsules represent the most advantageous oral dosage unit form. If desired, tablets may be sugar coated or enteric coated by standard techniques.

For parenteral formulations, the carrier will usually comprise sterile water, though other ingredients may be included, such as ingredients that aid solubility or for preservation. Injectable solutions may also be prepared in which case appropriate stabilizing agents may be employed.

In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

Methods of treatment using formulations suitable for oral administration may be presented as discrete units such as capsules, cachets, tablets, or lozenges, each containing a predetermined amount of the active ingredient. Optionally, a suspension in an aqueous liquor or a non-aqueous liquid may be employed, such as a syrup, an elixir, an emulsion, or a draught.

A tablet may be made by compression or molding, or wet granulation, optionally with one or more accessory ingredients. Compressed tablets may be prepared by compressing in a suitable machine, with the active compound being in a free-flowing form such as a powder or granules which optionally is mixed with, for example, a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration may comprise a sterile aqueous preparation of the active compound, which preferably is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

Nasal and other mucosal spray formulations (e.g., inhalable forms) can comprise purified aqueous solutions of the active compounds with preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

Formulations for rectal administration may be presented as a suppository with a suitable carrier such as cocoa butter, hydrogenated fats, or hydrogenated fatty carboxylic acids.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, formulations of this invention may further include one or more ingredient selected from diluents, buffers, flavoring agents, binders, disintegrants, surface active agents, thickeners, lubricants, preservatives (including antioxidants), and the like.

The formulations may be of immediate release, sustained release, delayed-onset release or any other release profile known to one skilled in the art.

For administration to mammals, and particularly humans, it is expected that the physician will determine the actual dosage and duration of treatment, which will be most suitable for an individual and can vary with the age, weight, genetics and/or response of the particular individual.

The methods of the invention comprise administration of a compound at a therapeutically effective amount. The therapeutically effective amount may include various dosages.

In one embodiment, a compound of this invention is administered at a dosage of 1-3000 mg per day. In additional embodiments, a compound of this invention is administered at a dose of 1-10 mg per day, 3-26 mg per day, 3-60 mg per day, 3-16 mg per day, 3-30 mg per day, 10-26 mg per day, 15-60 mg, 50-100 mg per day, 50-200 mg per day, 100-250 mg per day, 125-300 mg per day, 20-50 mg per day, 5-50 mg per day, 200-500 mg per day, 125-500 mg per day, 500-1000 mg per day, 200-1000 mg per day, 1000-2000 mg per day, 1000-3000 mg per day, 125-3000 mg per day, 2000-3000 mg per day, 300-1500 mg per day or 100-1000 mg per day. In one embodiment, a compound of this invention is administered at a dosage of 25 mg per day. In one embodiment, a compound of this invention is administered at a dosage of 40 mg per day. In one embodiment, a compound of this invention is administered at a dosage of 50 mg per day. In one embodiment, a compound of this invention is administered at a dosage of 67.5 mg per day. In one embodiment, a compound of this invention is administered at a dosage of 75 mg per day. In one embodiment, a compound of this invention is administered at a dosage of 80 mg per day. In one embodiment, a compound of this invention is administered at a dosage of 100 mg per day. In one embodiment, a compound of this invention is administered at a dosage of 125 mg per day. In one embodiment, a compound of this invention is administered at a dosage of 250 mg per day. In one embodiment, a compound of this invention is administered at a dosage of 300 mg per day. In one embodiment, a compound of this invention is administered at a dosage of 500 mg per day. In one embodiment, a compound of this invention is administered at a dosage of 600 mg per day. In one embodiment, a compound of this invention is administered at a dosage of 1000 mg per day. In one embodiment, a compound of this invention is administered at a dosage of 1500 mg per day. In one embodiment, a compound of this invention is administered at a dosage of 2000 mg per day. In one embodiment, a compound of this invention is administered at a dosage of 2500 mg per day. In one embodiment, a compound of this invention is administered at a dosage of 3000 mg per day.

The methods may comprise administering a compound at various dosages. For example, the compound may be administered at a dosage of 3 mg, 10 mg, 30 mg, 40 mg, 50 mg, 80 mg, 100 mg, 120 mg, 125 mg, 200 mg, 250 mg, 300 mg, 450 mg, 500 mg, 600 mg, 900 mg, 1000 mg, 1500 mg, 2000 mg, 2500 mg or 3000 mg.

Alternatively, the compound may be administered at a dosage of 0.1 mg/kg/day. The compound may be administered at a dosage between 0.2 to 30 mg/kg/day, or 0.2 mg/kg/day, 0.3 mg/kg/day, 1 mg/kg/day, 3 mg/kg/day, 5 mg/kg/day, 10 mg/kg/day, 20 mg/kg/day, 30 mg/kg/day, 50 mg/kg/day or 100 mg/kg/day.

The pharmaceutical composition may be a solid dosage form, a solution, or a transdermal patch. Solid dosage forms include, but are not limited to, tablets and capsules.

The following examples are presented in order to more fully illustrate the preferred embodiments of the invention. They should in no way, however, be construed as limiting the broad scope of the invention.

### EXAMPLES

### Example 1: Synthesis of SARCA Compounds

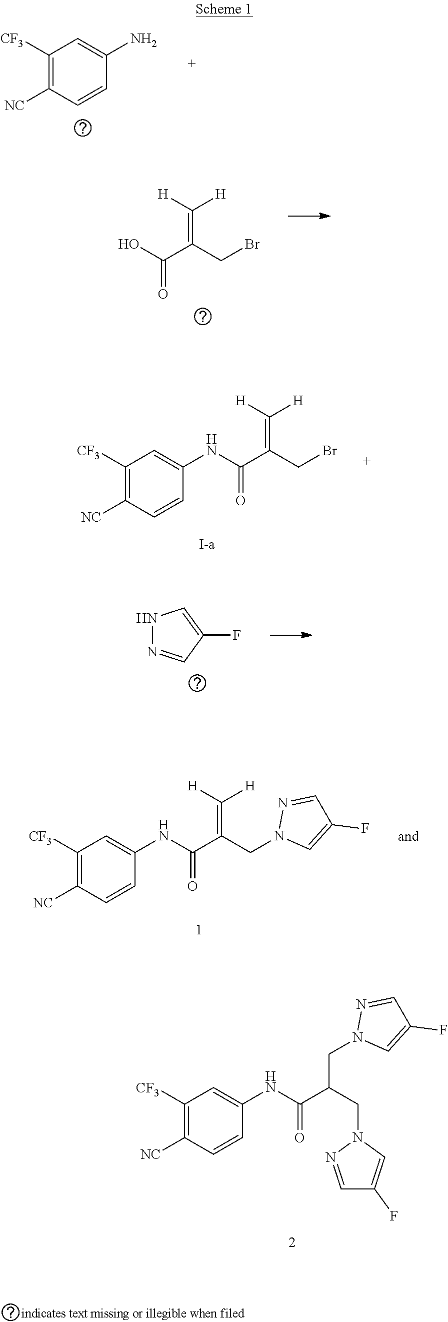 [SMILES: N#Cc1ccc(N)cc1C(F)(F)F ; C=C(CBr)C(=O)O ; C=C(CBr)C(=O)Nc1ccc(C#N)c(C(F)(F)F)c1 ; Fc1cn[nH]c1 ; C=C(Cn1cc(F)cn1)C(=O)Nc1ccc(C#N)c(C(F)(F)F)c1 ; N#Cc1ccc(NC(=O)C(Cn2cc(F)cn2)Cn2cc(F)cn2)cc1C(F)(F)F]

### 2-(Bromemethyl)-N-(4-cyano-3-(trifluoromethyl)phenyl)acrylamide (C₁₂H₈BrF₃N₂O) (1-a)

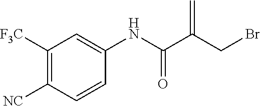 [SMILES: C=C(CBr)C(=O)Nc1ccc(C#N)c(C(F)(F)F)c1]

2-(Bromomethyl)acrylic acid (3.00 g, 0.0181829 mol) reacted with thionyl chloride (2.60 μg, 0.02182 mol), trimethylamine (2.39 g, 0.023638 mol), and 4-amino-2-(trifluoromethyl)benzonitrile (3.38 g, 0.0181829 mol) to afford the titled compound. The product was purified by a silica gel column using DCM and ethyl acetate (19:1) as eluent to afford 5.16 g (84%) of the titled compound as light brown solid.

¹H NMR (400 MHz, CDCl₃) δ 8.36 (s, 1H, NH), 8.10 (s, 1H, ArH), 8.02-8.00 (m, 1H, ArH), 7.83-7.80 (m, 1H, ArH), 6.11 (s, 1H, C═CH), 5.96 (s, 1H, C═CH), 4.41 (s, 2H, CH₂). Mass (ESI, Positive): 333.04 [M+H]⁺.

### N-(4-Cyano-3-(trifluoromethyl)phenyl)-2-((4-fluoro-1H-pyrazol-1-yl)methyl)acrylamide (C₁₅H₁₀F₄N₄O) (1)

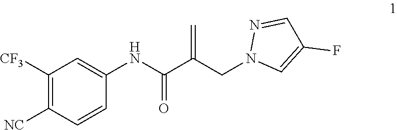 [SMILES: C=C(Cn1cc(F)cn1)C(=O)Nc1ccc(C#N)c(C(F)(F)F)c1]

To a solution of 4-fluoro-1H-pyrazole (0.41 g, 0.004803 mol) in anhydrous THF (20 mL), which was cooled in an ice water bath under an argon atmosphere, was added sodium hydride (60% dispersion in oil, 0.58 g, 0.01441 mol). After addition, the resulting mixture was stirred for 3 h. 2-(Bromomethyl)-N-(4-cyano-3-(trifluoromethyl) phenyl)acrylamide (1-a) (1.60 g, 0.004803 mol) was added to above solution, and the resulting reaction mixture was allowed to stir overnight at room temperature (RT) under argon. The reaction was quenched by water, and extracted with ethyl acetate. The organic layer was washed with brine, dried with MgSO₄, filtered, and concentrated under vacuum. The product was purified by a silica gel column using DCM and ethyl acetate (9:1) as eluent to afford 0.10 g (6%) of the titled compound as white solid.

¹H NMR (400 MHz, DMSO-d₆) δ 10.80 (s, 1H, NH), 8.34 (s, 1H, ArH), 8.14-8.13 (m, 2H, ArH), 7.91-7.90 (m, 1H, Pyrazole-H), 7.52-7.51 (m, 1H, Pyrazole-H), 6.15 (s, 1H, C═CH), 5.59 (s, 1H, C═CH), 4.49 (s, 2H, CH₂). HRMS [C₁₅H₁₁F₄N₄O⁺]: calcd 339.0869, found 339.0892 [M+H]⁺. Purity: 97.18% (HPLC).

### N-(4-Cyano-3-(trifluoromethyl)phenyl)-3-(4-fluoro-1H-pyrazol-1-yl)-2-((4-fluoro-1H-pyrazol-1-yl)methyl)propanamide (C₁₈H₁₃F₅N₆O) (2)

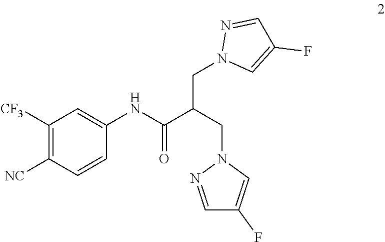 [SMILES: N#Cc1ccc(NC(=O)C(Cn2cc(F)cn2)Cn2cc(F)cn2)cc1C(F)(F)F]

To a solution of 4-fluoro-1H-pyrazole (0.41 g, 0.004803 mol) in anhydrous THF (20 mL), which was cooled in an ice water bath under an argon atmosphere, was added sodium hydride (60% dispersion in oil, 0.58 g, 0.01441 mol). After addition, the resulting mixture was stirred for 3 h. 2-(Bromomethyl)-N-(4-cyano-3-(trifluoromethyl)phenyl)acrylamide (1-a) (1.60 g, 0.004803 mol) was added to above solution, and the resulting reaction mixture was allowed to stir overnight at RT under argon. The reaction was quenched by water, and extracted with ethyl acetate. The organic layer was washed with brine, dried with MgSO₄, filtered, and concentrated under vacuum. The product was purified by a silica gel column using DCM and ethyl methanol (19:1) as eluent to afford 0.20 g (10%) of the titled compound as white solid.

¹H NMR (400 MHz, DMSO-d₆) δ 10.81 (s, 1H, NH), 8.17 (d, J=2.0 Hz, 1H, ArH), 8.09 (d, J=8.2 Hz, 1H, ArH), 7.87 (dd, J=8.2 Hz, J=2.0 Hz, 1H, ArH), 7.85-7.84 (m, 2H, Pyrazole-H), 7.49-7.48 (m, 2H, Pyrazole-H), 4.41-4.36 (m, 1H, CH₂), 4.26-4.21 (m, 1H, CH₂), 3.61-3.57 (m, 1H, CH). HRMS [C₁₈H₁₄F₅N₆O⁺]: calcd 524.1149, found 425.1157 [M+H]⁺. Purity: 95.50% (HPLC).

### N-(4-Cyano-3-(trifluoromethyl)phenyl)-2-(((4-cyano-3-(trifluoromethyl)phenyl)amino) methyl)acrylamide (C₂₀H₁₂F₆N₄O) (3)

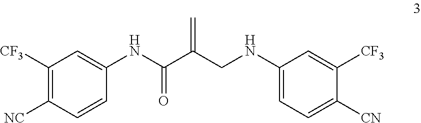 [SMILES: C=C(CNc1ccc(C#N)c(C(F)(F)F)c1)C(=O)Nc1ccc(C#N)c(C(F)(F)F)c1]

To a solution of 4-fluoro-1H-pyrazole (0.41 g, 0.004803 mol) in anhydrous THF (20 mL), which was cooled in an ice water bath under an argon atmosphere, was added sodium hydride (60% dispersion in oil, 0.58 g, 0.01441 mol). After addition, the resulting mixture was stirred for 3 h. 1-a (1.60 g, 0.004803 mol) was added to above solution, and the resulting reaction mixture was allowed to stir overnight at RT under argon. The reaction was quenched by water, and extracted with ethyl acetate. The organic layer was washed with brine, dried with MgSO₄, filtered, and concentrated under vacuum. The product was purified by a silica gel column using DCM and ethyl acetate (9:1) as eluent to afford 0.10 g (5%) of the titled compound as white solid.

¹H NMR (400 MHz, DMSO-d₆) δ 10.74 (s, 1H, NH), 8.40 (d, J=1.6 Hz, 1H, ArH), 8.19-8.12 (m, 2H, ArH), 7.76 (d, J=8.4 Hz, 1H, ArH), 7.65-7.62 (m, 1H, ArH), 7.10 (br s, 1H, NH), 6.89 (d, J=8.0 Hz, 1H, ArH), 6.07 (s, 1H, C═CH), 5.76 (s, 1H, C═CH), 4.18 (d, J=6.0 Hz, 2H, CH₂). HRMS [C₂₀H₁₂F₆N₄O⁺]: calcd 439.0999, found 439.0999 [M+H]⁺. Purity: 95.55% (HPLC).

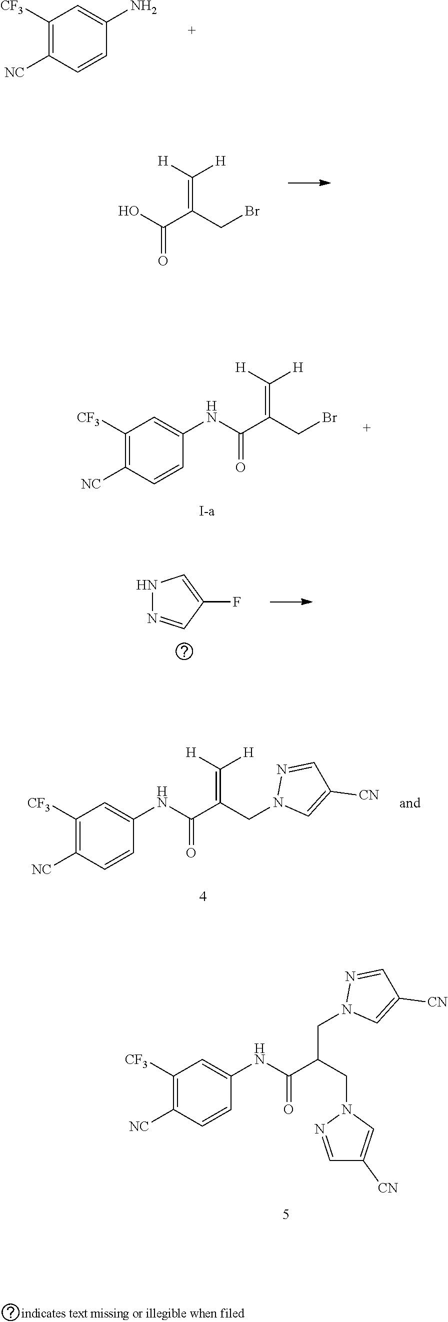 [SMILES: N#Cc1ccc(N)cc1C(F)(F)F ; C=C(CBr)C(=O)O ; C=C(CBr)C(=O)Nc1ccc(C#N)c(C(F)(F)F)c1 ; Fc1cn[nH]c1 ; C=C(Cn1cc(C#N)cn1)C(=O)Nc1ccc(C#N)c(C(F)(F)F)c1 ; N#Cc1cnn(CC(Cn2cc(C#N)cn2)C(=O)Nc2ccc(C#N)c(C(F)(F)F)c2)c1]

### 2-((4-Cyano-1H-pyrazol-1-yl)methyl)-N-(4-cyano-3-(trifluoromethyl)phenyl)acrylamide (C₁₆H₁₀F₃N₅O) (4)

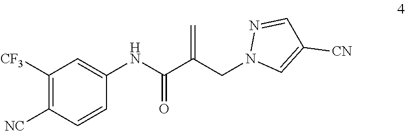 [SMILES: C=C(Cn1cc(C#N)cn1)C(=O)Nc1ccc(C#N)c(C(F)(F)F)c1]

To a solution of 4-cyano-1H-pyrazole (0.45 g, 0.004833 mol) in anhydrous THF (20 mL), which was cooled in an ice water bath under an argon atmosphere, was added sodium hydride (60% dispersion in oil, 0.58 g, 0.01450 mol). After addition, the resulting mixture was stirred for 3 h. 1-a (1.61 g, 0.004833 mol) was added to above solution, and the resulting reaction mixture was allowed to stir overnight at RT under argon. The reaction was quenched by water, and extracted with ethyl acetate. The organic layer was washed with brine, dried with MgSO₄, filtered, and concentrated under vacuum. The product was purified by a silica gel column using DCM and methanol (19:1) as eluent to afford 0.060 g (3.6%) of the titled compound as yellowish solid.

¹H NMR (400 MHz, DMSO-d₆) δ 10.82 (s, 1H, NH), 8.62 (s, 1H, Pyrazole-H), 8.33 (s, 1H, ArH), 8.15-8.13 (m, 2H, ArH), 8.10 (s, 1H, Pyrazole-H), 6.23 (s, 1H, C═CH), 5.73 (s, 1H, C═CH), 5.14 (s, 2H, CH₂). HRMS [C₁₆H₁₁F₃N₅O⁺]: calcd 346.0916, found 346.0927 [M+H]⁺. Purity:% (HPLC).

### 3-(4-Cyano-1H-pyrazol-1-yl)-2-((4-cyano-1H-pyrazol-1-yl)methyl)-N-(4-cyano-3-(trifluoromethyl)phenyl)propanamide (C₂₀H₁₃F₃N₈O) (5)

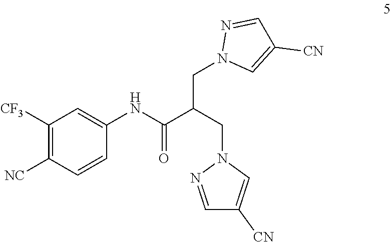 [SMILES: N#Cc1cnn(CC(Cn2cc(C#N)cn2)C(=O)Nc2ccc(C#N)c(C(F)(F)F)c2)c1]

To a solution of 4-cyano-1H-pyrazole (0.45 g, 0.004833 mol) in anhydrous THF (20 mL), which was cooled in an ice water bath under an argon atmosphere, was added sodium hydride (60% dispersion in oil, 0.58 g, 0.01450 mol). After addition, the resulting mixture was stirred for 3 h. 1-a (1.61 g, 0.004833 mol) was added to above solution, and the resulting reaction mixture was allowed to stir overnight at RT under argon. The reaction was quenched by water, and extracted with ethyl acetate. The organic layer was washed with brine, dried with MgSO₄, filtered, and concentrated under vacuum. The product was purified by a silica gel column using DCM and ethyl methanol (19:1) as eluent to afford 0.155 g (7.35%) of the titled compound as yellowish solid.

¹H NMR (400 MHz, DMSO-d₆) δ 10.87 (s, 1H, NH), 8.57 (m, 2H, Pyrazole-H), 8.12 (d, J=1.6 Hz, 1H, ArH), 8.11 (d, J=8.2 Hz, 1H, ArH), 8.05 (m, 2H, Pyrazole-H), 7.85 (dd, J=8.2 Hz, J=1.6 Hz, 1H, ArH), 4.58-4.53 (m, 1H, CH₂), 4.48-4.43 (m, 1H, CH₂), 3.71-3.67 (m, 1H, CH). HRMS [C₂₀H₁₄F₃N₈O⁺]: calcd 439.1243, found 439.1244 [M+H]+. Purity: 86.17% (HPLC).

### (S)-Methyl 2-(((3-(4-cyano-1H-pyrazol-1-yl)-1-((6-cyano-5-(trifluoromethyl)pyridine-3-yl)amino)-2-methyl-1-oxopropan-2-yl)oxy)methyl)acrylate (C₂₀H₁₇F3N₆O₄) (6)

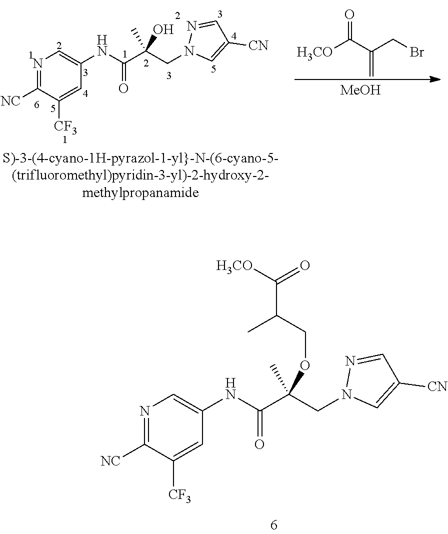

A solution of methyl 2-(bromomethyl)acrylate (0.2 mL, 0.74 mmol) in 5 mL of methanol was treated with (S)-3-(4-cyano-1H-pyrazol-1-yl)-N-(6-cyano-5-(trifluoromethyl)pyridin-3-yl)-2-hydroxy-2-methylpropanamide (200 mg, 0.54 mmol) portion wise over 10 min at RT. The solution was then stirred at RT. The solution was then stirred overnight at RT and the solution concentrated in vacuo. The residue was then taken up in water and extracted four times with ethyl acetate. The combined ethyl acetate solution was washed with saturated sodium chloride, dried over anhydrous magnesium sulfate, filtered and concentrated. The residue was then purified by silica gel column chromatography eluting with hexane/ethyl acetate 1:1, to give desired product as white solid (Yield 52%).

¹H NMR (CDCl₃, 400 MHz) δ 10.61 (bs, 1H, NH—C(O)), 9.17 (s, 1H), 8.89 (s, 1H), 7.85 (s, 1H), 7.72 (s, 1H), 6.52 (s, 1H), 6.08 (s, 1H), 4.55 (d, J=13.6 Hz, 1H), 4.41 (d, J=13.6 Hz, 1H), 4.36 (d, J=9.2 Hz, 1H), 4.09 (d, J=9.2 Hz, 1H), 3.77 (s, 3H, O—CH₃), 1.59 (s, 3H, CH₃); ¹³C NMR (CDCl₃, 100 MHz) δ 171.61, 167.86, 144.47, 142.90, 142.00, 137.52, 136.30, 132.23, 131.14 (q, J=33.5 Hz), 125.00, 123.87 (d, J=4.8 Hz), 123.02, 120.29, 114.42, 113.13, 92.78, 80.96, 65.63, 59.73, 53.11, 18.27. ¹⁹F NMR (CDCl₃, 400 MHz) δ −62.15. MS (ESI) m/z 461.23 [M−H]⁻; 463.27 [M+H]⁺; 485.21 [M+Na]⁺; HRMS (ESI) m/z calcd for C₂₀H₁₇F₃N₆O₄ 463.1342 [M+H]⁺ found 463.1342 [M+Ht.

### (S)-Methyl 2-((3-(4-cyano-1H-pyrazol-1-yl)-N-(6-cyano-5-(trifluoromethyl)pyridin-3-yl)-2-hydroxy-2-methylpropanamido)methyl)acrylate (C₂₀H₁₇F₃N₆O₄) (7)

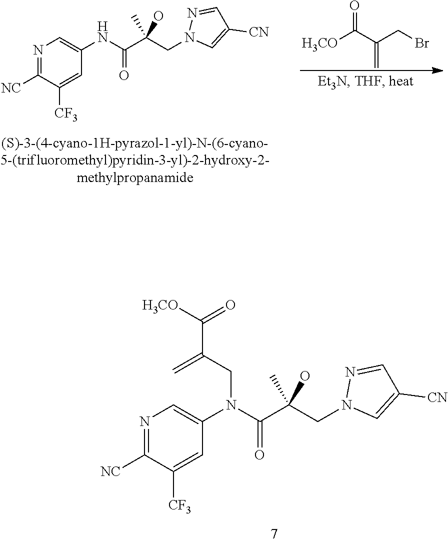

A solution of methyl 2-(bromomethyl)acrylate (0.2 mL, 0.74 mmol) in 5 mL of THF was treated with (S)-3-(4-cyano-1H-pyrazol-1-yl)-N-(6-cyano-5-(trifluoromethyl) pyridin-3-yl)-2-hydroxy-2-methylpropanamide (200 mg, 0.54 mmol) portion wise over 10 min at RT. The solution was then stirred at RT. The solution was then stirred overnight at RT and the solution concentrated in vacuo. The residue was then taken up in water and extracted four times with ethyl acetate. The combined ethyl acetate solution was washed with saturated sodium chloride, dried over anhydrous magnesium sulfate, filtered and concentrated. The residue was then purified by silica gel column chromatography eluting with hexane/ethyl acetate 1:1, to give desired product as yellowish oil (Yield 48%).

¹H NMR (CDCl₃, 400 MHz) δ 7.96 (s, 1H), 7.82 (s, 1H), 6.37 (s, 1H), 6.10 (s, 1H), 5.79 (s, 1H), 5.31 (s, 1H) 4.78 (d, J=14.4 Hz, 1H), 4.67 (d, J=15.4 Hz, 1H), 4.25 (d, J=14.4 Hz, 1H), 3.96 (bs, 1H, OH), 3.79 (s, 3H, O—CH₃), 1.67 (s, 3H, CH₃); ¹⁹F NMR (CDCl₃, 400 MHz) δ −62.07; MS (ESI) m/z 461.20 [M−H]⁻; 463.23 [M+H]⁺; HRMS (ESI) m/z calcd for C₂₀H₁₇F₃N₆O₄ 463.1342 [M+H]⁺ found 463.1326 [M+H]⁺; 485.1152 [M+Na]+.

### N-(4-Cyano-3-(trifluoromethyl)phenyl)-2-((5-fluoro-1H-indol-1-yl)methyl)acrylamide (C₂₀H₁₃F₄N₃O) (8)

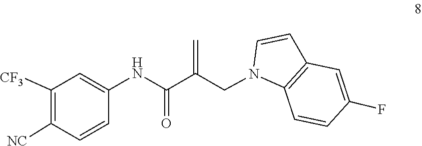 [SMILES: C=C(Cn1ccc2cc(F)ccc21)C(=O)Nc1ccc(C#N)c(C(F)(F)F)c1]

To a solution of 5-fluoro-indole (0.33 g, 0.002462 mol) in anhydrous THF (10 mL), which was cooled in an ice water bath under an argon atmosphere, was added sodium hydride (60% dispersion in oil, 0.30 g, 0.007385 mol). After addition, the resulting mixture was stirred for 3 h. 1-a (0.82 g, 0.002462 mol) was added to above solution, and the resulting reaction mixture was allowed to stir overnight at RT under argon. The reaction was quenched by water, and extracted with ethyl acetate. The organic layer was washed with brine, dried with MgSO₄, filtered, and concentrated under vacuum. The product was purified by a silica gel column using DCM and hexanes (2:1) as eluent to afford 30 mg (3.2%) of the titled compound as yellowish solid.

¹H NMR (400 MHz, DMSO-d₆) δ 10.74 (s, 1H, NH), 8.32 (s, 1H, ArH), 8.31-8.09 (m, 2H, ArH), 7.50-7.46 (m, 2H, ArH), 7.43 (d, J=3.2 Hz, 1H, ArH), 7.32 (dd, J=10.0 Hz, J=1.8 Hz, 1H, ArH), 7.00-6.95 (m, 2H, ArH), 6.45 (d, J=3.2 Hz, 1H, ArH), 6.05 (s, 1H, C═CH), 5.35 (s, 1H, C═CH), 5.14 (s, 2H, CH₂). HRMS [C₂₀H₁₄F₄N₃O⁺]: calcd 338.1073, found 338.1070 [M+H]⁺. Purity: 91.87% (HPLC).

### 4-(((5-Fluoro-1H-indol-1-yl)methyl)amino)-2-(trifluoromethyl)benzonitrile (C₁₇H₁₁F₄N₃) (15)

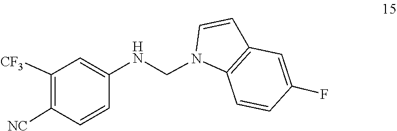 [SMILES: N#Cc1ccc(NCn2ccc3cc(F)ccc32)cc1C(F)(F)F]

Following the same synthesis as for 8, 15 and 16 were also synthesized as by-products. ¹H NMR (400 MHz, DMSO-d₆) δ 8.28 (t, J=6.4 Hz, 1H, NH), 7.77 (d, J=8.8 Hz, 1H, ArH), 7.71-7.68 (m, 1H, Indole-H), 7.66 (d, J=3.2 Hz, 1H, Indole-H), 7.31 (dd, J=9.6 Hz, J=1.8 Hz, 1H, Indole-H), 7.22 (d, J=2.0 Hz, 1H, ArH), 7.13 (dd, J=8.8 Hz, J=2.0 Hz, 1H, ArH), 7.02 (dt, J=9.2 Hz, J=2.8 Hz, 1H, Indole-H), 6.42 (d, J=2.8 Hz, 1H, Indole-H), 5.73 (d, J=6.8 Hz, 2H, CH₂). HRMS [C₁₇H₁₁F₄N₃Na⁺]: calcd 356.0787, found 356.0789 [M+H]⁺. Purity: 96.79% (HPLC).

### N-(4-Cyano-3-(trifluoromethyl)phenyl)-3-(5-fluoro-1H-indol-1-yl)-2-((5-fluoro-1H-indol-1-yl)methyl)propanamide (C₂₈H₁₉F₅N₄O) (16)

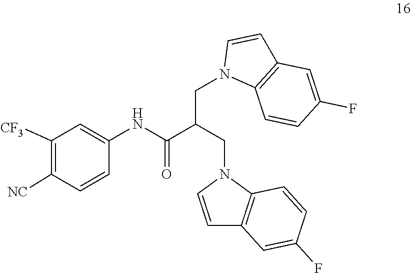 [SMILES: N#Cc1ccc(NC(=O)C(Cn2ccc3cc(F)ccc32)Cn2ccc3cc(F)ccc32)cc1C(F)(F)F]

Following the same synthesis as for 8, 15 and 16 were also synthesized as by-products. ¹H NMR (400 MHz, DMSO-d₆) δ 10.87 (s, 1H, NH), 8.57 (m, 2H, Pyrazole-H), 8.12 (d, J=1.6 Hz, 1H, ArH), 8.11 (d, J=8.2 Hz, 1H, ArH), 8.05 (m, 2H, Pyrazole-H), 7.85 (dd, J=8.2 Hz, J=1.6 Hz, 1H, ArH), 4.58-4.53 (m, 1H, CH₂), 4.48-4.43 (m, 1H, CH₂), 3.71-3.67 (m, 1H, CH). HRMS [C₂₈H₂₀F₅N₄O⁺]: calcd 523.1557, found [M+H]⁺. Purity:% (HPLC).

### (Z)—N-(4-Cyano-3-(trifluoromethyl)phenyl)-3-iodobut-2-enamide (C₁₂H₈F₃IN₂O) (1-b)

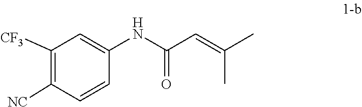 [SMILES: CC(C)=CC(=O)Nc1ccc(C#N)c(C(F)(F)F)c1]

(Z)-3-Iodobut-2-enoic acid (2.50 g, 0.011973 mol) reacted with thionyl chloride (1.68 g, 0.014152 mol), trimethylamine (1.55 g, 0.01533 mol), and 4-amino-2-(trifluoromethyl)benzonitrile (2.20 g, 0.011973 mol) to afford the titled compound. The product was purified by a silica gel column using hexanes and ethyl acetate (2:1) as eluent to afford 2.54 g (56.7%) of the titled compound as light brown oil.

¹H NMR (400 MHz, DMSO-d₆) δ 10.98 (s, 1H, NH), 8.31 (d, J=2.0 Hz, 1H, ArH), 8.09 (d, J=8.2 Hz, 1H, ArH), 7.98 (dd, J=8.2 Hz, J=2.0 Hz, 1H, ArH), 6.65 (d, J=1.6 Hz, 1H, C═CH), 2.71 (s, 3H, CH₃). HRMS [C₁₂H₉F₃IN₂O+]: calcd 380.9712, found 380.9704 [M+H]⁺. Purity: 95.89% (HPLC).

### (E)-N-(4-Cyano-3-(trifluoromethyl)phenyl)-3-(4-fluoro-1H-pyrazol-1-yl)but-2-enamide (C₁₅H₁₀F₄N₄O) (9)

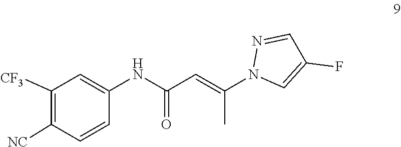 [SMILES: C/C(=C\C(=O)Nc1ccc(C#N)c(C(F)(F)F)c1)n1cc(F)cn1]

To a mixture of 4-fluoro-1H-pyrazole (0.103 g, 0.0012 mol) in anhydrous toluene (5 mL) was added 1-b (0.228 g, 0.0006 mol), KOBu-t (0.081 g, 0.00072 mol), Pd(OAc)₂ (14 mg, 0.00006 mol), and (R)-(+)-2,2′-bis(diphenylphosphino)-1,1′-binaphthalene (BINAP, 38 mg, 0.00006 mol) at RT under the argon atmosphere. The reaction mixture was heated at reflux for 5-6 h under the argon atmosphere. After the end of the reaction was established by TLC, the reaction was quenched by water, and extracted with ethyl acetate. The organic layer was dried with MgSO₄, filtered, and concentrated under vacuum. The product was purified by a silica gel column using DCM and ethyl acetate (19:1 to 9:1) as eluent to afford 15 mg (7.4%) of the desired compound as yellowish solid.

¹H NMR (400 MHz, DMSO-d₆) δ 10.97 (s, 1H, NH), 8.47 (d, J=8.2 Hz, 1H, Pyrazole-H), 8.36 (d, J=1.6 Hz, 1H, ArH), 8.10 (d, J=8.4 Hz, 1H, ArH), 7.99 (dd, J=8.4 Hz, J=1.6 Hz, 1H, ArH), 7.96 (d, J=8.2 Hz, 1H, Pyrazole-H), 6.81 (s, 1H, C═CH), 2.71 (s, 3H, CH₃). HRMS [C₁₅H₁₁F₄N₄O⁺]: calcd 339.0869, found 339.0868 [M+H]⁺. Purity: 99.30% (HPLC).

### (Z)—N-(4-Cyano-3-(trifluoromethyl)phenyl)-3-(4-fluoro-1H-pyrazol-1-yl)but-2-enamide (C₁₅H₁₀F₄N₄O) (10)

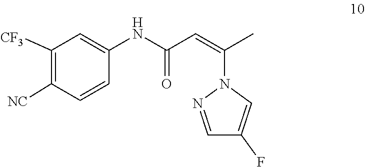 [SMILES: C/C(=C/C(=O)Nc1ccc(C#N)c(C(F)(F)F)c1)n1cc(F)cn1]

To a mixture of 4-fluoro-1H-pyrazole (0.103 g, 0.0012 mol) in in anhydrous toluene (5 mL) was added 1-b (0.228 g, 0.0006 mol), KOBu-t (0.081 g, 0.00072 mol), Pd(OAc)₂ (14 mg, 0.00006 mol), and (R)-(+)-2,2′-bis(diphenylphosphino)-1,1′-binaphthalene (BINAP, 38 mg, 0.00006 mol) at RT under the argon atmosphere. The reaction mixture was heated at reflux for 5-6 h under the argon atmosphere. After the end of the reaction was established by TLC, the reaction was quenched by water, and extracted with ethyl acetate. The organic layer was dried with MgSO₄, filtered, and concentrated under vacuum. The product was purified by a silica gel column using DCM and ethyl acetate (19:1 to 9:1) as eluent to afford 37 mg (18.2%) of the desired compound as pinkish solid.

¹H NMR (400 MHz, DMSO-d₆) δ 10.83 (s, 1H, NH), 8.31 (d, J=8.0 Hz, 1H, Pyrazole-H), 8.24 (s, 1H, ArH), 8.08 (d, J=8.2 Hz, 1H, ArH), 7.93 (dd, J=8.2 Hz, J=1.6 Hz, 1H, ArH), 7.73 (d, J=8.2 Hz, 1H, Pyrazole-H), 5.91 (d, J=1.2 Hz, 1H, C═CH), 2.30 (s, 3H, CH₃). HRMS [C₁₅H₁₁F₄N₄O⁺]: calcd 339.0869, found 339.0876 [M+H]⁺. Purity: 99.81% (HPLC).

### (S)-Methyl 2-(((1-((4-cyano-3-(trifluoromethyl)phenyl)amino)-3-(4-fluoro-1H-pyrazol-1-yl)-2-methyl-1-oxopropan-2-yl)oxy)methyl)acrylate (C₂₀H₁₈F₄N₄O₄) (11)

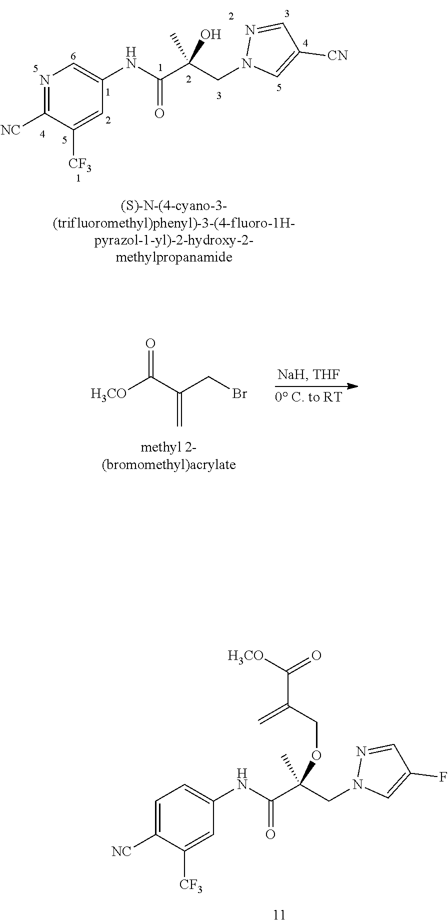

A solution of methyl 2-(bromomethyl)acrylate (0.61 mL, 4.9 mmol) in 10 mL of THF was treated with (S)—N-(4-cyano-3-(trifluoromethyl)phenyl)-3-(4-fluoro-1H-pyrazol-1-yl)-2-hydroxy-2-methylpropanamide (529 mg, 1.48 mmol) portion wise over 10 min at RT. The solution was then stirred at RT. The solution was then stirred overnight at RT and the solution concentrated in vacuo. The residue was then taken up in water and extracted four times with ethyl acetate. The combined ethyl acetate solution was washed with saturated sodium chloride, dried over anhydrous magnesium sulfate, filtered and concentrated. The residue was then purified by silica gel column chromatography eluting with hexane/ethyl acetate 1:1, to give desired product as a colorless oil.

¹H NMR (CDCl₃, 400 MHz) δ 10.27 (bs, 1H, NH—C(O)), 8.29 (d, J=2.0 Hz, 1H), 8.21 (dd, J=8.8, 2.0 Hz, 1H), 7.79 (d, J=2.0 Hz, 1H), 7.29 (d, J=4.8 Hz, 1H), 7.25 (d, J=4.8 Hz, 1H), 6.45 (s, 1H), 6.02 (s, 1H), 4.39 (d, J=14.4 Hz, 1H), 4.32 (d, J=14.4 Hz, 1H), 4.36 (d, J=9.6 Hz, 1H), 4.07 (d, J=9.6 Hz, 1H), 3.91 (s, 3H, O—CH₃), 1.52 (s, 3H, CH₃); ¹⁹F NMR (CDCl₃, 400 MHz) δ −62.30, −176.86. MS (ESI) m/z 455.13 [M+H]⁺; HRMS (ESI) m/z calcd for C20H1sF4N₄Q4 455.1342 [M+H]⁺ found 463.1333 [M+H]⁺.

### (E)-N-(4-Cyano-3-(trifluoromethyl)phenyl)-3-(4-fluoro-1H-pyrazol-1-yl)acrylamide (C₁₄H₈F₄N₄O) (12)

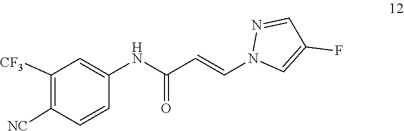 [SMILES: N#Cc1ccc(NC(=O)/C=C/n2cc(F)cn2)cc1C(F)(F)F]

To a solution of 4-fluoro-1H-pyrazole (0.103 g, 0.00116 mol) in anhydrous THF (10 mL), which was cooled in an ice water bath under an argon atmosphere, was added sodium hydride (60% dispersion in oil, 0.14 g, 0.003479 mol). After addition, the resulting mixture was stirred for 3 h. (E)-3-Bromo-N-(4-cyano-3-(trifluoromethyl)phenyl)acrylamide (0.37 g, 0.00116 mol) was added to above solution, and the resulting reaction mixture was allowed to stir overnight at RT under argon. The reaction was quenched by water, and extracted with ethyl acetate. The organic layer was washed with brine, dried with MgSO₄, filtered, and concentrated under vacuum. The product was purified by a silica gel column using DCM and ethyl acetate (19:1) as eluent to afford 0.143 g (38%) of the titled compound as white solid.

¹H NMR (400 MHz, DMSO-d₆) δ 11.00 (s, 1H, NH), 8.39 (d, J=4.4 Hz, 1H, Pyrazole-H), 8.32 (d, J=2.0 Hz, 1H, ArH), 8.13 (d, J=8.4 Hz, 1H, ArH), 8.08 (d, J=13.6 Hz, 1H, CH═C), 8.04 (dd, J=8.4 Hz, J=2.0 Hz, 1H, ArH), 7.98 (d, J=4.0 Hz, 1H, Pyrazole-H), 6.72 (d, J=13.6 Hz, 1H, C═CH).

### (E)-N-(4-Cyano-3-(trifluoromethyl)phenyl)-3-(4-fluorophenyl)-2-methylacrylamide (C₁₅H₁₂F₄N₂O) (13)

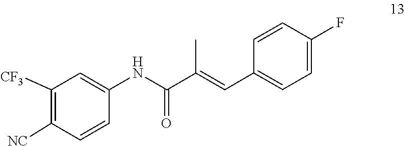 [SMILES: C/C(=C\c1ccc(F)cc1)C(=O)Nc1ccc(C#N)c(C(F)(F)F)c1]

(E)-3-(4-Fluorophenyl)-2-methylacrylic acid (1.00 g, 5.55 mmol) was dissolved in 10 mL of dry THF. Thionyl chloride (0.99 g, 0.61 mL, 8.325 mmol) was slowly added to the reaction mixture over 10 minutes while maintaining the reaction temperature below 10° C. The reaction mixture was stirred for 2 h. The reaction was cooled to 0° C. Triethylamine (1.68 g, 2.32 mL, 0.01665 mol) was slowly added to the reaction mixture, keeping the temperature below 10° C. 4-Amino-2-(trifluoromethyl) benzonitrile (1.03 g, 5.55 mmol) and THF (5 mL) were then charged to the batch. The batch was then heated to 50±5° C. and agitated for 2 h. The batch was then cooled to 20±5° C. followed by the addition of water (20 mL) and ethyl acetate (20 mL). After brief agitation the layers were separated. The organic layer was washed with water (15 mL). The batch was then concentrated to dryness and purified via silica gel column using DCM and ethyl acetate (19:1) as eluent to afford 1.22 g (63.2%) of title compound as yellow solid.

¹H NMR (400 MHz, DMSO-d₆) δ 10.57 (s, 1H, NH), 8.45 (d, J=2.0 Hz, 1H, ArH), 8.29 (dd, J=8.8 Hz, J=2.0 Hz, 1H, ArH), 8.21 (d, J=8.8 Hz, 1H, ArH), 7.64-7.61 (m, 2H, ArH), 7.46 (s, 1H, C═CH), 7.39-7.34 (m, 2H, ArH), 2.18 (d, J=0.8 Hz, 3H, CH₃).

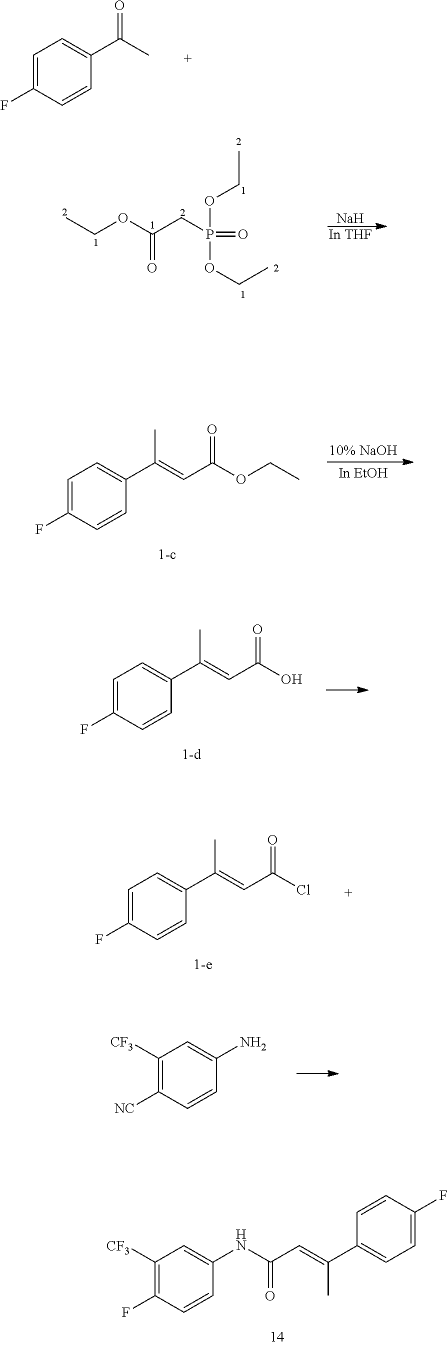

Sodium hydride (1.30 g, 0.032576 mol, 1.5 equiv, 60% in mineral oil) was dissolved in THF (100 mL), and triethyl phosphonoacetate (6.846 g, 0.032576 mol, 1.5 equiv) was added dropwise to the suspension at 0° C. under argon. The mixture was stirred until gas evolution had ceased. Then, 1-(4-fluorophenyl)ethanone (3.00 g, 0.21717 mol, 1.0 equiv) in THF (10 mL) was added by syringe. The reaction was stirred at RT and monitored by TLC. The reaction mixture was quenched with saturated aqueous NH₄Cl solution. The organic phase was separated, and the aqueous layer was extracted with EtOAc. The combined organic phases were washed with saturated aqueous NaCl solution, dried over anhydrous MgSO₄, and concentrated under vacuum pressure. Product was purified by silica gel chromatography using hexanes and ethyl acetate (4:1) to give 4.30 g (95%) of ethyl 3-(4-fluorophenyl)but-2-enoate as an oil.

### 3-(4-Fluorophenyl)but-2-enoic acid (C₁₀H₉FO₂) (1-d)

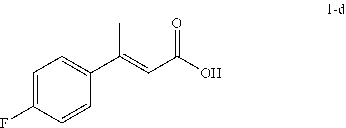

To a solution of 1-c (2.00 g, 0.009605 mol) in 20 mL of EtOH was added an aqueous NaOH solution (10%, 40 mL) at RT under argon. The resulting reaction mixture was stirred until no raw material was monitored by TLC. The mixture was acidified with 1 N HCl, and then extracted with diethyl ether. The combined organic phase was washed with saturated aqueous NaCl solution, dried over MgSO₄, and concentrated under vacuum pressure. Product was purified by recrystallization (CH₂Cl₂ vs Et₂O) to afford 1.56 g (90%) of 3-(4-fluorophenyl)but-2-enoic acid as white solid.

### N-(4-Cyano-3-(trifluoromethyl)phenyl)-3-(4-fluorophenyl)but-2-enamide (C₁₈H₁₂F₄N₂O) (14)

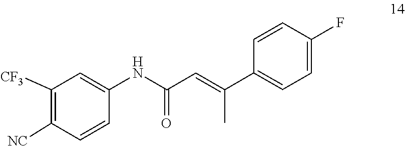 [SMILES: C/C(=C\C(=O)Nc1ccc(C#N)c(C(F)(F)F)c1)c1ccc(F)cc1]

1-d (1.00 g, 5.55 mmol) was dissolved in 10 mL of dry THF. Thionyl chloride (0.99 g, 0.61 mL, 8.325 mmol) was slowly added to the reaction mixture over 10 minutes while maintaining the reaction temperature below 10° C. to produce 1-e. The reaction mixture was stirred for 2 h. The reaction was cooled to 0° C. Without isolation of 1-e, triethylamine (1.68 g, 2.32 mL, 0.01665 mol) was slowly added to the reaction mixture, keeping the temperature below 10° C. 4-Amino-2-(trifluoromethyl) benzonitrile (1.03 g, 5.55 mmol) and THF (5 mL) were then charged to the batch. The batch was then heated to 50±5° C. and agitated for 2 h. The batch was then cooled to 20±5° C. followed by the addition of water (20 mL) and ethyl acetate (20 mL). After brief agitation the layers were separated. The organic layer was then washed with water (15 mL). The batch was then concentrated to dryness and purified by a silica gel column using hexanes and ethyl acetate (3:1) as eluent to afford 0.44 g (23%) of title compound as yellow solid.

¹H NMR (400 MHz, DMSO-d₆) δ 10.85 (s, 1H, NH), 8.35 (d, J=2.0 Hz, 1H, ArH), 8.10 (d, J=8.8 Hz, 1H, ArH), 7.99 (dd, J=8.8 Hz, J=2.0 Hz, 1H, ArH), Use Gap Code 7.64-7.61 (m, 2H, ArH), 7.31-7.27 (m, 2H, ArH), 6.39 (d, J=1.2 Hz, 1H, C═CH), 2.56 (d, J=0.8 Hz, 3H, CH₃).

### Preparation of Compound 16

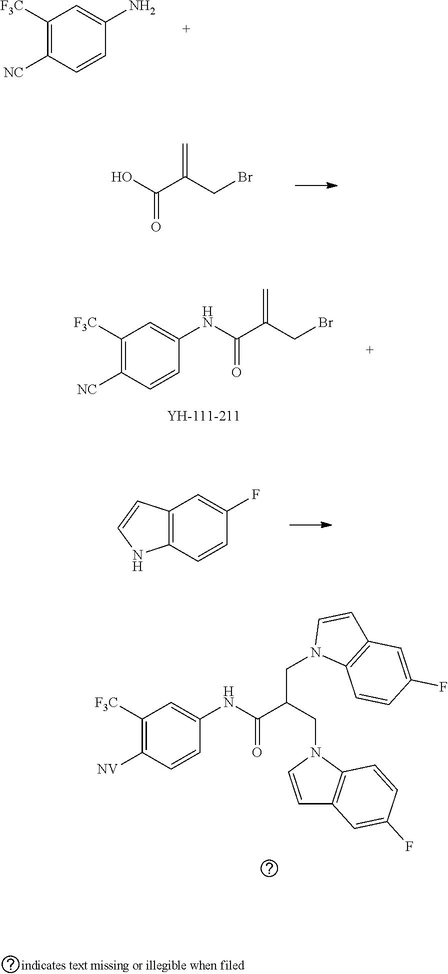

To a solution of 5-fluoro-indole (0.33 g, 0.002462 mol) in anhydrous THF (10 mL), which was cooled in an ice water bath under an argon atmosphere, was added sodium hydride (60% dispersion in oil, 0.30 g, 0.007385 mol). After addition, the resulting mixture was stirred for three hours. 2-(bromomethyl)-N-(4-cyano-3-(trifluoromethyl) phenyl)acrylamide (0.82 g, 0.002462 mol) was added to above solution, and the resulting reaction mixture was allowed to stir overnight at room temperature under argon. The reaction was quenched by water, extracted with ethyl acetate. The organic layer was washed with brine, dried with MgSO₄, filtered, and concentrated under vacuum. The product was purified by a silica gel column using DCM and hexanes (2:1) as eluent to afford 26 mg (2.05%) of the titled compound as yellowish solid.

¹H NMR (400 MHz, DMSO-d₆) δ 10.87 (s, 1H, NH), 8.57 (m, 2H, Pyrazole-H), 8.12 (d, J=1.6 Hz, 1H, ArH), 8.11 (d, J=8.2 Hz, 1H, ArH), 8.05 (m, 2H, Pyrazole-H), 7.85 (dd, J=8.2 Hz, J=1.6 Hz, 1H, ArH), 4.58-4.53 (m, 2H, CH₂), 4.48-4.43 (m, 2H, CH₂), 3.71-3.67 (m, 1H, CH); HRMS [C₂₈H₂₀F₅N₄O⁺]: calcd 523.1557, found [M+H]⁺.

### Preparation of (S)-Methyl 2-(((1-((6-cyano-5-(trifluoromethyl)pyridin-3-yl)amino)-3-(4-fluoro-1H-pyrazol-1-yl)-2-methyl-1-oxopropan-2-yl)oxy)methyl)acrylate (17) and (S)-Methyl 2-(((3-(4-cyano-1H-pyrazol-1-yl)-1-((4-cyano-3-(trifluoromethyl)phenyl) amino)-2-methyl-1-oxopropan-2-yl)oxy)methyl)acrylate (18)

A solution of methyl 2-(bromomethyl)acrylate (0.71 mL, 5.7 mmol) in 10 mL of THF was treated with aryl propanamide (620 mg, 1.27 mmol) portion wise over 10 min at ice bath and the solution was raised to room temperature then stirred overnight at the room temperature and the solution concentrated in vacuo. The residue was then taken up in water and extracted three times with ethyl acetate. The combined ethyl acetate solution was washed with saturated sodium chloride, dried over anhydrous magnesium sulfate (MgSO₄), filtered and concentrated. The residue was then purified by silica gel column chromatography eluting with hexane/ethyl acetate (1:1, v/v) to give desired product.

### (S)-Methyl 2-(((1-((6-cyano-5-(trifluoromethyl)pyridin-3-yl)amino)-3-(4-fluoro-1H-pyrazol-1-yl)-2-methyl-1-oxopropan-2-yl)oxy)methyl)acrylate (17)

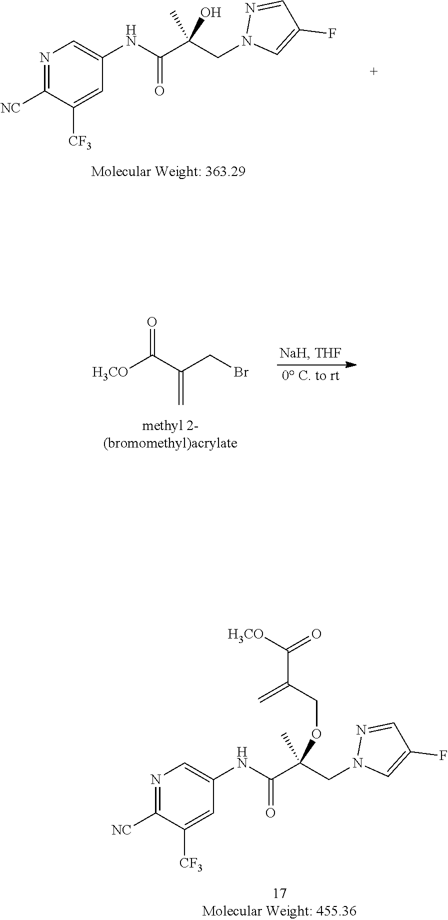

For aryl propanamide: (S)—N-(6-cyano-5-(trifluoromethyl) pyridin-3-yl)-3-(4-fluoro-1H-pyrazol-1-yl)-2-hydroxy-2-methylpropanamide-yield=49% (as colorless oil); UV max: 196.45, 275.45; HPLC: t_{R} 3.25 min, purity 98.57%; MS (ESI) m/z 456.07 [M+H]⁺; 478.05 [M+Na]⁺; [00442] HRMS (ESI) m/z calcd for C₁₉H₁₇F₄N₅O₄ Exact Mass: 456.1295 C₁₉H₁₇F₄N₅O₄ found 456.1295 [M+H]⁺;

¹H NMR (CDCl₃, 400 MHz) δ 10.53 (bs, 1H, NH—C(O)), 9.14 (d, J=2.4 Hz, 1H), 8.88 (d, J=2.4 Hz, 1H), 7.29 (d, J=4.8 Hz, 1H), 7.23 (d, J=4.8 Hz, 1H), 6.47 (s, H), 6.05 (s, 1H), 4.39 (d, J=14.4 Hz, 1H), 4.32 (d, J=9.6 Hz, 1H), 4.29 (d, J=14.4 Hz, 1H), 4.08 (d, J=9.6 Hz, 1H), 4.91 (s, 3H, O—CH₃), 1.54 (s, 3H, CH₃);

¹⁹F NMR (CDCl₃, 400 MHz) δ −62.16, −176.77;

¹³C NMR (CDCl₃, 100 MHz) δ172.13, 167.80, 150.88, 148.43, 144.47, 137.67, 134.95, 131.75, 131.11 (q, J=34 Hz), 126.71, 126.58, 124.76 (d, J=2.0 Hz), 123.75 (q, J=4.0 Hz), 121.68 (q, J=275.0 Hz), 117.05, 116.77, 114.42, 81.52, 65.49, 60.19, 52.91, 18.14.

### (S)-Methyl 2-(((3-(4-cyano-1H-pyrazol-1-yl)-1-((4-cyano-3-(trifluoromethyl)phenyl)amino)-2-methyl-1-oxopropan-2-yl)oxy)methyl)acrylate (18)

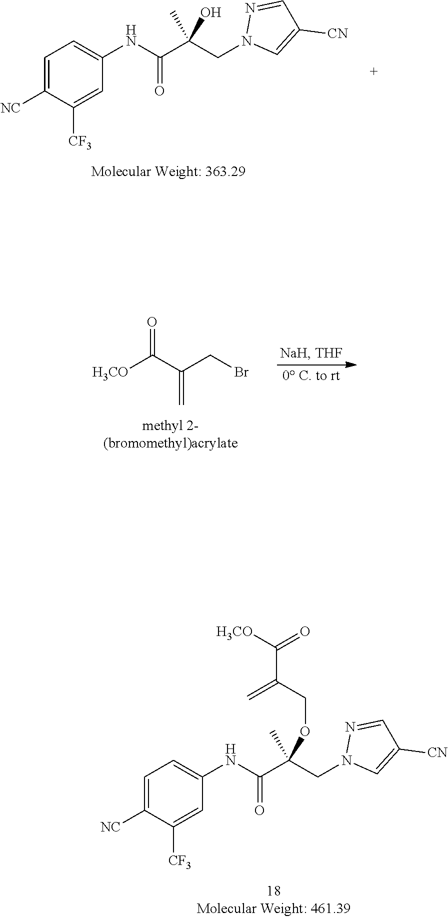

For aryl propanamide: (S)-3-(4-cyano-1H-pyrazol-1-yl)-N-(4-cyano-3-(trifluoromethyl)phenyl)-2-hydroxy-2-methylpropanamide-yield=52% (as colorless oil); UV max: 196.45, 271.45; HPLC: t_{R} 3.16 min, purity 96.38%; MS (ESI) m/z 462.07 [M+H]⁺; 484.06 [M+Na]⁺; HRMS (ESI) m/z calcd for C₂₁H₁₈F₃N₅O₄ 462.1389 [M+H]⁺ found 462.1396 [M+H]⁺; 484.1215 [M+Na]⁺;

¹H NMR (CDCl₃, 400 MHz) δ 10.31 (bs, 1H, NH—C(O)), 8.30 (s, 1H), 8.17 (d, J=8.8 Hz, 1H), 7.83 (s, 1H), 7.80 (d, J=8.8 Hz, 1H), 7.70 (s, H), 6.48 (s, 1H), 6.04 (s, 1H), 4.53 (d, J=14.4 Hz, 1H), 4.42 (d, J=14.4 Hz, 1H), 4.35 (d, J=9.2 Hz, 1H), 4.06 (d, J=9.2 Hz, 1H), 4.94 (s, 3H, O—CH₃), 1.56 (s, 3H, CH₃);

¹⁹F NMR (CDCl₃, 400 MHz) δ −62.30;

¹³C NMR (CDCl₃, 100 MHz) δ 170.85, 167.56, 142.10, 141.95, 136.15, 135.76, 134.85, 133.59 (q, J=36 Hz), 131.76, 122.23 (q, J=272 Hz), 122.20, 117.91 (q, J=5 Hz), 115.69, 133.20, 104.45, 92.70, 81.01, 65.46, 59.59, 52.89, 18.34.

### Example 2: Androgen Receptor Binding, Transactivation, Degradation, and Metabolism Ligand Binding Assay (Kᵢ Values)

Objective: To determine SARCAs binding affinity to the AR-LBD.

Method: Ligand binding assay (ki): hAR-LBD (633-919) was cloned into pGex4t.1. Large scale GST-tagged AR-LBD was prepared and purified using a GST column. Recombinant AR-LBD was combined with [3H]mibolerone (PerkinElmer, Waltham, MA) in buffer A (10 mM Tris, pH 7.4, 1.5 mM disodium EDTA, 0.25 M sucrose, 10 mM sodium molybdate, 1 mM PMSF) to determine the equilibrium dissociation constant (K_{d}) of [³H]mibolerone. Protein was incubated with increasing concentrations of [³H]mibolerone with and without a high concentration of unlabeled mibolerone at 4° C. for 18 h in order to determine total and non-specific binding. Non-specific binding was then subtracted from total binding to determine specific binding and non-linear regression for ligand binding curve with one site saturation to determine the K_{d} of mibolerone.

Increasing concentrations of SARCAs or DHT (range: 10⁻¹² to 10⁻² M) were incubated with [³H]mibolerone and AR LBD using the conditions described above. Following incubation, the ligand bound AR-LBD complex was isolated using Bio Gel HT® hydroxyapatite, washed and counted in a scintillation counter after adding scintillation cocktail. Values are expressed as Kᵢ.

Transactivation Assay with wt AR (IC₅₀ values): to determine the effect of SARCAs on androgen-induced transactivation of AR wildtype (wt).

Method: HEK-293 cells were plated at 125,000 cells/well of a 24 well plate in DME+5% csFBS without phenol red. Cells were transfected with 0.25 μg GRE-LUC, 10 ng CMV-renilla LUC, and 50 ng CMV-hAR(wt) using Lipofectamine transfection reagent in optiMEM medium. Medium was changed 24 h after transfection to DME+5% csFBS without phenol red and treated with a dose response of various drugs (1 pM to 10 μM). SARCAs and antagonists were treated in combination with 0.1 nM R1881. Luciferase assay was performed 24 h after treatment on a Biotek synergy 4 plate reader. Firefly luciferase values were normalized to renilla luciferase values.

Plasmid constructs and transient transfection.

Human AR cloned into CMV vector backbone was used for the transactivation study. HEK-293 cells were plated at 120,000 cells per well of a 24 well plate in DME+5% csFBS. The cells were transfected using Lipofectamine (Invitrogen, Carlsbad, CA) with 0.25 μg GRE-LUC, 0.01 kg CMV-LUC (renilla luciferase) and 25 ng of the AR. The cells were treated 24 h after transfection as indicated in the figures and the luciferase assay performed 48 h after transfection. Data are represented as IC₅₀ obtained from four parameter logistics curve.

LNCaP Gene Expression Assay.

Method: LNCaP cells were plated at 15,000 cells/well of a 96 well plate in RPMI+1% csFBS without phenol red. Forty-eight hours after plating, cells were treated with a dose response of SARCAs. Twenty four hours after treatment, RNA was isolated using cells-to-ct reagent, cDNA synthesized, and expression of various genes was measured by realtime rtPCR (ABI 7900) using taqman primers and probes. Gene expression results were normalized to GAPDH. (See results at Example 14 below.) LNCaP growth assay.

Method: LNCaP cells were plated at 10,000 cells/well of a 96 well plate in RPMI+1% csFBS without phenol red. Cells were treated with a dose response of SARCAs. Three days after treatment, cells were treated again. Six days after treatment, cells were fixed and cell viability was measured by SRB assay.

LNCaP or ADl degradation (AR FL).

Method: LNCaP or ADl cells expressing full length AR were plated at 750,000-1,000,000 cells/well of a 6 well plate in growth medium (RPMI+10% PBS). Twenty four hours after plating, medium was changed to RPMI+1% csFBS without phenol red and maintained in this medium for 2 days. Medium was again changed to RPMI+1% csPBS without phenol red and cells were treated with SARCAs (1 nM to 10 μM) in combination with 0.1 nM R1881. After 24 h of treatment, cells were washed with cold PBS and harvested. Protein was extracted using salt-containing lysis buffer with three free-thaw cycles. Protein concentration was estimated and five microgram of total protein was loaded on a SDS-PAGE, fractionated, and transferred to a PVDF membrane. The membrane was probed with AR N-20 antibody from SantaCruz and actin antibody from Sigma.

22RV1 and D567es degradation (AR SV).

Method: 22RV1 and D567es cells expressing AR splice variants were plated at 750,000-1,000,000 cells/well of a 6 well plate in growth medium (RPMI+10% PBS). Twenty four hours after plating, medium was changed and treated. After 24-30 h of treatment, cells were washed with cold PBS and harvested. Protein was extracted using salt-containing lysis buffer with three free-thaw cycles. Protein concentration was estimated and five microgram of total protein was loaded on a SDS-PAGE, fractionated, and transferred to a PVDF membrane. The membrane was probed with AR N-20 antibody from SantaCruz and actin antibody from Sigma.

22RV1 growth and gene expression.

Methods: Cell growth was evaluated as described before by SRB assay. Cells were plated in a 96 well plate in full serum and treated for 6 days with medium change after day 3. Gene expression studies were performed in 22RV1 cells plated in 96 well plate at 10,000 cells/well in RPMI+10% PBS. Twenty four hours after plating, cells were treated for 3 days and gene expression studies were performed as described before.

Transient Transfection (IC₅₀)

Methods: Human AR cloned into CMV vector backbone was used for the transactivation study. COS7 cells were plated at 30,000 cells per well of a 24 well plate in DME+5% csFBS. The cells were transfected using Lipofectamine (Invitrogen, Carlsbad, CA) with 0.25 μg GRE-LUC, 0.02 μg CMV-LUC (renilla luciferase) and 25 ng of the AR. The cells were treated 24 hrs after transfection as indicated in the figures and the luciferase assay performed 48 hrs after transfection. Data are represented as IC₅₀ obtained from four parameter logistics curve.

AR and AR-SV Degradation

Methods: LNCaP cells (AR) and 22RV1 cells (AR-SV) were plated in RPMI+1% csFBS w/o phenol red medium. Cells were treated 2 days after plating and the cells were harvested 24 hours after treatment. Protein was extracted and Western blot for AR and AR-SV was performed. The numbers under each lane represents the % change from vehicle. The bands were quantified using Image software. For each lane, the AR band was divided by GAPDH band and the % difference from vehicle was calculated and represented under each lane. The numbers shown are 0 (no degradation) or represented as decreases in AR levels normalized for GAPDH levels (some values are represented as positive but still indicate degradation).

Determination of Metabolic Stability (In Vitro CLᵢₙₜ) of Test Compounds

Phase I Metabolism

The assay was done in a final volume of 0.5 ml in duplicates (n=2). Test compound (1 μM) was pre-incubated for 10 minutes at 37° C. in 100 mM Tris-HCl, pH 7.5 containing 0.5 mg/ml liver microsomal protein. After pre-incubation, reaction was started by addition of 1 mM NADPH (pre-incubated at 37° C.). Incubations were carried out in triplicate and at various time-points (0, 5, 10, 15, 30 and 60 minutes) 100 μl aliquots were removed and quenched with 100 μl of acetonitrile containing internal standard. Samples were vortex mixed and centrifuged at 4000 rpm for 10 minutes. The supernatants were transferred to 96 well plates and submitted for LC-MS/MS analysis. As control, sample incubations done in absence of NADPH were included. From % PCR (% Parent Compound Remaining), rate of compound disappearance is determined (slope) and in vitro CLᵢₙₜ (μL/min/mg protein) was calculated.

Metabolic Stability in Phase I & Phase II Pathways

In this assay, test compound was incubated with liver microsomes and disappearance of drug was determined using discovery grade LC-MS/MS. To stimulate Phase II metabolic pathway (glucuronidation), UDPGA and alamethicin was included in the assay.

LC-MS/MS Analysis

The analysis of the compounds under investigation was performed using LC-MS/MS system consisting of Agilent 1100 HPLC with an MDS/Sciex 4000 Q-Trap™ mass spectrometer. The separation was achieved using a Cis analytical column (Alltima™, 2.1×100 mm, 3 m) protected by a Cis guard cartridge system (SecurityGuard™ ULTRA Cartridges UHPLC for 4.6 mm ID columns, Phenomenex). Mobile phase was consisting of channel A (95% acetonitrile+5% water+0.1% formic acid) and channel C (95% water+5% acetonitrile+0.1% formic acid) and was delivered at a flow rate of 0.4 mL/min. The volume ratio of acetonitrile and water was optimized for each of the analytes. Multiple reaction monitoring (MRM) scans were made with curtain gas, collision gas, nebulizer gas, and auxiliary gas optimized for each compound, and source temperature at 550° C. Molecular ions were formed using an ion spray voltage of −4200 V (negative mode). Declustering potential, entrance potential, collision energy, product ion mass, and cell exit potential were optimized for each compound.

LC-MS/MS Analysis for Determining Rat Serum Concentrations

Serum was collected 24-30 h after last dose. 100 μL of serum was mixed with 200 μL of acetonitrile/internal standard. Standard curves were prepared by serial dilution of standards in nM with 100 μL of rat serum, concentrations were 1000, 500, 250, 125, 62.5, 31.2, 15.6, 7.8, 3.9, 1.9, 0.97, and 0. Standards were with extracted with 200 μL of acetonitrile/internal standard. The internal standard for these experiments was (S)-3-(4-cyanophenoxy)-N-(3-(chloro)-4-cyanophenyl)-2-hydroxy-2-methylpropanamide.

The instrumental analysis of the analyte SARCA was performed using LC-MS/MS system consisting of Agilent 1100 HPLC with an MDS/Sciex 4000 Q-Trap™ mass spectrometer. The separation was achieved using a Cis analytical column (Alltima™, 2.1×100 mm, 3 m) protected by a C1s guard column (Phenomenex™ 4.6 mm ID cartridge with holder). Mobile phase was consisting of channel A (95% acetonitrile+5% water+0.1% formic acid) and channel C (95% water+5% acetonitrile+0.1% formic acid) and was delivered isocratically at a flow rate of 0.4 mL/min at 70% A and 30% B. The total runtime for analyte SARCA was optimized but generally 2-4 minutes, and the volume injected was 10 μL. Multiple reaction monitoring (MRM) scans were made with curtain gas at 10; collision gas at medium; nebulizer gas at 60.0 and auxiliary gas at 60.0 and source temperature at 550° C. Molecular ions were formed using an ion spray voltage (IS) of 4200 (negative mode). Declustering potential (DP), entrance potential (EP), collision energy (CE), product ion mass, and cell exit potential (CXP) were optimized for each analyte SARCA for the mass pair observed.

Log P: Octanol-Water Partition Coefficient (Log P)

Log P is the log of the octanol-water partition coefficient, commonly used early in drug discovery efforts as a rough estimate of whether a particular molecule is likely to cross biological membranes. Log P was calculated using ChemDraw Ultra version is 12.0.2.1016 (Perkin-Elmer, Waltham, Massachusetts 02451). Calculated Log P values are reported in Table 1 in the column labeled ‘Log P (−0.4 to +5.6)’. Lipinski's rule of five is a set of criteria intended to predict oral bioavailability. One of these criteria for oral bioavailability is that the Log P is between the values shown in the column heading (−0.4 (relatively hydrophilic) to +5.6 (relatively lipophilic) range), or more generally stated <5.

TABLE 1
In vitro screening of LBD binding (Kᵢ), AR antagonism (IC₅₀) SARD activity, and metabolic stability

| | | | | Binding/Wt. | | SARD Activity | | |
|---|---|---|---|---|---|---|---|---|
| | | | | | | Full Length | S.V. | DMPK(MLM) |
| | | | | Kᵢ (nm) | | % | (22RV1) % | T_{1/2} (min) |
| Compd ID | | Log P | | (DHT= | IC₅₀ | degradation | degradation | CLᵢₙₜ |
| (Scaffold) | Structure | (−0.4 to +5.6) | M.W | 1nM) | (nM) | at 1,10 μm | at 10 uM | (μg/min/mg) |
| Enzalutamide | 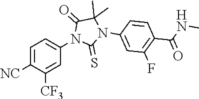 [SMILES: CNC(=O)c1ccc(N2C(=S)N(c3ccc(C#N)c(C(F)(F)F)c3)C(=O)C2(C)C)cc1F] | 4.56 | 464.44 | 3641.29 | 216.3 | | | |
| Enobosarm | 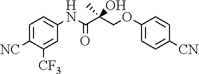 [SMILES: C[C@](O)(COc1ccc(C#N)cc1)C(=O)Nc1ccc(C#N)c(C(F)(F)F)c1] | 3.44 | 389.89 | 20.21 | ~20 (EC₅₀ value) | | | |
| R-Bicalutamide | 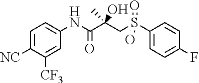 [SMILES: C[C@](O)(CS(=O)(=O)c1ccc(F)cc1)C(=O)Nc1ccc(C#N)c(C(F)(F)F)c1] | 2.57 | 430.37 | 508.84 | 248.2 | | | |
| Enzalutamide | 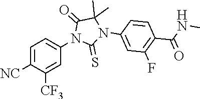 [SMILES: CNC(=O)c1ccc(N2C(=S)N(c3ccc(C#N)c(C(F)(F)F)c3)C(=O)C2(C)C)cc1F] | 4.56 | 464.44 | 3641.29 | 216.3 | | | |
| ARN-509 | 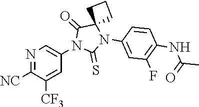 | 3.47 | 477.43 | 1452.29 | | 0 | 0 | |
| UT-34 | 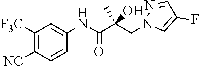 [SMILES: C[C@](O)(Cn1cc(F)cn1)C(=O)Nc1ccc(C#N)c(C(F)(F)F)c1] | 2.03 | 356.27 | No binding | 199.36 | 100 | 100 | 77.96/0.89 |
| 1 | 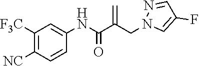 [SMILES: C=C(Cn1cc(F)cn1)C(=O)Nc1ccc(C#N)c(C(F)(F)F)c1] | 2.67 | 338.26 | 3276.78 | 1330 | 0 | 57 | Infinity 0.000 |
| 2 | 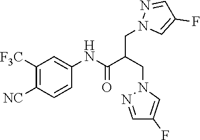 [SMILES: N#Cc1ccc(NC(=O)C(Cn2cc(F)cn2)Cn2cc(F)cn2)cc1C(F)(F)F] | 2.60 | 424.33 | 562.99 | 84.93 | 35 | 0 | 26.98 258.7 |
| 3 | 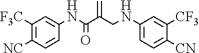 [SMILES: C=C(CNc1ccc(C#N)c(C(F)(F)F)c1)C(=O)Nc1ccc(C#N)c(C(F)(F)F)c1] | 4.63 | 438.33 | — | 36.850 | | | |
| 4 | 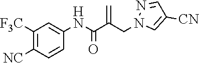 [SMILES: C=C(Cn1cc(C#N)cn1)C(=O)Nc1ccc(C#N)c(C(F)(F)F)c1] | 2.54 | 345.28 | 1229.75 | 1418 | 0 | 51 | 18.19 73.08 |
| 5 | 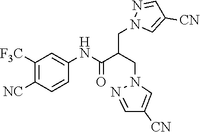 [SMILES: N#Cc1cnn(CC(Cn2cc(C#N)cn2)C(=O)Nc2ccc(C#N)c(C(F)(F)F)c2)c1] | 2.35 | 438.37 | 10.500 | 356 | 77 | 0 | |
| 6 | 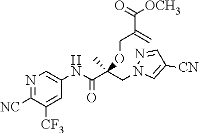 [SMILES: C=C(CO[C@@](C)(Cn1cc(C#N)cn1)C(=O)Nc1cnc(C#N)c(C(F)(F)F)c1)C(=O)OC] | 1.55 | 462.38 | No binding | 177 | 13, 49 | | Infinity 0 |
| 7 | 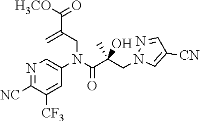 [SMILES: C=C(CN(C(=O)[C@@](C)(O)Cn1cc(C#N)cn1)c1cnc(C#N)c(C(F)(F)F)c1)C(=O)OC] | 1.43 | 462.38 | 1530 | 460.5 | | | |
| 8 | 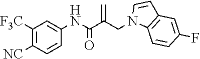 [SMILES: C=C(Cn1ccc2cc(F)ccc21)C(=O)Nc1ccc(C#N)c(C(F)(F)F)c1] | 4.11 | 387.33 | — | 4954 | | | |
| 16 | 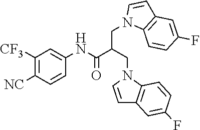 [SMILES: N#Cc1ccc(NC(=O)C(Cn2ccc3cc(F)ccc32)Cn2ccc3cc(F)ccc32)cc1C(F)(F)F] | 5.48 | 522.48 | | | | | |
| 1-b | 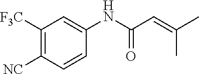 [SMILES: CC(C)=CC(=O)Nc1ccc(C#N)c(C(F)(F)F)c1] | 3.4 | 380.10 | — | — | | | |
| 9 | 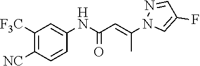 [SMILES: C/C(=C\C(=O)Nc1ccc(C#N)c(C(F)(F)F)c1)n1cc(F)cn1] | 2.2 | 338.26 | 319 | 364.2 | 8, 60 | 45 | |
| 10 | 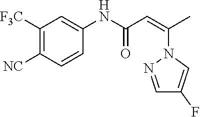 [SMILES: C/C(=C/C(=O)Nc1ccc(C#N)c(C(F)(F)F)c1)n1cc(F)cn1] | 2.2 | 338.26 | — | — | 92 | 26 | |
| 11 | 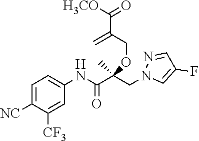 [SMILES: C=C(CO[C@@](C)(Cn1cc(F)cn1)C(=O)Nc1ccc(C#N)c(C(F)(F)F)c1)C(=O)OC] | 2.59 | 454.37 | 2416 | 400.2 | | | |
| 12 | 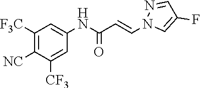 [SMILES: N#Cc1c(C(F)(F)F)cc(NC(=O)/C=C/n2cc(F)cn2)cc1C(F)(F)F] | 2.58 | 324.23 | | 1137 | | | |
| 13 | 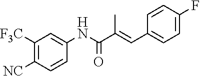 [SMILES: C/C(=C\c1ccc(F)cc1)C(=O)Nc1ccc(C#N)c(C(F)(F)F)c1] | 4.64 | 348.29 | | 731.7 | | | |
| 14 | 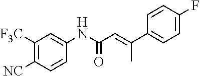 [SMILES: C/C(=C\C(=O)Nc1ccc(C#N)c(C(F)(F)F)c1)c1ccc(F)cc1] | 4.47 | 348.29 | | 18.2 | | | |
| 15 | 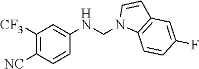 [SMILES: N#Cc1ccc(NCn2ccc3cc(F)ccc32)cc1C(F)(F)F] | 4.49 | 333.28 | No binding | 726 | 16, 80 | | |
| 17 | 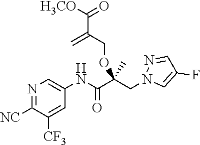 [SMILES: C=C(CO[C@@](C)(Cn1cc(F)cn1)C(=O)Nc1cnc(C#N)c(C(F)(F)F)c1)C(=O)OC] | 2.45 | 461.39 | No binding | 182.2 | 58 | | |
| 18 | 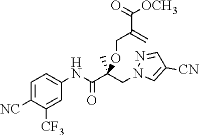 [SMILES: C=C(CO[C@@](C)(Cn1cc(C#N)cn1)C(=O)Nc1ccc(C#N)c(C(F)(F)F)c1)C(=O)OC] | 1.68 | 455.36 | No binding | 257 | 37 | | |

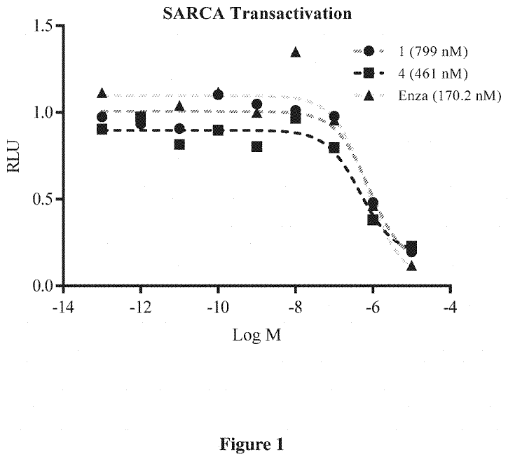

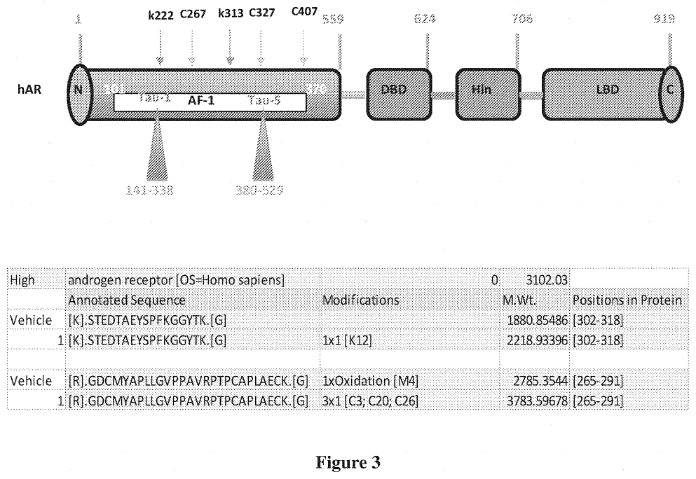

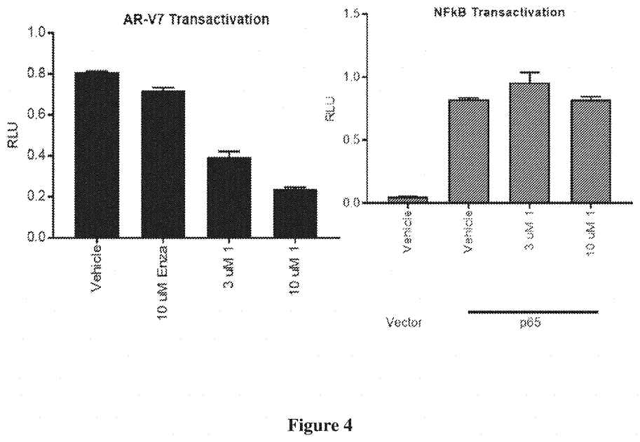

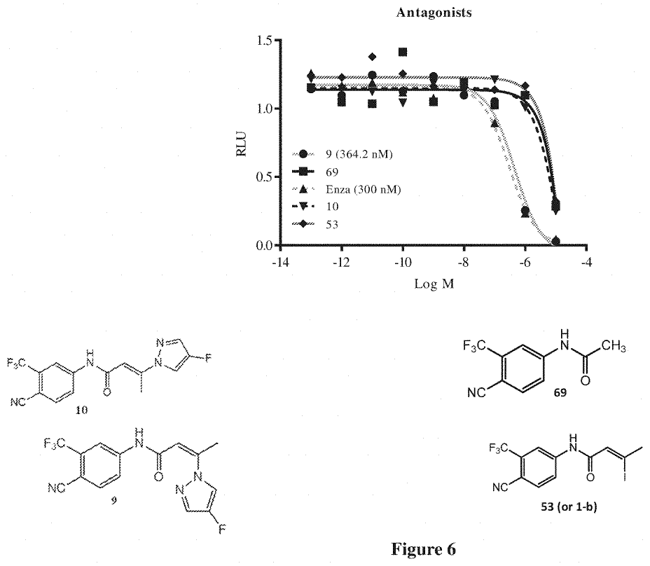

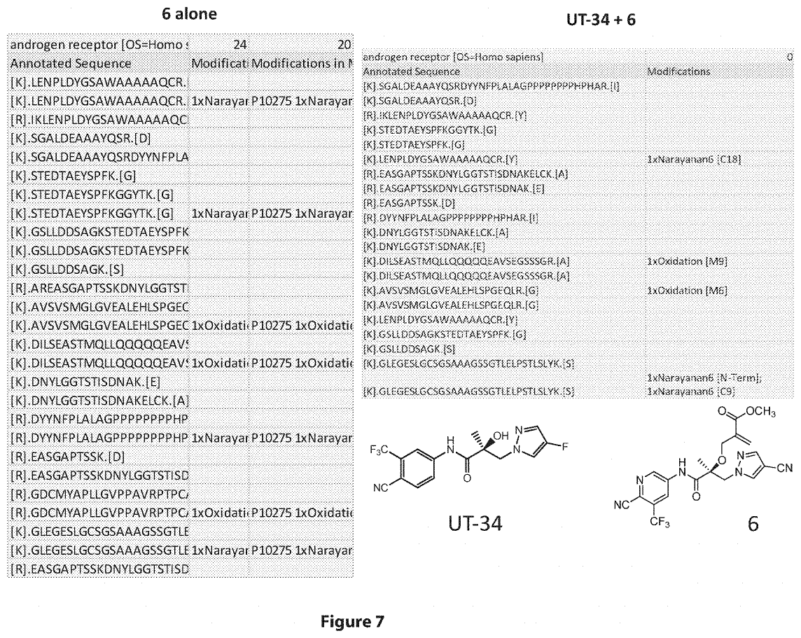

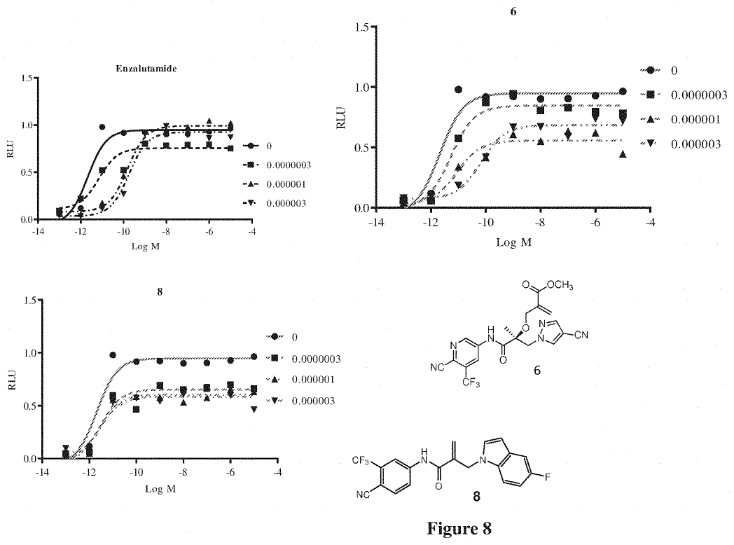

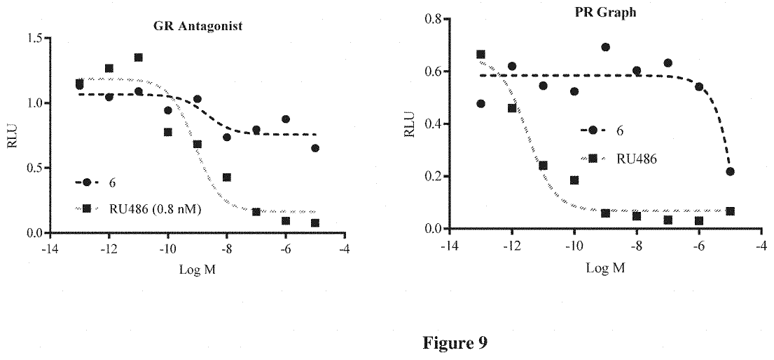

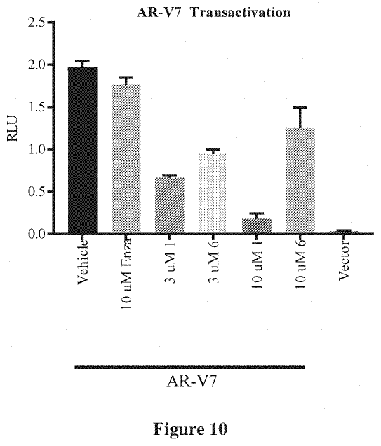

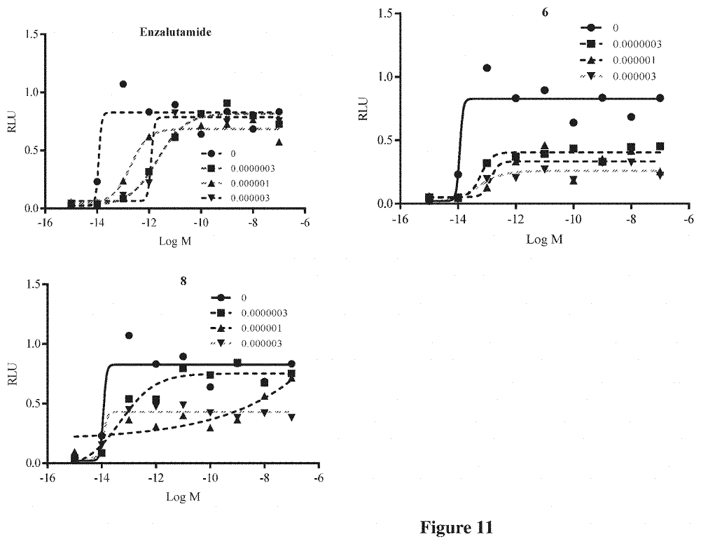

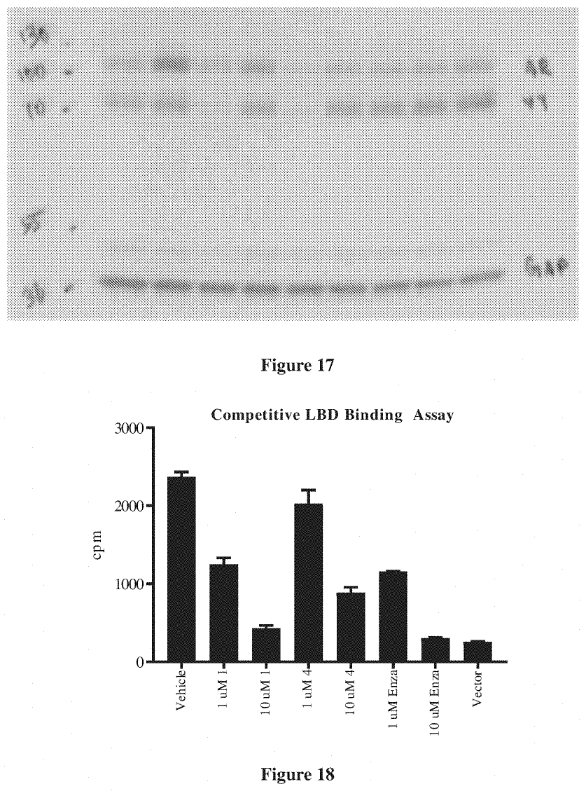

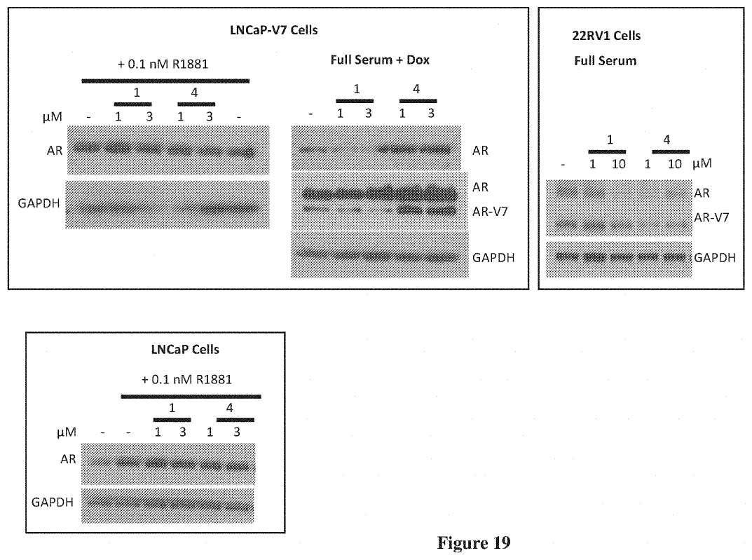

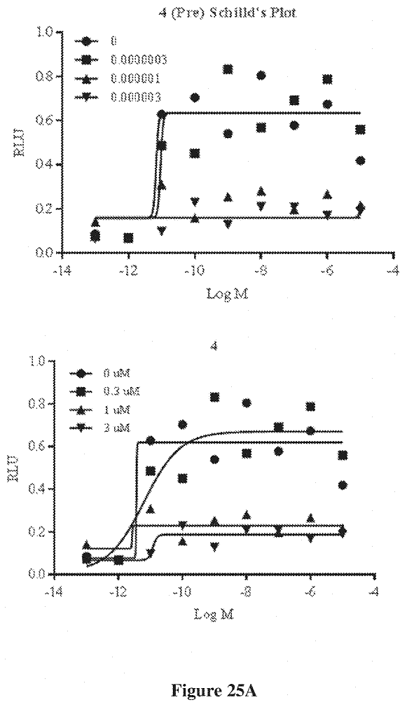

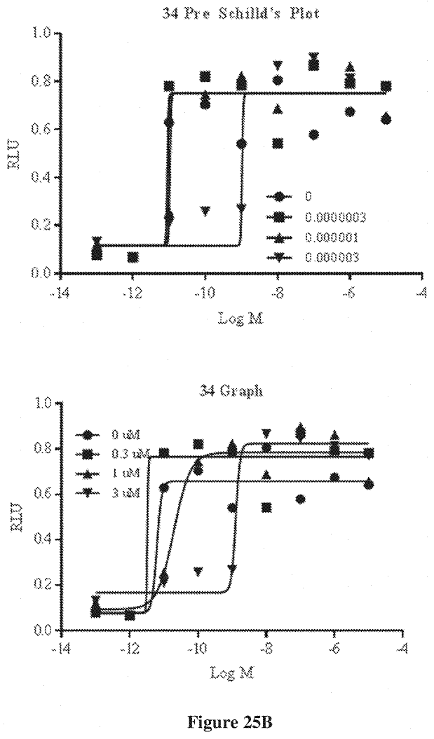

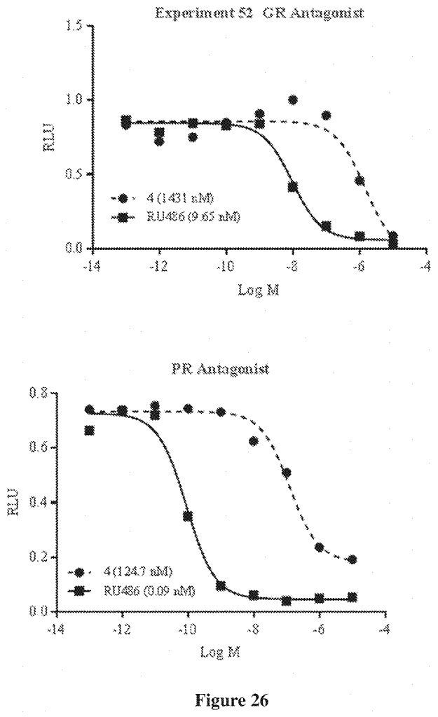

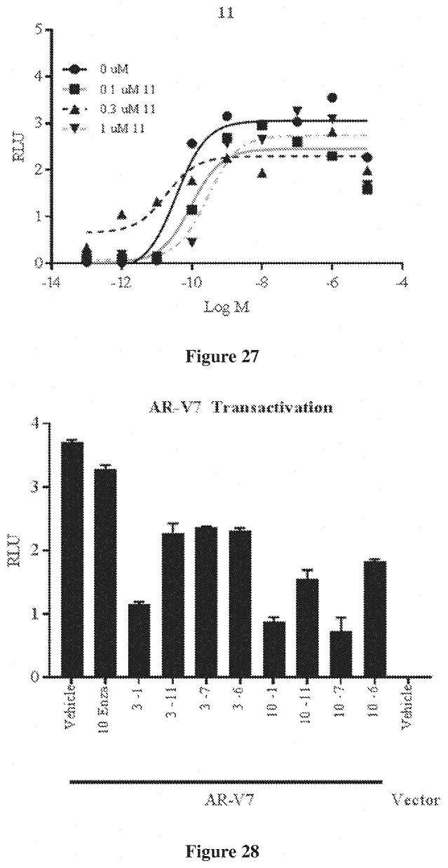

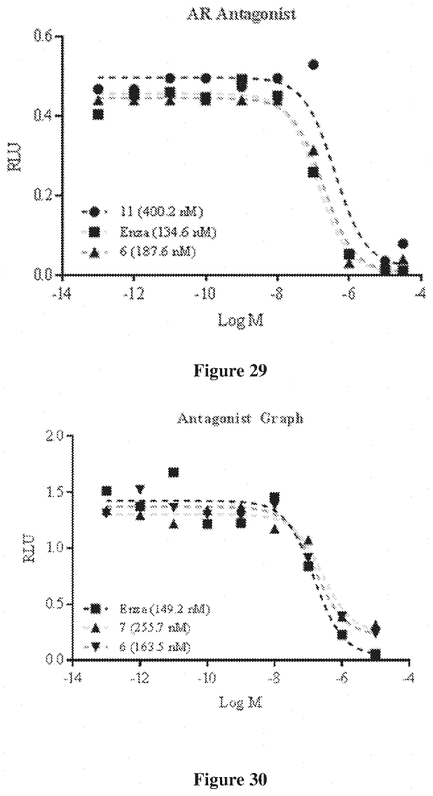

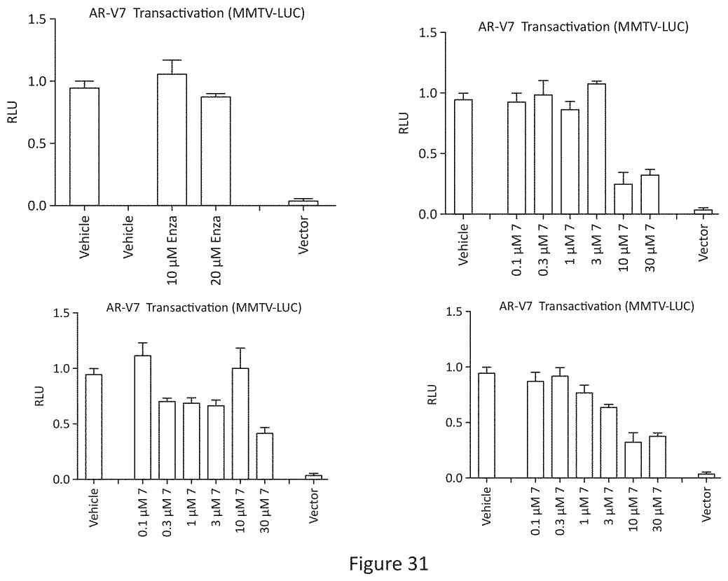

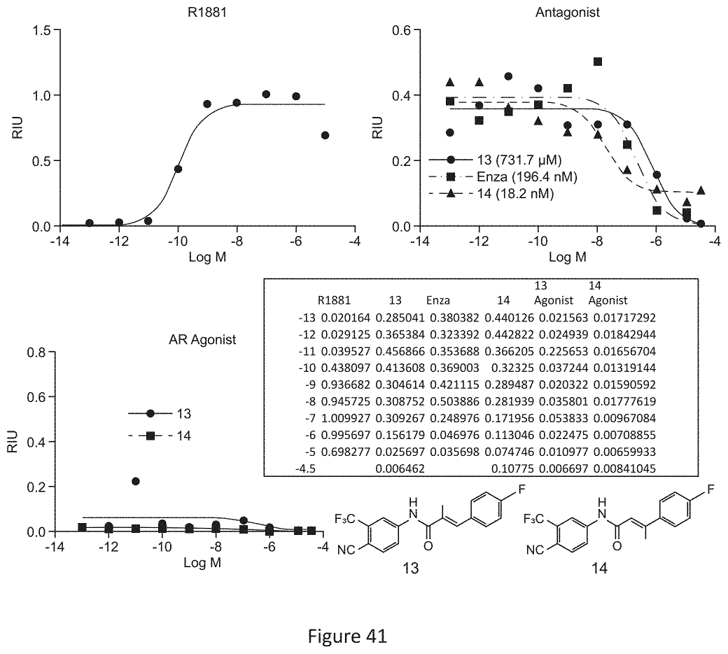

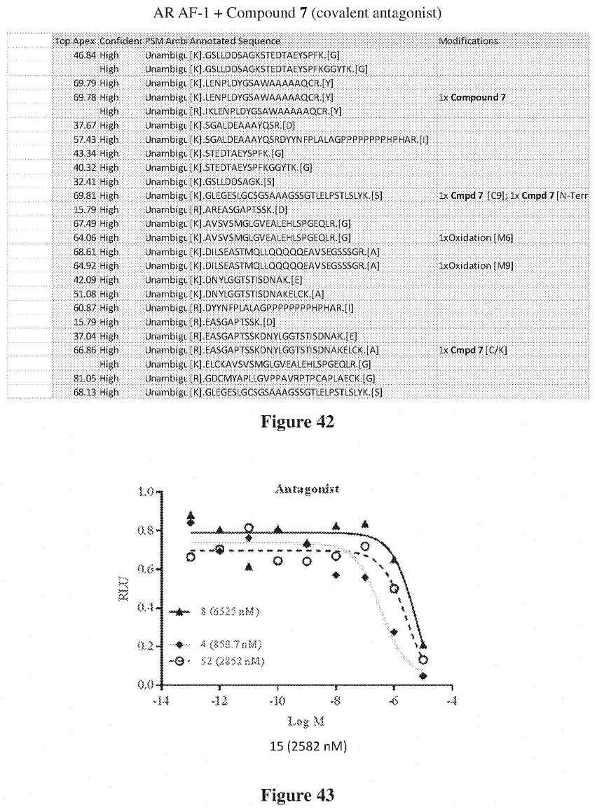

TABLE 2

| | | | | | | | MassSpec |
|---|---|---|---|---|---|---|---|
| | AR | GR | PR | | | | Binding/ |
| | antagonism | antagonism | antagonism | AR-V7 | AR | AR-V7 | Schild's |
| SARCA | (IC₅₀ nM) | (IC₅₀ nM) | (IC₅₀ nM) | antagonism | degradation | degradation | plot |
| 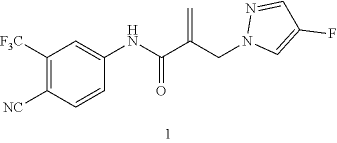 [SMILES: C=C(Cn1cc(F)cn1)C(=O)Nc1ccc(C#N)c(C(F)(F)F)c1] | 1330 see also Figure 1 where IC₅₀ was 799 nM) | 1036 (see also Figure 23 where IC₅₀ was 776 nM) | 745 | Yes (see Figures 4, 28, 31 | Yes (see Figures 17 and 19) | Yes (see Figures 17 and 19) | Yes/Yes (see Figures 3/2) |
| 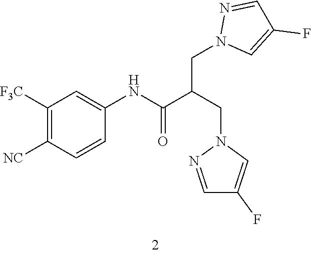 [SMILES: N#Cc1ccc(NC(=O)C(Cn2cc(F)cn2)Cn2cc(F)cn2)cc1C(F)(F)F] | 84.93 | >1000 (see Figure 23) | | | Yes (see Figure 17) | Yes (see Figure 17) | |
| 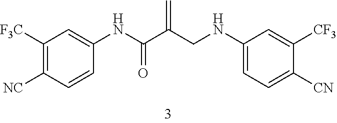 [SMILES: C=C(CNc1ccc(C#N)c(C(F)(F)F)c1)C(=O)Nc1ccc(C#N)c(C(F)(F)F)c1] | 36.850 | — | — | | | | N.D./N.D. |
| 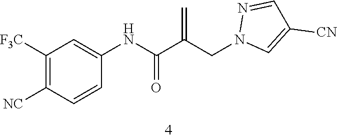 [SMILES: C=C(Cn1cc(C#N)cn1)C(=O)Nc1ccc(C#N)c(C(F)(F)F)c1] | 1418 | 1431 (see Figures 23 and 26) | 124.7 (see Figure 26) | | Yes (see Figures 17 and 19) | Yes (see Figures 17 and 19) | Yes/Yes (see Figures 25A/2) |
| 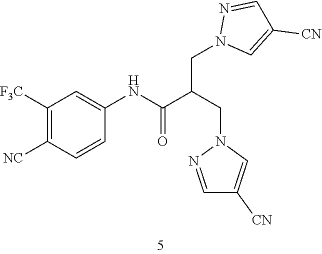 [SMILES: N#Cc1cnn(CC(Cn2cc(C#N)cn2)C(=O)Nc2ccc(C#N)c(C(F)(F)F)c2)c1] | 356 | | | | Yes (see Figure 17) | | |
| 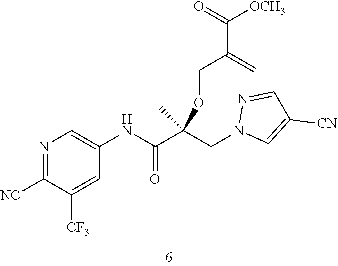 [SMILES: C=C(CO[C@@](C)(Cn1cc(C#N)cn1)C(=O)Nc1cnc(C#N)c(C(F)(F)F)c1)C(=O)OC] | 177 (see also Figures 29, 30) | 6223 (see Figures 9 and 23) | — (see Figure 9) | Yes (see Figures 10, 28, 31) | | | Yes/Yes (see Figures 7/8, 11) |
| 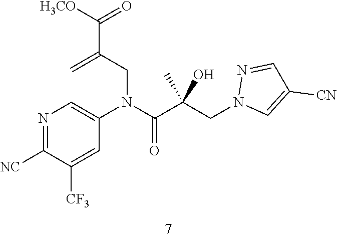 [SMILES: C=C(CN(C(=O)[C@@](C)(O)Cn1cc(C#N)cn1)c1cnc(C#N)c(C(F)(F)F)c1)C(=O)OC] | 460.5 (see also Figure 30) | | | Yes (see Figures 28, 31) | | | Figure 42/N.D |
| 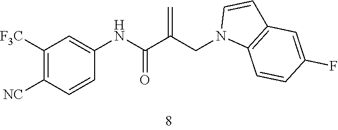 [SMILES: C=C(Cn1ccc2cc(F)ccc21)C(=O)Nc1ccc(C#N)c(C(F)(F)F)c1] | 4954 | 5755 | — | | | | N.D./Yes (N.D.)/(see Figures 8, 11) |
| 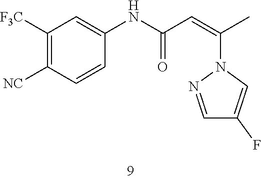 [SMILES: C/C(=C/C(=O)Nc1ccc(C#N)c(C(F)(F)F)c1)n1cc(F)cn1] | 364.2 (see Figure 6) | 193.1 | 465 | | | | N.D./N.D. |
| 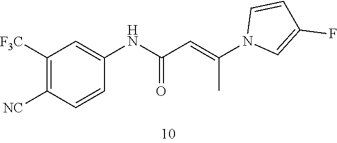 [SMILES: C/C(=C\C(=O)Nc1ccc(C#N)c(C(F)(F)F)c1)n1ccc(F)c1] | — (see Figure 6) | N.D. | N.D. | | | | N.D./N.D. |
| 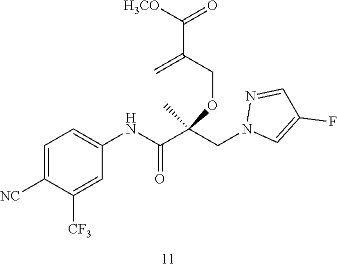 [SMILES: C=C(CO[C@@](C)(Cn1cc(F)cn1)C(=O)Nc1ccc(C#N)c(C(F)(F)F)c1)C(=O)OC] | 400 (see Figure 29) | | | Yes (see Figure 28) | | | ((N.D.)/(see Figure 27) |
| 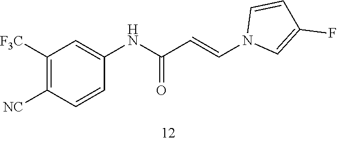 [SMILES: N#Cc1ccc(NC(=O)/C=C/n2ccc(F)c2)cc1C(F)(F)F] | 1137 | | | | | | |
| 13 | 732 (see | | | | | | |
| | Figure 41) | | | | | | |
| 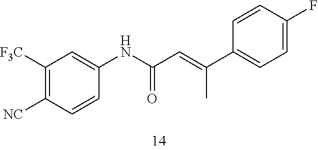 [SMILES: C/C(=C\C(=O)Nc1ccc(C#N)c(C(F)(F)F)c1)c1ccc(F)cc1] | 18 (see Figure 41) | | | | | | |
| 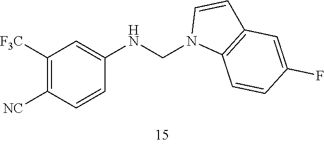 [SMILES: N#Cc1ccc(NCn2ccc3cc(F)ccc32)cc1C(F)(F)F] | 726 (see Figure 43) | | | | | | |
| 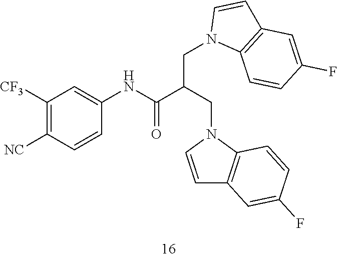 [SMILES: N#Cc1ccc(NC(=O)C(Cn2ccc3cc(F)ccc32)Cn2ccc3cc(F)ccc32)cc1C(F)(F)F] | N.D. | | | | | | |

### Example 3: SARCAs of this Invention are AR Antagonists (IC₅₀) and May Reversibly Bind the LBD (Kᵢ)

1 and 4 were evaluated in an AR transactivation assay. AR transactivation assay was performed in COS cells with AR, GRE-LUC, and CMV-renilla-LUC. The two compounds have a carbon-carbon double-bond moiety that they would need as covalent irreversible antagonists. The molecules were evaluated as to whether they have any effect on AR function. wtAR transactivation assay suggested that these two molecules have IC₅₀ values in submicromolar range (799 nM and 461 nM, respectively, in this experiment) (FIG. 1).

FIG. 6 demonstrated that 9 inhibited wtAR (364 nM), whereas its isomer 10 was a much weaker inhibitor of wtAR (micromolar range).

FIG. 29 demonstrated that 6 and 11 inhibited wtAR with IC₅₀ values in the low to mid nM range (177 nM and 400 nM, respectively).

FIG. 30 demonstrated that 6 and its isomer 7, in a separate experiment, inhibited wtAR with IC₅₀ values in the low to mid nM range (164 nM and 256 nM, respectively).

FIG. 41 demonstrated that 13 and its isomer 14 inhibited wtAR with IC₅₀ values of 732 nM and 18 nM, respectively, and demonstrated no intrinsic agonist activity. This data suggests that the left side N-atom as in the pyrazoles is not necessary for inhibition.

FIG. 43 demonstrated that 15, 8 and 4 inhibited wtAR with IC₅₀ values of 2852 nM, 6525 nM, and 850.7 nM, respectively.

FIG. 18 demonstrated that in addition to inhibition of wtAR, SARCAs of this invention in some cases bound reversibly with the LBD of AR (Ki column of Table 1). This competitive binding is also demonstrated in FIG. 18, for 1, 4, and enzalutamide (positive control). Pyrazoles and indoles lacking the warhead of the SARCAs of this invention were previously demonstrated to bind reversibly to AF-1. SARCAs of this invention, with the warhead, have been demonstrated herein to bind irreversible to AR-1 (or possibly LBD). It can be expected that irreversible AR inhibition will confer new properties to the SARCAs of this invention such as AR-V7 inhibition and the ability to inhibit cells whose growth is dependent of AR-V7 or another AR SV or genes whose expression are dependent on AR-V7 or another AR SV.

### Example 4: Compounds 1 and 4 are Covalent Irreversible AR Antagonists

Schild's plot was used to determine whether a molecule is a competitive antagonist or an irreversible covalent antagonist.

Schild's Plot: COS cells plated in 24 well plates in DME+5% csFBS without phenol red at 40,000 cells/well were transfected with 0.25 μg GRE-LUC, 25 ng CMV-hAR, and 10 ng CMV-renilla LUC using Lipofectamine reagent in OptiMEM medium. Cells were treated 24 h after transfection with a dose-response of R1881 (10⁻¹² M to 10⁻⁵ M) in the absence or presence of various doses of AR antagonist. Twenty-four hours after treatment, the cells were lysed and luciferase assay was performed using Dual luciferase assay kit (Promega, Madison, WI). Firefly luciferase values were normalized to Renilla luciferase values. The data were plotted in GraphPad prism and a Schild's plot was plotted.

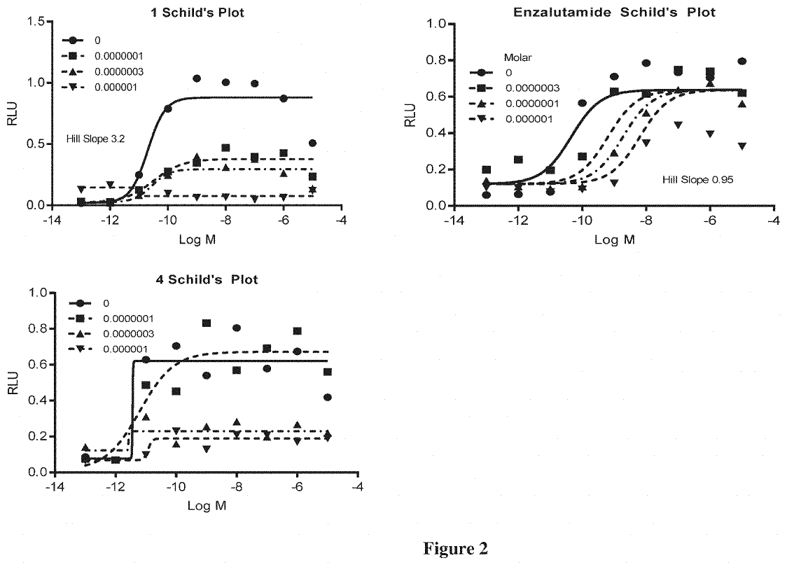

In the experiment, a compound is tested at a few doses with a dose-response of a competing agonist. If the curve shifts to the right with increasing agonist dose and if the slope is close to 1, then the molecule is a competitive antagonist. On the other hand, if the curve does not shift to the right, but if the Eₘₐₓ shifts downwards and if the slope is not close to 1, then the molecule is a covalent irreversible antagonist. An AR transactivation assay as described above was used and a Schild's plot was created to evaluate if 1 and 4 are covalent antagonists. The enzalutamide curve shifts to the right with increasing R1881 dose, indicative of competitive non-covalent antagonism. On the other hand, the Eₘₐₓ values of R1881 decreases in the presence of increasing dose of 1 and 4 (FIG. 2). The Schild's plots suggest that 1 and 4 are covalent irreversible antagonists. Similarly, FIG. 25 demonstrated reduced Eₘₐₓ for 4.

### Example 5: Compounds 1 and 4 Covalently Bind to AF-1 Domain of AR

Alkylation via Mass Spectrometry of Tryptic Digests

Mass Spectrometry: ARAF-1 (A.A. 141-486) was cloned in pGEX 6p and was expressed in E. coli. Protein was purified from a large bacterial culture through GST resin and then through 25 FPLC. The purified AF-1 protein was incubated at 4° C. for overnight in the presence of the SARCAs. After overnight incubation, the protein was incubated for overnight at room temperature (RT) in the presence of mass spectrometry grade trypsin. The protein was analyzed using HPLC (Ultimate 3000RSLCnano, Thermo Fisher) attached to a mass spectrometer (Orbitrap Fusion Lumas, Thermo Fisher). Acclaim PepMap 100 column was used for HPLC. The instrument conditions and analysis information are provided below. Sample amount per injection: 0.1 μg of digested protein.

HPLC: Ultimate 3000RSLCnano, Thermo Fisher; Column: Acclaim PepMap RSLC, 75 m×500 mm (ID×Length), C-18, 2 m, 100 A, Thermo Fisher; Trap column: Acclaim PepMap 100, 75 m×20 mm, C18, 3 m, 100 Å, Thermo Fisher; Solvent A: 0.1% formic acid in water, LC/MS grade, Thermo Fisher; Solvent B: 0.1% formic acid in acetonitrile, LC/MS grade, Thermo Fisher; Flow rate: 300 nL/min; Column temperature: 40° C.; Injection volume/mode: 5 μL/μL PickUp; LC Gradient: 0 min-3% B, 4 min-3% B, 5 min-5% B, 55 min-25% B, 60 min-30% B, 63 min-90% B, 73 min-90% B, 76 min-3% B, 100 min-3% B

MS: Orbitrap Fusion Lumas, Thermo Fisher; Data dependent analysis (DDA): 3 sec cycles; MS scan (full): Analyzer—Orbitrap, resolution-120,000 (FWHM, at m/z=200); Scan Filters: MIPS mode—Peptide; Intensity ≥10,000; Charge state—2-6; Dynamic exclusion—30 sec; MS2 scan (full): Quadrupole isolation window—0.7 m/z, Activation—HCD (30%); Analyzer—Ion Trap, Rapid scan

Post-Acquisition Analysis

Proteome Discoverer 2.2, Thermo Fisher; Peptide/protein identification; Search engine: Sequest HT; Database: SwissProt, TaxID 9606 (Homo sapiens), v. 2017-10-25, 42252 entries; Enzyme: Trypsin (full); Dynamic modification: Oxidation of Met; Modification of Cys and/or Lys with UT-34 (a non-covalent binder of AF-1), or SARCAs 1 or 4; Precursor and fragment ion mass tolerance: 10 ppm and 0.6 Da, respectively; Validation and filtering of PSM (q value): Percolator, FDR ≤0.01; Validation and filtering of peptide sequence (q value): Qvality algorithm, FDR ≤0.01; Identification of protein or protein group: At least one validated peptide sequence unique to a protein or a protein group; Protein groups: Strict parsimony principle applied

Validation of protein ID: Quality algorithm, strict—FDR ≤0.01, relaxed—FDR ≤0. Feature Detection-Min Trace Length: 5; Min #Isotopes: 2; Max ΔRT of Isotope Pattern: 0.2 min; Peptide Abundance: MS Peak Area

To determine if these molecules bind to the AF-1 domain of the AR, AR-AF-1 purified protein was incubated with 1 for 16 h at 4° C. and trypsin digested. The peptides were evaluated using MALDI TOF mass spectrometer to determine the binding of 1 to AF-1. 1 bound to the peptides indicated in the panel (FIG. 3). The M.Wt. shift by 338.08 Dalton of the top peptide corresponds to the M.Wt. of 1. Similarly, three molecules of 1 covalently interacted with the bottom peptide with M.Wt. corresponding shift of 998.75. The results indicate that 1 covalently attached itself to cysteines and lysine in the AF-1 domain of the AR (FIG. 3). While 1 bound to the AF-1, negative control enzalutamide failed to show any binding.

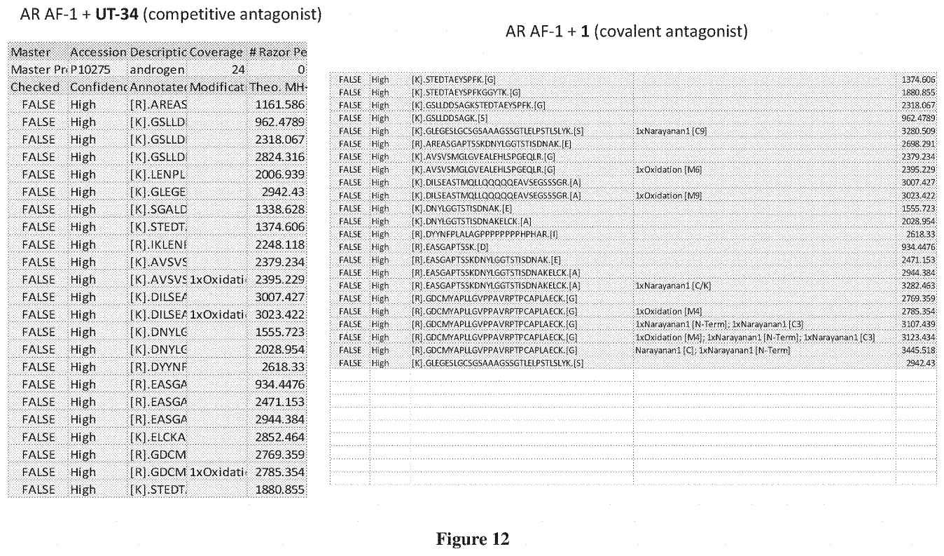

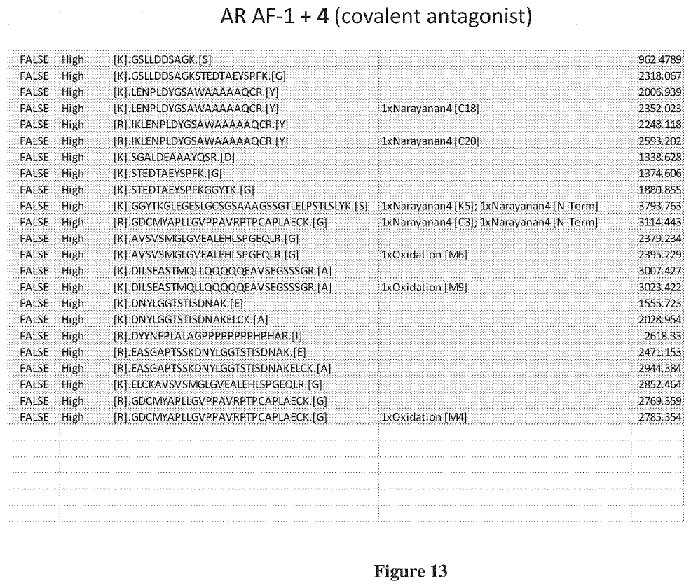

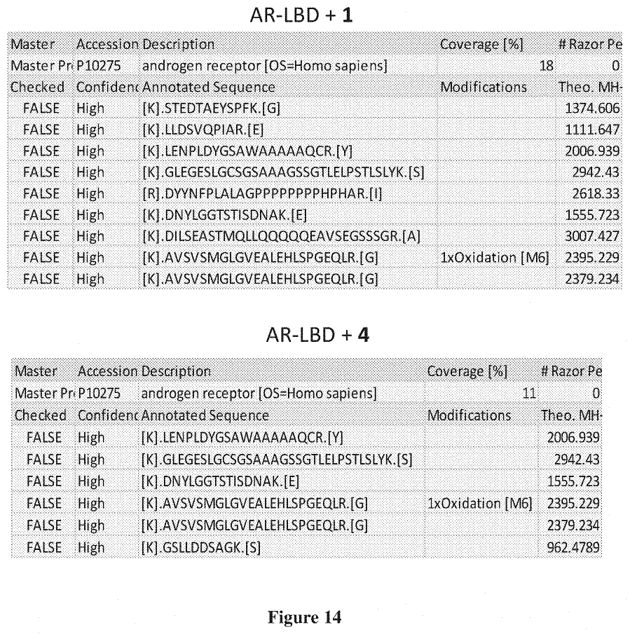

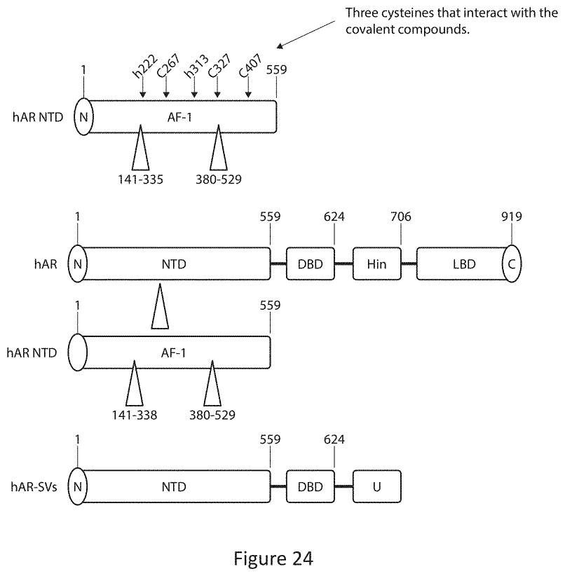

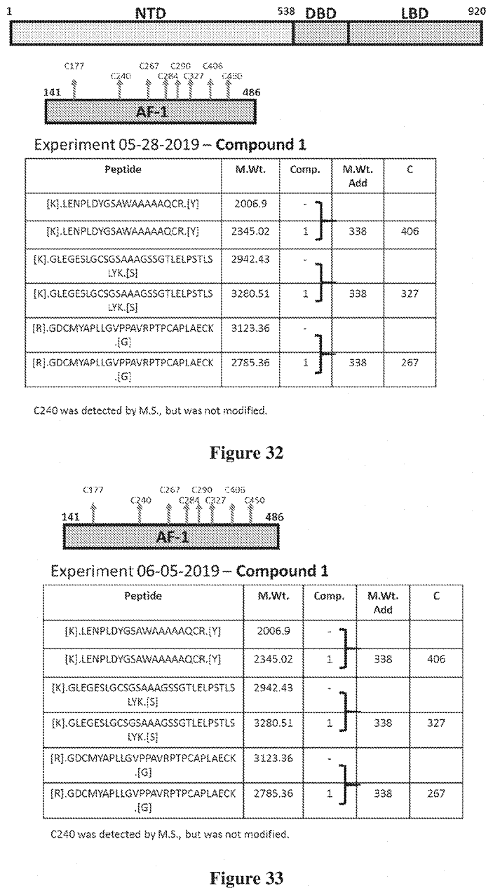

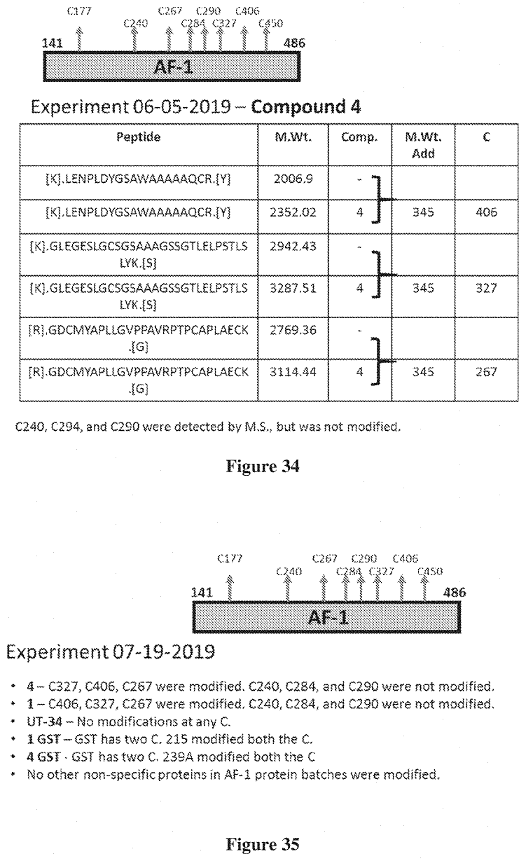

The alkylation of AR at AF-1 by 1 or 4 was demonstrated multiple times in variations of this same methology such as in FIGS. 12, 13, and 32-36. In each case, the amino acids that were alkylated (covalently modified by the SARCA) were in the AF-1 region of the NTD. Further, FIG. 14 suggests that 1 and 4 do not alkylate the LBD. An overview of the lysine (K) and cysteine (C) residues in the NTD of human androgen receptor (hAR NTD) is shown in FIG. 24 (top), and the domain topology of full length hAR and splice variant hAR (hAR SVs) is also shown. DBD is DNA binding domain; Hin is the hinge region; LBD is ligand binding domain; Tau is the transcriptional activation unit, two Taus are annotated in the figure (Tau-1 and Tau-5); U is an unknown region of cryptic structure that is found in splice variant ARs. The same three C residues are covalently modified by multiple SARCAs of this invention.

### Example 6: Compound 1 Inhibited AR-V7 Function

If 1 covalently binds to the AF-1 domain of the AR, then it should inhibit the AR-V7 activity. A transactivation study was performed with AR-V7 in COS cells. While 1 significantly inhibited the ability of AR-V7 to activate GRE-LUC, enzalutamide was inactive (FIG. 4). NF-kB transactivation was included as a negative control. As expected, 1 was unable to (bind or) inhibit NF-kB induced transactivation.

AR-V7 transactivation: COS cells plated in 24 well plates in DME+5% csFBS without 20 phenol red at 40,000 cells/well were transfected with 0.25 μg GRE-LUC, 25 ng pCDN3 AR-V7, and 10 ng CMV-renilla LUC using Lipofectamine reagent in OptiMEM medium. Cells were treated 24 h after transfection. Twenty-four hours after treatment, the cells were lysed and luciferase assay was performed using Dual luciferase assay kit (Promega, Madison, WI). Firefly luciferase values were normalized to renilla luciferase values. The data were plotted in GraphPad Prism.

To determine the cross-reactivity of 1 with another constitutively active protein, 1 was tested in NFkB transactivation. 1 did not inhibit NFkB transactivation, indicating its selectivity (FIG. 4).

### Example 7: Compound 1 but not Compound 6 Cross-Reacted with Other Receptors

The Michael addition accepting functional group in 1 and 4 is exposed and hence has the potential to randomly bind to other proteins. To confirm this, 1 and 4 were tested for their ability to inhibit the activity of GR and PR (Table 2), and PPAR-γ (not shown)). 1 and 4 (FIG. 26) inhibited the transactivation of all three receptors confirming their cross-reactivity (Table 2). See also FIG. 23 where 1 and 4 have 776 nm and 630 nM IC₅₀ values in GR and FIG. 26 where IC₅₀ values for 4 were 1431 nM (GR) and 125 nM (PR). Whereas 6 demonstrated very little cross-reactivity with GR and PR, respectively, as shown in FIGS. 9 and 23.

Objective: To determine the effect of SARCAs on glucocorticoid-induced transactivation of GR wildtype (wt).

Method: HEK-293 cells were plated at 125,000 cells/well of a 24 well plate in DME+5% csFBS without phenol red. Cells were transfected with 0.25 μg GRE-LUC, 10 ng CMV-renilla LUC, and 50 ng pCR3.1-rat GR(wt) using Lipofectamine transfection reagent in optiMEM medium. Medium was changed 24 h after transfection to DME+5% csFBS without phenol red and treated with a dose response of various drugs (1 pM to 10 mM). SARCAs and antagonists were treated in combination with 0.1 nM dexamethasone. Luciferase assay was performed 24 h after treatment on a Biotek synergy 4 plate reader. Firefly luciferase values were normalized to renilla luciferase values.

Objective: To determine the effect of SARCAs on progesterone-induced transactivation of PR wildtype (wt).

Method: HEK-293 cells were plated at 125,000 cells/well of a 24 well plate in DME+5% csFBS without phenol red. Cells were transfected with 0.25 μg GRE-LUC, 10 ng CMV-renilla LUC, and 50 ng pCR3. 1-hPR(wt) using Lipofectamine transfection reagent in optiMEM medium. Medium was changed 24 h after transfection to DME+5% csFBS without phenol red and treated with a dose response of various drugs (1 pM to 10 mM). SARCAs and antagonists were treated in combination with 0.1 nM progesterone. Luciferase assay was performed 24 h after treatment on a Biotek synergy 4 plate reader. Firefly luciferase values were normalized to renilla luciferase values.

### Example 8: Compound 1 Inhibited Proliferation of PCa Cell Lines

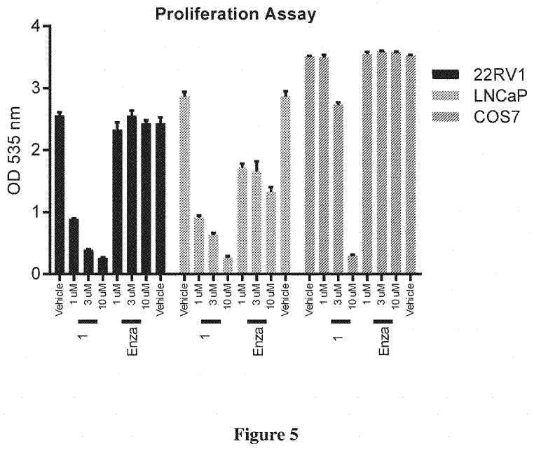

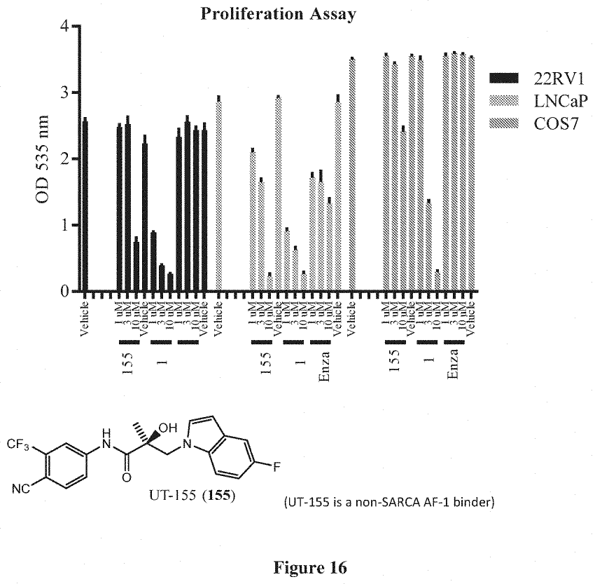

LNCaP and 22RV1 cells were cultured in full serum and treated as indicated in FIG. 5. Cells were treated for 6 days and SRB assay was performed to measure the number of viable cells. 1 inhibited the proliferation of LNCaP and 22RV1 cells, while enzalutamide had modest effects on only LNCaP cells (FIGS. 5 and 16).

The covalent-binding irreversible AR antagonists of the present invention have been synthesized with much lower IC₅₀ values, which are highly selective to the AR. Further, through mass spectrometry studies, it was found that the compounds of the invention as disclosed herein, e.g., 1 and 4 did bind to the AR in the AF-1 region. The Schild plots in FIG. 2 suggest that 1 and 4 were irreversible antagonists of the AR and these agents also blocked AR-SV.

### Example 9: Mass Spectrometry Experiments to Determine Covalent Binding of 6 and 7

AR AF-1 protein was incubated with a molecule overnight at 4° C. The protein was digested with trypsin overnight at RT and was evaluated using mass spectrometry. Covalent molecules bind to cysteine and lysine. If a molecule is attached covalently to a peptide, the molecular weight of the peptide will increase by the molecular weight of the molecule. For example, if a tryptic digested peptide's M.Wt. is 1000 Dalton and the incubated molecule's M.Wt. is 250 Dalton, then the covalently-bound peptide's M.Wt. will be ˜1250 Dalton. If two molecules are attached to a peptide, then the M.Wt. will increase correspondingly to ˜1500 Dalton.

AR AF-1 was incubated with 6 (covalent binder) alone or 6+UT-34 (UT-34 is a noncovalent AF-1 binder). AF-1 was pre-incubated for 2 h with 200 μM UT-34 and then with 6 (100 μM).

As illustrated in FIG. 7, 6 is a SARCA which bound irreversibly to the tryptic peptides.

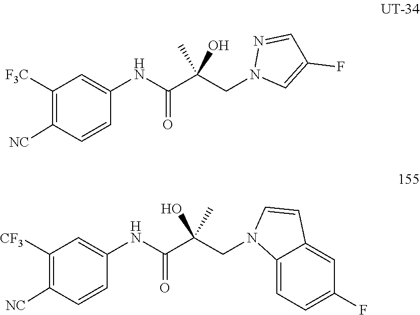 [SMILES: C[C@](O)(Cn1cc(F)cn1)C(=O)Nc1ccc(C#N)c(C(F)(F)F)c1 ; C[C@](O)(Cn1ccc2cc(F)ccc21)C(=O)Nc1ccc(C#N)c(C(F)(F)F)c1]

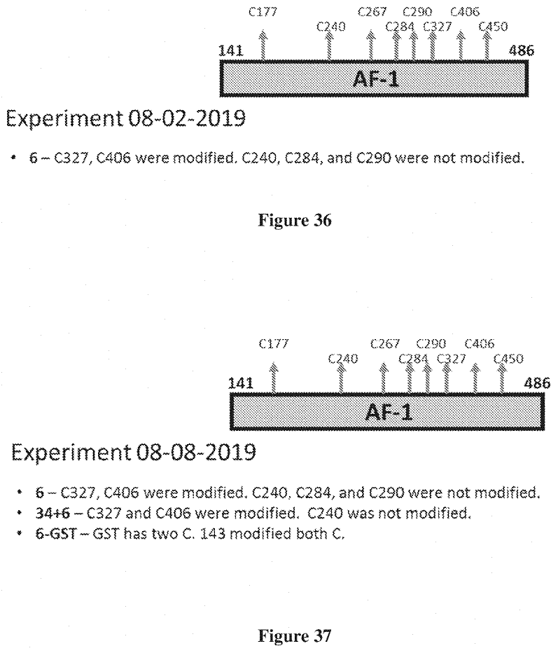

As confirmed in FIGS. 36 and 37 from separate experiment, 6 again covalently bound (alkylated) to AF-1, but also alkylated GST, suggesting the selectivity of irreversible binding still needs to be improved.

FIG. 42 demonstrates incontrovertibly that 7 also binds irreversibly to AF-1.

### Example 10: Compounds 6, 8, and 11 Irreversibly Bound to AR

Schild's plot is an assay to detect irreversible antagonism. If a molecule like enzalutamide is a competitive antagonist, increasing its dose will shift the curve of R1881 or an agonist to the right. If a molecule is an irreversible antagonist, the curve will shift downward with reduced Eₘₐₓ.

AR trans activation was performed with 0.25 μg GRE-LUC, 0.01 μg CMV-LUC, and 0.025 25 μg CMV-hAR. Cells were treated with a dose-response of R1881 in the presence of the indicated concentrations (Molar) of the compounds). Cells were harvested and luciferase assay was performed.

FIG. 8 depicts that enzalutamide was a reversible AR inhibitor whereas the SARCAs 6 and 8 were irreversible AR inhibitors using a Schild's plot analysis.

FIG. 8 top left panel demonstrates that R1881 agonist activity was shifted right (less potent, i.e., increased EC₅₀) by increasing enzalutamide concentration without reducing the Eₘₐₓ of R1881. This confirms that enzalutamide was an AR inhibitor that competed for reversible binding with R1881 (agonist) to the AR (full length). The result was expected from the known LBD binding site of these agents. The increased EC₅₀ value demonstrates that the inhibition was surmountable (i.e., reversible). Correspondingly, it serves as a control experiment to demonstrate that the Schild's plot can demonstrate reversible competitive inhibition with AR full length.

FIG. 8 top right panel demonstrates that R1881 agonist activity is shifted right (higher EC₅₀ value) but also that the Eₘₐₓ value is decreased with increasing concentration of the SARCA 6. Similarly, FIG. 8 bottom panel demonstrates that 8 decreased the Eₘₐₓ with increasing concentrations of SARCA. Lowered Eₘₐₓ values demonstrate that the inhibition is insurmountable (i.e., irreversible). Correspondingly, 6 and 8 exhibited the behavior of an irreversible inhibitor according to the Schild's plot. Similarly, FIG. 11 demonstrated a reduced Eₘₐₓ for 6 and 8. FIG. 27 suggests that 11 also demonstrated reduced Eₘₐₓ values.

### Example 12: SARCAs are Unprecedently Potent at Inhibition of AR-V7

AR-V7 transactivation. COS7 cells were plated in 24 well plates at 40,000 cells/well in DME+5% cs FBS without phenol red. Twenty-four hours after plating, the cells were transfected 20 with 0.25 μg GRE-LUC, 0.01 μg CMV-LUC, 0.025 μg pCR3.1 hAR-V7 using Lipofectamine reagents in optiMEM medium. Twenty-four hours after transfection, the cells were treated with the compounds. Twenty-four hours after treatment, the cells were harvested, and luciferase assay was performed using Dual-luciferase reagent. Firefly values were divided by Renilla numbers and the values are represented as relative light units (RLU).

AR-V7 was transfected into the cells instead of full length wildtype AR. As shown in FIG. 10, the right bar (Vector) in the figure, demonstrates that in the absence of AR-V7 the assay did not activate transcription (no light produced or 0 relative light units (RLU)). This serves as a negative control experiment. The bar below the graphic indicates that AR-V7 was transfected into each of these cells. The left bar (Vehicle) indicates that in the absence of an inhibitor, AR-V7 was able to activate transcription and addition of 10 μM enzalutamide (Enza), an LBD binding antiandrogen, did not significantly decrease this transcription (since AR-V7 lacks the LBD). In contrast, SARCAs of this invention that irreversibly bound to the NTD (present in AR-V7) and these SARCAs, e.g., 1 and 6 were able to significantly inhibit the transcriptional activation of AR-V7. 1 was dose-dependent (inhibition at 3 μM is greater than at 10 μM) whereas 6 did not demonstrate dose-dependent behavior in the experiment.

FIG. 28 describes an inhibition of AR-V7 transactivation experiment which showed significant inhibition with 1 at 3 and 10 μM, partial inhibition with 11 and 6 at M, and significant inhibition with 7 at 10 μM. It demonstrates that AR-V7 inhibition is a generalizable activity of SARCAs whereas enzalutamide and vehicle fail, and no activation was seen in the absence of AR-V7 (vector).

FIG. 31 describes an inhibition of AR-V7 transcriptional activation experiment. Enzalutamide (FIG. 31A) failed to inhibit AR-V7 but SARCA 7, 1, and 6 each dose-dependently inhibited AR-V7. 1 was the most potent and demonstrated activity at concentrations as low as 0.3 μM, and 6 and 7 demonstrated greater maximum efficacy at 10 μM.

### Example 13: Effect on AR and AR-V7 Degradation in 22RV1 Cells

LNCaP, LNCaP-V7 (LNCaP cells stably transfected with AR-V7), 22RV1 cells were plated in 60 mm dishes. Cells were treated in growth medium or RPMI supplemented with 0.1 nM R1881 for 24 h. Cells were harvested, protein extracted, and Western blot for AR and AR-V7 was performed.

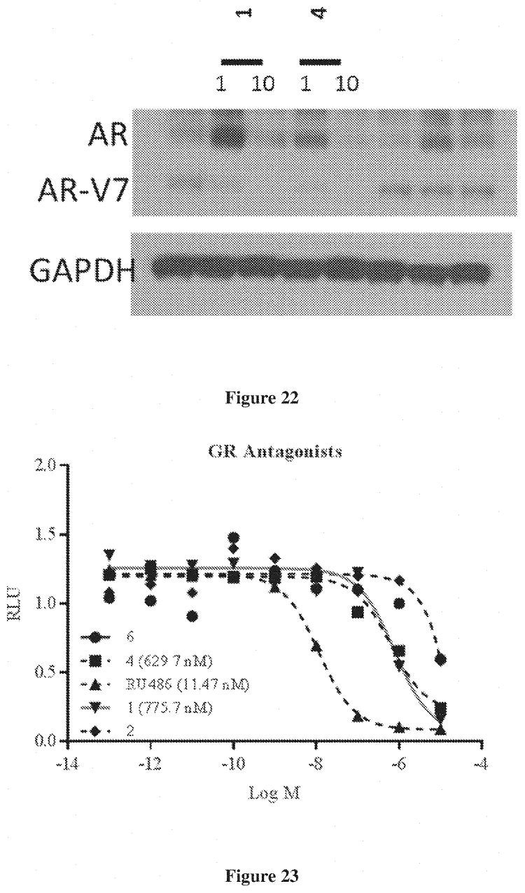

FIG. 17 demonstrates that 1 and 4 at 10 μM acted as degraders of AR (full length) and ARSV (AR-V7), whereas AR degradation activity of 2 and 5 was less robust in this experiment. Similarly, in FIG. 22, 1 and 4 were confirmed to be AR and AR-V7 degraders in 22RV1 cells.

LNCaP-V7 cells inducibly express AR-V7 by the addition of doxycycline (Dox). FIG. 19 demonstrates that in the absence of Dox, no AR-V7 was expressed (left panel), but upon addition of Dox then AR-V7 expression was seen (see gels to the right in the top left panel labeled as ‘Full Serum+Dox’). The gels to the right further demonstrate that 1 degraded AR (see top blot) and AR-V7 (see top blot) at 1 and 3 μMin LNCaP-V7 cells induced by Dox. In 22RV1 cells (top right panel) where AR-V7 was endogenously co-expressed with AR, 1 and 4 both degraded both AR and AR-V7.

### Example 14: SARCAs Inhibited AR-Dependent LNCaP Proliferation

Proliferation Assay: LNCaP cells were plated in 96 well plates in growth medium. Cells were treated with the indicated doses of the compounds for 6 days with the indicated nM of R1881 and AR antagonists of the invention, with medium change and retreatment after 3 days. Cells were fixed and stained with sulforhodamineblue (SRB). The stain color that is proportional to the number of cells was determined using a colorimeter.

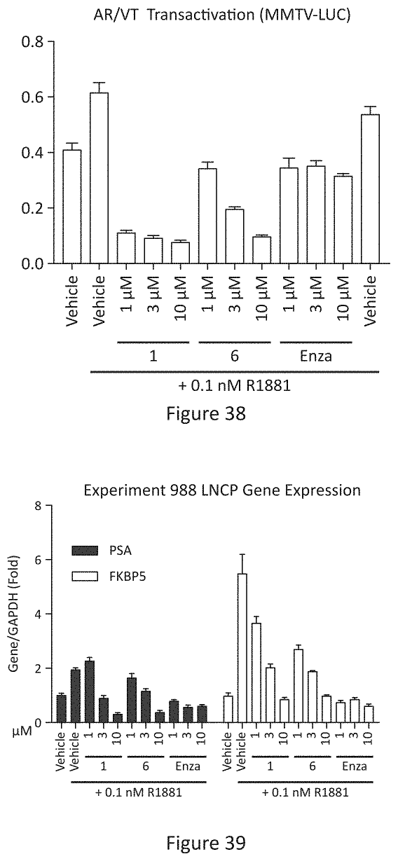

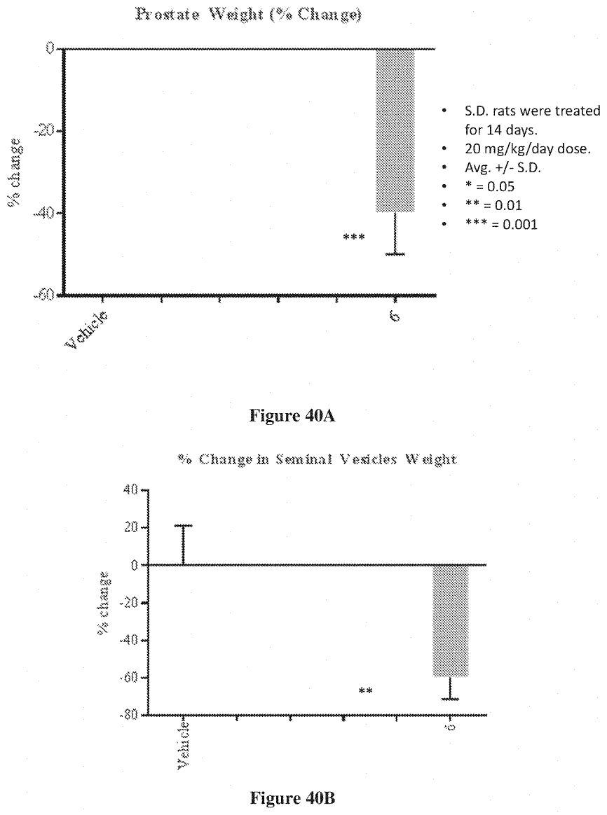

As shown in FIGS. 38, 1 and 6), and to some extent enzalutamide, were able to overcome 0.1 nM R1881 induced AR-dependent LNCaP proliferation. 1 and 6 demonstrated dose-dependent inhibition with full efficacy antiproliferation at 1 μM and 10 μM, respectively, whereas enzalutamide only reached approximately 40% efficacy at 1, 3, and M.

FIG. 39 describes that AR dependent gene expressions of PSA and FKBP5 in LNCaP cells were dose-dependently decreased by 1 and 6, like enzalutamide. This data confirms that AR antagonism observed in transcriptional activation assays translated into AR antagonism in AR dependent prostate cancer cells. (See methodology as described in Example 2 above.)

### Example 15: In Vitro Metabolic Stability in Mouse & Rat Liver Microsomes (MLM and RLM)

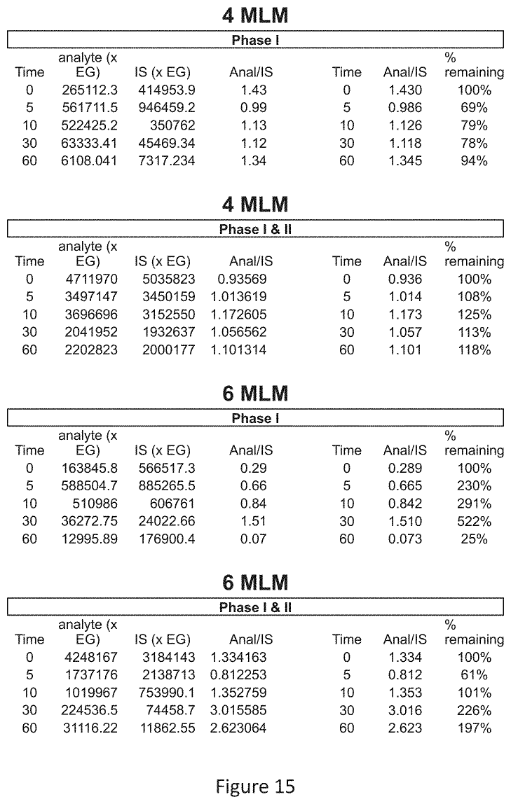

FIG. 15 depicts that 4 and 6 are stable for at least 60 minutes when incubated in vitro with mouse liver microsomes (MLM) under conditions that mimic Phase I and II metabolism. (See description of the methodology in Example 2.)

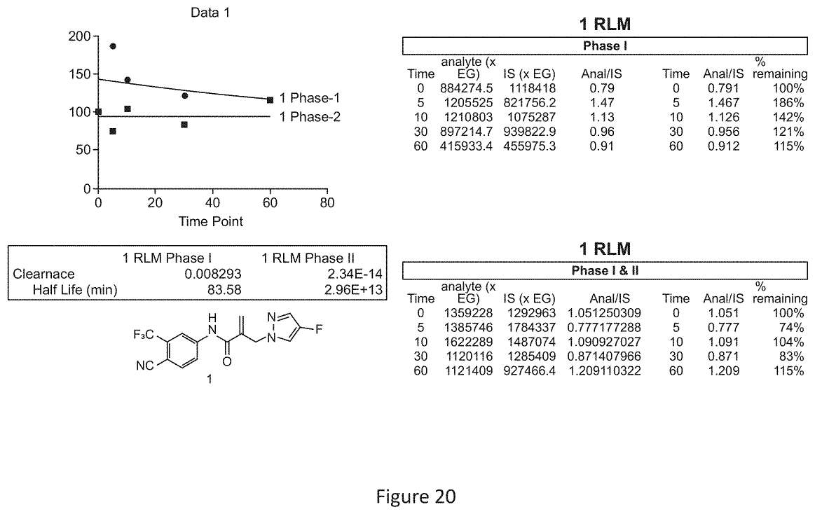

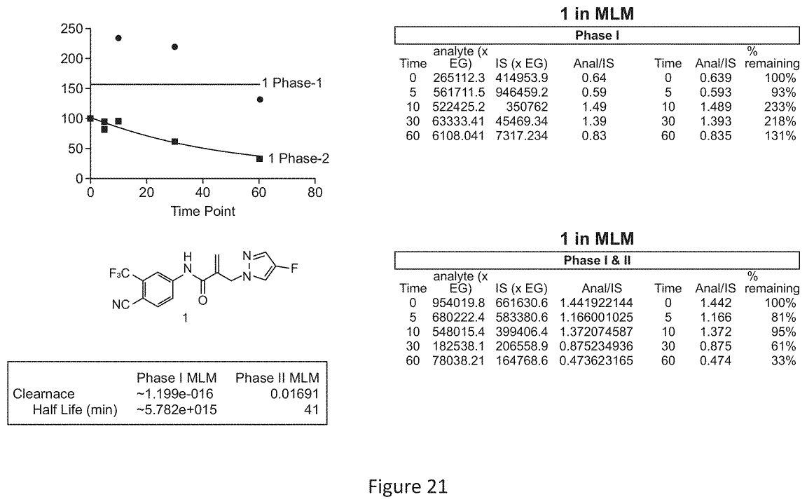

FIG. 20 depicts that 1 was stable in rat liver microsome (RLM) for >60 minutes. Estimated half-life for phase I stability was about 84 min, whereas FIG. 21 depicts that 1 had a half-life of 41 min in MLM in Phase I and II conditions.

Unexpectedly, despite possessing intrinsically reactive warhead functional groups, these stability data suggest that SARCAs of this invention are stable enough in rodent models to allow them to be tested for AR antagonism in vivo. If SARCAs are stable in the bloodstream and only react following binding to AR, then these SARCAs can be expected to have an unprecedented AR antagonist pharmacodynamics profile in vivo.

### Example 16: In Vivo AR Antagonism

In vivo AR antagonism was demonstrated in intact Sprague Dawley rats with SARCA 6 (FIGS. 41A and 41B). 20 mg/kg of 6 dosed daily for 14 days was sufficient to reduce the weight of androgen-dependent secondary sex organs. Prostate weights were reduced by ˜40% and seminal vesicles weights by ˜60%, and such reductions were statistically significant. It suggests that SARCA compounds of the invention are orally bioavailable and stable enough in the bloodstream to reach the prostate and seminal vesicles, and further confirms that SARCAs are potent enough to exert pharmacodynamics effects on AR target organs. Accordingly, SARCAs will be able to suppress the AR-axis in a wide variety of cell types thought the body and exert therapeutic antiandrogen effects in a wide variety of AR-dependent or androgen-dependent diseases and conditions as described herein. Further SARCAs of this invention are expected to suppress a broad spectrum of castrate resistant prostate cancer tumors or refractory breast cancer tumors including those whose growth is AR-V7 dependent or dependent on other AR mutations or truncations.

### Example 17: Mass Spectrometry Experiments to Determine Covalent Binding of SARCA

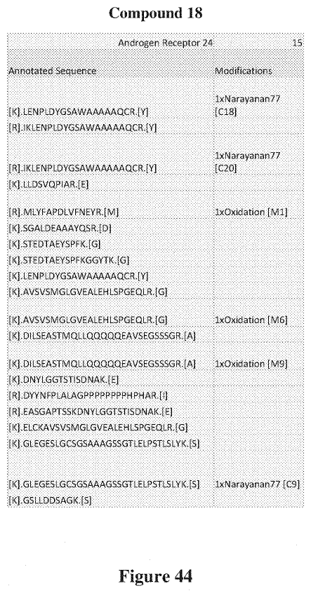

AR AF-1 protein was incubated with a molecule overnight at 4° C. The protein was digested with trypsin overnight at room temperature and was evaluated using mass spectrometry. Covalent molecules bind to cysteine and lysine, although interaction with amino acids has been detected. If a molecule is attached covalently to a peptide, the molecular weight of the peptide will increase by the molecule's molecular weight. For example, if a tryptic digested peptide's M.Wt. is 1000 Dalton and the incubated molecule's M.Wt. is 250 Dalton, then the covalently-bound peptide's M.Wt. will be ˜1250 Dalton. If two molecules are attached to a peptide, then the M.Wt. will increase correspondingly to ˜1500 Dalton. FIG. 44 depicts that compound 18 bound covalently to AR AF-1, with table showing that compound 18 bound to the peptides that contained select cysteines.

### Example 18: SARCA Compounds Activity

Methods: COS7 cells were plated in 24 well plates at 40,000 cells/well in DME+5% csFBS w/o phenol red. Twenty-four hours after plating, the cells were transfected with 0.25 ug GRE-LUC, 0.01 ug CMV-LUC, 0.025 ug CMV-hAR using lipofectamine reagents in optiMEM medium. Twenty-four hours after transfection, the cells were treated with a dose-response of the compounds in the presence of 0.1 nM R1881. Twenty-four hours after treatment, the cells were harvested, and luciferase assay was performed using Dual-luciferase reagent. Firefly values were divided by renilla numbers and the values are represented as relative light units (RLU).

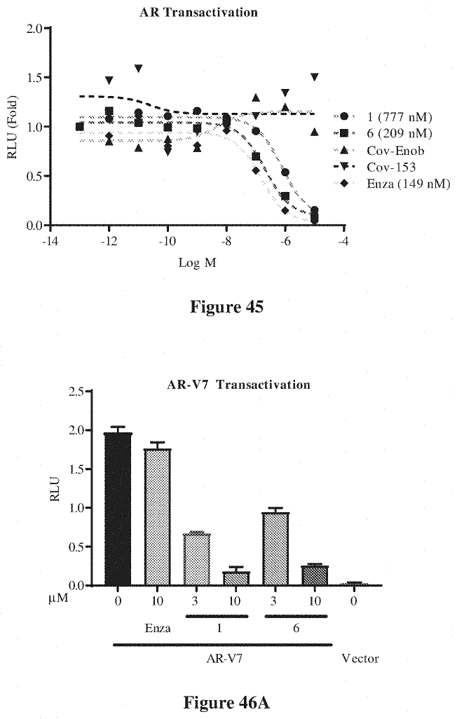

Results: FIG. 45 depicts AR antagonist activity of compounds 1 and 6.

Methods: COS7 cells were plated in 24 well plates at 40,000 cells/well in DME+5% csFBS w/o phenol red. Twenty-four hours after plating, the cells were transfected with 0.25 ug GRE-LUC, 0.01 ug CMV-LUC, 0.025 ug pCR3.1 hAR-V7 using lipofectamine reagents in optiMEM medium. Twenty-four hours after transfection, the cells were treated with the compounds. Twenty-four hours after treatment, the cells were harvested, and luciferase assay was performed using Dual-luciferase reagent. Firefly values were divided by renilla numbers and the values are represented as relative light units (RLU).

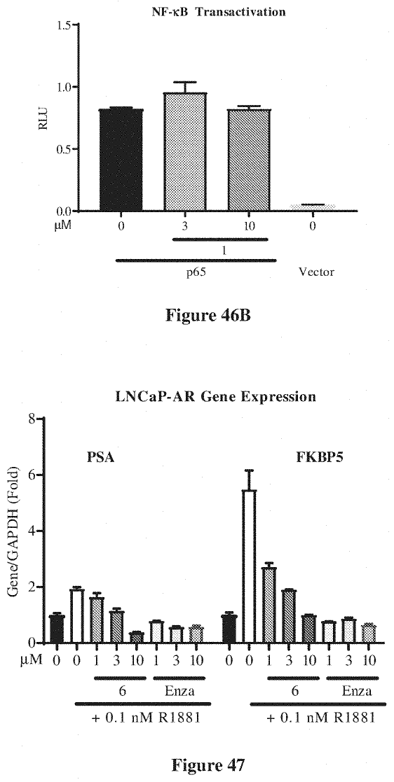

Results: As shown in FIGS. 46A and 46B, compounds 1 and 6 inhibited AR-V7 (FIG. 46A), but not NFkB (FIG. 46B), transactivation.

Methods: LNCaP cells over-expressing AR were plated in 96 well plates in RPMI+1% csFBS w/o phenol red medium. Cells were maintained in this medium for two days and then treated as indicated in the figure. Twenty-four hours after treatment, the cells were harvested, RNA isolated, and expression of the genes was quantified using real-time PCR.

Results: Compound 6 inhibited AR-target gene expression in prostate cancer cells as 1 o demonstrated in FIG. 47.

Methods: LNCaP-AR cells were plated in 96 well plates in RPMI+1% csFBS w/o phenol red medium. Cells were treated with the indicated doses of the compounds for 6 days, with medium change and retreatment after 3 days. Cells were fixed and stained with sulforhodamine blue (SRB). The stain color that is proportional to the number of cells was determined using a colorimeter.

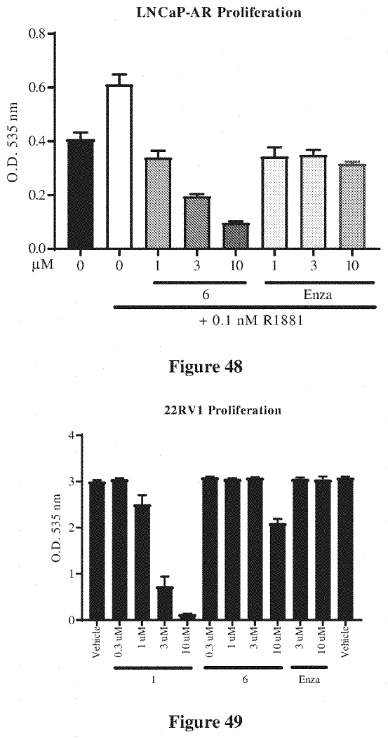

Results: As shown in FIG. 48, compound 6 inhibited prostate cancer cell proliferation.

Methods: 22RV1 cells were plated in 96 well plates in growth medium. Cells were treated with the indicated doses of the compounds for 6 days, with medium change and retreatment after 3 days. Cells were fixed and stained with sulforhodamine blue (SRB). The stain color that is proportional to the number of cells was determined using a colorimeter.

Results: As shown in FIG. 49, compounds 1 and 6 inhibited proliferation of prostate cancer cells that expressed AR-splice variants (AR-SVs).

Methods: Indicated cells were plated in 96 well plates in growth medium. Cells were treated with the indicated doses of the compounds for 6 days, with medium change and retreatment after 3 days. Cells were fixed and stained with sulforhodamine blue (SRB). The stain color that is proportional to the number of cells was determined using a colorimeter.

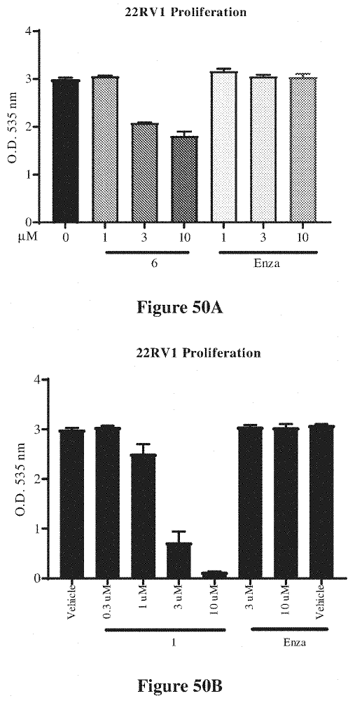

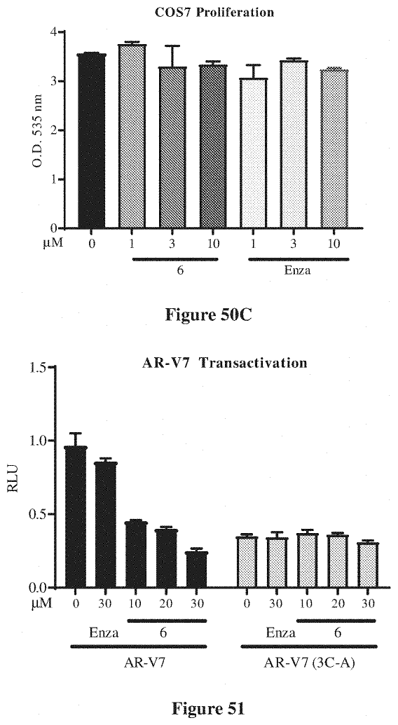

Results: Compounds 1 and 6 inhibited proliferation of prostate cancer cells that expressed AR-SVs, but not non-cancerous cells (FIGS. 50A-50C).

### Example 19: Transactivation of AR-V7 with Mutated Cysteines C267, C327, and C406

Methods: COS7 cells were plated in 24 well plates at 40,000 cells/well in DME+5% csFBS w/o phenol red. Twenty-four hours after plating, the cells were transfected with 0.25 ug GRE-LUC, 0.01 ug CMV-LUC, 0.025 ug pCDNA3.1 hAR-V7 or mutant AR-V7 (in which three cysteines (C267, C327, and C406) were mutated) using lipofectamine reagents in optiMEM medium. Twenty-four hours after transfection, the cells were treated with the compounds. Twenty-four hours after treatment, the cells were harvested, and luciferase assay was performed using Dual-luciferase reagent. Firefly values were divided by renilla numbers and the values are represented as relative light units (RLU).

Results: As demonstrated in FIG. 51, compounds 6 inhibited wildtype AR-V7 transactivation, but not transactivation of AR-V7 where three cysteines (C267, C327, and C406) were mutated. This data confirms that binding to the three cysteines is important for the SARCAs' function. Also, these three cysteines are important for AR-V7 function.

### Example 20: Mutating Individual Cysteines Did Not Affect SAR CA Activity

Methods: COS7 cells were plated in 24 well plates at 40,000 cells/well in DME+5% csFBS w/o phenol red. Twenty-four hours after plating, the cells were transfected with 0.25 ug GRE-LUC, 0.01 ug CMV-LUC, 0.025 ug pCDNA3.1 hAR-V7 or mutant AR-V7 (in which cysteines (C327, and C406) were mutated) using lipofectamine reagents in optiMEM medium. Twenty-four hours after transfection, the cells were treated with the compounds. Twenty-four hours after treatment, the cells were harvested, and luciferase assay was performed using Dual-luciferase reagent. Firefly values were divided by renilla numbers and the values were represented as relative light units (RLU).

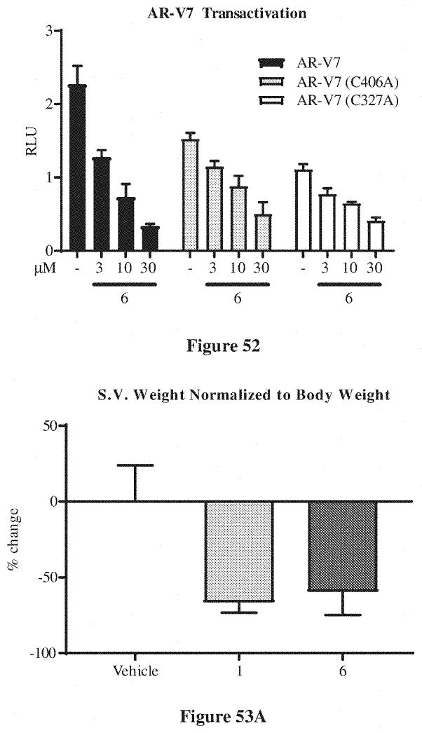

Results: FIG. 52 demonstrates that mutating individual cysteines did not affect compound 6 activity, but affected AR-V7 function. Mutating the cysteines individually to alanines, reduces ARV7 activity, but has minimum to no effect on SARCA inhibitory activity.

### Example 21: SARCAs Inhibited AR-Target Tissues Prostate and Seminal Vesicles

Methods: Hershberger assay results to study the body weight changes of representative compound. Intact Sprague Dawley rats (100-120 g body weight) (n=6/group) were dosed by 20 mg/kg for 13 days. Dosing solutions were prepared in 20% DMSO+80% PEG. Fourteen days after the initiation of treatment, animals were sacrificed and tissue weights were recorded. Body weights were measured on day 1 and at the time of sacrifice. Tissue weights were normalized to body weight and represented as percent change from vehicle-treated animals.

Results: As provided in FIGS. 53A and 53B, compounds 1 and 6 inhibited AR-target tissues prostate and seminal vesicles.

### Example 22: SARCAs Inhibited Growth of Prostate Cancer and TNBC

Methods: LNCaP cells over-expressing AR (5 million; 1:1 with matrigel) were implanted subcutaneously in male NSG mice (n=8-10/group). Once the tumors grow to 100-300 mm3, the animals were randomized and treated with vehicle, 30 mpk enza, or 60 mpk SARCA. Tumor volume was measured twice daily. Twenty-eight days after treatment initiation, the animals were sacrificed and tumors processed for further analysis. TNBC: MDA-MB-453 cells (5 million; 1:1 with matrigel) were implanted subcutaneously in female NSG mice (n=8-10/group). Once the tumors grow to 100-300 mm3, the animals were randomized and treated with vehicle or 60 mpk SARCA. Tumor volume was measured twice daily. Twenty-eight days after treatment initiation, the animals were sacrificed and tumors processed for further analysis.

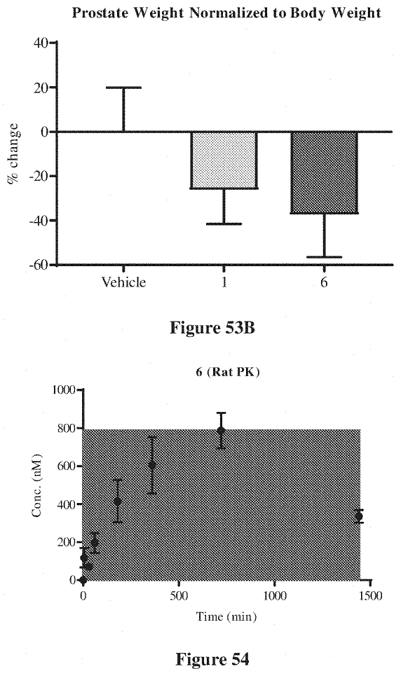

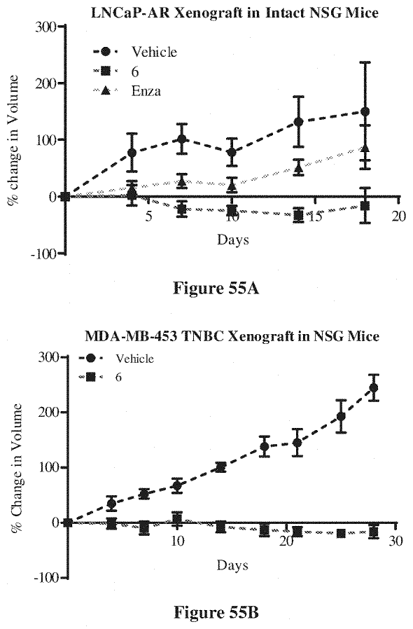

Results: Compound 6 inhibited growth of prostate cancer and triple-negative breast cancer xenograft growth in NSG mice (FIGS. 55A and 55B).

### Example 23: Quantification of Peptides Modified By SARCAs

Methods: Purified AF-1 protein was incubated with vehicle or 100 μM 1 and 6 overnight and the protein was trypsinized. The trypsinized peptides were analyzed by HPLC-mass spectrometer (LC-MS). Since covalent compounds irreversibly bind to a protein, the harsh conditions of MS will not dissociate a molecule from proteins. Analyzing the peptides in LC-MS showed that 1 and 6 bound strongly to two cysteines (C406 and C327) and very weakly and inconsistently to one cysteine (C267) in the AF-1 domain. The advantage of covalent binding is that the binding can be easily detected by molecular weight change of the peptides corresponding to the molecule's molecular weight. Despite the presence of 8 cysteines and 11 lysines in AF-1, the molecules selectively bound to C406 and C327. While 1 and 6 bound to AF-1 covalently, other non-specific compounds (covalent modification of enobosarm) failed to bind to the AF-1, providing a structure activity relationship for the interaction with the AF-1. Despite over 75% homology in the structure between 1 and 6 and covalent-enobosarm, the striking difference in binding to AF-1 is a clear indication of the importance of the pyrazole ring for this scaffold's binding to AF-1. Quantification of modified residues indicated that 1 and 6 modified 60-80% of the C406 and C327 coding peptides (and a small percent of C267). The cross-reactivity of 1 and 6 with other purified proteins was evaluated. While 1 cross-reacted with LBD at approximately 50% and with glutathione S-transferase (GST) at about 10% of the AF-1 modifications, 6 was selective to AF-1 with a very modest 2-5% modification observed in LBD and GST. All these experiments were conducted at 100 μM. These results again confirm that 6 is highly selective to AF-I, especially to C327 and C406 amino acids.

A dose response of compounds 1 and 6 was performed with purified AF-I protein. Both 1 and 6 demonstrated significant binding both at 30 and 100 μM to C406 and C327 and a modest modification at 10 μM concentration. At concentrations lower than 100 M, no modification of proteins other than AF-I (PR-LBD, GST, or AR-LBD) was observed with 6.

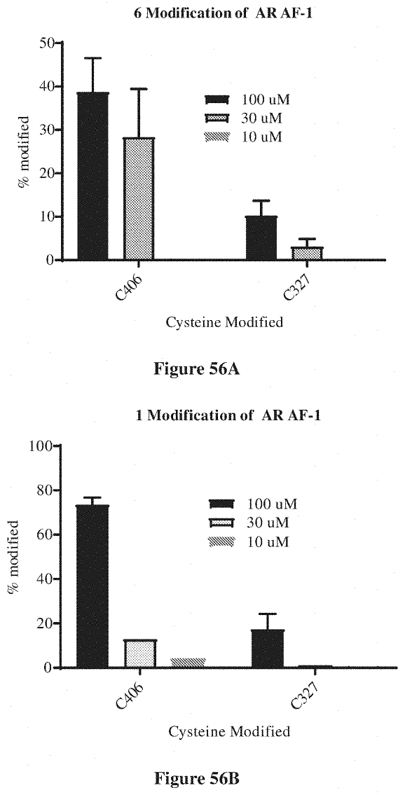

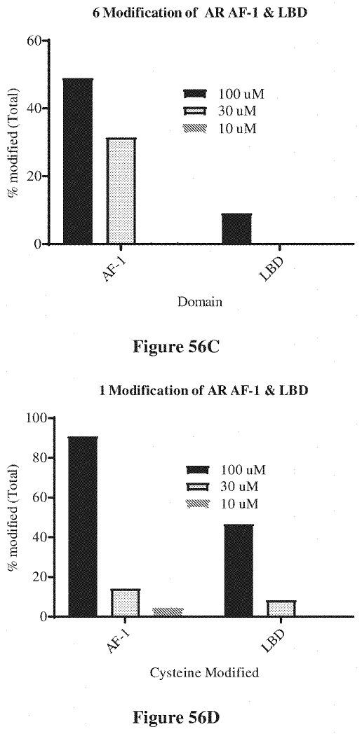

Results: FIGS. 56A-56D describes quantification of peptides modified by compounds 1 and 6.

### Example 24: Single Point Mutations of C406 and C327 Reduced AR-V7 Activity and Stability

As demonstrated in the examples herein, selective binding of 1 and 6 to C406, C327, and C267 that resulted in the inhibition of AR and AR-V7 function suggests the importance of these three amino acids and this region for AR and AR-V7 function.

The three amino acids were mutated (3C-A) and the effect of the mutation on AR-V7 expression was evaluated. Wild type or 3C-A (where C406, C327, and C267 were mutated to alanines) AR-V7 were expressed in COS7 cells and the expression of AR-V7 at the protein and mRNA levels was measured by Western blot and real-time PCR, respectively. Interestingly, mutating the three amino acids completely destabilized the AR-V7 protein, with no AR-V7 protein detected in the 3CA AR-V7 transfected cells. AR-V7 mRNA was detected at a higher level in the 3C-A AR-V7 transfected cells than the wildtype AR-V7 transfected cells. These results suggest that these three amino acids are extremely critical for AR-V7 stability, but not for AR stability.

Single point mutations of C406 and C327 reduced AR-V7 activity and stability. Since the triple C-A mutation caused a greater than 50% decrease in AR-V7 transactivation, single point mutations of C406 and C327 were created and the stability of AR-V7 and point mutant AR-V7 was evaluated by Western blot analysis. Single point mutation of C406 and C327 resulted in over 80-90% reduction in AR-V7 protein levels, without much alteration in AR-V7 mRNA. These results clearly demonstrate that C406 and C327 are extremely important for the stability of AR-V7 and mutating or blocking any one of them will result in its destabilization and functional loss.

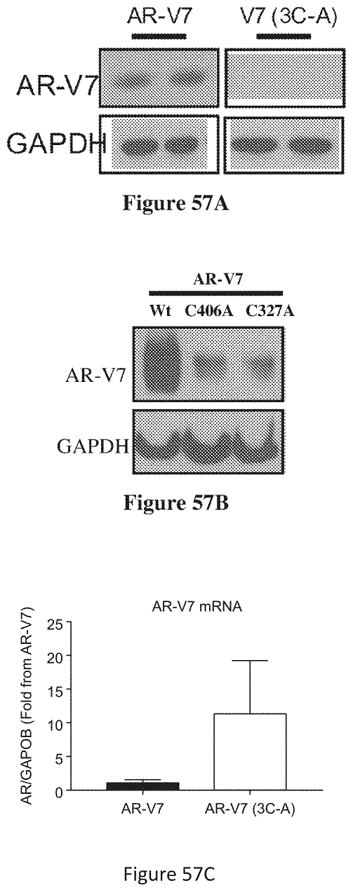

Results: FIGS. 57A-57C demonstrate that C406, C327, and C267 were important for the AR-V7 stability.

### Example 25: SARCAs Minimally Cross-Reacted with GST

The cross-reactivity of 1 and 6 was evaluated with other purified proteins. While 1 cross-reacted with LBD at approximately 50% and with glutathione S-transferase (GST) at about 10% of the AF-1 modifications, 6 was selective to AF-1 with a very modest 2-5% modification observed in LBD and GST. All these experiments were conducted at 100 μM. These results again confirm that 6 is highly selective to AF-1, especially to C327 and C406 amino acids.

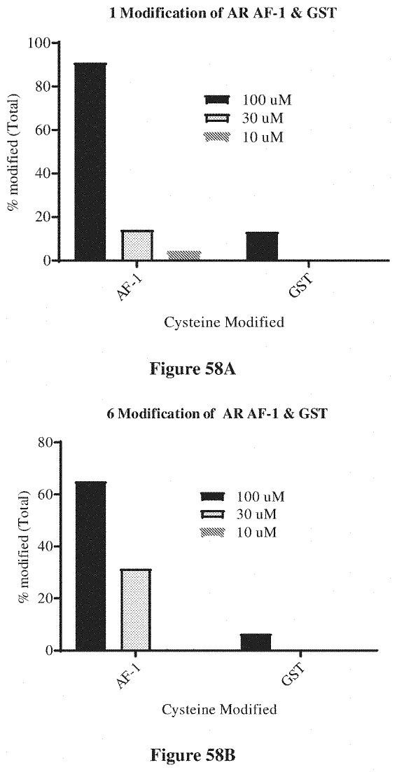

Results: FIGS. 58A and 58B demonstrate that compounds 1 and 6 minimally cross-reacted with GST.

### Example 26: SARCAs Competed with UT-105 and UT-34

The potential of UT-34 and UT-105 to compete with 6 for binding was evaluated.

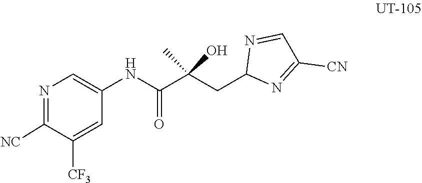 [SMILES: C[C@](O)(CC1N=CC(C#N)=N1)C(=O)Nc1cnc(C#N)c(C(F)(F)F)c1]

Methods: AF-1 protein was pre-incubated with 100 μM UT-34 or UT-105 for 2 hours and then with 30 μM 6. The trypsin-digested peptides were analyzed by LC-MS. 6-dependent C406 and C327 modifications were significantly reversed by UT-34. This suggests that these molecules have comparable binding conformation to the AF-1 that involves C406 and C327 or a pocket that engages these two cysteines. Mutation of C407, C327, and C267 resulted in complete loss of 6 binding to the AF-1, suggesting that 6 does not bind to other cysteines or lysines in the absence of these three amino acids. Collectively, these results convincingly demonstrate the existence of a binding region in the AF-1 that can be utilized with appropriate chemical scaffold to target AR and AR-SVs. Considering that the three cysteines are not adjacent to each other, the covalent molecules should create a three-dimensional structure in the AF-1 that result in binding to these amino acids.

M.S. studies were performed as indicated above. The percent modified cysteines to unmodified was quantified and plotted as graph.

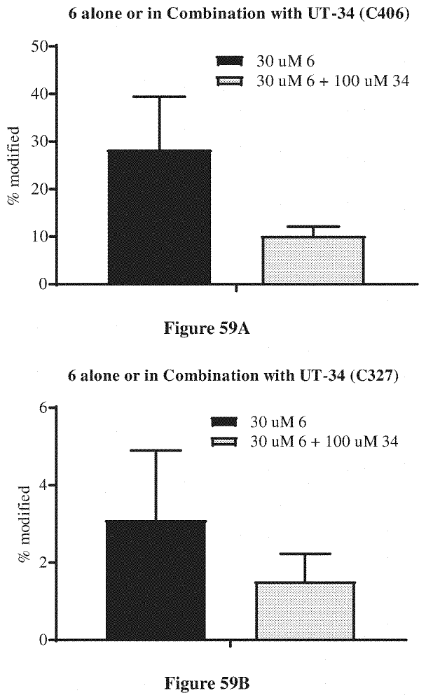

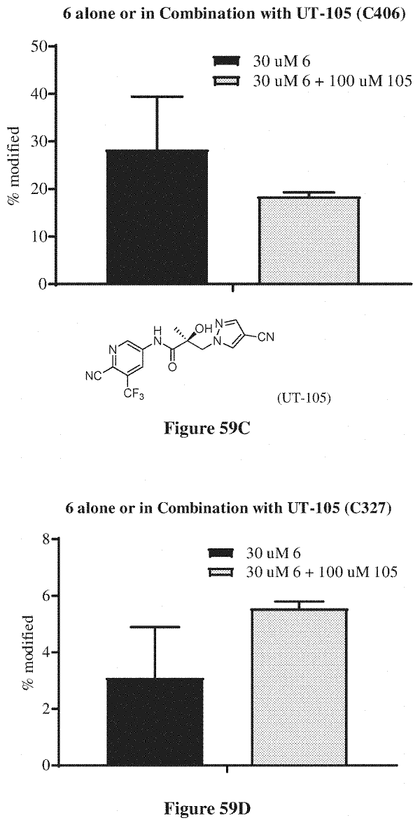

Results: As demonstrated in FIGS. 59A-59D, UT-105 and UT-34 competed with 1 and 6 for binding to AF-1. (Both UT-105 and UT-34 are non-covalent binders of AF-1.)

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound represented by the structure of formula I 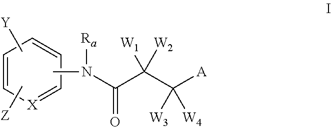
wherein
X is CH or N;
Y is H, CF₃, F, Br, Cl, I, CN, or C(R)₃;
Z is H, NO₂, CN, F, Br, Cl, I, COOH, COR, NHCOR, or CONHR;
or Y and Z form a 5 to 8 membered fused ring;
R is H, alkyl, alkenyl, CH₂CH₂OH, CF₃, CH₂Cl, CH₂CH₂Cl, aryl, F, Cl, Br, I, or OH;
Rₐ is H, alkyl-NCO, alkyl-NCS, alkyl-SCN, alkyl-OCN, alkyl-N₃, alkyl-SO₂F, alkyl-CH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂, wherein halide is F, Cl, Br, or I;
W₁ is H or OR_{d}, wherein R_{d} is H, alkyl-NCO, alkyl-NCS, alkyl-SCN, alkyl-OCN, alkyl-N₃, alkyl-SO₂F, alkyl-CH₂halide, -alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
W₂ is CH₃, CH₂F, CHF₂, CF₃, CH₂CH₃, CF₂CF₃, or CH₂A;
or W₁ and W₂, together with the carbon atom to which they are attached, form a C═CW₅W₆ group, wherein W₅ and W₆ are each H or alkyl;
W₃ and W₄ are individually H, OH, alkyl, wherein the alkyl is optionally substituted with OR, NO₂, CN, F, Br, Cl, I, COR, NHCOR, CONHR, —NCO, —NCS, —SCN, —OCN, —N₃, —SO₂F, —CH₂halide, —NHCOCH₂halide, —NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, C(CONHCOR)═CH₂, —CH₂—CH═CH—CON(R)₂, or —CH₂—C(CON(R)₂)═CH₂;
or one of W₁ and W₂ with one of W₃ and W₄, together with the carbon atoms to which they are attached, form a C═C bond;
A is NR_{b}R_{c} or a 5 to 10-membered aryl or heteroaryl group, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
R_{b} is H or alkyl, wherein the alkyl is optionally substituted with OR, NO₂, CN, F, Br, Cl, I, COR, NHCOR, or CONHR;
R_{c} is alkyl, haloalkyl, cycloalkyl, heterocycloalkyl, aryl or heteroaryl, wherein said alkyl, haloalkyl, cycloalkyl, heterocycloalkyl, aryl and heteroaryl groups are optionally substituted with CN, NO₂, CF₃, F, Cl, Br, I NHCOOR, N(R)₂, NHCOR, COR, alkyl, or alkoxy;
or R_{b} and R_{c}, together with the nitrogen atom to which they are attached, form a 5 to 10-membered saturated or unsaturated heterocyclic ring having at least one nitrogen atom and 0, 1, or 2 double bonds, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
or its isomer, optical isomer, racemic mixture, pharmaceutically acceptable salt, pharmaceutical product, hydrate or any combination thereof.

2. The compound of claim 1, wherein said compound is represented by the structure of formula II 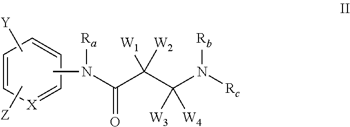
wherein
X is CH or N;
Y is H, CF₃, F, Br, Cl, I, CN, or C(R)₃;
Z is H, NO₂, CN, F, Br, Cl, I, COOH, COR, NHCOR, or CONHR;
or Y and Z form a 5 to 8 membered fused ring;
R is H, alkyl, alkenyl, CH₂CH₂OH, CF₃, CH₂Cl, CH₂CH₂Cl, aryl, F, Cl, Br, I, or OH;
Rₐ is H, alkyl-NCO, alkyl-NCS, alkyl-SCN, alkyl-OCN, alkyl-N₃, alkyl-SO₂F, alkylCH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂, wherein halide is F, Cl, Br, or I;
W₁ is H or OR_{d}, wherein R_{d} is H, alkyl-NCO, alkyl-NCS, alkyl-SCN, alkyl-OCN, alkyl-N₃, alkyl-SO₂F, alkyl-CH₂halide, alkyl-NHCOCH₂halide, alkyl-NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
W₂ is CH₃, CH₂F, CHF₂, CF₃, CH₂CH₃, CF₂CF₃, or CH₂A;
or W₁ and W₂, together with the carbon atom to which they are attached, form a C═CW₅W₆ group, wherein W₅ and W₆ are each H or alkyl;
W₃ and W₄ are individually H, OH, alkyl, wherein the alkyl is optionally substituted with OR, NO₂, CN, F, Br, Cl, I, COR, NHCOR, CONHR, —NCO, —NCS, —SCN, —OCN, —N₃, —SO₂F, —CH₂halide, —NHCOCH₂halide, —NHSO₂CH₂halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
or one of W₁ and W₂ with one of W₃ and W₄, together with the carbon atoms to which they are attached, form a C═C bond;
A is NR_{b}R_{c} or a 5 to 10-membered aryl or heteroaryl group, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CHCONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
R_{b} is H or alkyl, wherein the alkyl is optionally substituted with OR, NO₂, CN, F, Br, Cl, I, COR, NHCOR, or CONHR;
R_{c} is alkyl, haloalkyl, cycloalkyl, heterocycloalkyl, aryl or heteroaryl, wherein said alkyl, haloalkyl, cycloalkyl, heterocycloalkyl, aryl and heteroaryl groups are optionally substituted with CN, NO₂, CF₃, F, Cl, Br, I NHCOOR, N(R)₂, NHCOR, COR, alkyl, or alkoxy;
or R_{b} and R_{c}, together with the nitrogen atom to which they are attached, form a 5 to 10-membered saturated or unsaturated heterocyclic ring having at least one nitrogen atom and 0, 1, or 2 double bonds, optionally substituted with at least one of Q¹, Q², Q³ and Q⁴, each independently selected from hydrogen, keto, substituted or unsubstituted alkyl, substituted or unsubstituted cycloalkyl, substituted or unsubstituted heterocycloalkyl, haloalkyl, CF₃, substituted or unsubstituted aryl, F, Cl, Br, I, CN, NO₂, hydroxyl, alkoxy, OR, benzyl, NCS, maleimide, NHCOOR, N(R)₂, NHCOR, CONHR, COOR, COR, —NCO, —NCS, —SCN, —OCN, —N₃, —SO₂F, —CH₂halide, —NHCOCH₂-halide, —NHSO₂CH₂-halide, —CH₂—CH═CH—COOR, —CH₂—C(COOR)═CH₂, —CH₂—CH═CH—CONHR, —CH₂—C(CONHR)═CH₂, —CH₂—CH═CH—CONHCOR, —CH₂—C(CONHCOR)═CH₂, —CH₂—CH═CH—CON(R)₂, or —CH₂—C(CON(R)₂)═CH₂;
or its isomer, optical isomer, racemic mixture, pharmaceutically acceptable salt, pharmaceutical product, hydrate or any combination thereof.

3. The compound of claim 1, wherein said compound is represented by the structure of any one of the following compounds:

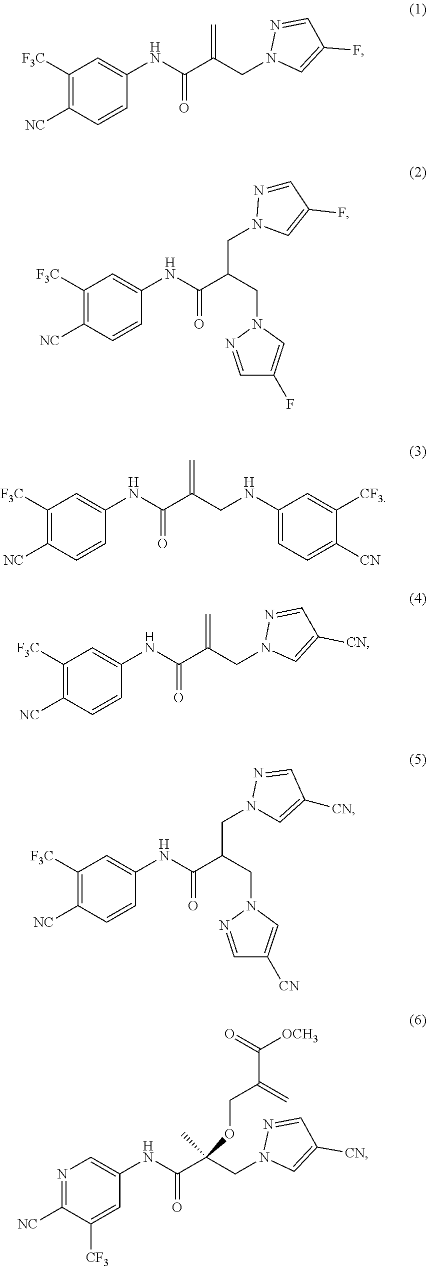 [SMILES: C=C(Cn1cc(F)cn1)C(=O)Nc1ccc(C#N)c(C(F)(F)F)c1 ; N#Cc1ccc(NC(=O)C(Cn2cc(F)cn2)Cn2cc(F)cn2)cc1C(F)(F)F ; C=C(CNc1ccc(C#N)c(C(F)(F)F)c1)C(=O)Nc1ccc(C#N)c(C(F)(F)F)c1 ; C=C(Cn1cc(C#N)cn1)C(=O)Nc1ccc(C#N)c(C(F)(F)F)c1 ; N#Cc1cnn(CC(Cn2cc(C#N)cn2)C(=O)Nc2ccc(C#N)c(C(F)(F)F)c2)c1 ; C=C(CO[C@@](C)(Cn1cc(C#N)cn1)C(=O)Nc1cnc(C#N)c(C(F)(F)F)c1)C(=O)OC]

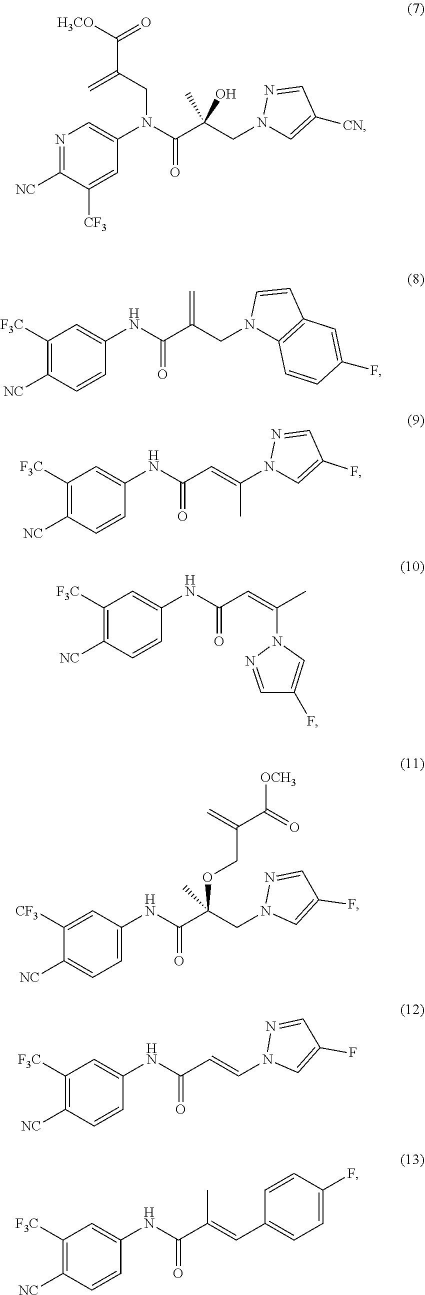

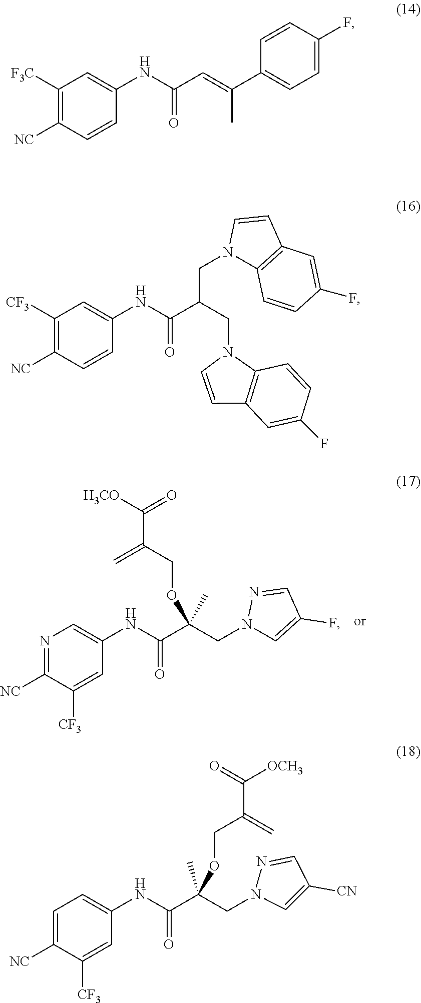 [SMILES: C/C(=C\C(=O)Nc1ccc(C#N)c(C(F)(F)F)c1)c1ccc(F)cc1 ; N#Cc1ccc(NC(=O)C(Cn2ccc3cc(F)ccc32)Cn2ccc3cc(F)ccc32)cc1C(F)(F)F ; C=C(CO[C@@](C)(Cn1cc(F)cn1)C(=O)Nc1cnc(C#N)c(C(F)(F)F)c1)C(=O)OC ; C=C(CO[C@@](C)(Cn1cc(C#N)cn1)C(=O)Nc1ccc(C#N)c(C(F)(F)F)c1)C(=O)OC]

4. The compound of claim 1, wherein said compound is a selective androgen receptor covalent antagonist (SARCA) compound containing at least one nucleophile acceptor group.

5. The compound of claim 4, where said nucleophile acceptor group is a Michael addition reaction acceptor or at least one of —NCO, —NCS, —N₃, 2-haloacetyl, or halomethyl.

6. The compound of claim 1, wherein Rₐ and R_{d} are not H at the same time.

7. A compound represented by the structure of compound 15

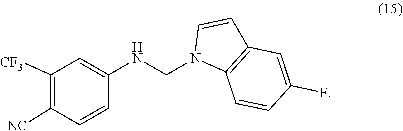 [SMILES: N#Cc1ccc(NCn2ccc3cc(F)ccc32)cc1C(F)(F)F]

8. (canceled)

9. A method of treating an androgen receptor dependent disease or condition in a subject in need thereof, comprising administering to the subject a therapeutically effective amount of a compound of claim 1.

10. The method of claim 9, wherein said compound binds irreversibly to androgen receptor (AR).

11. The method of claim 9, wherein said androgen receptor dependent disease or condition in said subject responds to at least one of AR-splice variant (AR-SV) degradation activity, full length (AR-FL) degradation activity, AR-SV inhibitory, or AR-FL inhibitory activity.

12. The method of claim 9, wherein said androgen receptor dependent disease or condition is breast cancer in said subject.

13. The method of claim 9, wherein said subject has AR expressing breast cancer, AR-SV expressing breast cancer, and/or AR-V7 expressing breast cancer.

14. (canceled)

15. (canceled)

16. The method of claim 1, wherein said androgen receptor dependent disease or condition is a hormonal disease or condition in a female is selected from the group consisting of precocious puberty, dysmenorrhea, amenorrhea, multilocular uterus syndrome, endometriosis, hysteromyoma, abnormal uterine bleeding, early menarche, fibrocystic breast disease, fibroids of the uterus, ovarian cysts, polycystic ovary syndrome, pre-eclampsia, eclampsia of pregnancy, preterm labor, premenstrual syndrome, or vaginal dryness.

17. The method of claim 9, wherein said androgen receptor dependent disease or condition is hormonal disease or condition in a male in said subject.

18. The method of claim 17, wherein said hormonal disease or condition in a male is at least one of hypergonadism, hypersexuality, sexual dysfunction, gynecomastia, precocious puberty in a male, alterations in cognition and mood, depression, hair loss, hyperandrogenic dermatological disorders, pre-cancerous lesions of the prostate, benign prostate hyperplasia, prostate cancer and/or other androgen-dependent cancers.

19. The method of claim 9, wherein said androgen receptor dependent disease or condition is sexual perversion, hypersexuality, paraphilias, androgen psychosis, virilization, or androgen insensitivity syndrome in said subject.

20. The method of claim 9, wherein said androgen receptor dependent disease or condition is AR-expressing cancer in said subject.

21. The method of claim 9, wherein said androgen receptor dependent disease or condition is amyotrophic lateral sclerosis (ALS), uterine fibroids, or abdominal aortic aneurysm (AAA) in said subject.

22. The method of claim 9, wherein said androgen receptor dependent disease or condition is caused by polyglutamine (polyQ) AR polymorphs in a subject.

23. A method of treating prostate cancer (PCa) or increasing the survival of a male subject suffering from prostate cancer comprising administering to the subject a therapeutically effective amount of a compound of claim 1, or its isomer, optical isomer, or any mixture of optical isomers, pharmaceutically acceptable salt, pharmaceutical product, hydrate or any combination thereof.

24. (canceled)


SEQUENCE LISTING (199 sequences)

SEQ ID NO: 1 - protein, 17 aa, Homo sapiens
STEDTAEYSPFKGGYTK

SEQ ID NO: 2 - protein, 17 aa, Homo sapiens
STEDTAEYSPFKGGYTK

SEQ ID NO: 3 - protein, 27 aa, Homo sapiens
GDCMYAPLLGVPPAVRPTPCAPLAECK

SEQ ID NO: 4 - protein, 27 aa, Homo sapiens
GDCMYAPLLGVPPAVRPTPCAPLAECK

SEQ ID NO: 5 - protein, 19 aa, Homo sapiens
LENPLDYGSAWAAAAAQCR

SEQ ID NO: 6 - protein, 19 aa, Homo sapiens
LENPLDYGSAWAAAAAQCR

SEQ ID NO: 7 - protein, 20 aa, Homo sapiens
IKLENPLDYGSAWAAAAAQC

SEQ ID NO: 8 - protein, 13 aa, Homo sapiens
SGALDEAAAYQSR

SEQ ID NO: 9 - protein, 21 aa, Homo sapiens
SGALDEAAAYQSRDYYNFPLA

SEQ ID NO: 10 - protein, 12 aa, Homo sapiens
STEDTAEYSPFK

SEQ ID NO: 11 - protein, 17 aa, Homo sapiens
STEDTAEYSPFKGGYTK

SEQ ID NO: 12 - protein, 16 aa, Homo sapiens
STEDTAEYSPFKGYTK

SEQ ID NO: 13 - protein, 22 aa, Homo sapiens
GSLLDDSAGKSTEDTAEYSPFK

SEQ ID NO: 14 - protein, 22 aa, Homo sapiens
GSLLDDSAGKSTEDTAEYSPFK

SEQ ID NO: 15 - protein, 10 aa, Homo sapiens
GSLLDDSAGK

SEQ ID NO: 16 - protein, 21 aa, Homo sapiens
AREASGAPTSSKDNYLGGTST

SEQ ID NO: 17 - protein, 21 aa, Homo sapiens
AVSVSMGLGVEALEHLSPGEC

SEQ ID NO: 18 - protein, 21 aa, Homo sapiens
AVSVSMGLGVEALEHLSPGEC

SEQ ID NO: 19 - protein, 21 aa, Homo sapiens
DILSEASTMQLLQQQQQEAVS

SEQ ID NO: 20 - protein, 21 aa, Homo sapiens
DILSEASTMQLLQQQQQEAVS

SEQ ID NO: 21 - protein, 15 aa, Homo sapiens
DNYLGGTSTISDNAK

SEQ ID NO: 22 - protein, 19 aa, Homo sapiens
DNYLGGTSTISDNAKELCK

SEQ ID NO: 23 - protein, 21 aa, Homo sapiens
DYYNFPLALAGPPPPPPPPHP

SEQ ID NO: 24 - protein, 21 aa, Homo sapiens
DYYNFPLALAGPPPPPPPPHP

SEQ ID NO: 25 - protein, 10 aa, Homo sapiens
EASGAPTSSK

SEQ ID NO: 26 - protein, 22 aa, Homo sapiens
EASGAPTSSKDNYLGGTSTISD

SEQ ID NO: 27 - protein, 21 aa, Homo sapiens
GDCMYAPLLGVPPAVRPTPCA

SEQ ID NO: 28 - protein, 21 aa, Homo sapiens
GDCMYAPLLGVPPAVRPTPCA

SEQ ID NO: 29 - protein, 21 aa, Homo sapiens
GLEGESLGCSGAAAGSSGTLE

SEQ ID NO: 30 - protein, 21 aa, Homo sapiens
GLEGESLGCSGAAAGSSGTLE

SEQ ID NO: 31 - protein, 22 aa, Homo sapiens
EASGAPTSSKDNYLGGTSTISD

SEQ ID NO: 32 - protein, 38 aa, Homo sapiens
SGALDEAAAYQSRDYYNFPLALAGPPPPPPPPHPPHAR

SEQ ID NO: 33 - protein, 13 aa, Homo sapiens
SGALDEAAAYQSR

SEQ ID NO: 34 - protein, 21 aa, Homo sapiens
IKLENPLDYGSAWAAAAAQCR

SEQ ID NO: 35 - protein, 17 aa, Homo sapiens
STEDTAEYSPFKGGYTK

SEQ ID NO: 36 - protein, 12 aa, Homo sapiens
STEDTAEYSPFK

SEQ ID NO: 37 - protein, 19 aa, Homo sapiens
LENPLDYGSAWAAAAAQCR

SEQ ID NO: 38 - protein, 29 aa, Homo sapiens
EASGAPTSSKDNYLGGTSTISDNAKELCK

SEQ ID NO: 39 - protein, 25 aa, Homo sapiens
EASGAPTSSKDNYLGGTSTISDNAK

SEQ ID NO: 40 - protein, 10 aa, Homo sapiens
EASGAPTSSK

SEQ ID NO: 41 - protein, 24 aa, Homo sapiens
DYYNFPLALAGPPPPPPPPHPHAR

SEQ ID NO: 42 - protein, 19 aa, Homo sapiens
DNYLGGTSTISDNAKELCK

SEQ ID NO: 43 - protein, 15 aa, Homo sapiens
DNYLGGTSTISDNAK

SEQ ID NO: 44 - protein, 28 aa, Homo sapiens
DILSEASTMQLLQQQQQEAVSEGSSSGR

SEQ ID NO: 45 - protein, 28 aa, Homo sapiens
DILSEASTMQLLQQQQQEAVSEGSSSGR

SEQ ID NO: 46 - protein, 23 aa, Homo sapiens
AVSVSMGLGVEALEHLSPGEQLR

SEQ ID NO: 47 - protein, 23 aa, Homo sapiens
AVSVSMGLGVEALEHLSPGEQLR

SEQ ID NO: 48 - protein, 19 aa, Homo sapiens
LENPLDYGSAWAAAAAQCR

SEQ ID NO: 49 - protein, 22 aa, Homo sapiens
GSLLDDSAGKSTEDTAEYSPFK

SEQ ID NO: 50 - protein, 10 aa, Homo sapiens
GSLLDDSAGK

SEQ ID NO: 51 - protein, 31 aa, Homo sapiens
GLEGESLGCSGSAAAGSSGTLELPSTLSLYK

SEQ ID NO: 52 - protein, 31 aa, Homo sapiens; identical to SEQ ID NO: 51

SEQ ID NO: 53 - protein, 5 aa, Homo sapiens
AREAS

SEQ ID NO: 54 - protein, 5 aa, Homo sapiens
GSLLD

SEQ ID NO: 55 - protein, 5 aa, Homo sapiens
GSLLD

SEQ ID NO: 56 - protein, 5 aa, Homo sapiens
GSLLD

SEQ ID NO: 57 - protein, 5 aa, Homo sapiens
LENPL

SEQ ID NO: 58 - protein, 5 aa, Homo sapiens
GLEGE

SEQ ID NO: 59 - protein, 5 aa, Homo sapiens
SGALD

SEQ ID NO: 60 - protein, 5 aa, Homo sapiens
STEDT

SEQ ID NO: 61 - protein, 5 aa, Homo sapiens
IKLEN

SEQ ID NO: 62 - protein, 5 aa, Homo sapiens
AVSVS

SEQ ID NO: 63 - protein, 5 aa, Homo sapiens
AVSVS

SEQ ID NO: 64 - protein, 6 aa, Homo sapiens
DILSEA

SEQ ID NO: 65 - protein, 6 aa, Homo sapiens
DILSEA

SEQ ID NO: 66 - protein, 5 aa, Homo sapiens
DNYLG

SEQ ID NO: 67 - protein, 5 aa, Homo sapiens
DNYLG

SEQ ID NO: 68 - protein, 5 aa, Homo sapiens
DYYNP

SEQ ID NO: 69 - protein, 5 aa, Homo sapiens
EASGA

SEQ ID NO: 70 - protein, 5 aa, Homo sapiens
EASGA

SEQ ID NO: 71 - protein, 5 aa, Homo sapiens
EASGA

SEQ ID NO: 72 - protein, 5 aa, Homo sapiens
ELCKA

SEQ ID NO: 73 - protein, 4 aa, Homo sapiens
GDCM

SEQ ID NO: 74 - protein, 4 aa, Homo sapiens
GDCM

SEQ ID NO: 75 - protein, 5 aa, Homo sapiens
STEDT

SEQ ID NO: 76 - protein, 12 aa, Homo sapiens
STEDTAEYSPFK

SEQ ID NO: 77 - protein, 17 aa, Homo sapiens
STEDTAEYSPFKGGYTK

SEQ ID NO: 78 - protein, 22 aa, Homo sapiens
GSLLDDSAGKSTEDTAEYSPFK

SEQ ID NO: 79 - protein, 10 aa, Homo sapiens
GSLLDDSAGK

SEQ ID NO: 80 - protein, 31 aa, Homo sapiens; identical to SEQ ID NO: 51

SEQ ID NO: 81 - protein, 27 aa, Homo sapiens
AREASGAPTSSKDNYLGGTSTISDNAK

SEQ ID NO: 82 - protein, 23 aa, Homo sapiens
AVSVSMGLGVEALEHLSPGEQLR

SEQ ID NO: 83 - protein, 23 aa, Homo sapiens
AVSVSMGLGVEALEHLSPGEQLR

SEQ ID NO: 84 - protein, 28 aa, Homo sp.
DILSEASTMQLLQQQQQEAVSEGSSSGR

SEQ ID NO: 85 - protein, 28 aa, Homo sapiens
DILSEASTMQLLQQQQQEAVSEGSSSGR

SEQ ID NO: 86 - protein, 15 aa, Homo sapiens
DNYLGGTSTISDNAK

SEQ ID NO: 87 - protein, 19 aa, Homo sapiens
DNYLGGTSTISDNAKELCK

SEQ ID NO: 88 - protein, 24 aa, Homo sapiens
DYYNFPLALAGPPPPPPPPHPHAR

SEQ ID NO: 89 - protein, 10 aa, Homo sapiens
EASGAPTSSK

SEQ ID NO: 90 - protein, 25 aa, Homo sapiens
EASGAPTSSKDNYLGGTSTISDNAK

SEQ ID NO: 91 - protein, 29 aa, Homo sapiens
EASGAPTSSKDNYLGGTSTISDNAKELCK

SEQ ID NO: 92 - protein, 29 aa, Homo sapiens
EASGAPTSSKDNYLGGTSTISDNAKELCK

SEQ ID NO: 93 - protein, 27 aa, Homo sapiens
GDCMYAPLLGVPPAVRPTPCAPLAECK

SEQ ID NO: 94 - protein, 27 aa, Homo sapiens
GDCMYAPLLGVPPAVRPTPCAPLAECK

SEQ ID NO: 95 - protein, 27 aa, Homo sapiens
GDCMYAPLLGVPPAVRPTPCAPLAECK

SEQ ID NO: 96 - protein, 27 aa, Homo sapiens
GDCMYAPLLGVPPAVRPTPCAPLAECK

SEQ ID NO: 97 - protein, 27 aa, Homo sapiens
GDCMYAPLLGVPPAVRPTPCAPLAECK

SEQ ID NO: 98 - protein, 31 aa, Homo sapiens; identical to SEQ ID NO: 51

SEQ ID NO: 99 - protein, 10 aa, Homo sapiens
GSLLDDSAGK

SEQ ID NO: 100 - protein, 22 aa, Homo sapiens
GSLLDDSAGKSTEDTAEYSPFK

SEQ ID NO: 101 - protein, 19 aa, Homo sapiens
LENPLDYGSAWAAAAAQCR

SEQ ID NO: 102 - protein, 19 aa, Homo sapiens
LENPLDYGSAWAAAAAQCR

SEQ ID NO: 103 - protein, 21 aa, Homo sapiens
IKLENPLDYGSAWAAAAAQCR

SEQ ID NO: 104 - protein, 21 aa, Homo sapiens
IKLENPLDYGSAWAAAAAQCR

SEQ ID NO: 105 - protein, 13 aa, Homo sapiens
SGALDEAAAYQSR

SEQ ID NO: 106 - protein, 12 aa, Homo sapiens
STEDTAEYSPFK

SEQ ID NO: 107 - protein, 17 aa, Homo sapiens
STEDTAEYSPFKGGYTK

SEQ ID NO: 108 - protein, 36 aa, Homo sapiens
GGYTKGLEGESLGCSGSAAAGSSGTLELPSTLSLYK

SEQ ID NO: 109 - protein, 27 aa, Homo sapiens
GDCMYAPLLGVPPAVRPTPCAPLAECK

SEQ ID NO: 110 - protein, 23 aa, Homo sapiens
AVSVSMGLGVEALEHLSPGEQLR

SEQ ID NO: 111 - protein, 23 aa, Homo sapiens
AVSVSMGLGVEALEHLSPGEQLR

SEQ ID NO: 112 - protein, 28 aa, Homo sapiens
DILSEASTMQLLQQQQQEAVSEGSSSGR

SEQ ID NO: 113 - protein, 28 aa, Homo sapiens
DILSEASTMQLLQQQQQEAVSEGSSSGR

SEQ ID NO: 114 - protein, 15 aa, Homo sapiens
DNYLGGTSTISDNAK

SEQ ID NO: 115 - protein, 19 aa, Homo sapiens
DNYLGGTSTISDNAKELCK

SEQ ID NO: 116 - protein, 24 aa, Homo sapiens
DYYNFPLALAGPPPPPPPPHPHAR

SEQ ID NO: 117 - protein, 25 aa, Homo sapiens
EASGAPTSSKDNYLGGTSTISDNAK

SEQ ID NO: 118 - protein, 29 aa, Homo sapiens
EASGAPTSSKDNYLGGTSTISDNAKELCK

SEQ ID NO: 119 - protein, 27 aa, Homo sapiens
ELCKAVSVSMGLGVEALEHLSPGEQLR

SEQ ID NO: 120 - protein, 27 aa, Homo sapiens
GDCMYAPLLGVPPAVRPTPCAPLAECK

SEQ ID NO: 121 - protein, 27 aa, Homo sapiens
GDCMYAPLLGVPPAVRPTPCAPLAECK

SEQ ID NO: 122 - protein, 12 aa, Homo sapiens
STEDTAEYSPFK

SEQ ID NO: 123 - protein, 10 aa, Homo sapiens
LLDSVQPIAR

SEQ ID NO: 124 - protein, 19 aa, Homo sapiens
LENPLDYGSAWAAAAAQCR

SEQ ID NO: 125 - protein, 31 aa, Homo sapiens; identical to SEQ ID NO: 51

SEQ ID NO: 126 - protein, 24 aa, Homo sapiens
DYYNFPLALAGPPPPPPPPHPHAR

SEQ ID NO: 127 - protein, 15 aa, Homo sapiens
DNYLGGTSTISDNAK

SEQ ID NO: 128 - protein, 28 aa, Homo sapiens
DILSEASTMQLLQQQQQEAVSEGSSSGR

SEQ ID NO: 129 - protein, 23 aa, Homo sapiens
AVSVSMGLGVEALEHLSPGEQLR

SEQ ID NO: 130 - protein, 23 aa, Homo sapiens
AVSVSMGLGVEALEHLSPGEQLR

SEQ ID NO: 131 - protein, 19 aa, Homo sapiens
LENPLDYGSAWAAAAAQCR

SEQ ID NO: 132 - protein, 31 aa, Homo sapiens; identical to SEQ ID NO: 51

SEQ ID NO: 133 - protein, 15 aa, Homo sapiens
DNYLGGTSTISDNAK

SEQ ID NO: 134 - protein, 23 aa, Homo sapiens
AVSVSMGLGVEALEHLSPGEQLR

SEQ ID NO: 135 - protein, 23 aa, Homo sapiens
AVSVSMGLGVEALEHLSPGEQLR

SEQ ID NO: 136 - protein, 10 aa, Homo sapiens
GSLLDDSAGK

SEQ ID NO: 137 - protein, 19 aa, Homo sapiens
LENPLDYGSAWAAAAAQCR

SEQ ID NO: 138 - protein, 19 aa, Homo sapiens
LENPLDYGSAWAAAAAQCR

SEQ ID NO: 139 - protein, 31 aa, Homo sapiens; identical to SEQ ID NO: 51

SEQ ID NO: 140 - protein, 31 aa, Homo sapiens; identical to SEQ ID NO: 51

SEQ ID NO: 141 - protein, 27 aa, Homo sapiens
GDCMYAPLLGVPPAVRPTPCAPLAECK

SEQ ID NO: 142 - protein, 27 aa, Homo sapiens
GDCMYAPLLGVPPAVRPTPCAPLAECK

SEQ ID NO: 143 - protein, 19 aa, Homo sapiens
LENPLDYGSAWAAAAAQCR

SEQ ID NO: 144 - protein, 19 aa, Homo sapiens
LENPLDYGSAWAAAAAQCR

SEQ ID NO: 145 - protein, 31 aa, Homo sapiens; identical to SEQ ID NO: 51

SEQ ID NO: 146 - protein, 31 aa, Homo sapiens; identical to SEQ ID NO: 51

SEQ ID NO: 147 - protein, 27 aa, Homo sapiens
GDCMYAPLLGVPPAVRPTPCAPLAECK

SEQ ID NO: 148 - protein, 27 aa, Homo sapiens
GDCMYAPLLGVPPAVRPTPCAPLAECK

SEQ ID NO: 149 - protein, 19 aa, Homo sapiens
LENPLDYGSAWAAAAAQCR

SEQ ID NO: 150 - protein, 19 aa, Homo sapiens
LENPLDYGSAWAAAAAQCR

SEQ ID NO: 151 - protein, 31 aa, Homo sapiens; identical to SEQ ID NO: 51

SEQ ID NO: 152 - protein, 31 aa, Homo sapiens; identical to SEQ ID NO: 51

SEQ ID NO: 153 - protein, 27 aa, Homo sapiens
GDCMYAPLLGVPPAVRPTPCAPLAECK

SEQ ID NO: 154 - protein, 27 aa, Homo sapiens
GDCMYAPLLGVPPAVRPTPCAPLAECK

SEQ ID NO: 155 - protein, 22 aa, Homo sapiens
GSLLDDSAGKSTEDTAEYSPFK

SEQ ID NO: 156 - protein, 27 aa, Homo sapiens
GSLLDDSAGKSTEDTAEYSPFKGGYTK

SEQ ID NO: 157 - protein, 19 aa, Homo sapiens
LENPLDYGSAWAAAAAQCR

SEQ ID NO: 158 - protein, 19 aa, Homo sapiens
LENPLDYGSAWAAAAAQCR

SEQ ID NO: 159 - protein, 21 aa, Homo sapiens
IKLENPLDYGSAWAAAAAQCR

SEQ ID NO: 160 - protein, 13 aa, Homo sapiens
SGALDEAAAYQSR

SEQ ID NO: 161 - protein, 35 aa, Homo sapiens
SGALDEAAAYQSRDYYNFPLALAGPPPPPPPPHAR

SEQ ID NO: 162 - protein, 12 aa, Homo sapiens
STEDTAEYSPFK

SEQ ID NO: 163 - protein, 17 aa, Homo sapiens
STEDTAEYSPFKGGYTK

SEQ ID NO: 164 - protein, 10 aa, Homo sapiens
GSLLDDSAGK

SEQ ID NO: 165 - protein, 31 aa, Homo sapiens; identical to SEQ ID NO: 51

SEQ ID NO: 166 - protein, 12 aa, Homo sapiens
AREASGAPTSSK

SEQ ID NO: 167 - protein, 23 aa, Homo sapiens
AVSVSMGLGVEALEHLSPGEQLR

SEQ ID NO: 168 - protein, 23 aa, Homo sapiens
AVSVSMGLGVEALEHLSPGEQLR

SEQ ID NO: 169 - protein, 28 aa, Homo sapiens
DILSEASTMQLLQQQQQEAVSEGSSSGR

SEQ ID NO: 170 - protein, 28 aa, Homo sapiens
DILSEASTMQLLQQQQQEAVSEGSSSGR

SEQ ID NO: 171 - protein, 15 aa, Homo sapiens
DNYLGGTSTISDNAK

SEQ ID NO: 172 - protein, 19 aa, Homo sapiens
DNYLGGTSTISDNAKELCK

SEQ ID NO: 173 - protein, 24 aa, Homo sapiens
DYYNFPLALAGPPPPPPPPHPHAR

SEQ ID NO: 174 - protein, 10 aa, Homo sapiens
EASGAPTSSK

SEQ ID NO: 175 - protein, 25 aa, Homo sapiens
EASGAPTSSKDNYLGGTSTISDNAK

SEQ ID NO: 176 - protein, 29 aa, Homo sapiens
EASGAPTSSKDNYLGGTSTISDNAKELCK

SEQ ID NO: 177 - protein, 27 aa, Homo sapiens
ELCKAVSVSMGLGVEALEHLSPGEQLR

SEQ ID NO: 178 - protein, 27 aa, Homo sapiens
GDCMYAPLLGVPPAVRPTPCAPLAECK

SEQ ID NO: 179 - protein, 31 aa, Homo sapiens; identical to SEQ ID NO: 51

SEQ ID NO: 180 - protein, 19 aa, Homo sapiens
LENPLDYGSAWAAAAAQCR

SEQ ID NO: 181 - protein, 21 aa, Homo sapiens
IKLENPLDYGSAWAAAAAQCR

SEQ ID NO: 182 - protein, 21 aa, Homo sapiens
IKLENPLDYGSAWAAAAAQCR

SEQ ID NO: 183 - protein, 10 aa, Homo sapiens
LLDSVQPIAR

SEQ ID NO: 184 - protein, 14 aa, Homo sapiens
MLYFAPDLVFNEYR

SEQ ID NO: 185 - protein, 13 aa, Homo sapiens
SGALDEAAAYQSR

SEQ ID NO: 186 - protein, 12 aa, Homo sapiens
STEDTAEYSPFK

SEQ ID NO: 187 - protein, 17 aa, Homo sapiens
STEDTAEYSPFKGGYTK

SEQ ID NO: 188 - protein, 19 aa, Homo sapiens
LENPLDYGSAWAAAAAQCR

SEQ ID NO: 189 - protein, 23 aa, Homo sapiens
AVSVSMGLGVEALEHLSPGEQLR

SEQ ID NO: 190 - protein, 23 aa, Homo sapiens
AVSVSMGLGVEALEHLSPGEQLR

SEQ ID NO: 191 - protein, 28 aa, Homo sapiens
DILSEASTMQLLQQQQQEAVSEGSSSGR

SEQ ID NO: 192 - protein, 28 aa, Homo sapiens
DILSEASTMQLLQQQQQEAVSEGSSSGR

SEQ ID NO: 193 - protein, 15 aa, Homo sapiens
DNYLGGTSTISDNAK

SEQ ID NO: 194 - protein, 24 aa, Homo sapiens
DYYNFPLALAGPPPPPPPPHPHAR

SEQ ID NO: 195 - protein, 25 aa, Homo sapiens
EASGAPTSSKDNYLGGTSTISDNAK

SEQ ID NO: 196 - protein, 27 aa, Homo sapiens
ELCKAVSVSMGLGVEALEHLSPGEQLR

SEQ ID NO: 197 - protein, 31 aa, Homo sapiens; identical to SEQ ID NO: 51

SEQ ID NO: 198 - protein, 31 aa, Homo sapiens; identical to SEQ ID NO: 51

SEQ ID NO: 199 - protein, 10 aa, Homo sapiens
GSLLDDSAGK